SEQUENCE LISTING (259 sequences)

SEQ ID NO: 1 - DNA, 1134 bp, Artificial Sequence
  Synthetic
atgtcgaccagcgcgtttttcatcccgagccttaatctgatgggtgccgggtgcttacagcaggcggtagacgcgatgcgcggccatggcttccgccgcgccctgattgttaccgatcaaggcctggttaaagcaggtctggccgcaaaagtggcagatatgttaggcaaagcggacattgagccggtaatttttgacggcgtgcatccgaacccgagctgtgccaatgtcaacgcgggcctggccttactgaaagaaaaacagtgtgatgttgtggtaagcctcggcgggggcagcccgcatgactgcgccaaaggcattgcattagttgccgtcaacggcggcaaaattcaagattatgaaggcgttgataaaagcgcaaagccgcagctcccgctggtggcgattaacaccacggcaggcaccgcttcggaaatgacccgcttctgcattattaccgatgaaagccgccatattaaaatggcaattgttgataaacataccaccccgattctcagcgtcaatgatccggaaaccatggcgggcatgccggcaagcctgaccgcggctaccggcatggacgcactgacccatgccgttgaagcatatgttagcaccattgcaaccccgattaccgatgcctgtgcactgaaagcagttgaactgattgcgggctttctgcgccgcgcagtcaaggacggcaaggatatggaggctcgcgaacagatggcgtacgctcagtttctggccggcatggcctttaacaatgcaagcttaggttacgtgcatgcgatggctcatcagctgggcgggttctacgatctgccgcatggcgtttgcaacgcggtactgctgccgcatgttcaagcgtttaacgccgcgagcgcgggcgagcgcctgggcgatgtggccattgcgctgggcgagaaaacccgcagcgcgcaagcggccattgccgcgattaaacgcctggccgcggatgtgggcattccggccggcctgcgcgaactcggcgtgaaagaagcggatattccgaccctcgcggataacgccctgaaagacgcgtgcggcttcaccaacccgcgcaaaggcagccatgaagacgtttgtgcgatcttccgcgcagcgatgtaa

SEQ ID NO: 2 - DNA, 1173 bp, Artificial Sequence
  Synthetic
atgactcatttgaatattgcaaaccgtgtcgacagtttctttattccttgcgttacattattcgggcctggctgtgtccgtgaaacgggagttcgcgcacgctctcttggcgcacgcaaagcgctgattgttacggatgcaggattgcataagatgggtctttccgaggttgtggctggtcacattcgtgaggccggactgcaagccgttattttccctggagcggagcctaatccaactgacgtaaatgtgcacgatggagtaaaactgttcgaacgtgaggaatgtgactttattgtatcgctgggcggcgggtcgagtcacgactgcgccaaaggaattggacttgtcactgcgggcggcggtcacattcgtgattacgagggcattgataagtccacagtgccaatgactccgttaatctccattaatactaccgccggaaccgcagctgagatgacacgtttttgcatcattactaattcctctaaccatgttaagatggtgatcgtagattggcgttgtaccccgcttatcgcaatcgatgaccctagtctgatggtagcgatgcctccggccttaactgcagcgaccggtatggacgcattaacccacgctatcgaggcctacgtaagtacagcagctactccgattactgatgcttgtgctgagaaggctatcgtactgatcgctgaatggttacccaaagcagtcgcaaatggtgatagtatggaagcacgcgcagcaatgtgctacgcccagtacctggctggtatggctttcaataacgcaagtcttggctacgtccacgcgatggcacaccaattggggggtttctacaatctgcctcacggtgtgtgtaacgcaatcttactgccccacgtatctgagtttaatttaatcgcagcgcccgagcgttatgcacgtatcgcggaattgttgggcgagaacatcggcggactgagcgctcacgatgcggcaaaggctgcggtgtccgcaattcgcaccctgtcaaccagtatcggcatccccgcagggttagccggactgggcgtgaaggcggatgaccacgaagttatggcgagtaatgcccaaaaagacgcctgcatgttgaccaacccacgtaaagccaccctggcacaagttatggcaatcttcgctgcagcgatgtga

SEQ ID NO: 3 - DNA, 1152 bp, Artificial Sequence
  Synthetic
atgacgaaaaccaagttctttatcccctcatcgacagtgttcggtcgtggcgcggtaaaagaagtcggtgcacgtttgaaggccattggtgcgactaaagccttaattgtaacagacgcatttttacattctacaggtttatcagaggaagttgcaaaaaacattcgtgaggcaggattagatgtcgtgatttttccaaaagctcagccggaccctgcggatacccaggttcacgagggtgttgaagtatttaagcaggagaaatgcgatgccctggtttctatcggaggcggatcatcgcacgataccgcaaaaggcatcgggctggtggcagccaacggcgggcgtatcaatgattaccagggggtaaactctgtagagaaacaggttgtaccccagattgccatcaccaccacggctgggactggttccgagaccacctcgcttgcagtcatcaccgatagcgctcgtaaagtaaaaatgcctgtcatcgatgagaaaatcacacccacagtcgccatcgtggacccagagttaatggtcaagaaaccagctggcttgacaattgcaaccggcatggacgcattaagccacgcaatcgaagcctatgtggctaagcgcgccacgcctgtgacagacgccttcgccatccaagctatgaaactgattaacgagtacttacctaaagcagtcgctaacggtgaggatattgaagctcgtgaggcgatggcgtatgcccagtatatggcgggagttgcttttaataatggtggcttagggttagtgcatagtatctcgcaccaggtaggtggcgtttacaagttacaacacggcatttgcaattcggtagtgatgccgcatgtatgccaattcaacctgattgcccgtacagaacgcttcgctcacattgcggagctgttaggggagaacgtttcgggcctgtcgaccgcgtcggccgcagaacgtacaattgccgctttagagcgctacaatcgtaattttggtatcccgtccggctacaaggcgatgggtgtgaaggaagaggacattgagttgttggcaaataacgcgatgcaagatgtctgtacgctggataatccgcgcgtcccaaccgtgcaggacatccaacagattattaagaatgccctttga

SEQ ID NO: 4 - DNA, 1152 bp, Artificial Sequence
  Synthetic
atgacgaaaaccaagttctttatcccctcatcgacagtgttcggtcgtggcgcggtaaaagaagtcggtgcacgtttgaaggccattggtgcgactaaagccttaattgtaacagacgcatttttacattctacaggtttatcagaggaagttgcaaaaaacattcgtgaggcaggattagatgtcgtgatttttccaaaagctcagccggaccctgcggatacccaggttcacgagggtgttgaagtatttaagcaggagaaatgcgatgccctggtttctatcggaggcggatcatcgcacgataccgcaaaaggcatcgggctggtggcagccaacggcgggcgtatcaatgattaccagggggtaaactctgtagagaaacaggttgtaccccagattgccatcaccaccacggctgggactggttccgagaccacctcgcttgcagtcatcaccgatagcgctcgtaaagtaaaaatgcctgtcatcgatgagaaaatcacacccacagtcgccatcgtggacccagagttaatggtcaagaaaccagctggcttgacaattgcaaccggcatggacgcattaagccacgcaatcgaagcctatgtggctaagcgcgccacgcctgtgacagacgccttcgccatccaagctatgaaactgattaacgagtacttacctaaagcagtcgctaacggtgaggatattgaagctcgtgaggcgatggcgtatgcccagtatatggcgggagttgcttttaataatggtggcttagggttagtgcatagtatctcgcaccaggtaggtggcgtttacaagttacaacacggcatttgcaattcggtagtgatgccgcatgtatgccaattcaacctgattgcccgtacagaacgcttcgctcacattgcggagctgttaggggagaacgtttcgggcctgtcgaccgcgtcggccgcagaacgtacaattgccgctttagagcgctacaatcgtaattttggtatcccgtccggctacaaggcgatgggtgtgaaggaagaggacattgagttgttggcaaataacgcgatgcaagatcgttgtacgctggataatccgcgcgtcccaaccgtgcaggacatccaacagattattaagaatgccctttga

SEQ ID NO: 5 - DNA, 1134 bp, Artificial Sequence; identical to SEQ ID NO: 1
  Synthetic

SEQ ID NO: 6 - DNA, 1173 bp, Artificial Sequence
  Synthetic
atggcctttaaaaatatcgcggatcaaaccaatggcttttacataccctgcgtgtctctgttcggtccgggtagcgccaaggaagttggttcaaaagcccagaacttgggggcgaaaaaagccttaatcgtgaccgatgcgggcttatacaagttcggcgtcgcggacatcattgcgggttatctgaaagaagcacaggtggaatcatatattttcgctggcgctgaaccgaacccgaccgatatcaatgttcacgacggcgtagaagcttataacaataatgcctgcgactttatcatttcccttggcggcggctcctcacacgactgcgcgaaaggcattgggctggttaccgccggaggcggccatatccgcgattatgaaggcatcgataagtccacagtaccgatgacgccgttaatcgccatcaacaccacagccggtactgcgtccgaaatgacccgcttttgcatcataaccaacaccgagacgcacgtgaagatggcaatcgtagattggcgctgtaccccattaattgctatcgatgatccgaagctgatgatcgctaaacctgcggccctgaccgccgccacggggatggatgctcttacccatgcagtggaggcgtatgtgtcaaccgcagccaaccctataaccgatgcgtgcgcggaaaaagcgattagcatgatttcacagtggctgtcgccggctgtcgcgaacggcgaaaacatagaagcgcgcgatgcgatgtcgtatgcccagtatttggctggtatggccttcaataatgcatcgctgggctatgtgcatgcgatggcgcatcaattaggcggattttataatctgccacatggtgtgtgcaacgcgattcttcttcctcacgtgtgcgaatttaatttaattgcgtgtcctgaccgttatgcgaaaattgcagaattaatgggtgtgaatattgaagggctaacgataaatgaagcggcgtacgcagccatcgacgcgatcaaaatcctctcccaatccatcggcatcccgaccggcctgaaagaactcagcgtcaaagaagaagacctagaagtgatggcgcagaatgcccagaaagacgcctgtatgttaacgaacccacgcaaagcagatctgcaacaggttatcaacattttcaaagccgccatgtga

SEQ ID NO: 7 - DNA, 1149 bp, Artificial Sequence
  Synthetic
atgaccgtctccgaattttttattccaagccacaatatcctggggccgggtgcgttggatcaagcgatgccgatcattggtaaaatgggcttcaaaaaagccctgattatcaccgatgccgatctggctaagttgggcatggcacagctggtggctgataaattaaccgcgcaaggcattgataccgccatttttgacaaagtccagccgaaccctactgtcggtaatgtgaacgcggggcttgacgccttgaaggcacacggcgcggatttgatcgttagtctgggtggcggctcatctcatgactgtgcgaaaggagttgcattagtggcaagcaatggcggcaagatcgcggactacgaaggcgtcgacaaatcggcaaaaccgcagttgccgctgctggccatcaacaccaccgccggcaccgcgtcggaaatgacacgtttcacgataattaccgatgaaacgcgccacgttaaaatggccattattgatcgccacattactccatttctgtccgtaaacgatagtgatcttatggaaggtatgccggcgtctctgaccgcggcgacaggcatggatgcccttacacacgctgtggaggcatacgtgtcaacaattgctacccctatcaccgacgcatgcgcagtgaaagtcgtcgaactgatcgcaaaatatcttcccactgcggttcgtgagccccacaacaaaaaagcacgcgaacagatggcctacgcgcagttcttggccgggatggcgtttaacaacgccagtttagggtatgtgcatgccatggctcatcagctgggaggattctacgatttgccgcacggtgtctgtaacgcgttgctgctgcctcatgttcaagccttcaacatgcaggttgccggtgagcgtttaaatgaaattgggaagctgctgagtgataacaatgccgatctcaaaggcttggatgttattgctgcaattaaaaagcttgcggacattgtgggcattcccaaatcgttggaagaactcggcgtgaagcgtgaagactttcctgtcctggccgataacgccctgaaagatgtctgcggggcgacaaatccgattcagaccgacaaaaagacgattatgggtatatttgaagaagcctttggagtgcgctga

SEQ ID NO: 8 - DNA, 1173 bp, Artificial Sequence
  Synthetic
atggcccatattgcgcttgcagatcatacggatagctttttcatcccttgcgtgaccctgataggcccggggtgcgccaagcaagcgggcgaccgcgccaaggcattaggcgcacgtaaagcactgattgtaaccgatgcgggccttaagaagatgggagtagcagacattattagcgggtaccttctggaggacggtctgcaaactgtgatctttgacggggcagagcctaatccgacggataaaaatgtacacgatggtgtcaaaatttatcaggataacggatgtgattttatcgtgtcacttggcggcgggtcggcgcacgattgtgcgaaaggaatagggctggttaccgccggcggcggaaacatccgtgattatgaaggcgtggataaatcacgtgtcccgatgaccccactcattgcaattaacacgacggccggcaccgcttcggaaatgactcgcttctgcattattactaactcccagacccacgtcaaaatggcgattgttgattggcgttgcaccccgctgattgccattgatgacccgaatttaatggtggccatgccgccagcgttaaccgcggccacaggtatggatgccctgacccacgcgatcgaagcatatgtgtctaccgctgcgaccccgattacggatgcgtgtgccgaaaaagcgatttcactcattggagagtttctgccgaaggcggtagggaacggggaaaatatggaagcgcgcgttgcgatgtgctatgcccagtacttagcgggcatggcgtttaataacgcctctctgggctatgtacacgcgatggcgcatcagttaggtggtttttataacctgccgcacggtgtgtgcaacgcggttctcttaccccatgtgtgtcgctttaatcttattgccgccgccgaccgctatgctcgcgtagctcgtcttctgggtgtcccgaccgatctgatgtcacgtgatgaggcagcagaagcggcgatagatgcgattacgcaaatggcccgctccgtgggaatcccttctggactgacagcacttggtgttaaagcggaagaccacaaaaccatggcggaaaacgcgcagaaagacgcctgtatgcttaccaatccgcgtaaagcgacactggcacagattattggcgtgttcgaagccgcaatgtga

SEQ ID NO: 9 - DNA, 1146 bp, Artificial Sequence
  Synthetic
atggccacccagtttttcatgccggtgcaaaatattctcggtgcgggcgccctggcggaagcaatggatgttattgccgcattgggtctgaaaaaagccctgattatcaccgacgctggcttgagcaaactcggggtcgcagagcagattgggagcttgcttaaaggcaaagggattgattatgcagtgttcgataaggcgcaaccgaacccgaccgtgagcaatgtgaacgccggtcttgaacagctgaagaacagcggcgcagaatttattgtaagcctgggcggcgggagcagccatgattgtgcgaaagcagtggcgattgtggccgcgaacggcggcaagattgaagattacgaaggcctgaataaagccaagaagccgcagctgccgctcattagcattaacaccaccgccggcaccgcaagcgagatgacccgcttcgcggtgattaccgatgaaagccgccatgtgaaaatggccattgttgataaaaacgtcaccccgctgctgagcgttaacgatccgagcctgatggagaacatgccggcgccgctcaccgcagccacgggtatggacgcactgacccatgcggtcgaagcgtacgttagcaccggcgcgagcccgattaccgacgcgtgtgcagtcaaagcgattgaacttattgcccgctacctgccgaccgctgtccatgaaccgaaaaacaaagaagcacgcgaacagatggcctatgcgcaattcttggcgggcatggcttttaataacgcttcgctgggctacgttcatgcgatggcccatcaactgggcggcttttatgacttaccgcatggtgtgtgtaatgcgctgctgctgccgcatgtggagcgctttaaccagcaagcggccaaagaacgcttggatgaaattggccaaattctgaccaaaaataacaaggatctggccggcctggatgtgattgatgcgattaccaaactggctggcattgtaggcattccgaaaagcctgaaagagctgggtgtcaaagaagaagattttgacgttctcgcggataacgcgctgaaagatgtgtgcggcttcaccaacccgattcaggctgataaacagcagattattggcattttcaaagccgcattcgatccggcctga

SEQ ID NO: 10 - DNA, 1149 bp, Artificial Sequence
  Synthetic
atgtcgtcaaccttttatattcccgcggtcaatattattggcgaaaacgcactaaaagatgcggccacccagatggataactatggattcaaacaggccctgatcgtcacggatccaggtatgaccaagttgggagtaactgccgaaattgaggcgctgctcaaagaacacggcattgattccttaatttacgatggcgtccagcctaaccccaccgtgacaaacgtaaaggcggggttagatgttcttcaaaaacaccagtgtgattgcgttatttctctagggggcggcagtgctcatgactgtgcgaaaggtatcgcgctggtagcgacgaatggcggtcacatcagcgattatgaaggagttgacgttagcaagaaaccgcagcttccattgatttccatcaataccaccgctggaacggccagtgaaatgacccgtttttgcattattaccgacccagaacgccatattaaaatggcaattgtagatcagaatgttacccctattctttcagttaacgatccgcgtttgatggttggcatgcctgcgtctctgaccgctgccaccggcatggatgcattaacccatgcggttgaggcctatgtatcaaccgatgctacccctataacagatgcttgcgccattaaagcgatcgaaattattcgtgacaatctgcacgaggccgtgcacaatggcgcaaacatggaggctcgcgagcagatggcgtatgcccagttcctggccggcatggcctttaacaacgcttcgctgggctatgttcatgcgatggcgcaccagctgggtggtttctatgacttaccgcacggcgtttgcaacgccgtactgttaccgcacgtgcaacgctataacagccaggttgtcgcgccacgtctcaaagatataggtaaagcactgggtgctgaagtgcaaggcctgacggaaaaagagggcgcggatgccgcgatcgctgccatcgtgaaactctcccagagcgtgaacatccccgctggcctcgaggagctgggcgctaaagaagaagatttcaacaccctggcggataacgctatgaaagatgcctgcggcttaaccaacccgatccagccgtcacacgaggacattgtgaccattttcaaagccgccttctga

SEQ ID NO: 11 - DNA, 1149 bp, Artificial Sequence
  Synthetic
atgaccagcaccttttttatgccggcagtcaacctgatgggcagcggcagcctgggcgaagcgatgcaggctgtaaaaggcctgggctatcgcaaagctctgattgttacggacgcaatgctgaacaaactcggcctcgcggataaagtggcgaagctgcttaatgaacttcaaattgctaccgttgtctttgatggtgctcaaccgaacccgaccaaaggcaacgtacgcgccggtctggccctgttacgcgcgaaccagtgcgattgtgtggtcagcctgggcggcggcagcagccatgattgtgcaaagggcattgctctgtgcgcgaccaacggcggcgaaattagcgattacgagggcgttgaccgcagcgttaagccgcaattgccgctggttgccattaataccaccgcaggcaccgccagcgagatgacccgcttctgcattattaccgatgaagaaacccatattaaaatggctattgtggaccgcaacgttaccccgattctgagcgtgaacgatccggacctgatgctggccaaaccgaaagccttgaccgccgcgaccggcatggacgcactcacccatgccgtagaagcgtatgtgagcaccgcagctaccccgattaccgacgcgtgtgccctgaaggcggttgagcttattgcgcgccatctccgcaccgcagtggcaaagggcgatgatctgcatgcgcgcgaacaaatggcttatgcccagttcctggcgggcatggccttcaacaacgccagcctcggctatgtgcatgccatgagccatcaactgggcggcttctacgacctgccgcatggcgtttgcaatgcgctgctgcttccgcatgttgaggcctttaatgtgaaaaccagcgcggcacgcctccgcgatgtggcgcaggcgatgggtgagaatgtacagggtctggacgcgcaagcgggcgcccaagcgtgcctggccgccattcgcaaacttagcagcgatattggcattccgaaaagcctgggcgaactgggcgttaaacgcgcggacattccgaccttagccgccaacgcaatgaaagacgcctgcggctttaccaacccgcgcagcgccacccagaccgaaattgaagcaatttttgagggcgcgatgtga

SEQ ID NO: 12 - DNA, 1149 bp, Artificial Sequence
  Synthetic
atgtcgagcactttttttatcccggccgttaatatcatgggaatcggttgtctggacgaagcgatgactgcgattgtgggttatggtttccgtaaagcactgattgtaactgacggtggtttagcaaaagcgggtgttgcacagcgtattgcagagcaactagccgtgcgcgatatcgatagtcgcgtctttgacgatgcgaagccgaatccgtctattgcgaacgtagaacagggtctggcgctgctgcaacgcgaaaaatgcgatttcgtgatttcgctgggcggtggctcgccgcatgactgcgcgaaaggcattgcgctgtgcgcgaccaatggtggccgtatcgctgattacgagggtgtggaccgttcgacgaaacctcagcttcctctggttgccattaatacgaccgctgggaccgcctcggaaatgacacgcttctgcattatcaccgatgaagcgcgtcatgttaaaatggccatcgttgatcgcaacgtaactccaattctgtctgtgaacgacccggcgctcatggtcgcgatgcccaaagcccttaccgccgccacaggtatggatgctctgactcacgcggtggaggcatacgtgtcaaccgcggcaaccccgattaccgatgcttgcgctttaaaagcaatcgaactcatatctggtaacttacgccaggccgtcgcaaatggtcaggaccttttggcgcgcgaagcgatggcctatgcacaattcctagcgggcatggccttcaataacgcgagcctggggtacgtgcacgcaatggctcatcagctaggcggtttctacgatctcccccacggcgtgtgcaatgctgtgctgctgccgcacgttcagcgctttaatgctaaagtcagcgccgcccgccttcgcgatgttgcagcggcgctgggcgttgaagtggcggaattgaacgcggaacagggggcagctgccgcgatcgaagcgattgagcagctcagtcgcgatattgacatcccacctggcttggccgtgctgggggcgaaggtggaggacgttccgattctggcgggcaacgccctgaaagatgcgtgcggcctgaccaatccacgcccggcgtcacaggccgaaattgaggcagtctttaaagcggcgttctga

SEQ ID NO: 13 - DNA, 1152 bp, Artificial Sequence
  Synthetic
atggccgcgagcaccttttacattccgagcgtgaacgtcattggcgccgatagcttgaaaagcgcaatggataccatgcgcgactatggctaccgccgcgcgctgatcgtgaccgatgcgattttaaacaaattgggtatggcgggcgacgtacagaaaggccttgccgaacgcgatattttcagcgttatttacgatggcgtgcagccgaatccgaccaccgcaaacgtgaatgcgggtctggctattttaaaggagaacaattgtgattgtgtcattagcctgggcgggggtagcccgcatgactgtgccaaagggatcgccctggttgcgagcaatggtggtcagattagcgactacgagggggttgatcgcagcgcgaaaccgcaactgccgatgattgcaatcaacaccaccgcgggcaccgcttcggaaatgacccgcttttgtattattacggatgaagcgcgccatattaaaatggccattgtggacaagcatgtgaccccgattctgagcgtaaacgatagcagcttaatgaccggcatgccgaaaagccttaccgcggctaccggcatggatgcgttgacccatgccattgaagcgtatgtgagcattgccgcaacgccgattaccgacgcgtgcgcgctgaaggctattaccatgattgcagaaaatctgagcgtggcggtagcagatggcgccaacgcggaagcgcgcgaagccatggcgtatgcccagtttctggccggcatggcgttcaataacgcgagcctgggttatgtgcatgccatggcgcatcagttgggcgggttttacgatttgccgcatggcgtgtgcaacgccgtccttctgccgcatgtgcaggcgttcaacagcaaggttgcagcagcgcgcctccgcgattgcgcgcaggcaatgaaggttaatgtcgcgggcctgagcgatgagcagggcgccaaagcgtgcattgatgctatttgtaaactggcacgcgaagtgaatattccggcgggtctgcgcgatcttaacgtaaaagaggaagacattccggtcctggccaccaacgccctgaaggacgcgtgcggcttcaccaacccgattcaggcgacccatgacgagattatggctatttaccgcgcggcgatgtga

SEQ ID NO: 14 - DNA, 1149 bp, Artificial Sequence
  Synthetic
atgtcgtccacttttttcatcccggcagtcaacatgattggttcgggctgtttacaggaagcaatgcaggcgattcgcaaatatggatttttaaaagccctgattgttaccgatgcggggttagccaaggcgggtgttgcgacccaggtggcgggcctgctggtagagcagggcattgacagcgtgatctacgatggcgcacgccccaatccgacaattgctaacgttgaacaggggctggagctgctgcaagcgcaccagtgcgacttcgtgatttcactcggcggagggtcaccccatgactgcgccaaggggattgcgttatgcgcgagcaatgggggtcacatttcagactatgaaggcgttgaccgttctcaacagccgcagttaccgctggtggcaattaacaccaccgcaggcaccgcatcagagatgacccgcttttgtatcattacagatacggcgcgtcacgtcaagatggcgattattgatcgtaacgttacccccatcctgtcggtaaacgatcctcaaatgatggcaggcatgccgcgtagcttaactgccgccactggtatggatgcgttaacccacgccgtggaggcctacgttagtactgcggccacgcccatcacggatgcgtgtgccctgaaagcaattggtctgattgccggcaaccttcagcgtgccgtcgaacaaggagacgatctgcaagcgcgtgaaaatatggcgtatgcacagtttcttgcgggtatggcgtttaacaatgctagtctgggttacgtgcatgcgatggctcaccagctgggaggcttctacgatctgccgcacggcgtgtgcaatgccgtcttactgcctcacgtgcagcgttttaatgcgtcggtgagcgccgcgcgtctgaccgatgtcgcacatgcgatgggcgccaacattcgcggaatgtcacccgaagcgggtgctcaggccgcgattgatgcgatttcgcaactggcggcgtcagttgaaattccggctggcctcacccagctgggcgtgaaacagtcagatatcccgaccctggcggcaaacgcgctgaaggatgcgtgcggtttaaccaaccctcgccctgccgatcaacagcagattgaatcgatattccaggccgccctctaa

SEQ ID NO: 15 - DNA, 1173 bp, Artificial Sequence
  Synthetic
atgtcgtacttaagtatcgcagatcgcactgacagcttttttattccgtgtgttaccttaattggcgccggctgcgcccgcgaaacgggcacacgcgcgaaatccctcggcgcgaaaaaggctttgatcgtcaccgatgcgggcttacataaaatggggctgtcggcaaccattgcgggctacttacgcgaagccggcgtggatgcggtgattttcccgggtgccgaacccaaccccaccgacgtcaacgtgcacgatggagtaaaattgtaccaacagaatggttgtgattttatagttagccttggaggcgggagtagccacgattgcgccaaaggtattggccttgtcaccgctggcgggggacacattagccattacgaaggtgtagataaatccagcgttccgatgacgccgctgatctctatcaatacaacggctggcaccgccgccgaaatgacgcgtttttgcatcatcaccaattcgtccaaccacgtaaaaatggcaatcgttgactggcgttgtacccctctgattgctatcgacgaccctcgtctgatggtagcgatgccgcctgcccttaccgctgctacaggtatggatgcactgactcatgcggttgaagcctacgtcagcactgctgccaccccgatcactgacgcatgcgccgaaaaggcaatagcacttattggcgagtggctgccgaaagcagtggcaaatggcgagtcgatggaggcgcgcgccgccatgtgttatgcacagtacctggcaggcatggcatttaacaatgcaagcctgggctatgtacacgccatggcacatcagttaggtggtttctataacctgcctcacggcgtctgtaatgctattctgctcccgcacgtgtgcgagttcaacctgattgcggcgccggaacgttttgcacgcattgccgcattgctgggcgccaatacagcaggtctgagcgtaaccgatgctggtgcagccgcgattgccgcgattcgtgcgttatcggcctcgatcgatattccggcgggcctcgcgggcctgggtgtaaaagccgatgatcacgaagtcatggcccgtaacgcccagaaagatgcgtgcatgttaacgaatcctcgcaccgcaacccttaagcaagtgataggcatttttgaggcggcgatgtga

SEQ ID NO: 16 - DNA, 1152 bp, Artificial Sequence
  Synthetic
atggccacgttcaaattctacattccggccattaatttaatgggggcaggatgtttacaagaagcggcagctgacattcaaggacatggctatcgcaaagcgctgatcgttacagacaagattctgggccagattggcgtggtgggtcgtctggcggccctgctggccgaacatggtattgatgccgtagtgttcgatgaaacacgcccgaaccccactgtagcaaatgtcgaagccggtctggccatgatccgcgcacatggttgtgactgcgtcatttcactgggcggaggcagccctcatgactgtgcgaaagggattgcgctggttgcggcgaacggcgggtcaattaaagattatgaaggtgtggatcgctccgcgaagccgcaactgccgttgattgcgattaataccaccgccggcacggcgtccgaaatgacccgcttctgtatcatcacagacgaatctcgccaggtcaaaatggcgattatcgacaaacatgtgacaccgttaatgtcagtcaatgatccggaattaatgctcgcgaaacctgccggtctaaccgccgccacaggcatggacgccttaacacacgcgattgaagcatacgtgagcaccgctgctacccccgttacggatgcgagtgccgtgatggcaattgccctgattgcggaacatctgcgtaccgcggtgcaccaaggagaagatttgcacgcgcgcgaacaaatggcgtacgctcagtttctggccggcatggcgttcaacaacgcctcattgggctacgtgcatgcgatggcgcatcagttagggggtttttatgacctgccgcatggtgtgtgtaatgcggttctgctgccgcatgtgcaggcctacaatgcccgtgtctgcgcgggccgtctgaaggatgtcgcgcgtcacatgggcgttgatgtgagcgctatgagcgatgaacaaggtgcagcggcggccatcgacgcgattcgtcagttagcgagtgacgttaaaattccgacgggtttagagcaactaggtgtacgtgctgatgatctggacgttctggcaacgaatgccctgaaagatgcatgtggtcttacaaatccgcgccaggcgactcatgcggaaattgttgccatttttcgcgctgcgatgtga

SEQ ID NO: 17 - DNA, 1212 bp, Artificial Sequence
  Synthetic
atggccttcaagaacatcgcagaccagaccaacggcttctacatcccgtgcgtttcgctttttggtcctggctgcgcgaaagaaatcgggggcaaagcacagaatttaggcgctaaaaaagcgctgatcgttacggatgctggactttttaaattcggggtagccgataccattgcaggttatttgaaagatgcgggcgtcgattcacatatctttccgggcgcagaaccgaaccctaccgatattaacgtccacaacggcgttactgcgtacaatgagcagggatgtgatttcattgtctcattaggcgggggctccagccatgattgtgccaaaggtatagggctggtaaccgccggtggaggccacattcgtgattatgaaggtattgataagtcaaccgtgccgatgacgccactgatagccatcaacaccaccgccggcaccgcctctgaaatgacccgcttttgtatcatcacgaacaccgacacccatgtcaaaatggcgattgttgactggcgctgtaccccgttgatcgcgattgacgatcctaaactgatgattgcaaagccggcgtcacttaccgccgccactggcatggatgcgctgacccatgcggtggaagcatacgttagtacagcggcaaatccaattaccgacgcttgtgcagaaaaagcaattagtatgattagcgaatggctgtctccggcggttgcgaacggtgaaaatcttgaagcgcgtgatgcgatgagttacgcgcaataccttgcgggtatggcgtttaataatgcgtcattagggtacgtgcacgccatggcacaccagctgggaggcttttataatcttccgcatggagtatgcaatgcggtccttttaccacacgtctgtgaatttaatcttatcgcatgtcccgatcgttatgctcgtatagcagaattgatgggagttaacattaccggtctgaccgttacggaagccggctatgcggccattgatgccattcgcgaactttcggccagcatcggcattccgtcatctctgtcggaactcggtgttaaagaacaggatttaggtgttatgagcgaaaacgcacagaaagacgcgtgcatgttaaccaatccccgcaaagcgaaccacgcgcaggtcgtggatatttttaaagctgccctgaagtcgggcgcctcagtggtggattttaaagccgcagtatga

SEQ ID NO: 18 - DNA, 1149 bp, Artificial Sequence
  Synthetic
atggccgcgaagttttttattccgagcgtcaatgtcctgggcaaaggcgccgtagatgacgccattggcgacatcaagaccctgggcttcaaacgcgcgctgattgttaccgataaaccgctggtgaacattgggctcgtgggcgaggtagcggaaaaactggggcagaacggcattaccagcaccgtctttgatggcgttcaaccgaacccgacggtgggcaatgtggaggccggcctggcgctcctgaaagcgaatcagtgtgatttcgtaattagcctgggcggcggcagcccgcatgattgcgctaaaggtattgcgctggtcgccaccaacggcggcagcattaaggactatgaaggcctggataagagcacgaagccgcagttaccgctggtggcgattaacaccaccgcgggcaccgcgagcgaaatgacccgcttctgtattattacggacgaagcccgccatattaagatggcgattgtggataagcataccaccccgattctgagcgtgaacgatccggagctgatgcttaaaaaaccggccagcctgaccgcggccaccggcatggatgcgctgacccatgcggtcgaagcttatgttagcattgcagccaacccgattaccgacgcctgcgccattaaagcaattgaactgattcaaggtaatttggtgaacgcggtgaaacagggccaagatattgaagcgcgcgagcagatggcatatgcccaattcctggccggcatggcatttaataacgcttcgctgggctacgtgcatgcgatggcgcatcagctgggcggcttttacgatctgccgcatggggtgtgcaacgccctgctgctgccgcatgttcaagaatataatgccaaagtggtaccgcatcgccttaaagacattgcgaaggccatgggcgttgatgtagccaaaatgaccgacgaacaaggggccgctgcggcaattaccgcaattaaaaccctcagcgtagccgtgaacattccggagaacctcaccctgctgggtgtgaaagctgaagatattccgacgctggcggacaacgccctcaaagacgcttgtggttttaccaatccgaagcaggcaacccatgccgagatttgtcagatttttaccaatgcactctga

SEQ ID NO: 19 - DNA, 1149 bp, Artificial Sequence
  Synthetic
atgtcgaccacgtttttcattccgagcattaatgtggtgggcgaaaacgccctgaacgacgccgttccgcatattcttggtcatggcttcaaacatgggctgattgtaaccgatgagttcatgaataaaagcggtgtagcacagaaagtcagcgacctgcttgcaaaaagcggcattaataccagcatttttgacggcacccatccgaacccgacggtcagcaacgttaatgacggcctgaaaattctgaaggcaaataattgcgatttcgtgatcagcctgggcggcggcagcccgcatgattgcgctaaaggcattgcgttactggccagcaatggcggcgagattaaagactatgaaggcctggacgtaccgaaaaaaccgcagctcccgcttgtcagcattaacaccaccgcggggaccgcgagcgagattacccgcttctgcatcattaccgacgaagtgcgccatattaagatggctattgtgaccagcatggtcaccccgattctgagcgtgaatgatccggcactgatggcggcaatgccgccgggcctgaccgcggcaaccggcatggatgcgctgacccatgcaattgaagcgtacgtgagcaccgccgcttcgccgattacggacgcatgtgcattaaaagcagccaccatgattagcgagaatctgcgcaccgcggtgaaagatgggaaaaacatggcagcgcgcgaaagcatggcttacgcacagctcctggccggcatggcgtttaataatgccagcctcggctacgttcatgcaatggcccatcaactgggcggcttctacggtttgccgcatggcgtctgcaacgccgtactgttgccgcatgtgcaggaatataatctgccgacctgcgcgggccgcctgaaggatatggcaaaagccatgggggtgaatgttgataagatgagcgatgaggaaggcgggaaggcgtgtattgcagcgattcgcgccctgagcaaagatgtcaacattccggcgaacctcaccgaattaaaagtaaaagccgaggatattccgaccctggcagccaatgcgttgaaagacgcatgtggggtcaccaacccgcgccaaggcccgcagagcgaagtggaagccattttcaaaagcgctatgtga

SEQ ID NO: 20 - DNA, 1149 bp, Artificial Sequence
  Synthetic
atgtcgtcaaccttttttatccccgctgtcaatgtaatgggattgggctgtctggatgaagcaatgaccgcgattcgcaactacggatttcgtaaagcactcattgttaccgataccggattggctaaagcaggcgtggccagtaaagtggcaggtcttttggcgttacaggatattgattctgttatctttgacggcgcaaaaccgaacccgtcaattgctaatgtggaacttgggctgggtctgctgaaagaaagtcaatgtgatttcgttgtgtcgcttgggggcggttcgccgcatgattgtgcgaaaggcatcgcactttgcgcgacaaacggtggccacatcggtgattacgaaggggtagaccgttctactaaaccgcaacttccgctgattgcgattaacaccaccgcagggaccgcctctgagatgactcgcttctgcataattacggatgaatcacgtcatgtgaaaatggctattgtggatcgcaatgtgaccccgttgatgagtgtgaacgatccggcgctgatggtcgccatgcctaagggcctgacagcggccactggcatggatgcactgactcatgccattgaagcatacgtgtcaaccgtagccaaccccattacagatgcatgtgcgctgaaagcggtaactctgatctcgaataatctgcgcctggccgttcgcgatggcggtgacctagcagcccgcgagaatatggcatatgctcaattcctggcaggtatggcatttaataacgcatccctcggcttcgtacatgctatggcgcaccaactgggcggcttctacgatctgccccacggcgtgtgcaacgcggtcctgctgccgcacgtgcaaagcttcaacgcctccgtgtgcgcggaccgcctgaccgacgtggcgcatgctatgggaggcgatacccgcgggttgtcaccggaagaaggggcacaagccgcgattgccgcgatccgcagcctggcccgcgatgtggatattcctgcgggcctccgcgacctcggtgtccgcctgaacgatgtcccggtcctcgccactaacgcgctaaaagatgcatgtggcctgacgaacccccgcgccgctgaccagcgccagattgaggaaatattccgtagcgcctattga

SEQ ID NO: 21 - DNA, 1149 bp, Artificial Sequence
  Synthetic
atgtcgagcaccttttttattccggcggtcaacattatggggattggctgcctggatgaggccatgaacgctattcgcaattacggcttccgcaaagccctgattgttaccgatgcggggttagcgaaagccggcgtggcgagcatgattgctgagaaactggccatgcaggatattgatagccttgtctttgatggcgcaaaaccgaacccgagcattgacaacgtagaacaaggcctgctgcgcctgcgcgagggcaactgcgatttcgtgatcagcttaggcggcggcagcccgcatgactgcgctaaaggcattgcactgtgtgccacgaatggcggccatattcgcgattatgaaggcgtggatcagagcgccaaaccgcagttaccgctgattgcaattaacaccaccgctggcaccgcaagcgaaatgacccgcttctgtattattaccgacgaagcgcgccatgtgaaaatggctattgttgatcgcaacgttaccccgctgctgagcgttaatgatccggcgctcatggtagcgatgccgaagggcttgacggcagcgacgggcatggatgcgctgacccatgcaattgaagcctacgttagcaccgccgcgaatccgattaccgatgcatgtgcactcaaagcgattgacatgattagcaacaatttgcgccaggccgtacatgatggtagcgatttaaccgcccgcgaaaatatggcgtacgcacaattcctcgcaggcatggcattcaataacgcaagcctcggctttgtacatgctatggcccatcagctgggcgggttctacgatttgccgcatggcgtatgtaatgcggtgctgctgccgcatgtgcagagctttaacgcttcggtatgtgccgagcgcctgaccgatgtggcacatgccatgggcgcagatattcgcggctttagcccggaggaaggcgcccaagcagcgattgcggcaattcgcagcctggcccgcgatgtcgaaattccggcgggtctgcgcgagctcggcgcaaaactgccggatatcccgatcctggcggccaacgcgctcaaagatgcatgcggcctgaccaacccgcgcgctgccgatcagcgccagattgaagaaatttttcgcagcgccttctga

SEQ ID NO: 22 - DNA, 1182 bp, Artificial Sequence
  Synthetic
atgtcgctagttaattatctccagctggcagatcgcacggacggctttttcataccaagtgtgaccttggtgggaccaggctgtgtgaaagaagtgggcccgcgtgcgaaaatgctgggcgccaaacgcgcactcattgtgaccgacgccgggctgcataaaatgggtcttagccaagaaattgcggacctgctgcgctcggaaggcatcgatagcgtaatatttgccggcgcggaaccgaaccccacggacatcaacgtgcacgacggcgtgaaggtctaccagaaagagaaatgcgacttcatcgtctcgctagggggtggctctagccacgactgcgcgaaagggattggccttgtgactgccggcggtggccatatccgcgactatgaaggtgttgacaaatctaaagtccctatgacaccacttatcgctattaataccaccgcgggcaccgcgagcgagatgacgcgcttctgtattattaccaatactgatactcacgtgaaaatggcaattgttgattggcgttgcacgccgctggttgcgattgatgatccgcgtcttatggtcaaaatgccgcctgcgctcacagcggctaccggaatggatgcgctcacccatgcagtagaggcatatgtgagcacagcggcaacgcccatcaccgacacctgtgcggagaaagcaattgagctgataggtcagtggctcccgaaagcagtggcgaacggtgactggatggaggcgcgcgcggcgatgtgctatgcgcagtatctagcgggcatggcttttaacaatgccagcctagggtacgtgcatgcgatggcacatcagttgggtggattctataacctgccgcacggtgtctgtaacgcaattctgcttcctcatgtctgccagttcaatctgattgctgcaacggagcgctatgcgcgcattgctgctctgctcggcgtcgatacctcaggcatggaaacgcgcgaggcggccctggcggcgattgcggccattaaggaactgagctcatcaatagggatcccgcgtggcctcagcgaattgggcgtcaaagcagcggatcacaaagtgatggcagaaaatgcgcagaaggatgcgtgcatgttgaccaatccacgtaaagcaaccctggaacaagtcatcgggatttttgaggccgcgatgtga

SEQ ID NO: 23 - DNA, 1146 bp, Artificial Sequence
  Synthetic
atggccacccagttttttatgccggtccaaaacattctgggcgaaaatgcgctggctgaagccatggacgttattagcgccctgggcttaaaaaaagcactgattgttacggacggcggcctgagcaagatgggcgtggccgataaaattggcggtctgctgaaagaaaaaaacattgattatgccgtatttgataaagcgcaaccgaatccgaccgtgaccaatgtcaacgatgggctggcagctctgaaagaagccggcgcagattttattgtcagcctgggcggcgggagcagccatgattgtgccaaagccgtggcgattgtcacgaccaacggtggtaagattgaagactatgaaggcctggacaaaagcaaaaaaccgcagctgccgctgattgccattaacaccaccgcagggaccgcaagcgagatgacccgctttgccgtaattacggatgaagcccgccatgtgaaaatggccattgtcgataagaatgttaccccgctgttaagcgttaacgatccgagcctgatggaaggcatgccggctccgctgaccgccgccaccggcatggatgcgctgacccatgccgtggaagcgtatgtgagcaccattgccagcccgattaccgatgcgtgcgcgttaaaagcgatcgagctgattgcgggctatctgccgaccgcggtacatgaaccgaaaaacaaagaagcgcgcgaaaaaatggcctacgcgcagtttctggccggcatggcgtttaacaatgcgagccttgggtacgtacatgcgatggcacatcagttaggcggcttttacgatctgccgcatggcgtgtgcaacgccctgcttttaccgcatgtggaacgttttaaccaacaggcagccaaagaacgtcttgatgaaattggcgctattttaggcaagtataatagcgatttaaagggtttagatgtgattgatgcaattaccaaactggcacgtattgttggtattccgaaaagcttaaaagaactgggtgttaaacaagaggattttggggtgcttgccgataatgctttaaaagatgtgtgcggttttaccaatccgattcaagctaataaggaacagattatcggcatctatgaggccgcgtttgatccggcctga

SEQ ID NO: 24 - DNA, 1173 bp, Artificial Sequence
  Synthetic
atggccttcaagaatttggcggatcagactaatggcttctacattccgtgcgtttctctgttcggcccgggctgcgcgaaagaagtgggtgcgaaagcgcagaacctcggcgccaagaaagccctgattgtcacagacgcgggcctatttaagtttggcgttgcagacattattgtaggctacctgaaggacgccggggttgatagccatgtcttcccgggggcggaaccgaatccgacggatattaatgtgttgaacggcgtgcaggcatataacgacaatggctgcgacttcattgtctccctcggcggcggctcgagccacgactgcgcgaaaggcatcggcctcgtcacggcaggcggtggtaacatccgcgactacgaaggcatagataagagttctgttccgatgaccccgctgatcgcgatcaataccacagcgggcacggcctcggaaatgacccgcttctgcattattacgaatactgatacccatgtcaagatggcgatcgttgattggcgttgcacacccttagtagctatcgacgacccgaaactgatgatcgcgaaacccgcggcgttaaccgccgcgaccggcatggatgcgctgacccacgcggtggaagcgtatgtcagcaccgcagcaaatccgattaccgatgcctgcgcagaaaaggcaatttccatgatttcagagtggttaagcagcgcagtcgcaaatggcgagaatatcgaggcgcgcgacgcgatggcgtatgcccagtatttggccgggatggcttttaataacgcttccctgggctacgttcacgccatggcccaccaactgggtggtttctacaaccttcctcacggtgtgtgcaatgcaatcctattaccccacgtgtgtgaatttaatctgattgcgtgtcctgaccgcttcgcgaaaattgctcagcttatgggtgtggacaccactgggatgaccgtgaccgaggcaggatacgaagcgatcgccgcgattcgcgaactgagcgccagcattggcattccgtcagggcttaccgagctgggggtgaaagccgccgatcatgcggttatgaccagtaatgcccaaaaagatgcctgtatgctgacgaaccctcgtaaggcgacggatgcgcaagtcattgcgatctttgaggccgcgatgtga

SEQ ID NO: 25 - DNA, 1164 bp, Artificial Sequence
  Synthetic
atgtcctaccgcatgtttgattatttagttccaaatgtgaacttctttggaccgaacgcaatttctgtagtcggggaacgttgcaaacttctgggcggtaagaaagccctcttggtgacggacaaaggcctgcgagctatcaaagatggtgcggttgacaagacactgacccacctgagagaggcgggcatagatgtcgtggttttcgatggtgtagaacccaatcctaaagacaccaacgttcgtgatgggttagaagtgtttcgcaaagagcattgtgatattatcgtgaccgtcggcggtggcagtcctcatgattgcggtaaaggcattggcatcgccgcgactcacgaaggtgacctgtatagctacgcagggattgaaactttgaccaacccgctcccgccgattgtggcggtaaatacgacagccggaacggcgtcagaagtgacccggcattgtgtcctgactaacaccaagacgaaagtcaagtttgtaatcgtgtcgtggcgtaatctaccaagcgttagtattaatgatccgctgctgatgcttggtaaacctgcgccgctaacagccgctaccggaatggacgcacttacacacgccgttgaggcatatatctccaaagatgctaacccggtcaccgacgccgctgcgatccaagcaattaggctgattgcccgcaacttacgtcaggcggttgctttaggcagcaatctgaaagcccgcgagaatatggcttacgcctcgctcctggcgggcatggcgttcaacaacgcaaatttgggatatgtgcatgcaatggctcaccagttgggtgggctgtatgacatgccgcatggggtggcgaacgccgtactgctcccccatgttgcgagatacaatcttatcgcgaacccagaaaaatttgctgatattgcggaatttatgggcgaaaacacggatggactatctactatggatgcggccgaattagccatccacgcgattgcgcgcctgtcggcagacataggtatcccgcagcatctgcgtgatctgggcgtcaaggaagccgatttcccctatatggctgagatggcgctgaaagacgggaatgcattcagcaacccacgcaaaggcaacgaaaaagagatagcagaaattttccggcaagctttttga

SEQ ID NO: 26 - DNA, 1173 bp, Artificial Sequence
  Synthetic
atggcctttaaaaatatcgcggatcaaaccaatggcttttacataccctgcgtgtctctgttcggtccgggtagcgccaaggaagttggtgtaaaagcccagaacttgggggcgaaaaaagccttaatcgtgaccgatgcgggcttatacaagttcggcgtcgcggacatcattgcgggttatctgaaagaagcacaggtggaatcatatattttcgctggcgctgaaccgaacccgaccgatatcaatgttcacgacggcgtagaagcttataacaataatgcctgcgactttatcatttcccttggcggcggctcctcacacgactgcgcgaaaggcattgggctggttaccgccggaggcggccatatccgcgattatgaaggcatcgataagtccacagtaccgatgacgccgttaatcgccatcaacaccacagccggtactgcgtccgaaatgacccgcttttgcatcataaccaacaccgagacgcacgtgaagatggtaatcgtagattggcgctgtaccccattaattgctatcgatgatccgaagctgatgatcgctaaacctgcggccctgaccgccgccacggggatggatgctcttacccatgcagtggaggcgtatgtgtcaaccgcagccaaccctataaccgatgcgtgcgcggaaaaagcgattagcatgatttcacagtggctgtcgccggctgtcgcgaacggcgaaaacatagaagcgcgcgatgcgatgtcgtatgcccagtatttggctggtatggccttcaataatgcatcgctgggctatgtgcatgcgatggcgcatcaattaggcggattttataatctgccacatggtgtgtgcaacgcgattcttcttcctcacgtgtgcgaatttaatttaattgcgtgtcctgaccgttatgcgaaaattgcagaattaatgggtgtgaatattgaagggctaacgataaatgaagcggcgtacgcagccatcgacgcgatcaaaatcctctcccaatccatcggcatcccgaccggcctgaaagaactcagcgtcaaagaagaagacctagaagtgatggcgcagaatgcccagaaagaccgctgtatgttaacgaacccacgcaaagcagatctgcaacaggttatcaacattttcaaagccgccatgtga

SEQ ID NO: 27 - DNA, 1173 bp, Artificial Sequence
  Synthetic
atggcctttaaaaatatcgcggatcaaaccaatggcttttacataccctgcgtgtctctgttcggtccgggtagcgtcaaggaagttggttcaaaagcccagaacttgggggcgaaaaaagccttaatcgtgaccgatgcgggcttatacaagttcggcgtcgcggacatcattgcgggttatctgaaagaagcacaggtggaatcatatattttcgctggcgctgaaccgaacccgaccgatatcaatgttcacgacggcgtagaagcttataacaataatgcctgcgactttatcatttcccttggcggcggctcctcacacgactgcgcgaaaggcattgggctggttaccgccggaggcggccatatccgcgattatgaaggcatcgataagtccacagtaccgatgacgccgttaatcgccatcaacaccacagccggtactgcgtccgaaatgacccgcttttgcatcataaccaacaccgagacgcacgtgaagatggtaatcgtagattggcgctgtaccccattaattgctatcgatgatccgaagctgatgatcgctaaacctgcggccctgaccgccgccacggggatggatgctcttacccatgcagtggaggcgtatgtgtcaaccgcagccaaccctataaccgatgcgtgcgcggaaaaagcgattagcatgatttcacagtggctgtcgccggctgtcgcgaacggcgaaaacatagaagcgcgcgatgcgatgtcgtatgcccagtatttggctggtatggccttcaataatgcatcgctgggctatgtgcatgcgatggcgcatcaattaggcggattttataatctgccacatggtgtgtgcaacgcgattcttcttcctcacgtgtgcgaatttaatttaattgcgtgtcctgaccgttatgcgaaaattgcagaattaatgggtgtgaatattgaagggctaacgataaatgaagcggcgtacgcagccatcgacgcgatcaaaatcctctcccaatccatcggcatcccgaccggcctgaaagaactcagcgtcaaagaagaagacctagaagtgatggcgcagaatgcccagaaagaccgctgtatgttaacgaacccacgcaaagcagatctgcaacaggttatcaacattttcaaagccgccatgtga

SEQ ID NO: 28 - DNA, 1173 bp, Artificial Sequence
  Synthetic
atggcctttaaaaatatcgcggatcaaaccaatggcttttacataccctgcgtgtctctgttcggtccgggtagcgtcaaggaagttggtgtaaaagcccagaacttgggggcgaaaaaagccttaatcgtgaccgatgcgggcttatacaagttcggcgtcgcggacatcattgcgggttatctgaaagaagcacaggtggaatcatatattttcgctggcgctgaaccgaacccgaccgatatcaatgttcacgacggcgtagaagcttataacaataatgcctgcgactttatcatttcccttggcggcggctcctcacacgactgcgcgaaaggcattgggctggttaccgccggaggcggccatatccgcgattatgaaggcatcgataagtccacagtaccgatgacgccgttaatcgccatcaacaccacagccggtactgcgtccgaaatgacccgcttttgcatcataaccaacaccgagacgcacgtgaagatggtaatcgtagattggcgctgtaccccattaattgctatcgatgatccgaagctgatgatcgctaaacctgcggccctgaccgccgccacggggatggatgctcttacccatgcagtggaggcgtatgtgtcaaccgcagccaaccctataaccgatgcgtgcgcggaaaaagcgattagcatgatttcacagtggctgtcgccggctgtcgcgaacggcgaaaacatagaagcgcgcgatgcgatgtcgtatgcccagtatttggctggtatggccttcaataatgcatcgctgggctatgtgcatgcgatggcgcatcaattaggcggattttataatctgccacatggtgtgtgcaacgcgattcttcttcctcacgtgtgcgaatttaatttaattgcgtgtcctgaccgttatgcgaaaattgcagaattaatgggtgtgaatattgaagggctaacgataaatgaagcggcgtacgcagccatcgacgcgatcaaaatcctctcccaatccatcggcatcccgaccggcctgaaagaactcagcgtcaaagaagaagacctagaagtgatggcgcagaatgcccagaaagaccgctgtatgttaacgaacccacgcaaagcagatctgcaacaggttatcaacattttcaaagccgccatgtga

SEQ ID NO: 29 - protein, 385 aa, Bacillus methanolicus MGA3
MKNTQSAFYMPSVNLFGAGSVNEVGTRLAGLGVKKALLVTDAGLHSLGLSEKIAGIIREAGVEVAIFPKAEPNPTDKNVAEGLEAYNAENCDSIVTLGGGSSHDAGKAIALVAANGGTIHDYEGVDVSKKPMVPLIAINTTAGTGSELTKFTIITDTERKVKMAIVDKHVTPTLSINDPELMVGMPPSLTAATGLDALTHAIEAYVSTGATPITDALAIQAIKIISKYLPRAVANGKDIEAREQMAFAQSLAGMAFNNAGLGYVHAIAHQLGGFYNFPHGVCNAILLPHVCRFNLISKVERYAEIAAFLGENVDGLSTYEAAEKAIKAIERMARDLNIPKGFKELGAKEEDIETLAKNAMNDACALTNPRKPKLEEVIQIIKNAM

SEQ ID NO: 30 - protein, 390 aa, Artificial Sequence
  Synthetic
MTHLNIANRVDSFFIPCVTLFGPGCVRETGVRARSLGARKALIVTDAGLHKMGLSEVVAGHIREAGLQAVIFPGAEPNPTDVNVHDGVKLFEREECDFIVSLGGGSSHDCAKGIGLVTAGGGHIRDYEGIDKSTVPMTPLISINTTAGTAAEMTRFCIITNSSNHVKMVIVDWRCTPLIAIDDPSLMVAMPPALTAATGMDALTHAIEAYVSTAATPITDACAEKAIVLIAEWLPKAVANGDSMEARAAMCYAQYLAGMAFNNASLGYVHAMAHQLGGFYNLPHGVCNAILLPHVSEFNLIAAPERYARIAELLGENIGGLSAHDAAKAAVSAIRTLSTSIGIPAGLAGLGVKADDHEVMASNAQKDACMLTNPRKATLAQVMAIFAAAM

SEQ ID NO: 31 - protein, 383 aa, Bacillus methanolicus
MTKTKFFIPSSTVFGRGAVKEVGARLKAIGATKALIVTDAFLHSTGLSEEVAKNIREAGLDVVIFPKAQPDPADTQVHEGVEVFKQEKCDALVSIGGGSSHDTAKGIGLVAANGGRINDYQGVNSVEKQVVPQIAITTTAGTGSETTSLAVITDSARKVKMPVIDEKITPTVAIVDPELMVKKPAGLTIATGMDALSHAIEAYVAKRATPVTDAFAIQAMKLINEYLPKAVANGEDIEAREAMAYAQYMAGVAFNNGGLGLVHSISHQVGGVYKLQHGICNSVVMPHVCQFNLIARTERFAHIAELLGENVSGLSTASAAERTIAALERYNRNFGIPSGYKAMGVKEEDIELLANNAMQDVCTLDNPRVPTVQDIQQIIKNAL

SEQ ID NO: 32 - protein, 383 aa, Artificial Sequence
  Synthetic
MTKTKFFIPSSTVFGRGAVKEVGARLKAIGATKALIVTDAFLHSTGLSEEVAKNIREAGLDVVIFPKAQPDPADTQVHEGVEVFKQEKCDALVSIGGGSSHDTAKGIGLVAANGGRINDYQGVNSVEKQVVPQIAITTTAGTGSETTSLAVITDSARKVKMPVIDEKITPTVAIVDPELMVKKPAGLTIATGMDALSHAIEAYVAKRATPVTDAFAIQAMKLINEYLPKAVANGEDIEAREAMAYAQYMAGVAFNNGGLGLVHSISHQVGGVYKLQHGICNSVVMPHVCQFNLIARTERFAHIAELLGENVSGLSTASAAERTIAALERYNRNFGIPSGYKAMGVKEEDIELLANNAMQDRCTLDNPRVPTVQDIQQIIKNAL

SEQ ID NO: 33 - protein, 377 aa, Chromobacterium violaceum
MSTSAFFIPSLNLMGAGCLQQAVDAMRGHGFRRALIVTDQGLVKAGLAAKVADMLGKADIEPVIFDGVHPNPSCANVNAGLALLKEKQCDVVVSLGGGSPHDCAKGIALVAVNGGKIQDYEGVDKSAKPQLPLVAINTTAGTASEMTRFCIITDESRHIKMAIVDKHTTPILSVNDPETMAGMPASLTAATGMDALTHAVEAYVSTIATPITDACALKAVELIAGFLRRAVKDGKDMEAREQMAYAQFLAGMAFNNASLGYVHAMAHQLGGFYDLPHGVCNAVLLPHVQAFNAASAGERLGDVAIALGEKTRSAQAAIAAIKRLAADVGIPAGLRELGVKEADIPTLADNALKDACGFTNPRKGSHEDVCAIFRAAM

SEQ ID NO: 34 - protein, 390 aa, Acinetobacter sp.
MAFKNIADQTNGFYIPCVSLFGPGSAKEVGSKAQNLGAKKALIVTDAGLYKFGVADIIAGYLKEAQVESYIFAGAEPNPTDINVHDGVEAYNNNACDFIISLGGGSSHDCAKGIGLVTAGGGHIRDYEGIDKSTVPMTPLIAINTTAGTASEMTRFCIITNTETHVKMAIVDWRCTPLIAIDDPKLMIAKPAALTAATGMDALTHAVEAYVSTAANPITDACAEKAISMISQWLSPAVANGENIEARDAMSYAQYLAGMAFNNASLGYVHAMAHQLGGFYNLPHGVCNAILLPHVCEFNLIACPDRYAKIAELMGVNIEGLTINEAAYAAIDAIKILSQSIGIPTGLKELSVKEEDLEVMAQNAQKDACMLTNPRKADLQQVINIFKAAM

SEQ ID NO: 35 - protein, 382 aa, Achromobacter sp.
MTVSEFFIPSHNILGPGALDQAMPIIGKMGFKKALIITDADLAKLGMAQLVADKLTAQGIDTAIFDKVQPNPTVGNVNAGLDALKAHGADLIVSLGGGSSHDCAKGVALVASNGGKIADYEGVDKSAKPQLPLLAINTTAGTASEMTRFTIITDETRHVKMAIIDRHITPFLSVNDSDLMEGMPASLTAATGMDALTHAVEAYVSTIATPITDACAVKVVELIAKYLPTAVREPHNKKAREQMAYAQFLAGMAFNNASLGYVHAMAHQLGGFYDLPHGVCNALLLPHVQAFNMQVAGERLNEIGKLLSDNNADLKGLDVIAAIKKLADIVGIPKSLEELGVKREDFPVLADNALKDVCGATNPIQTDKKTIMGIFEEAFGVR

SEQ ID NO: 36 - protein, 390 aa, Asaia platycodi SF2.1
MAHIALADHTDSFFIPCVTLIGPGCAKQAGDRAKALGARKALIVTDAGLKKMGVADIISGYLLEDGLQTVIFDGAEPNPTDKNVHDGVKIYQDNGCDFIVSLGGGSAHDCAKGIGLVTAGGGNIRDYEGVDKSRVPMTPLIAINTTAGTASEMTRFCIITNSQTHVKMAIVDWRCTPLIAIDDPNLMVAMPPALTAATGMDALTHAIEAYVSTAATPITDACAEKAISLIGEFLPKAVGNGENMEARVAMCYAQYLAGMAFNNASLGYVHAMAHQLGGFYNLPHGVCNAVLLPHVCRFNLIAAADRYARVARLLGVPTDLMSRDEAAEAAIDAITQMARSVGIPSGLTALGVKAEDHKTMAENAQKDACMLTNPRKATLAQIIGVFEAAM

SEQ ID NO: 37 - protein, 381 aa, Neisseria wadsworthii
MATQFFMPVQNILGAGALAEAMDVIAALGLKKALIITDAGLSKLGVAEQIGSLLKGKGIDYAVFDKAQPNPTVSNVNAGLEQLKNSGAEFIVSLGGGSSHDCAKAVAIVAANGGKIEDYEGLNKAKKPQLPLISINTTAGTASEMTRFAVITDESRHVKMAIVDKNVTPLLSVNDPSLMENMPAPLTAATGMDALTHAVEAYVSTGASPITDACAVKAIELIARYLPTAVHEPKNKEAREQMAYAQFLAGMAFNNASLGYVHAMAHQLGGFYDLPHGVCNALLLPHVERFNQQAAKERLDEIGQILTKNNKDLAGLDVIDAITKLAGIVGIPKSLKELGVKEEDFDVLADNALKDVCGFTNPIQADKQQIIGIFKAAFDPA

SEQ ID NO: 38 - protein, 382 aa, Idiomarina loihiensis
MSSTFYIPAVNIIGENALKDAATQMDNYGFKQALIVTDPGMTKLGVTAEIEALLKEHGIDSLIYDGVQPNPTVTNVKAGLDVLQKHQCDCVISLGGGSAHDCAKGIALVATNGGHISDYEGVDVSKKPQLPLISINTTAGTASEMTRFCIITDPERHIKMAIVDQNVTPILSVNDPRLMVGMPASLTAATGMDALTHAVEAYVSTDATPITDACAIKAIEIIRDNLHEAVHNGANMEAREQMAYAQFLAGMAFNNASLGYVHAMAHQLGGFYDLPHGVCNAVLLPHVQRYNSQVVAPRLKDIGKALGAEVQGLTEKEGADAAIAAIVKLSQSVNIPAGLEELGAKEEDFNTLADNAMKDACGLTNPIQPSHEDIVTIFKAAF

SEQ ID NO: 39 - protein, 382 aa, Comamonadaceae bacterium
MTSTFFMPAVNLMGSGSLGEAMQAVKGLGYRKALIVTDAMLNKLGLADKVAKLLNELQIATVVFDGAQPNPTKGNVRAGLALLRANQCDCVVSLGGGSSHDCAKGIALCATNGGEISDYEGVDRSVKPQLPLVAINTTAGTASEMTRFCIITDEETHIKMAIVDRNVTPILSVNDPDLMLAKPKALTAATGMDALTHAVEAYVSTAATPITDACALKAVELIARHLRTAVAKGDDLHAREQMAYAQFLAGMAFNNASLGYVHAMSHQLGGFYDLPHGVCNALLLPHVEAFNVKTSAARLRDVAQAMGENVQGLDAQAGAQACLAAIRKLSSDIGIPKSLGELGVKRADIPTLAANAMKDACGFTNPRSATQTEIEAIFEGAM

SEQ ID NO: 40 - protein, 382 aa, Pseudomonas putida
MSSTFFIPAVNIMGIGCLDEAMTAIVGYGFRKALIVTDGGLAKAGVAQRIAEQLAVRDIDSRVFDDAKPNPSIANVEQGLALLQREKCDFVISLGGGSPHDCAKGIALCATNGGRIADYEGVDRSTKPQLPLVAINTTAGTASEMTRFCIITDEARHVKMAIVDRNVTPILSVNDPALMVAMPKALTAATGMDALTHAVEAYVSTAATPITDACALKAIELISGNLRQAVANGQDLLAREAMAYAQFLAGMAFNNASLGYVHAMAHQLGGFYDLPHGVCNAVLLPHVQRFNAKVSAARLRDVAAALGVEVAELNAEQGAAAAIEAIEQLSRDIDIPPGLAVLGAKVEDVPILAGNALKDACGLTNPRPASQAEIEAVFKAAF

SEQ ID NO: 41 - protein, 383 aa, Enterobacteriaceae bacterium
MAASTFYIPSVNVIGADSLKSAMDTMRDYGYRRALIVTDAILNKLGMAGDVQKGLAERDIFSVIYDGVQPNPTTANVNAGLAILKENNCDCVISLGGGSPHDCAKGIALVASNGGQISDYEGVDRSAKPQLPMIAINTTAGTASEMTRFCIITDEARHIKMAIVDKHVTPILSVNDSSLMTGMPKSLTAATGMDALTHAIEAYVSIAATPITDACALKAITMIAENLSVAVADGANAEAREAMAYAQFLAGMAFNNASLGYVHAMAHQLGGFYDLPHGVCNAVLLPHVQAFNSKVAAARLRDCAQAMKVNVAGLSDEQGAKACIDAICKLAREVNIPAGLRDLNVKEEDIPVLATNALKDACGFTNPIQATHDEIMAIYRAAM

SEQ ID NO: 42 - protein, 382 aa, Pseudomonas sp.
MSSTFFIPAVNMIGSGCLQEAMQAIRKYGFLKALIVTDAGLAKAGVATQVAGLLVEQGIDSVIYDGARPNPTIANVEQGLELLQAHQCDFVISLGGGSPHDCAKGIALCASNGGHISDYEGVDRSQQPQLPLVAINTTAGTASEMTRFCIITDTARHVKMAIIDRNVTPILSVNDPQMMAGMPRSLTAATGMDALTHAVEAYVSTAATPITDACALKAIGLIAGNLQRAVEQGDDLQARENMAYAQFLAGMAFNNASLGYVHAMAHQLGGFYDLPHGVCNAVLLPHVQRFNASVSAARLTDVAHAMGANIRGMSPEAGAQAAIDAISQLAASVEIPAGLTQLGVKQSDIPTLAANALKDACGLTNPRPADQQQIESIFQAAL

SEQ ID NO: 43 - protein, 390 aa, Burkholderia glumae
MSYLSIADRTDSFFIPCVTLIGAGCARETGTRAKSLGAKKALIVTDAGLHKMGLSATIAGYLREAGVDAVIFPGAEPNPTDVNVHDGVKLYQQNGCDFIVSLGGGSSHDCAKGIGLVTAGGGHISHYEGVDKSSVPMTPLISINTTAGTAAEMTRFCIITNSSNHVKMAIVDWRCTPLIAIDDPRLMVAMPPALTAATGMDALTHAVEAYVSTAATPITDACAEKAIALIGEWLPKAVANGESMEARAAMCYAQYLAGMAFNNASLGYVHAMAHQLGGFYNLPHGVCNAILLPHVCEFNLIAAPERFARIAALLGANTAGLSVTDAGAAAIAAIRALSASIDIPAGLAGLGVKADDHEVMARNAQKDACMLTNPRTATLKQVIGIFEAAM

SEQ ID NO: 44 - protein, 383 aa, Aeromonas hydrophila
MATFKFYIPAINLMGAGCLQEAAADIQGHGYRKALIVTDKILGQIGVVGRLAALLAEHGIDAVVFDETRPNPTVANVEAGLAMIRAHGCDCVISLGGGSPHDCAKGIALVAANGGSIKDYEGVDRSAKPQLPLIAINTTAGTASEMTRFCIITDESRQVKMAIIDKHVTPLMSVNDPELMLAKPAGLTAATGMDALTHAIEAYVSTAATPVTDASAVMAIALIAEHLRTAVHQGEDLHAREQMAYAQFLAGMAFNNASLGYVHAMAHQLGGFYDLPHGVCNAVLLPHVQAYNARVCAGRLKDVARHMGVDVSAMSDEQGAAAAIDAIRQLASDVKIPTGLEQLGVRADDLDVLATNALKDACGLTNPRQATHAEIVAIFRAAM

SEQ ID NO: 45 - protein, 403 aa, Acinetobacter johnsonii
MAFKNIADQTNGFYIPCVSLFGPGCAKEIGGKAQNLGAKKALIVTDAGLFKFGVADTIAGYLKDAGVDSHIFPGAEPNPTDINVHNGVTAYNEQGCDFIVSLGGGSSHDCAKGIGLVTAGGGHIRDYEGIDKSTVPMTPLIAINTTAGTASEMTRFCIITNTDTHVKMAIVDWRCTPLIAIDDPKLMIAKPASLTAATGMDALTHAVEAYVSTAANPITDACAEKAISMISEWLSPAVANGENLEARDAMSYAQYLAGMAFNNASLGYVHAMAHQLGGFYNLPHGVCNAVLLPHVCEFNLIACPDRYARIAELMGVNITGLTVTEAGYAAIDAIRELSASIGIPSSLSELGVKEQDLGVMSENAQKDACMLTNPRKANHAQVVDIFKAALKSGASVVDFKAAV

SEQ ID NO: 46 - protein, 382 aa, Shewanella oneidensis
MAAKFFIPSVNVLGKGAVDDAIGDIKTLGFKRALIVTDKPLVNIGLVGEVAEKLGQNGITSTVFDGVQPNPTVGNVEAGLALLKANQCDFVISLGGGSPHDCAKGIALVATNGGSIKDYEGLDKSTKPQLPLVAINTTAGTASEMTRFCIITDEARHIKMAIVDKHTTPILSVNDPELMLKKPASLTAATGMDALTHAVEAYVSIAANPITDACAIKAIELIQGNLVNAVKQGQDIEAREQMAYAQFLAGMAFNNASLGYVHAMAHQLGGFYDLPHGVCNALLLPHVQEYNAKVVPHRLKDIAKAMGVDVAKMTDEQGAAAAITAIKTLSVAVNIPENLTLLGVKAEDIPTLADNALKDACGFTNPKQATHAEICQIFTNAL

SEQ ID NO: 47 - protein, 382 aa, Commensalibacter intestini
MSTTFFIPSINVVGENALNDAVPHILGHGFKHGLIVTDEFMNKSGVAQKVSDLLAKSGINTSIFDGTHPNPTVSNVNDGLKILKANNCDFVISLGGGSPHDCAKGIALLASNGGEIKDYEGLDVPKKPQLPLVSINTTAGTASEITRFCIITDEVRHIKMAIVTSMVTPILSVNDPALMAAMPPGLTAATGMDALTHAIEAYVSTAASPITDACALKAATMISENLRTAVKDGKNMAARESMAYAQLLAGMAFNNASLGYVHAMAHQLGGFYGLPHGVCNAVLLPHVQEYNLPTCAGRLKDMAKAMGVNVDKMSDEEGGKACIAAIRALSKDVNIPANLTELKVKAEDIPTLAANALKDACGVTNPRQGPQSEVEAIFKSAM

SEQ ID NO: 48 - protein, 382 aa, Pseudomonas fluorescens
MSSTFFIPAVNVMGLGCLDEAMTAIRNYGFRKALIVTDTGLAKAGVASKVAGLLALQDIDSVIFDGAKPNPSIANVELGLGLLKESQCDFVVSLGGGSPHDCAKGIALCATNGGHIGDYEGVDRSTKPQLPLIAINTTAGTASEMTRFCIITDESRHVKMAIVDRNVTPLMSVNDPALMVAMPKGLTAATGMDALTHAIEAYVSTVANPITDACALKAVTLISNNLRLAVRDGGDLAARENMAYAQFLAGMAFNNASLGFVHAMAHQLGGFYDLPHGVCNAVLLPHVQSFNASVCADRLTDVAHAMGGDTRGLSPEEGAQAAIAAIRSLARDVDIPAGLRDLGVRLNDVPVLATNALKDACGLTNPRAADQRQIEEIFRSAY

SEQ ID NO: 49 - protein, 382 aa, Pseudomonas sp.
MSSTFFIPAVNIMGIGCLDEAMNAIRNYGFRKALIVTDAGLAKAGVASMIAEKLAMQDIDSLVFDGAKPNPSIDNVEQGLLRLREGNCDFVISLGGGSPHDCAKGIALCATNGGHIRDYEGVDQSAKPQLPLIAINTTAGTASEMTRFCIITDEARHVKMAIVDRNVTPLLSVNDPALMVAMPKGLTAATGMDALTHAIEAYVSTAANPITDACALKAIDMISNNLRQAVHDGSDLTARENMAYAQFLAGMAFNNASLGFVHAMAHQLGGFYDLPHGVCNAVLLPHVQSFNASVCAERLTDVAHAMGADIRGFSPEEGAQAAIAAIRSLARDVEIPAGLRELGAKLPDIPILAANALKDACGLTNPRAADQRQIEEIFRSAF

SEQ ID NO: 50 - protein, 393 aa, Artificial Sequence
  Synthetic
MSLVNYLQLADRTDGFFIPSVTLVGPGCVKEVGPRAKMLGAKRALIVTDAGLHKMGLSQEIADLLRSEGIDSVIFAGAEPNPTDINVHDGVKVYQKEKCDFIVSLGGGSSHDCAKGIGLVTAGGGHIRDYEGVDKSKVPMTPLIAINTTAGTASEMTRFCIITNTDTHVKMAIVDWRCTPLVAIDDPRLMVKMPPALTAATGMDALTHAVEAYVSTAATPITDTCAEKAIELIGQWLPKAVANGDWMEARAAMCYAQYLAGMAFNNASLGYVHAMAHQLGGFYNLPHGVCNAILLPHVCQFNLIAATERYARIAALLGVDTSGMETREAALAAIAAIKELSSSIGIPRGLSELGVKAADHKVMAENAQKDACMLTNPRKATLEQVIGIFEAAM

SEQ ID NO: 51 - protein, 381 aa, Neisseria weaveri
MATQFFMPVQNILGENALAEAMDVISALGLKKALIVTDGGLSKMGVADKIGGLLKEKNIDYAVFDKAQPNPTVTNVNDGLAALKEAGADFIVSLGGGSSHDCAKAVAIVTTNGGKIEDYEGLDKSKKPQLPLIAINTTAGTASEMTRFAVITDEARHVKMAIVDKNVTPLLSVNDPSLMEGMPAPLTAATGMDALTHAVEAYVSTIASPITDACALKAIELIAGYLPTAVHEPKNKEAREKMAYAQFLAGMAFNNASLGYVHAMAHQLGGFYDLPHGVCNALLLPHVERFNQQAAKERLDEIGAILGKYNSDLKGLDVIDAITKLARIVGIPKSLKELGVKQEDFGVLADNALKDVCGFTNPIQANKEQIIGIYEAAFDPA

SEQ ID NO: 52 - protein, 390 aa, Acinetobacter gerneri
MAFKNLADQTNGFYIPCVSLFGPGCAKEVGAKAQNLGAKKALIVTDAGLFKFGVADIIVGYLKDAGVDSHVFPGAEPNPTDINVLNGVQAYNDNGCDFIVSLGGGSSHDCAKGIGLVTAGGGNIRDYEGIDKSSVPMTPLIAINTTAGTASEMTRFCIITNTDTHVKMAIVDWRCTPLVAIDDPKLMIAKPAALTAATGMDALTHAVEAYVSTAANPITDACAEKAISMISEWLSSAVANGENIEARDAMAYAQYLAGMAFNNASLGYVHAMAHQLGGFYNLPHGVCNAILLPHVCEFNLIACPDRFAKIAQLMGVDTTGMTVTEAGYEAIAAIRELSASIGIPSGLTELGVKAADHAVMTSNAQKDACMLTNPRKATDAQVIAIFEAAM

SEQ ID NO: 53 - protein, 387 aa, Citrobacter freundii
MSYRMFDYLVPNVNFFGPNAISVVGERCKLLGGKKALLVTDKGLRAIKDGAVDKTLTHLREAGIDVVVFDGVEPNPKDTNVRDGLEVFRKEHCDIIVTVGGGSPHDCGKGIGIAATHEGDLYSYAGIETLTNPLPPIVAVNTTAGTASEVTRHCVLTNTKTKVKFVIVSWRNLPSVSINDPLLMLGKPAPLTAATGMDALTHAVEAYISKDANPVTDAAAIQAIRLIARNLRQAVALGSNLKARENMAYASLLAGMAFNNANLGYVHAMAHQLGGLYDMPHGVANAVLLPHVARYNLIANPEKFADIAEFMGENTDGLSTMDAAELAIHAIARLSADIGIPQHLRDLGVKEADFPYMAEMALKDGNAFSNPRKGNEKEIAEIFRQAF

SEQ ID NO: 54 - protein, 390 aa, Acinetobacter sp.
MAFKNIADQTNGFYIPCVSLFGPGSAKEVGVKAQNLGAKKALIVTDAGLYKFGVADIIAGYLKEAQVESYIFAGAEPNPTDINVHDGVEAYNNNACDFIISLGGGSSHDCAKGIGLVTAGGGHIRDYEGIDKSTVPMTPLIAINTTAGTASEMTRFCIITNTETHVKMVIVDWRCTPLIAIDDPKLMIAKPAALTAATGMDALTHAVEAYVSTAANPITDACAEKAISMISQWLSPAVANGENIEARDAMSYAQYLAGMAFNNASLGYVHAMAHQLGGFYNLPHGVCNAILLPHVCEFNLIACPDRYAKIAELMGVNIEGLTINEAAYAAIDAIKILSQSIGIPTGLKELSVKEEDLEVMAQNAQKDRCMLTNPRKADLQQVINIFKAAM

SEQ ID NO: 55 - protein, 390 aa, Acinetobacter sp.
MAFKNIADQTNGFYIPCVSLFGPGSVKEVGSKAQNLGAKKALIVTDAGLYKFGVADIIAGYLKEAQVESYIFAGAEPNPTDINVHDGVEAYNNNACDFIISLGGGSSHDCAKGIGLVTAGGGHIRDYEGIDKSTVPMTPLIAINTTAGTASEMTRFCIITNTETHVKMVIVDWRCTPLIAIDDPKLMIAKPAALTAATGMDALTHAVEAYVSTAANPITDACAEKAISMISQWLSPAVANGENIEARDAMSYAQYLAGMAFNNASLGYVHAMAHQLGGFYNLPHGVCNAILLPHVCEFNLIACPDRYAKIAELMGVNIEGLTINEAAYAAIDAIKILSQSIGIPTGLKELSVKEEDLEVMAQNAQKDRCMLTNPRKADLQQVINIFKAAM

SEQ ID NO: 56 - protein, 390 aa, Acinetobacter sp.
MAFKNIADQTNGFYIPCVSLFGPGSVKEVGVKAQNLGAKKALIVTDAGLYKFGVADIIAGYLKEAQVESYIFAGAEPNPTDINVHDGVEAYNNNACDFIISLGGGSSHDCAKGIGLVTAGGGHIRDYEGIDKSTVPMTPLIAINTTAGTASEMTRFCIITNTETHVKMVIVDWRCTPLIAIDDPKLMIAKPAALTAATGMDALTHAVEAYVSTAANPITDACAEKAISMISQWLSPAVANGENIEARDAMSYAQYLAGMAFNNASLGYVHAMAHQLGGFYNLPHGVCNAILLPHVCEFNLIACPDRYAKIAELMGVNIEGLTINEAAYAAIDAIKILSQSIGIPTGLKELSVKEEDLEVMAQNAQKDRCMLTNPRKADLQQVINIFKAAM

SEQ ID NO: 57 - protein, 40 aa, Artificial Sequence
  Synthetic
  misc_feature (18)..(18): Xaa can be any naturally occurring amino acid
  misc_feature (26)..(26): Xaa can be any naturally occurring amino acid
  misc_feature (35)..(35): Xaa can be any naturally occurring amino acid
LAGMAFNNASLGYVHAMXHQLGGFYXLPHGVCNAXLLPHV

SEQ ID NO: 58 - protein, 40 aa, Artificial Sequence; identical to SEQ ID NO: 57
  Synthetic
  MISC_FEATURE (18)..(18): may be Alanine or Serine
  MISC_FEATURE (26)..(26): may be Asparagine or Aspartic Acid
  MISC_FEATURE (35)..(35): may be Leucine, Valine, or Isoleucine

SEQ ID NO: 59 - protein, 6 aa, Artificial Sequence
  Synthetic
KMAIVD

SEQ ID NO: 60 - protein, 6 aa, Artificial Sequence
  Synthetic
KMAIID

SEQ ID NO: 61 - protein, 6 aa, Artificial Sequence
  Synthetic
KFVIVS

SEQ ID NO: 62 - protein, 6 aa, Artificial Sequence
  Synthetic
KMAIVT

SEQ ID NO: 63 - protein, 6 aa, Artificial Sequence
  Synthetic
KMPVID

SEQ ID NO: 64 - protein, 6 aa, Artificial Sequence
  Synthetic
KMPVID

SEQ ID NO: 65 - protein, 6 aa, Artificial Sequence
  Synthetic
KMVIVD

SEQ ID NO: 66 - protein, 4 aa, Artificial Sequence
  Synthetic
KDAC

SEQ ID NO: 67 - protein, 4 aa, Artificial Sequence
  Synthetic
KDVC

SEQ ID NO: 68 - protein, 4 aa, Artificial Sequence
  Synthetic
KDGN

SEQ ID NO: 69 - protein, 4 aa, Artificial Sequence
  Synthetic
QDVC

SEQ ID NO: 70 - protein, 4 aa, Artificial Sequence
  Synthetic
QDRC

SEQ ID NO: 71 - protein, 4 aa, Artificial Sequence
  Synthetic
NDAC

SEQ ID NO: 72 - protein, 4 aa, Artificial Sequence
  Synthetic
KDRC

SEQ ID NO: 73 - DNA, 1152 bp, Artificial Sequence
  Synthetic
atgtcgattagcaccttcttcattccgccggtgaacatgattggcaccggctgcttagcggatgcgatcaaaagcatgaaagattacggctaccataacgccttaattgttacggatagcgtgttaaaccagattggcgtagtgggcgaagttcagaacttactgcgcgaggcggggattcgcagccgcatttacgatggcacccatccgaatccgaccaccgttaatgttagcgaaggtctggccattctgcaagaacatcagtgtgattgtgtgattagccttggcggcggcagcccgcatgattgtgcaaaggggattgccctggtggcgagcaacggcggcgacattcgcgactatgagggcgtagatcgcagcgcgaaaccgcagctgccgctgattgccattaataccaccgccggtaccgccagcgaaatgacccgcttctgcattattaccgatgtcgaccgccatattaaaatggcgattgtggataagcatgtgaccccgattttaagcgtaaacgatagcggcttaatggcgggcatgccgaaaggcctgaccgccgcgaccggtatggatgccttaacccatgcaattgaagcctacgtaagcattgccgcgaacccgattaccgacgcctgcgcgctgaaagcggtgaccatgattagccagtacttagcgcgtgcggtcgcccagggcgatgatatggaagcgcgtgaaatgatggcgtatgcgcagtttcttgccggcatggcctttaataacgccagcttaggttatgttcatgcgatggctcatcagctgggaggcttctacgacctgccgcatggtgtctgtaacgccgtgctgctgccgcatgtagagagctttaatgcaaaggcatgcgccccgcgtcttaaagatattgcggtggcgatgggtgtggacaccaaaggtatgaatgacgaacagggtgcagctgcgtgtattgcagaaattcgtaagttaagtaagactgttggtattccaagtggtttagttgagttaaatgtaaaggaagaagatctcccggttctcgcgaccaatgcgctgaaagatgcctgtggcctgaccaacccgattcaggccacccatgaagaaattgtggcaatttttaagagcgcgatgtga

SEQ ID NO: 74 - DNA, 1158 bp, Artificial Sequence
  Synthetic
atgaaaaatacccaaagcgccttctacatgccgtctgttaatctgttcggcgcgggctcggtaaacgaggtgggtacccgcctagcgggcctgggagtgaagaaagcgctgctggtaacggacgcaggattacactctctgggcttaagcgaaaaaattgcaggtattattcgcgaagcgggggtagaagttgcgatttttcctaaagcggagccgaatccgaccgataaaaacgttgcagagggcctagaggcatacaacgcagaaaattgtgactcaattgtcacattaggcggtggctctagccatgacgcgggtaaggcgattgctttagtcgccgctaacgggggtaccattcatgactatgaaggtgttgatgtttctaaaaaacctatggtgccgctgattgcgattaacaccaccgccggcacggggagcgaactgacgaaattcactattattactgatactgaacgtaaagttaaaatggcgatagttgacaaacatgttacgcctacactgtcgatcaacgatccggagctaatggtgggtatgcctccgtcgctcaccgctgctacaggcctggacgcgctgacgcatgcgatcgaagcgtatgtgagtaccggcgctacccccattacagatgcgcttgccattcaggccattaaaataatctcaaaatatctgccgcgtgctgtggcgaacggcaaagatattgaggcccgcgaacagatggcgttcgcacagtcgcttgcgggtatggcctttaacaacgccggtctgggctatgtccacgcgattgcacaccagcttggcggcttttataattttcctcacggcgtttgcaatgcgatcctgctgcctcatgtatgccgttttaatttaatcagcaaagtggaacgttatgcagaaattgcggcgtttttaggtgaaaacgttgatggtttaagtacgtatgaagctgccgagaaagcgatcaaggctattgagcgtatggcccgtgacctgaatatcccgaaaggtttcaaagaactgggtgcgaaggaagaagacattgaaactctggcgaaaaatgctatgaatgatgcttgtgcattaactaatccgcgtaaaccaaaattagaggaagttatccagattattaaaaatgccatgtga

SEQ ID NO: 75 - DNA, 945 bp, Artificial Sequence
  Synthetic
atgcaggaacatatccaggctgtgctgaagaatattgagaaagtgatgattggcaagcgcgaagtcgcggaactgagcattgtcgcgttgctgaccggtggccatgtgcttctggaagatgtgccgggtgttggcaagaccatgatggtacgcagcctggccaaaagcgtgggcgcgaatttcaaacgcattcagtttaccccggatttgttaccgagcgatgtagtgggcgtaagcatttataacccgaagaccctccagtttgagtttcgcccggggccgattgtaggcaacattattttggccgatgaaattaatcgcacgagcccgaaaacccaggcggcactcctcgaagctatggaagaagcgagcattaccgtcgatggcgaaaccctgagcattccgaagccgtttttcgtaatggccacccagaacccgattgagtacgaaggtacctatccgttgccggaagcccaactggatcgctttctgctgaagattcgcatgggttacccgagcgtacaacaggagattgaagtgctgcgccgcgccgagaacaagcagccgattgaagaaattaaggccgtgatgaccgtagaagaactgctggcgctgcaacgcgcggtgcagcaagtttacattgaagatagcgtgaaaggctacattgttgacatcgcacgcgcaacccgcgaaaatccgcgcgtttacttaggtgtgagcccgcgcgcgagcgttgccctgatgaaggcaagccaggcatatgcgtttattcaggggcgcgatttcgtgaaaccggatgatattaagtacctcgccccgtttgtgtttggccatcgcctgatcctcaccccggatacccgctacgaaggcgtaaccccggaacagattattagccagattatcgagcagacgtacgtgccggttcgccgcttcaccgactcgtga

SEQ ID NO: 76 - DNA, 1149 bp, Artificial Sequence
  Synthetic
atgtcgagtactttttttattccagcagtaaatattattggtagtggttgtattgaggaagccatgcaggcaattcgcaagtatggcttcttaaaagccctgattgttaccgacgcggggctggcgaaagccggcattgcggcgcaagtcgcgggcctgttactggaacagggcattgatgcggtcgtgtatgacggcgcaaaaccgaatccgaccattagcaacgtggaaaagggcttagcgctcttacaagagcgccaatgtgattttgtcattagcttgggtggcggcagcccgcatgattgcgccaaggggattgcgctgtgtgcgagcaatggcgggcatattagcgattacgaaggcgttgaccgcagcgaaaaaccgcagctgccgttaattgcaattaacaccaccgcgggcaccgcaagcgaaatgacccgcttttgtatcattaccgacgaggtgcgccatgtgaagatggctattattgatcgcaacgtgaccccgattctgagcgttaacgatccgaaaatgatggttggcatgccgcgcagcctcaccgccgccaccggcatggacgcgctcacccatgcaattgaagcctatgtaagcaccgcagccaccccgattaccgatgcatgtgcgattaaagcggtgaatctgattgcaggtaatctgtacaaagcagttgtcgatggcaccgatattgtcgcccgtgagaatatggcatatgcgcagttcttagccggtatggcattcaacaatgccagccttggctacgtccatgcgatggctcatcagctgggaggcttctatgatcttccgcatggcgtgtgcaacgccgtcctgctgccgcatgttcagagctttaatgccaccgtgagcgccgcacgcctgaccgatgtggcacatgcgatgggtgccgacattcgcggcctcagcccgcaggatggcgcgcgcgcggcagtagcggccatccgcaaactgagcaccagcgtcgaaattccgagcgggttagttgccctgggcgttaaagaggaagatattccgaccctggctgcaaacgctttgaaagatgcctgcggcctgaccaatccgcgcccggcgacgcaggaacagattgaaggcattttccgccaagccctctga

SEQ ID NO: 77 - DNA, 1152 bp, Artificial Sequence
  Synthetic
atggccacctctacattctacatcccgagcgtgaacttgatgggcgccggttgtctccgcgatgcggtcaaagcgattcagagccacggctggcgcaaagcactcattgtgactgacctgccgctcgtgcgcgcgggcctcgccgggcaagtcgtagaacgcctgggcgagcagggcatcggcgctgccgtgttcgatggcgtgaaaccgaatcccaacgtggccaacgtggaagcaggcctggcgttactgcgcgccgaaggctgtgatttcgtgattagtctcggtggcgggtccccgcatgattgtgcgaagggcattgcactggttgctgccaatggcggaaccattgctgactatgagggcgtggatcgttcggctcgcccgcagttaccgctggttgctatcaacacaaccgcgggcaccgcaagcgaaatgacccgcttctgcatcattacggacgaaacccgtcatgtcaaaatggccattgtagacaaaaatgtcacgcctgtcctttccgtgaatgatccggaaatgatggctgggatgccaccgggcctaaccgcggcgacgggcatggatgccctcacccatgcagtggaagcttatgtgagcaccgcagcgaccccgatcactgacgcctgtgctctgcaagcggtaacgctggtcagtcgccatttacgtgcggctgtggcggacggtcgcgacatggcggcccgtgaacagatggcgtatgccgaatttttagcgggcatggcttttaataacgcttcgcttggctatgtccacgcaatggcacaccagcttggaggcttttacgatctgccgcatggggtgtgtaatgcaatccttttaccgcacgtgcaggcctttaatgcgagtgtggcagcggcacgtcttggggaagttgcgcgtgcgatgggtgttcatactgctggtttagacgatgcggcagccgcggaggcttgcgtgcaggcgatccgccgtttggcggcggatgttggtattccggccggagtgggcccgctcggcgccaaggaagaagacattccgaccttggcggccaacgccatgaaagacgcgtgcggtcttacgaatcctcgcaaaccgagctttgaagaagtttgcgcgcttttcaaagcggcactctga

SEQ ID NO: 78 - DNA, 1149 bp, Artificial Sequence
  Synthetic
atgtcgtccacgttctttatcccggcggtgaatattatgggcattggctgcctggatgaggctatgtcagcgattcgcaactacggctttcgtaaagcgcttatcgtaacggacaccggcctggcaaaagcgggcgtggcttcgatggtggcggagaagcttgcgatgcaggatattgattctgtgatctttgatggcgccaaaccaaatccttccattgccaacgtcgaacaaggcctggcacagctgcaacaggcgcagtgcgatttcgtcattagtctgggaggcggcagcccgcatgactgcgctaaaggcattgcgctgtgtgctacaaacggcggtcaaattcgcgattacgaaggtgttgaccaatccgcgaaaccacagcttcctctgatcgcaattaatactacggccgggacagcgagcgagatgacccgtttctgcattattaccgacgaatcacgtcacgttaaaatggcaattgttgaccgcaatgttaccccgctgctgtcagtgaatgacccagccctgatggtcgcaatgccgaaaggcttgaccgcagcgaccggaatggacgcgctcacgcacgctgttgaagcatatgtatcgactgccgcgaatccgattacggatgcctgcgcgctcaaagcggtagagatgatctcagcgaacttacgtcaagcggttcacgatggcaatgatctgctggcgcgcgaaaacatggcgtatgcccagtttctggcgggcatggcatttaacaatgcttcgcttggttttgtgcacgcgatggcgcatcaactgggaggcttttatgaccttccgcatggagtctgcaacgcggtgctgttaccccacgtgcagagtttcaatgctaccgtttgtgcgcagcgtctgaccgatgtagcgcacgccctgggtgccgatatccgtggtttcagtcctgaagaaggtgcgcaggccgcgattgccgccattcgtaccttagcacgcgatgtcgagattcccgctggcctgcgtgaacttggtgcgaaattgcaggatatcccgctgctggcggcgaatgcgctgaaagacgcgtgcggcctgaccaacccccgtccggcggatcagcgtcagattgaagaaattttccgcaatgcgttctga

SEQ ID NO: 79 - DNA, 1149 bp, Artificial Sequence
  Synthetic
atggccaccaagttttttattccgagcgtgaacgttttaggtcagggcggggttgatgaagccattaacgacatcaaaaccctgggctttaagcgcgcgctcattgtgaccgacaccccgcttgtcaatattggcctggtcgataaagtagcggcaaaacttattgataacggcattaccgtttttattttcgatggcgtgcagccgaacccgaccgtgagcaatgtggaagctggcctggcaatgctgaatgcccatgagtgtgactttgttattagcctgggcggcggcagcccgcatgactgcgccaaagggattgccttggtggcaaccaacggcggcaatattagcgattacgaaggcctggacgtgagcacccgcccgcagttaccgctggttgcgattaacaccaccgccggcaccgccagcgaaatgacccgcttttgcattattaccgatgaaacgcgccatattaaaatggccattgtagataagaacaccaccccgattctgagcgtaaacgatccggaattaatgattgaaaaaccggctgcgctgaccgcagccaccgggatggatgcgctcacccatgcgattgaagcgtatgtaagcattgcagccacgccgattaccgatgcctgtgccattaaagcgattgaactgattaaggcaaacttagttaatgccgtggaacaaggggacaatattgacgcgcgcgaacagatggcctacgcccagttcctggcgggcatggcctttaacaacgcgagcctgggctatgtgcatgcgatggctcatcagctgggcggcttctatgacctgccgcatggcgtgtgcaatgccctgctgctgccgcatgtgcaagcgtacaacgcgaaagtggtcccgggcaaactgaaagatattgccaaggcaatgggcgtagatgtggcacagttaagcgacgaacagggcgcggagagcgccattgaagcgattaaagcactgagcgtggccgtaaatattccggcgaatctcaccgaactgggtgtgaatccggaggacattccggtgcttgctgataacgcgctgaaagatgcatgtgggttaaccaatccgcagcaggctacccatgcggaaatttgcgagattttcaccaacgcgctctga

SEQ ID NO: 80 - DNA, 1149 bp, Artificial Sequence
  Synthetic
atgtcggtaagcgaatttcatatcccggcgctcaacctcatgggtgccggggccctgaaacaagctatcgggaacattcaaaaacaaggttttagccgcgcattaattgtgactgatgcaggccttgttagcgccgggctagttgacgaggttacccagctgctgcaacaggccggcgttgcgacctgtgtatttgccgatgttcagcctaatccgacgaccgccaacgttgcagcgggtctggcgctgctgcaacagcagcaatgcgatctggttatcagcctgggcggaggatcgccgcacgattgcgcaaaaggcatcgcgctggtggctaccaatgggggcgacatccgcgattacgagggcgtagataaatcagcaaaaccgcaactgccgctgatcagtattaacacgaccgcaggtacggcctcagaaatgacgcgcttttgtattattacagatgaaacccgccatattaaaatggcaattgttgacaaacacaccacgccgattttaagtgtgaacgacccgttgaccatggttggtatgcctacacagctgactgcggcgacgggcatggacgcacttacccatgcagttgaagcctatgtgagcacagccgctacgcctatcaccgatgcctgcgcgctgaaagcggtggaattgatcacccgttttctgcctcgtgcagttcagcagggtgatgatctggaggcgcgcgagcaaatggcatacgcccagtttttagcaggtatggcgttcaataacgcaagtctgggttacgtgcacgcaatggcacaccagctgggcggtttttatgatttgccgcatggcgtctgcaatgctgtgttgttaccgcatgttcaggtttttaacagccaagtcgcagcggaacgcttggcacaggtaggggtagctatgggcctagcggcgagcgataatgcccaagccggcgcagacgcctgtatcgcagcgattaaagccctcaaagatcaggtaggcattcctcgtggtctggctgatctgggtgcgaaagcagaagacattccagtgcttgccgcgaacgcgctaaaagatgcatgcggcttcacaaacccgattcaggccaatcagtcccagattgaggcaatttttcaacaggcctggtga

SEQ ID NO: 81 - protein, 383 aa, Pragia fontium
MSISTFFIPPVNMIGTGCLADAIKSMKDYGYHNALIVTDSVLNQIGVVGEVQNLLREAGIRSRIYDGTHPNPTTVNVSEGLAILQEHQCDCVISLGGGSPHDCAKGIALVASNGGDIRDYEGVDRSAKPQLPLIAINTTAGTASEMTRFCIITDVDRHIKMAIVDKHVTPILSVNDSGLMAGMPKGLTAATGMDALTHAIEAYVSIAANPITDACALKAVTMISQYLARAVAQGDDMEAREMMAYAQFLAGMAFNNASLGYVHAMAHQLGGFYDLPHGVCNAVLLPHVESFNAKACAPRLKDIAVAMGVDTKGMNDEQGAAACIAEIRKLSKTVGIPSGLVELNVKEEDLPVLATNALKDACGLTNPIQATHEEIVAIFKSAM

SEQ ID NO: 82 - protein, 385 aa, Bacillus methanolicus MGA3; identical to SEQ ID NO: 29

SEQ ID NO: 83 - protein, 314 aa, Lysinibacillus odysseyi 34hs-1 = NBRC 100172
MQEHIQAVLKNIEKVMIGKREVAELSIVALLTGGHVLLEDVPGVGKTMMVRSLAKSVGANFKRIQFTPDLLPSDVVGVSIYNPKTLQFEFRPGPIVGNIILADEINRTSPKTQAALLEAMEEASITVDGETLSIPKPFFVMATQNPIEYEGTYPLPEAQLDRFLLKIRMGYPSVQQEIEVLRRAENKQPIEEIKAVMTVEELLALQRAVQQVYIEDSVKGYIVDIARATRENPRVYLGVSPRASVALMKASQAYAFIQGRDFVKPDDIKYLAPFVFGHRLILTPDTRYEGVTPEQIISQIIEQTYVPVRRFTDS

SEQ ID NO: 84 - protein, 382 aa, Pseudomonas cichorii JBC1
MSSTFFIPAVNIIGSGCIEEAMQAIRKYGFLKALIVTDAGLAKAGIAAQVAGLLLEQGIDAVVYDGAKPNPTISNVEKGLALLQERQCDFVISLGGGSPHDCAKGIALCASNGGHISDYEGVDRSEKPQLPLIAINTTAGTASEMTRFCIITDEVRHVKMAIIDRNVTPILSVNDPKMMVGMPRSLTAATGMDALTHAIEAYVSTAATPITDACAIKAVNLIAGNLYKAVVDGTDIVARENMAYAQFLAGMAFNNASLGYVHAMAHQLGGFYDLPHGVCNAVLLPHVQSFNATVSAARLTDVAHAMGADIRGLSPQDGARAAVAAIRKLSTSVEIPSGLVALGVKEEDIPTLAANALKDACGLTNPRPATQEQIEGIFRQAL

SEQ ID NO: 85 - protein, 383 aa, Rubrivivax gelatinosus
MATSTFYIPSVNLMGAGCLRDAVKAIQSHGWRKALIVTDLPLVRAGLAGQVVERLGEQGIGAAVFDGVKPNPNVANVEAGLALLRAEGCDFVISLGGGSPHDCAKGIALVAANGGTIADYEGVDRSARPQLPLVAINTTAGTASEMTRFCIITDETRHVKMAIVDKNVTPVLSVNDPEMMAGMPPGLTAATGMDALTHAVEAYVSTAATPITDACALQAVTLVSRHLRAAVADGRDMAAREQMAYAEFLAGMAFNNASLGYVHAMAHQLGGFYDLPHGVCNAILLPHVQAFNASVAAARLGEVARAMGVHTAGLDDAAAAEACVQAIRRLAADVGIPAGVGPLGAKEEDIPTLAANAMKDACGLTNPRKPSFEEVCALFKAAL

SEQ ID NO: 86 - protein, 382 aa, Pseudomonas fluorescens
MSSTFFIPAVNIMGIGCLDEAMSAIRNYGFRKALIVTDTGLAKAGVASMVAEKLAMQDIDSVIFDGAKPNPSIANVEQGLAQLQQAQCDFVISLGGGSPHDCAKGIALCATNGGQIRDYEGVDQSAKPQLPLIAINTTAGTASEMTRFCIITDESRHVKMAIVDRNVTPLLSVNDPALMVAMPKGLTAATGMDALTHAVEAYVSTAANPITDACALKAVEMISANLRQAVHDGNDLLARENMAYAQFLAGMAFNNASLGFVHAMAHQLGGFYDLPHGVCNAVLLPHVQSFNATVCAQRLTDVAHALGADIRGFSPEEGAQAAIAAIRTLARDVEIPAGLRELGAKLQDIPLLAANALKDACGLTNPRPADQRQIEEIFRNAF

SEQ ID NO: 87 - protein, 382 aa, Shewanella sp. P1-14-1
MATKFFIPSVNVLGQGGVDEAINDIKTLGFKRALIVTDTPLVNIGLVDKVAAKLIDNGITVFIFDGVQPNPTVSNVEAGLAMLNAHECDFVISLGGGSPHDCAKGIALVATNGGNISDYEGLDVSTRPQLPLVAINTTAGTASEMTRFCIITDETRHIKMAIVDKNTTPILSVNDPELMIEKPAALTAATGMDALTHAIEAYVSIAATPITDACAIKAIELIKANLVNAVEQGDNIDAREQMAYAQFLAGMAFNNASLGYVHAMAHQLGGFYDLPHGVCNALLLPHVQAYNAKVVPGKLKDIAKAMGVDVAQLSDEQGAESAIEAIKALSVAVNIPANLTELGVNPEDIPVLADNALKDACGLTNPQQATHAEICEIFTNAL

SEQ ID NO: 88 - protein, 382 aa, Nitrincola lacisaponensis
MSVSEFHIPALNLMGAGALKQAIGNIQKQGFSRALIVTDAGLVSAGLVDEVTQLLQQAGVATCVFADVQPNPTTANVAAGLALLQQQQCDLVISLGGGSPHDCAKGIALVATNGGDIRDYEGVDKSAKPQLPLISINTTAGTASEMTRFCIITDETRHIKMAIVDKHTTPILSVNDPLTMVGMPTQLTAATGMDALTHAVEAYVSTAATPITDACALKAVELITRFLPRAVQQGDDLEAREQMAYAQFLAGMAFNNASLGYVHAMAHQLGGFYDLPHGVCNAVLLPHVQVFNSQVAAERLAQVGVAMGLAASDNAQAGADACIAAIKALKDQVGIPRGLADLGAKAEDIPVLAANALKDACGFTNPIQANQSQIEAIFQQAW

SEQ ID NO: 89 - DNA, 624 bp, Artificial Sequence
  Synthetic
atgaaactgcaagtagccatggatctgctgaccgtggaagatgccctggagctggccaaccaggtggcagaatacgtcgatattattgagttgggcaccccgctgattaaagctgccggtttagcggccgttaccgctgtaaaaaatgctcatccggacaaaattgtctttgcggatatgaaaaccatggatgccggcgaactggaagcggatattgcgtttaaggcgggcgcggatctgatgaccgtgctgggcaccgctgacgatagcaccattgcgggcgccgtgaaagcagccaaggcacataataaaggcgttgttgtggacctcattggtgtcgcggataaagttacccgcgcaaaagaagtgcgcgcgcttggtgctaaattcgtggaaatgcatgccggcctggacgaacaggccaaaccgggctttgatctgcgcggcctgcttaccgcgggcgaagaagcccgcgtcccgtttagcgtggcgggtggtgtcaacctgagcaccattgaggcggtacaacgcgcgggtgccgatgttgcagtagccggcgggtttatttacagcgcgcaggacccggctctggcagcgaaacagctgcgcgccgcaattatctga

SEQ ID NO: 90 - DNA, 645 bp, Artificial Sequence
  Synthetic
atggccaagaaagtgatgatccagtttgctctggattctctggacccgcaggttaccttagaccttgcagctaaggccgcgccctacgtcgatattttagagattggaaccccgtgcatcaaatataatggaatttctttggtgaaagagatgaaatcccgttttcctgataagaaggtgctggtggatctaaaaaccatggatgctggcgaatatgaggcaaagccgttctttgaagcgggcgcggatattaccacggttctaggagtagctgaactggccactatcaaaggggttattaaagctgcccatgcccacaatggctgggcgcaggttgatctaatgaatgtaccggataaagccgcgtgtgccaaggccgtagtcgaagccggcgccgatattgtgggcgttcatactggccttgaccaacaagccgcaggaatgaccccttttaccgacctgaatctgatcagctcacttggtctgaatgttatgatctcgtgtgcgggcggcgttaagcatgaaaccgtgcaggatgtggtccgtgccggcgcgaatattgtagtggtcggcggcgccatttacggcgctcctgatccggcagctgcggcgaaaaaattccgcgaattagtggatgccgtatga

SEQ ID NO: 91 - DNA, 633 bp, Artificial Sequence
  Synthetic
atgaaattacagctggcattagatctggttgacattccggaggctaaaaaagtagttcaggaagttgaagcatatattgacattgtagagattggtaccccggttgttattaatgaaggtttaagagcagttaaagagattaaggaagcgttcccgcatctgcaagtcctggcggatctgaaggtgatggacgcggccggctacgaagtcatgaaagccagcgaagctggcgccgatattgtgaccattctgggcgctgccgaggacgcgaccattcgcggcggggtagaagaagcccgccgcttaggcaagaaaattctggtggatatgattagcgtcaaaaatctcgaagaacgcgctaaagaagtggatgcaatgggcgttgattatatttgtgttcataccggctacgatctgcaagccgcgggcaaaaatagcttcgaagattttcgcaccattaaacgcgtggttaaaaatgctaagacggcagtggcgggtggcattaagctggcgaccctgccggaagtggtggccgccggcccggatctggtgattgttggcggcggcattacgggcgaagcggacaaaaaagcggctgccgcgcagatgcaacaactgattaaaggggcctga

SEQ ID NO: 92 - DNA, 648 bp, Artificial Sequence
  Synthetic
atggcaaggcccttgatccagttagcgctggatacgctggatattccgcagaccctgaaattagcaagcttaaccgccccatacgtggacatttttgagattggcaccccaagcattaaacataacggcattgcgctggttaaagaatttaagaagcgctttccaaacaaactgttactggtggatttaaagaccatggatgcgggggagtatgaggcgaccccattttttgcggcgggcgcggatattaccaccgtgttaggcgtggcaggactggcgaccattaaaggcgtgattaacgcggcgaacaaacataatgcggaagttcaggtggatctgattaacgtgccagataaagcggcgtgcgcgcgggaaagtgcgaaagcgggcgcgcagattgtgggcattcataccggcttagatgcgcaggcggcgggccagaccccatttgcggatttacaggcgattgcgaaattaggcttaccagtgcgcattagtgtggcgggcggcattaaagcgagtaccgcgcaacaggtggtgaaaaccggggcgaacattattgtggtgggagcggcgatttatggcgcggcgagtccagcggacgcggcccgcgagatttatgagcaggttgtggcggctagtgcgtaa

SEQ ID NO: 93 - DNA, 624 bp, Artificial Sequence
  Synthetic
atgaaactgcaagtagccattgatttactgaccaccgaagccgcactggagctggcaggcaaagtggcagagtatgtggatatcattgaactgggcaccccgctgattaaagcggaaggcttaagcgtaatcaccgccgtcaaagaagcgcatccggataaaattgtctttgcggacctgaaaacgatggacgccggcgaactggaagccgacattgcttttaaggccggtgcagacctggtgaccgtcctgggcgcggcagatgacagcaccattgccggcgcggtcaaagcggcgcaggcacataacaagggcgtggtagtggatctgattggcattgaggacaaggttacccgcgcgaaagaagtgcgcgcattgggcgctaaatttgtcgagatgcatgcggggctggatgagcaagccaaaccggggtttgacctgaatggcctgctgcgcgcgggcgccgaagcccgcgtcccgtttagcgtggcaggcggcgtgaagctggcgaccattggcgatgttcagaaagcgggcgcggatgtggcagttgcgggcggcgcaatttatggcgcggcggacccggcagtagcagctaaagaattacgcgcagcgattgtatga

SEQ ID NO: 94 - DNA, 684 bp, Artificial Sequence
  Synthetic
atggacgatcgctaccgcattgcgccgagcgttctgagcgccgattttgcccgcttaggggaagaagtgcgcgcggtcgaagcagctggcgcagacctgattcattttgatgtgatggataaccattatgtgccgaatctgaccgtgggcccgctggtctgtgcggcggtgcgcccgcatctccgcattccgatcgatgtgcatcttatggtagagccggtggacgggatggttgcggattttgctgatgcaggcgccaacctgattagctttcatccggaggccagccgccatgttgatcgcacccttggtctgattcgcgaacgcggctgcaaagccggccttgtgtttaatccggccaccccgcttgcctggttagatcataccttagataaggttgaccttgttttactgatgagcgtcaatccgggttttggtggtcagcgtttcattgacagcgttttaccgaaaattgctgaagctcgtcgtcgtattgatgcgcatggtggtgcacgtgaaatttggttagaggtagatggcggggtgaaaaccgataacatcgcgcagattgcggctgctggcgcagatacctttgttgcgggcagcgcgatttttggcagcaaagattacgcggcgaccattcgcgaaatgcgcacccgcctggcaggcgcacgccgcgcctga

SEQ ID NO: 95 - DNA, 636 bp, Artificial Sequence
  Synthetic
atgaaactgcaactggcaattgatctgctggatcaggttgaagccgccaaattggcccaggaagtagaagaatttattgatattgtggaaattgggaccccgattgtgattaatgaaggcctgagcgcggtcgaacatatgagcaagagcgtaaacaatacccaggtgctggccgatctgaaaattatggacgccgcgggctatgaggtgagccaggcgattaagtttggcgcggacattgttacgattctgggcgtcgcggaagatgcgagcattaagagcgcgattgaagaagcgcataaacatggcaaagaactgctggtcgacatgatcgcggtgcaaaaccttgaacaacgcgcggcagagttagataaaatgggtgctgattatattgcagtgcatacgggctatgacctgcaagccgagggcgtaagcccgctcgaaagcctgcgcacggtgaaaagcgtcattagcaatagcaaagttgcggtagcgggtggcattaaaccggataccattgagacggtagcagcagaaaaaccggatttaattatcgtgggtggcggcattgcaaatgccgatgacccgaaggccgccgccaaaaagtgtcgcgaaattgtcgatgctcatgcctga

SEQ ID NO: 96 - DNA, 633 bp, Artificial Sequence
  Synthetic
atgaaattacaattagcgctggatttagttgatattccgggtgcaaaagctttaattgaagaagttgagcagtttattgatgttgttgaaattggtaccccggttgttattaatgaaggtttaagagcagttaaggaagttaaagaagccttcccgaatctggatgtgctggcagacctgaaaattatggatgcggcggggtacgaagtgatgaaagcgagcgaagccggcgcagatattattaccattctgggtgtagcggaggatgccagcattaagggcgcagtggaggaagcgaaaaaacaggggaaaaaaattctggtggacatgattagcgtcaaggacattgcaacccgcgcgaaagaactggacgaatttggcgtggactacatctgtgtgcataccggttatgatttgcaggccgttggtcagaacagctttgaagatctgcgcaccattaaaagcgtggttaaaaacgccaaaaccgcggtcgctggcggtattaaattggatacccttccggaagttattgcagctaatccggatctggtgattgtgggtgggggcattaccggccaagatgataaaaaggcagtagccgcgaaaatgcaggaattgattaaacaggggtga

SEQ ID NO: 97 - DNA, 624 bp, Artificial Sequence
  Synthetic
atgaaactgcaagtggcgatggatgtactgacggtggaagctgcactggagctggccggcaaagtggctgaatatgtggacatcattgaacttggcaccccgctggtcaaaaacgcgggtttgagcgcggtgaccgcggttaaaaccgcgcatccggataaaattgtatttgctgatatgaaaaccatggacgcgggcgaattggaagcagaaatcgccttcggtgcaggggccgatctggtcagcgtcctgggcagcgcagacgatagcaccattgcaggcgcggtcaaagcagccaaagcgcataacaagggcattgtggtagatctcattggggttgctgataaagtgacccgcgccaaagaagcgcgcgctctgggcgcgaaatttattgagttccatgccggcctcgacgaacaggctaaaccgggctataatctcaatctgctgctgagcgccggggaagaagcacgcgtaccgtttagcgtcgcaggcggcgtgaacctgagcaccatcgaggcggtgcagcgcgcaggcgcggatgtagcagtggtcggcggcagcatttatagcgcagaagatccggcgctggcggctaagcagctgcgcgcggcgattatctga

SEQ ID NO: 98 - DNA, 642 bp, Artificial Sequence
  Synthetic
atggaattacaattagctttagatttagtaaatattccacaagcaaaagaagttgttaaggaagtcgaagggcatattgatattgtggaaattggtaccccggttgttattaatgagggtctgcgtgcggtgaaggagattaaacaagcgttcccgaatcttaaagttttagcagacctgaaaattatggacgccggtgcatatgaagttatgaaagcaagtgaagcaggagcagatattgtaactgttttaggtgcaactgatgatgcaactattaagggagctgttgaggaagctaaaaaacagggtacccaaattctggtagatatgattaatgttaaggaccttgaacagcgtgcgaaagaaattgatgcgctgggggtagactacatttgtgtgcataccggttacgatcttcaggcagcgggtgaaaatagctttcaacaattacaaaccattaagcgtgttgttaaaaatgcgaagacggcaattgcgggaggcattaaattagacaccctgagcgaagtggtggaaacccagccggatttggttattgtcggcggcggtattaccggccagcaggataaaaaagccgtagcagctaaaatggaaagcctgattaaacaggaaagcctggcctga

SEQ ID NO: 99 - DNA, 633 bp, Artificial Sequence
  Synthetic
atgaaacttcagttagcgattgatttggaagacgtagatggtgcaatcgagctgatcgaaaaaaccaaagacagtgtggatgtttttgaatatggcacgccgctggtaatcaacttcggattagaaggcttaaaaaaaatccgtgagcgttttccagatatcaccttactggcggatgtaaaaattatggatgtagccggttacgaagtcgaacaggccatcaattacggcgcggatatcgtgacgatcttagccgcggctgaggatcaatcgatcaaagatgcagtggcgaaagcccacgaacacggaaaagaactgctggttgatatgattggtatacaggatgtggagaaacgtgcaaaagaactggatgaaatgggtgccgactatattgcgacccataccggctatgacttacaggcgttagggcagacgccactggaaaatttcaataaaattaaggccacggtgcaacaaaccaaaacagcagtcgcgggtgggattaaagaggatagcgcgccgaccattatatcacaacagccggatttattgattgtcggcggcgcgattagcaccgacgataatcctgcggagaaagcaaaagtcttcaaagacatgatcgacaacgcctga

SEQ ID NO: 100 - DNA, 633 bp, Artificial Sequence
  Synthetic
atgaaacttcaactcgccttggacctggttaatattccggaagctaaagaagttgtaaaagaagtggaagaatatattgatattgtcgaaattggcaccccggttgtcattaacgagggcctgaaagcggttaaggaaattaaagaggcgtttccgagcctgagcgttttagcggacctgaaaattatggatgcggcgggttatgaagtaatgaaagcgagcgaagccggtgccgacattgtgacgattttgggcgtcgcggaagatgcttcgattcaaggtgcggtggaagaagcgaaaaaacagggcaaagaactcctggtcgatatgattggcgtcaaagacatcgagaaacgcgccaaagagttggaccagtttggcgcggactacatttgcgtgcataccggctatgatttacaagccgaaggcaagaacagctttgaggatttacatacgatcaaaagcgtggtgaagaatgccaaaaccgcgatcgcaggcggtattaaattagagactttaccagaggtgattaaagaaaatccggatctgattattgtgggaggcggcattaccagccaggatgataaagcggccaccgcggcgaaaattcgcgaattgattaataaagggtga

SEQ ID NO: 101 - DNA, 633 bp, Artificial Sequence
  Synthetic
atggaactgcaactggcgttagacttggtgaacattgaagaagcgaaagttctggttaaagaggtagaaagctttattgatattgttgaaattggcaccccgattgtaattaacgaggggctccatgccgttaaggcgattaaagaagctttcccgaatctgaaggttctggctgatctgaagattatggatgctggcggctatgaggtgatgaaagcaagcgaagcaggggcagacattattaccgtactgggcgtcagcgatgatagcaccattcgcggcgccgtggaagaagcgcgcaagcagggcaataagattatggttgatatgattaacgtgaaaaacattgaagcacgcgcggcagaaattgatgcgttaggcgtagattatatttgtgtccatagcggctatgatcatcaggctgagggcaaaaacagctttgaagaactcgcagcgattaaacgcgtagttaaacaggcgaaaaccgcgattgcgggcggcattaagattgataccctgcaagaggtgattagcgccaaaccggatctggtgattgtcggcggcgggattaccggcgtggaaaacaaaagcgcaaccgcgagccagatgcaacagtggatcaaacaagcctga

SEQ ID NO: 102 - DNA, 636 bp, Artificial Sequence
  Synthetic
atgaaacttcagctggccctcgatctggttgacattcaaggcgcgattgatatggtcaatgaagtcggccaagaaaacattgatgtggtagaaattggcacgccggttgttattaatgagggcctgcatgcagtgaaggccattaaagaggcgtttccgaatcttaccgtgctcgccgacctgaaaattatggacgcagccggctacgaagtgaatcaggccagcgccgcgggcgcggacattattaccattctgggtgccagcgaggatgagagcattaaaggcgcagttgccgaagcgaaaaaggacggcaaagaaattctcgtcgatatgattgctgtaaaggacctggcagcccgcgcaaaagaagtggatgaatttggcgtggactacatttgcgtgcataccggctacgatctgcaagcggtgggcaaaaatagctttgaagacttaaaaaccattaaagctgccgtgaaaaacgcgaaaaccgccattgcgggcgggattaaactcgacaccttaaaggaagcagtggaacaacatccggacctgattattgtgggcggcggcattaccaccgtggacaataaacaggaagtggcaaaagcaatgaaagcgatgattaatgaagggtga

SEQ ID NO: 103 - DNA, 633 bp, Artificial Sequence
  Synthetic
atgaaattgcagctggcactggatctggtggatattgcaggcgctaaagcgattgtggccgaagtggcggagttcattgatattgtagaaattggtaccccggttgttattaacgaaggcctgcatgccgtgaaagcaattaaggacgcatttccggcgctgacggtcctggccgatctgaaaattatggacgctgggggctatgaagtgatgaaagcggttgaagcgggcgcgggcattgtcaccgtcttgggcgtaagcgatgatagcaccatccgcggtgcggtggaagaagccaaaaagaccggcgctgaaattctggttgatctgattaacgtgaaagatctgaaagcacgcgcggcagaagtggatgccctgggggtagattacgtttgtgttcatagcggctacgatcatcaagctgaaggcaaaaacagctttgaagatctgcgcgcgattaaaagcgtagtgaccaaggccaaaaccgccattgccgggggcattaaattaggcaccctgccggaagttattgcggccaacccggatctggtgattgtaggtggtggtattacgggtgaagctgaccaacgtgcggcggcagctgaaatgaaacgcctggttagccaggcctga

SEQ ID NO: 104 - DNA, 624 bp, Artificial Sequence
  Synthetic
atgaaacttcagttcgccatggataccctgaccaccgatgcggctcttgagttagccgcggcggcagccccgagcgttgatattattgaactgggcaccccgctgattaaagccgagggctttcgcgcgattaccgcgatcaaagaagcccatccggacaaaattgttttcgccgatctgaagaccatggatgccggcgaactggaagcgggggaagcatttaaggccggcgccgatctcgtgaccgtgctgggcgtggccggtgacagcaccattgcaggcgccgtgaaagctgcgaaggcacatggtaaaggcattgtcgtcgatctgattggcgtgggcgataaggccgcccgcgctaaggaagtggtggccctgggtgccgaatttgtggagatgcatgcgggcctggacgaacaagcggaagaaggtttcaccttcgagaagctcttggaagcgggcaaggcgagcggggttccgtttagcgtcgccggcggcgtgaaagccgcgaccgtgggcagcgtacaggatgccggcgccgatgttgccgtggcgggtgccgcaatttacagcgcggatgatgttgctggtgcggcagctgaaattcgcgctgcaattaagtga

SEQ ID NO: 105 - DNA, 648 bp, Artificial Sequence
  Synthetic
atggcaaggcccttgatccagttagcgctggatacgctggatattccgcagaccctgaaattagcaagcttaaccgccccatacgtggacatttttgagattggcaccccaagcattaaacataacggcattgcgctggttaaagaatttaagaagcgctttccaaacaaactgttactggtggatttaaagaccatggatgcgggggagtatgaggcgaccccattttttgcggcgggcgcggatattaccaccgtgttaggcgtggcaggactggcgaccattaaaggcgtgattaacgcggcgaacaaacataatgcggaagttcaggtggatctgattaacgtgccagataaagcggcgtgcgcgcgggaaagtgcgaaagcgggcgcgcagattgtgggcattcataccggcttagatgcgcaggcggcgggccagaccccatttgcggatttacaggcgattgcgaaattaggcttaccagtgcgcattagtgtggcgggcggcattaaagcgagtaccgcgcaacaggtggtgaagaccggggcgaacattattgtggtgggagcggcgatttatggcgcggcgagtccagcggacgcggcccgcgagatttatgagcaggttgtggcggctagtgcgtga

SEQ ID NO: 106 - protein, 207 aa, Arthrobacter sp. ERGS101
MKLQVAMDLLTVEDALELANQVAEYVDIIELGTPLIKAAGLAAVTAVKNAHPDKIVFADMKTMDAGELEADIAFKAGADLMTVLGTADDSTIAGAVKAAKAHNKGVVVDLIGVADKVTRAKEVRALGAKFVEMHAGLDEQAKPGFDLRGLLTAGEEARVPFSVAGGVNLSTIEAVQRAGADVAVAGGFIYSAQDPALAAKQLRAAII

SEQ ID NO: 107 - protein, 214 aa, Methylothermus subterraneus
MAKKVMIQFALDSLDPQVTLDLAAKAAPYVDILEIGTPCIKYNGISLVKEMKSRFPDKKVLVDLKTMDAGEYEAKPFFEAGADITTVLGVAELATIKGVIKAAHAHNGWAQVDLMNVPDKAACAKAVVEAGADIVGVHTGLDQQAAGMTPFTDLNLISSLGLNVMISCAGGVKHETVQDVVRAGANIVVVGGAIYGAPDPAAAAKKFRELVDAV

SEQ ID NO: 108 - protein, 210 aa, Paenibacillus mucilaginosus
MKLQLALDLVDIPEAKKVVQEVEAYIDIVEIGTPVVINEGLRAVKEIKEAFPHLQVLADLKVMDAAGYEVMKASEAGADIVTILGAAEDATIRGGVEEARRLGKKILVDMISVKNLEERAKEVDAMGVDYICVHTGYDLQAAGKNSFEDFRTIKRVVKNAKTAVAGGIKLATLPEVVAAGPDLVIVGGGITGEADKKAAAAQMQQLIKGA

SEQ ID NO: 109 - protein, 215 aa, Methylococcus capsulatus
MARPLIQLALDTLDIPQTLKLASLTAPYVDIFEIGTPSIKHNGIALVKEFKKRFPNKLLLVDLKTMDAGEYEATPFFAAGADITTVLGVAGLATIKGVINAANKHNAEVQVDLINVPDKAACARESAKAGAQIVGIHTGLDAQAAGQTPFADLQAIAKLGLPVRISVAGGIKASTAQQVVKTGANIIVVGAAIYGAASPADAAREIYEQVVAASA

SEQ ID NO: 110 - protein, 207 aa, Arthrobacter globiformis
MKLQVAIDLLTTEAALELAGKVAEYVDIIELGTPLIKAEGLSVITAVKEAHPDKIVFADLKTMDAGELEADIAFKAGADLVTVLGAADDSTIAGAVKAAQAHNKGVVVDLIGIEDKVTRAKEVRALGAKFVEMHAGLDEQAKPGFDLNGLLRAGAEARVPFSVAGGVKLATIGDVQKAGADVAVAGGAIYGAADPAVAAKELRAAIV

SEQ ID NO: 111 - protein, 227 aa, Betaproteobacteria bacterium
MDDRYRIAPSVLSADFARLGEEVRAVEAAGADLIHFDVMDNHYVPNLTVGPLVCAAVRPHLRIPIDVHLMVEPVDGMVADFADAGANLISFHPEASRHVDRTLGLIRERGCKAGLVFNPATPLAWLDHTLDKVDLVLLMSVNPGFGGQRFIDSVLPKIAEARRRIDAHGGAREIWLEVDGGVKTDNIAQIAAAGADTFVAGSAIFGSKDYAATIREMRTRLAGARRA

SEQ ID NO: 112 - protein, 211 aa, Macrococcus caseolyticus
MKLQLAIDLLDQVEAAKLAQEVEEFIDIVEIGTPIVINEGLSAVEHMSKSVNNTQVLADLKIMDAAGYEVSQAIKFGADIVTILGVAEDASIKSAIEEAHKHGKELLVDMIAVQNLEQRAAELDKMGADYIAVHTGYDLQAEGVSPLESLRTVKSVISNSKVAVAGGIKPDTIETVAAEKPDLIIVGGGIANADDPKAAAKKCREIVDAHA

SEQ ID NO: 113 - protein, 210 aa, Bacillus akibai
MKLQLALDLVDIPGAKALIEEVEQFIDVVEIGTPVVINEGLRAVKEVKEAFPNLDVLADLKIMDAAGYEVMKASEAGADIITILGVAEDASIKGAVEEAKKQGKKILVDMISVKDIATRAKELDEFGVDYICVHTGYDLQAVGQNSFEDLRTIKSVVKNAKTAVAGGIKLDTLPEVIAANPDLVIVGGGITGQDDKKAVAAKMQELIKQG

SEQ ID NO: 114 - protein, 207 aa, Arthrobacter sp.
MKLQVAMDVLTVEAALELAGKVAEYVDIIELGTPLVKNAGLSAVTAVKTAHPDKIVFADMKTMDAGELEAEIAFGAGADLVSVLGSADDSTIAGAVKAAKAHNKGIVVDLIGVADKVTRAKEARALGAKFIEFHAGLDEQAKPGYNLNLLLSAGEEARVPFSVAGGVNLSTIEAVQRAGADVAVVGGSIYSAEDPALAAKQLRAAII

SEQ ID NO: 115 - protein, 213 aa, Bacillus sp.
MELQLALDLVNIPQAKEVVKEVEGHIDIVEIGTPVVINEGLRAVKEIKQAFPNLKVLADLKIMDAGAYEVMKASEAGADIVTVLGATDDATIKGAVEEAKKQGTQILVDMINVKDLEQRAKEIDALGVDYICVHTGYDLQAAGENSFQQLQTIKRVVKNAKTAIAGGIKLDTLSEVVETQPDLVIVGGGITGQQDKKAVAAKMESLIKQESLA

SEQ ID NO: 116 - protein, 210 aa, Lactobacillus floricola
MKLQLAIDLEDVDGAIELIEKTKDSVDVFEYGTPLVINFGLEGLKKIRERFPDITLLADVKIMDVAGYEVEQAINYGADIVTILAAAEDQSIKDAVAKAHEHGKELLVDMIGIQDVEKRAKELDEMGADYIATHTGYDLQALGQTPLENFNKIKATVQQTKTAVAGGIKEDSAPTIISQQPDLLIVGGAISTDDNPAEKAKVFKDMIDNA

SEQ ID NO: 117 - protein, 210 aa, Bacillus marisflavi
MKLQLALDLVNIPEAKEVVKEVEEYIDIVEIGTPVVINEGLKAVKEIKEAFPSLSVLADLKIMDAAGYEVMKASEAGADIVTILGVAEDASIQGAVEEAKKQGKELLVDMIGVKDIEKRAKELDQFGADYICVHTGYDLQAEGKNSFEDLHTIKSVVKNAKTAIAGGIKLETLPEVIKENPDLIIVGGGITSQDDKAATAAKIRELINKG

SEQ ID NO: 118 - protein, 210 aa, Paenibacillus sp.
MELQLALDLVNIEEAKVLVKEVESFIDIVEIGTPIVINEGLHAVKAIKEAFPNLKVLADLKIMDAGGYEVMKASEAGADIITVLGVSDDSTIRGAVEEARKQGNKIMVDMINVKNIEARAAEIDALGVDYICVHSGYDHQAEGKNSFEELAAIKRVVKQAKTAIAGGIKIDTLQEVISAKPDLVIVGGGITGVENKSATASQMQQWIKQA

SEQ ID NO: 119 - protein, 211 aa, Lactobacillus ceti
MKLQLALDLVDIQGAIDMVNEVGQENIDVVEIGTPVVINEGLHAVKAIKEAFPNLTVLADLKIMDAAGYEVNQASAAGADIITILGASEDESIKGAVAEAKKDGKEILVDMIAVKDLAARAKEVDEFGVDYICVHTGYDLQAVGKNSFEDLKTIKAAVKNAKTAIAGGIKLDTLKEAVEQHPDLIIVGGGITTVDNKQEVAKAMKAMINEG

SEQ ID NO: 120 - protein, 210 aa, Paenibacillus sp.
MKLQLALDLVDIAGAKAIVAEVAEFIDIVEIGTPVVINEGLHAVKAIKDAFPALTVLADLKIMDAGGYEVMKAVEAGAGIVTVLGVSDDSTIRGAVEEAKKTGAEILVDLINVKDLKARAAEVDALGVDYVCVHSGYDHQAEGKNSFEDLRAIKSVVTKAKTAIAGGIKLGTLPEVIAANPDLVIVGGGITGEADQRAAAAEMKRLVSQA

SEQ ID NO: 121 - protein, 207 aa, Frigoribacterium sp.
MKLQFAMDTLTTDAALELAAAAAPSVDIIELGTPLIKAEGFRAITAIKEAHPDKIVFADLKTMDAGELEAGEAFKAGADLVTVLGVAGDSTIAGAVKAAKAHGKGIVVDLIGVGDKAARAKEVVALGAEFVEMHAGLDEQAEEGFTFEKLLEAGKASGVPFSVAGGVKAATVGSVQDAGADVAVAGAAIYSADDVAGAAAEIRAAIK

SEQ ID NO: 122 - protein, 215 aa, Methylococcus capsulatus; identical to SEQ ID NO: 109

SEQ ID NO: 123 - DNA, 615 bp, Artificial Sequence
  Synthetic
atgaaaaaagatcaggtgaaggattgcaaagacgtgattctcagcatggagctgattgccgaaaatttgaatgaggtaattaaggtcttggatcgcgaagccattattagcatgctgcaagaaatccttgaaggggagcgcgtctttgtgatgggcgccggccgcagcgggctggttgcgaaagcatttgcgatgcgcctgatgcatttgggcttcaccgtatacgttgtgggcgaaaccacgaccccggccgttcgccaacaggatgtagtaattgcaattagcggcagcggtgaaacccgcagcattgcggatcttggcaaaatcgtaaaagacattggcagcaccctgattacggtgaccagcaaaaaagaaagcaccttaggccgcattagcgacattgcaatgattcttccgagcaaaaccaaaaacgaccatgatgcgggcggctacctggaaaaaaatatgcgcggcgattacaaaaatttgccgccgctgggcacggcattcgagattaccagcttggtgtttttggatagcattattgcgcagctcattaccttaacgggcgccagcgaagccgagctgaaaagccgccataccaacattgaatga

SEQ ID NO: 124 - DNA, 612 bp, Artificial Sequence
  Synthetic
atgaccaacagcacgccggatccgcgccctacgggcgatgccccagtagatgtggccaccgccttaactctaattgcggatgagaatgcacgcgttgcacgcgccttggccgagcctgatctggcggctcgcctagatgaagccgcgcgcgtgattcgtgatggccgccgtgtatttgccctgggggcgggacgcagcggcttggctttacgcatgactgcgatgcgctttatgcaccttggtcttgacgctcatgtagtgggcgaagcgacatcgccagcaatcgccgagggagatgtgctgttagtggcttcgggctctggtacgaccgcagggatcgttgcggcggcacagaccgcgcatgatgtaggtgcccgtatcgtggcactgacaaccgcagatgatagcccgctggcggatctggccgacgtcaccgttttgatccccgctgcggcaaagcaagatcatggcggcaccgtttcggcccagtatgcgggcggtttgttcgaactgtctgttgccctggttggcgatgcggtctttcatgccttatggcaggcctcgggcctgagcgcagacgaactgtggcctcgccacgccaatcttgaatga

SEQ ID NO: 125 - DNA, 612 bp, Artificial Sequence
  Synthetic
atggaaaaaaacgaaattctccagaaaggcaaaaaagttattgaaatggaacgctatgagctgggccgcctgatggatagcctcgatgataactttgtgaaagcggtcgacatgattaccgaatgcaagggcaaaattattctgaccggcaccggcaaaagcggcttaatcagccgcaaaatcgcagcgaccctgtgttgcaccggcaaaccggcgtttttcctgagcgcctataactgtgaaaatggtgatattggtgcaatccagccgaacgatcttattattgcgattagcaatagcggggaaaccaccattctgaaggaattagttattccgagtgcaaaaaccattggtgcaaaagcaatttgtttaactggtaataccgagagtaccttagcaaagttatgtgatgttgcattatatattggtgttgagaaggaagcgtgcccgaccggcgtaaacgccaccacgagcaccaccaataccttagcgatgggcgatgccctggcgatggtcagcgaagaaattcgcggcgtgacccgcgaacaagttctgttttaccatcagggtggggcgtggggtgaaaaactgaaagacgagttcgaaaagtga

SEQ ID NO: 126 - DNA, 534 bp, Artificial Sequence
  Synthetic
atgcaccagaagctgattatagataagattagtggcattttagcggcgaccgacgcgggctacgacgcaaagctgactgcgatgttagatcaggcgagtcgcatttttgtggccggtgcgggccgttcgggtctggtggcgaaattttttgcgatgcgcttaatgcatggcggctacgatgtgtttgtggtgggcgagattgtgaccccaagcattcgcaaaggcgatttgctgattgttattagtggcagtggggagacggagacgatgttagcgtttaccaagaaggcgaaagaacagggcgcgagtattgcgttaattagtacccgcgatagcagtagtttaggcgatttagcggatagtgtgtttcgcattggcagtcccgaattatttggaaaggtggtgggcatgccaatgggcaccgtgtttgaattaagtaccttattatttttagaagcgaccatttcacatattattcatgaaaagggcattccagaggaggagatgaggactcggcatgcgaacctggagtaa

SEQ ID NO: 127 - DNA, 609 bp, Artificial Sequence
  Synthetic
atgaaagagattcatctgaccgaatgtaaatatctcaccagcagcattctgcttatggctgaacatctggagacggtggccaataagttggataaggatagcgtgcgccagatgttggaggacattatgggcgcgaaacgcatttttgtgatgggcgccgggcgcagcggcttagtcggccgcgcattcgcgatgcgcctgatgcatttaggcctcaccagccatgttgtcggcgaaagcaccaccccggcagtcagcaaggacgacgtggtaattgccatcagcggcagcggccaaacccgcagcatcgccaatctgggccgcgtagccaaagaaattggcgcaaaactggtgaccattaccagcaacaaagaaagcgttctgggcgaaattagcgataccaccattgtactgccgggccgcagcaaagatgacgcgggcggctatgttgaacgccatatgcgcggtgaatacacctatctgaccccgctgggcaccagcttcgaaaccagcagcagcgtgttcctggatgcggttattgcagaattgatttttattaccggcgcaagcgaagaagatctgaagtcgcgccataccaatattgaatga

SEQ ID NO: 128 - DNA, 1029 bp, Artificial Sequence
  Synthetic
atggacgccgcgaccgttaacgcagaaatcgatcttagcgcaccgtcaccccttctggatgcggaggccatcacacgcaccgcccgtggcgttattgcgatagaagcactcgcgatcgccgtgcttgaaaaacgtatcgaagccgagttcattcgtgcatgcggtatgatgttagcgtgtccgggccgcattgtcgtgaccggtatgggcaaatctggtcacattgggcgcaagattgcggccacgctggcctccaccgggaccccggcgtttttcgtacaccctggcgaagccagtcacggggacttaggtatgattaccgataaggacgtggtgctggccctgtcaaattcaggcgagacggacgaactgctgacaatattacctgtgattaaacgtcagggcatccccttgatagcaatgacgggtaatccgggttctagccttgcccgtcaggccgacctgcacctcgatgtgtcggtgccggcggaagcttgcccactaggcctggcgccaactgcgagcaccaccgcggccctggttatgggcgacgccttagccattgccctgttagaagcccgtgggttcaccgccgaggacttcgcccgctcacacccggcaggtagtctgggccgtcgtttgttactgcgtatcgcagacatcatgcataccggcgataaagtccccaaggtgcgcgcggatgcatcactcaccgaagcgttagtggaaatgagtcgtaaaggtttgggtatgacagcggtggttgatgcggatgaccgtcttctgggcgtctataccgatggggatctgcgccgtaccctggatgatcatcaggttgatctgcgcggcgtgcgtgtcgctgagctgatgactcgcaatcctaaatcaatagctcctgacaaactggcagctgaagcggcgcaactgatggagacgtacaagatccactccttactggtggtagatggagaacgccgcgtggtcggcgccctgaatattcacgatcttttgcgcgcgaaagttgtatga

SEQ ID NO: 129 - DNA, 651 bp, Artificial Sequence
  Synthetic
atgcgcacccaattaaacaccttttggcgcacgagcatgaagaaagaccaggttaacgactgcaaggacgtgattctgagcatggagctgatggtagacaatctgagcgacgtcgtgaaaatgctggattgccaggcgattgaaagcatgttgcagaaaattatggaaggcgagcgcgtgttcgtgatgggcgcaggccgcagcggcttggtagctaaggcattcgccatgcgcctgatgcatctgggcttcagcgtttatgttgttggtgagacgaccaccccggcggtgcatccgcaggacgtggtgattgcaattagcggcagcggcgagacgcgcagcattgcgaatctggggcgcattgtaaaagaaattggcagcaccttgatcaccgtcacgagcaaaaaggacagcagcttaggcaaaattagcgacattaccatggttctgccgagcaaaacgaagaacgatcatgacgccggcgggagcttagaaaaaaatatgcgcggcgactataagaatctgccgccgcttggcaccgccttcgaaattaccagcctggtttttctggatagcgttattgcgcagttaattaccctgaccggcgccagcgaagccgaactgaaaagccgccataccaatattgaatga

SEQ ID NO: 130 - DNA, 903 bp, Artificial Sequence
  Synthetic
atgaaaatcgatctgacacagctggtgaccgagggccgtaacagtgcaagcgccgacattgataccctgccgaccctggagatgctgcaagtaatcaatcgtgaggaccagaaagtcgcgtttgccgtcgagaagaccctgcctcaggttgcacaggcggttgatgcgattgttctagcatttcaaacgggcggccgtctgatctacatgggcgccggtacgagcggccgtcttggtattctggacgcgagtgaatgcccgccgacatatggtagtcacccggatttagtggttggtttaattgcgggtggtcatcaagcgattttaaaagcagtagagaatgcggaagacaatacagaactgggtcaggatgatttaaaacatctgcaactgactgacaaagacgtcgtcgtaggcatcgcagcttcgggacgcaccccgtacgtcctgggtggcatggcctacgcaaaatcaatcggcgcgaccgtggtagccattgcgtgcaatcctcaatgtgccatgcagcagcaagcggatattgccatcatcccagtggtgggcgccgaagtagtaaccggcagctcacgtatgaaggcaggtacggcgcagaaacttatattaaacatgctgaccagcggggctatgatacgcagcggtaaagtgttcggcaatttaatggtggatgtagaagcgacaaatgccaaactcattcaacgccagaataatatagtggtggaagcgacaggttgtaactcagatcaagccgaacaggcactgaacgcgtgccaacgccattgcaaaacggccatattaatgattctagcggacatgaatgccgagcaggccacgcaaaaactcgcgaagcacaatggttttatccgcgccgccctgaacgatcagtga

SEQ ID NO: 131 - DNA, 987 bp, Artificial Sequence
  Synthetic
atgtcgcatatggaactgcaaccggattttgatttccagcaggcaggcaaagacgtgcttcgcattgagcgcgaaggcttagcgcatctggacttgttcattaatcaagactttagccgcgcctgtgatgcgatgctgcgctgccgcggcaaagtggttgttatgggcatgggtaaaagcgggcatatcggccgcaaaattgcagccacgctggcttcgaccggcaccagcgcgttttttgtgcatccgggcgaggccagccatggcgatttaggcatggtagaacagcgcgacgttgtgctggccattagcaacagcggcgaaagccaggaaattcaagcactgattccggtcttaaagcgtcagaatgtgaccctgatttgcatgacgaataatccggacagcgcgatggggcgtgcagcagacattcatctgtgtattcgtgtaccgcaagaggcttgtccgatgggcctcgctccgaccaccagcacgaccgctaccctggtgatgggcgacgcgctggcggtggcattactgcaagcacgcggctttaccgcagaggactttgcactgagccatccgggcggggccctgggccgcaaactgttgttgcgcgtaagcgatatcatgcatagcggcgatgaagtaccgatggttagcccgaccgcgagcctgcgcgacgcgctgctggagattacccgcaaaaatctgggcctgaccgtaatttgtggtccggacgcgcatattgatggcattttcaccgatggcgacttacgccgcattttcgacatgggcattaaccttaataacgcgaaaattgccgacgtcatgacccgcggcggcattcgcattcgcccgaccgcgctggctgtggatgcgctcaatctcatgcaggagcgccatatcaccagcctgctggtcgccgaaaacgatcgcctgattggcgtagtgcatatgcatgacatgctgcgcgccggcgttgtatga

SEQ ID NO: 132 - DNA, 963 bp, Artificial Sequence
  Synthetic
atgaactacaaagagatcgcacaggaaaccctgaagattgaagcgcagaccctgttggacagcgccgataaaattgatgatgtgttcgataaagcggtggaaattattctcacctgtaaaggcaagctcatcgtcaccggcgtgggcaagagcggccttattggcgcgaaaatggctgcgacctttgccagcaccggcaccccgagcttttttctgcatccgacggaagcgttgcatggtgatctggggatgattagccatagcgacgtagttattgccattagctatagcggcgagagcgaagaactgagcagcattttgccgcatattaagcgctttaacaccccgctgattggcatgacccgcgataaaaacagcacgctgggcaaatatagcgatttagtgattgatgtaattgtaaataaagaagcgtgcccgcttggcattgcgccgaccagcagcaccaccctgaccctcgccctgggtgatgcgctggcagtttgtctgatgcgcgccaaaaactttaaaaagagcgattttgcgagctttcatccgggcggcgccctcggcaagcagctgtttgtaaaagtgaaagatctgatgcgcgttaaagaactgccgattgtgaaagcggatacgaaggttaaagatgcgatttttaaaattagcgaaggtcgcctgggcaccgtactggtgaccgacgaacaaaatcgcttgctggctttaatgagcgacggcgatattcgccgcgcacttatgagcgaagactttagcctcgaagaaagcgtgttgaaatacgcgaccaagaatccgaaaaccattgaagatgaaaatatcctcgcgagcgaagcactggttattattgaagaaatgaagatccagctgctcgttgtgacggataaacatcgccgcgtactgggcgtgttacatattcataccctgattgaaaaaggcatttcgtga

SEQ ID NO: 133 - DNA, 969 bp, Artificial Sequence
  Synthetic
atggactttaatctgaaaacggaaaccgaagaacagaccctaattgatagcgtccgtaatactcttaccgaacaaggcgacgcgcttcgtcatctggctgaggtgattgatgctaatgagtacagtactgcactctcactaatgcttaattgtaaaggccacgtaatcgtatcaggtatgggcaagtccgggcacgtaggccgcaaaatgagcgcgactttagcctcgacggggacccccagcttctttatccacccggcggaggcgtttcacggagacttggggatgataaccccctacgatgtacttatcctcatttctgccagcggcgaaacggatgaagtgctgaaattggtgcccagcctgaaaaacttcggcaataaaattatcgccattactaacaacgctaatagcactttggcgaaacatgcggatgcgaccttagaacttcacatggccaacgaaacctgcccgaataacctggctccgaccacgtccactactctgacgatggcgatcggcaatgccttagcgattgcactgattcacaaacgccactttaagcctgatgactttgcgcgctatcaccctggaggctcgctggggcgtcgtttgcttactcgcgtcgccgatgtgatgcaggttcacgtgcctaacgtagacattaatgcgaccttccgccagataatccaagaacttacaagtgggtgccagggtatggtggtagtgaaagaaaatggtaaacttgccggcatcattaccgatggcgatttgcgccgctacatggagaaatgtgaagatttcgttaatggcacggcacagagcatgatgacccgcaatcctatcaccatgccgctggattcgatgattattgatgcggaagaaaaaatgacgaaacatcgtatctcaaccttacttatcactgacagtactcaagatgtaattgggttggttcgtatcttcgactga

SEQ ID NO: 134 - DNA, 534 bp, Artificial Sequence
  Synthetic
atgcaccagaagctgattatagataagattagtggcattttagcggcgaccgacgcgggctacgacgcaaagctgactgcgatgttagatcaggcgagtcgcatttttgtggccggtgcgggccgttcgggtctggtggcgaaattttttgcgatgcgcttaatgcatggcggctacgatgtgtttgtggtgggcgagattgtgaccccaagcattcgcaaaggcgatttgctgattgttattagtggcagtggggagacggagacgatgttagcgtttaccaagaaggcgaaagaacagggcgcgagtattgcgttaattagtacccgcgatagcagtagtttaggcgatttagcggatagtgtgtttcgcattggcagtcccgaattatttggaaaggtggtgggcatgccaatgggcaccgtgtttgaattaagtaccttattatttttagaagcgaccatttcacatattattcatgaaaagggcattccagaggaggagatgaggactcggcatgcgaacctggagtga

SEQ ID NO: 135 - protein, 204 aa, Methanosarcina horonobensis
MKKDQVKDCKDVILSMELIAENLNEVIKVLDREAIISMLQEILEGERVFVMGAGRSGLVAKAFAMRLMHLGFTVYVVGETTTPAVRQQDVVIAISGSGETRSIADLGKIVKDIGSTLITVTSKKESTLGRISDIAMILPSKTKNDHDAGGYLEKNMRGDYKNLPPLGTAFEITSLVFLDSIIAQLITLTGASEAELKSRHTNIE

SEQ ID NO: 136 - protein, 203 aa, Corynebacterium Sepedonicum
MTNSTPDPRPTGDAPVDVATALTLIADENARVARALAEPDLAARLDEAARVIRDGRRVFALGAGRSGLALRMTAMRFMHLGLDAHVVGEATSPAIAEGDVLLVASGSGTTAGIVAAAQTAHDVGARIVALTTADDSPLADLADVTVLIPAAAKQDHGGTVSAQYAGGLFELSVALVGDAVFHALWQASGLSADELWPRHANLE

SEQ ID NO: 137 - protein, 203 aa, Anaerofustis stercorihominis
MEKNEILQKGKKVIEMERYELGRLMDSLDDNFVKAVDMITECKGKIILTGTGKSGLISRKIAATLCCTGKPAFFLSAYNCENGDIGAIQPNDLIIAISNSGETTILKELVIPSAKTIGAKAICLTGNTESTLAKLCDVALYIGVEKEACPTGVNATTSTTNTLAMGDALAMVSEEIRGVTREQVLFYHQGGAWGEKLKDEFEK

SEQ ID NO: 138 - protein, 177 aa, Methylococcus capsulatus
MHQKLIIDKISGILAATDAGYDAKLTAMLDQASRIFVAGAGRSGLVAKFFAMRLMHGGYDVFVVGEIVTPSIRKGDLLIVISGSGETETMLAFTKKAKEQGASIALISTRDSSSLGDLADSVFRIGSPELFGKVVGMPMGTVFELSTLLFLEATISHIIHEKGIPEEEMRTRHANLE

SEQ ID NO: 139 - protein, 202 aa, Methanolobus tindarius
MKEIHLTECKYLTSSILLMAEHLETVANKLDKDSVRQMLEDIMGAKRIFVMGAGRSGLVGRAFAMRLMHLGLTSHVVGESTTPAVSKDDVVIAISGSGQTRSIANLGRVAKEIGAKLVTITSNKESVLGEISDTTIVLPGRSKDDAGGYVERHMRGEYTYLTPLGTSFETSSSVFLDAVIAELIFITGASEEDLKSRHTNIE

SEQ ID NO: 140 - protein, 342 aa, Mizugakiibacter sediminis
MDAATVNAEIDLSAPSPLLDAEAITRTARGVIAIEALAIAVLEKRIEAEFIRACGMMLACPGRIVVTGMGKSGHIGRKIAATLASTGTPAFFVHPGEASHGDLGMITDKDVVLALSNSGETDELLTILPVIKRQGIPLIAMTGNPGSSLARQADLHLDVSVPAEACPLGLAPTASTTAALVMGDALAIALLEARGFTAEDFARSHPAGSLGRRLLLRIADIMHTGDKVPKVRADASLTEALVEMSRKGLGMTAVVDADDRLLGVYTDGDLRRTLDDHQVDLRGVRVAELMTRNPKSIAPDKLAAEAAQLMETYKIHSLLVVDGERRVVGALNIHDLLRAKVV

SEQ ID NO: 141 - protein, 216 aa, Methanosarcina acetivorans
MRTQLNTFWRTSMKKDQVNDCKDVILSMELMVDNLSDVVKMLDCQAIESMLQKIMEGERVFVMGAGRSGLVAKAFAMRLMHLGFSVYVVGETTTPAVHPQDVVIAISGSGETRSIANLGRIVKEIGSTLITVTSKKDSSLGKISDITMVLPSKTKNDHDAGGSLEKNMRGDYKNLPPLGTAFEITSLVFLDSVIAQLITLTGASEAELKSRHTNIE

SEQ ID NO: 142 - protein, 300 aa, Vibrio alginolyticus
MKIDLTQLVTEGRNSASADIDTLPTLEMLQVINREDQKVAFAVEKTLPQVAQAVDAIVLAFQTGGRLIYMGAGTSGRLGILDASECPPTYGSHPDLVVGLIAGGHQAILKAVENAEDNTELGQDDLKHLQLTDKDVVVGIAASGRTPYVLGGMAYAKSIGATVVAIACNPQCAMQQQADIAIIPVVGAEVVTGSSRMKAGTAQKLILNMLTSGAMIRSGKVFGNLMVDVEATNAKLIQRQNNIVVEATGCNSDQAEQALNACQRHCKTAILMILADMNAEQATQKLAKHNGFIRAALNDQ

SEQ ID NO: 143 - protein, 328 aa, Edwardsiella ictaluri
MSHMELQPDFDFQQAGKDVLRIEREGLAHLDLFINQDFSRACDAMLRCRGKVVVMGMGKSGHIGRKIAATLASTGTSAFFVHPGEASHGDLGMVEQRDVVLAISNSGESQEIQALIPVLKRQNVTLICMTNNPDSAMGRAADIHLCIRVPQEACPMGLAPTTSTTATLVMGDALAVALLQARGFTAEDFALSHPGGALGRKLLLRVSDIMHSGDEVPMVSPTASLRDALLEITRKNLGLTVICGPDAHIDGIFTDGDLRRIFDMGINLNNAKIADVMTRGGIRIRPTALAVDALNLMQERHITSLLVAENDRLIGVVHMHDMLRAGVV

SEQ ID NO: 144 - protein, 320 aa, Sulfurimonas denitrificans
MNYKEIAQETLKIEAQTLLDSADKIDDVFDKAVEIILTCKGKLIVTGVGKSGLIGAKMAATFASTGTPSFFLHPTEALHGDLGMISHSDVVIAISYSGESEELSSILPHIKRFNTPLIGMTRDKNSTLGKYSDLVIDVIVNKEACPLGIAPTSSTTLTLALGDALAVCLMRAKNFKKSDFASFHPGGALGKQLFVKVKDLMRVKELPIVKADTKVKDAIFKISEGRLGTVLVTDEQNRLLALMSDGDIRRALMSEDFSLEESVLKYATKNPKTIEDENILASEALVIIEEMKIQLLVVTDKHRRVLGVLHIHTLIEKGIS

SEQ ID NO: 145 - protein, 322 aa, Enterobacter cloacae
MDFNLKTETEEQTLIDSVRNTLTEQGDALRHLAEVIDANEYSTALSLMLNCKGHVIVSGMGKSGHVGRKMSATLASTGTPSFFIHPAEAFHGDLGMITPYDVLILISASGETDEVLKLVPSLKNFGNKIIAITNNANSTLAKHADATLELHMANETCPNNLAPTTSTTLTMAIGNALAIALIHKRHFKPDDFARYHPGGSLGRRLLTRVADVMQVHVPNVDINATFRQIIQELTSGCQGMVVVKENGKLAGIITDGDLRRYMEKCEDFVNGTAQSMMTRNPITMPLDSMIIDAEEKMTKHRISTLLITDSTQDVIGLVRIFD

SEQ ID NO: 146 - protein, 177 aa, Methylococcus capsulatus; identical to SEQ ID NO: 138

SEQ ID NO: 147 - DNA, 924 bp, Artificial Sequence
  Synthetic
atgttagtgtccgggtcagaaatcttgcttaaggcgcataaagagaactatggtgtcggcgcttttaatttcgttaactttgaaatgctgaatgcaattttctgtgccgcgaacgaagcaaatagtcccataattgtacaggcctcggagggagctatcaaatacatgggcattgacatggcggtgggcatggttaaaatcctctctaagcgttatcctcacattccggtcgcgctgaacctggatcatggtactagctttgaaagctgccaaaaagccgtggaggccgggttcacaagtgtgatgatcgatgcaagccaccatccatttgaagaaaacttgcagctaacccaaaaagttgtagaaatggcgcacgctaaaggtgtgtcggtggaggcagaactgggccgcctgatgggcattgaggacaatatatcagtctctgaaaaagatgcggtacttattaatccggacgaagcggaagaatttgtttccaagaccaaagtcgattacctggcgccggcaatcggcacgtcgcatggagccttcaaatttaaaggtgagcctaagttggatttcgaacggttacaggaggtgaaacgccgaaccaacattccgctagtattacatggtgcctctagcatcccggagtatgttcgtgaagctttcctggcgacgggtggggatctcaaaggctccaagggagtgccatttgacttcctgaaagaagccatcaaaggaggcattaataagatcaacattgacactgatctgaggatcgcttttattgcggaagtccgccgcgttgcaaacgaagatccgacgcagtttgacttgcggaaattctttgcaccagccatggagagtatcacaaaagtgatggttgaacgcatgaatattcttggttccgccaataaaatatag

SEQ ID NO: 148 - DNA, 933 bp, Artificial Sequence
  Synthetic
atggctctggtcacgactaaagagatgtttaagaaagcatatgaaggaggctacgcgattggtgccttcaacatcaataaccttgaaataattcagggcgtattgcgcggggcgaaagcaaaaaattccgccgtgatcctgcaatgcagtacaggtgcgattaagtatgcgggcgcagcctacttaaaagctatggttgacgccgctatcgaagagacgggtattgatgtggcgctacacctggatcatggtccctcacttgacgctgttaaagaagtcatagatgcggggtttaccagcgtgatgtttgatggatcgcattatgactacgaagagaacgttcggctgaccaaagaagtagtggaatatgcgcacgcccgtggcgtggtagtcgaggcagaactcggcgtcctggctggtgtagaggatgacgtggttgccgcagaacatatttacaccgatcctgaacaggcggttgacttcgtcaatcgcaccggggtcgattctttggcaatcgcgatcggcacgagccatggcgcgttcaaatttccattagattttaagccgcaactgcgtttcgatattctggaagagatccaggccaaattgccgggtttcccgattgttttacacggcgctagcgccgtagaccccaaagcagtggagacttgtaaccaatatggtggcgatattgcgggggcgaagggtataccggtggatatgctgcgaaaagcatctggaatggcggtgtgcaaaatcaatatggacacggatctccgcctggcgtttaccgccgcggttcgtaagacctttggagacaaaccaaaggaatttgacccaagagcatatcttggggcaggcaggaacgcagttcagacaacagtggaatcgaaaattgatgaagttctcgggagtattgattccatgaaatag

SEQ ID NO: 149 - DNA, 981 bp, Artificial Sequence
  Synthetic
atgggttacaattataaagatttaggcctgagcaatacaaaggaaatgttcgcaaaagcgaacgccaacgggtatgctgttccagcgtttaactttaataacatggagatggcccttgcgatcgtagaagcatgcgctgaaatgggatccccggtcatactgcaatgtagtaaaggtgccctctcttacatgggccctgaggtgaccccgttgctggcgaaggcagcggtggaccgtgcccgctcaatgggttcggatattcccgtggctctgcacttggaccatggcccggatctcgcgacggttaaaacctgcattgaagctggcttcagctctgtcatgatcgatggttcgcattatgattttgcaaaaaacattgaagtcagcaaagaagtagtggagtttgcgcacgccaaggacgttactgttgaagcagaactgggggtacttgccgggatcgaagatgatgtgaaagcggagtcacatacgtataccaatccggacgaggtggaggaatttgtgactaaaaccggtgtcgattccctggcaattgccattgggacgtcccacggcgctcataaattcaaaccaggtgaagatcctaagttaagactggacatcttagaagaaatcgaacggcgcattccgggcttccctatagttctgcacggcagttcggcggtgccgcagcagtacaccaccatgattaaagaatttggcggtgaggttaaagacgcgatcggaatcccggatagcgagctacgtaaggcggcgaaaagcgctgtggcaaagattaacgtagatacagacggacgactggccttcactgctgcaatccgtcgcgtattgggcaccacacccaaagagttcgatccacgtaaatacctgggtgcggctaaagaagaaatgaaggcctattataaaacgaaaattgtggacgtctttgggtctgaaggggcgtacaagaaaggtactaaatag

SEQ ID NO: 150 - DNA, 858 bp, Artificial Sequence
  Synthetic
atgcctctggtcagtatgaaagagatgttaaacaaggccaaagcggaaggctatgcagttggtcaattcaatattaacaatctcgaatttacccaggctatccttcaggcggcagtagccgaaaaatccccagtgatactgggagtgtcggagggtgcggggcggtacatcggcggctttaaaactgtggttaaaatggtcgaaggtctgatggaagattataacgtaacagtgccggttgcaattcacttggaccatggctcttcgttcgagaagtgcaaagaagctattgatgccgggtttaccagcgttatgatcgacgcgtctcatcaccccttcgaagaaaacattgaaattacgtcaaaagtcgtggattacgctcatagcaagggagtgagcgtcgaggccgaactgggcaccgttggtgggcaagaggacgatgtagtcgcggaaggtgtgatctatgccgatccgaaagaatgtgaggaattggttaaacgaacgggcatcgattgcctggcgccggcgctaggatcggtacacggaccctacaaaggtgaaccgaatttaggctttgccgagatggaagaaattgggaagattaccggcatgccattagtgctgcatggtggtacaggcattccgactaaagacatccagcgtagtgtctcactgggaacggctaagatcaatgttaacaccgagaaccagatagcaagcgcgaaaaccgtgcgcgaagtcctggctgcgaaaccgaacgaatatgaccctcgtaaatacctcggcccagcaagggatgccatcaaggaaacagtgattggtaaaatgagagagttcggtagttccggccgtgcgtag

SEQ ID NO: 151 - DNA, 861 bp, Artificial Sequence
  Synthetic
atgaatgtgtccttcgttactccaaaagaaatcgtaatggatgcgtttgagaacggatatgctattggggcatttgccgtccacaacctggaaataatgaaggcggtgattcatggtgcagaacgcatgaatagtccggttatcctccagaccacacccgacaccgtgcgttacatgggcttagattatacggttgccgccgtcaaaaacttggcggagaaagcgaaaattccggtggctctgcatcttgatcacggcgacacgttccatattgcaatgcaatgtctgagggccggctacacctcgatcatgatcgacggttctagcctggattttgaagaaaacgtacatttagttaaaaaggtcaccgaggcgtcacacgctatgggcatccctgtggaagccgaactggggtcgattgcgagaaatgagggaaatggtgaaaaaacagatcgactaatgtatactgacccgtctctggcaggcgagtttgccaaacgtacgggcatagatttcctagcgcccagcttcggaaccgtacatggtgtctacgccgatgaaccggacttggattttcagttgctggaggctattaaggatgcgtccgggattccattagttatgcacggtgcgagtggcgtgagcaacgaagatattcggaaagctatcaattgcggtatcgcaaagataaactattccacggaactcaaactggcctttgccgcggaactgcgtcactaccttcaaagccatccgaccgcgtcagatcctcgcaagtatttcatgagcgcccgcgagaacgttgaagagctggtgaaagaaaaaattagtgtcctcatcgaaaaacagcgcgtactgtag

SEQ ID NO: 152 - DNA, 858 bp, Artificial Sequence
  Synthetic
atggctctggtcagtatgaaagagatgttagaaaagggcaaaaaagaaggatatgcagttggtcaattcaacattaataacctcgaatttacacaggcgatccttcaggccgcggaggaagaaaaatcgccagtgatattgggggtatcagaaggcgccgcgaaatacatgggcggttttactacggtggttcatatggtcaaggggctgatggaggattataaaaccagcgtgccggtagcaatccacttggaccatggttcctctttcgataagtgtaaagctgcgattgacgcaggatttacctctgttatgattgatgctagccaccatccctttgaagagaatgtcgaaattacgtcgaaagtggtggactacgcccacgcgcataacgtaagcgtcgaagccgagctgggcaccgtagggggccaggaggatgatgttatcgcagatggtgtgatttatgccgacccggctgaatgcgcggaacttgtaaagcgtactgcaatcgattgcctggcgcctgcgctgggtagtgtgcacggcccgtataaaggtgaaccaaatctcggcttcgaagaaatggaggaaatatcaaaactagcagatttaccgctggttttacatggcggaaccgggattccgacgcatgatattaaacgctcgatctcactgggtacagccaaaattaacgttaacaccgagaatcaaatcagcgccaccaaggccatccgagcgtacctggacgagaaccctaatcagtatgacccaaggaaatacctgacgccggctcgtgatgcgattaaaacgaccgtcatcgggaagatgagagaatttggctccagtaacaaagcctag

SEQ ID NO: 153 - protein, 307 aa, Helicobacter sp.
MLVSGSEILLKAHKENYGVGAFNFVNFEMLNAIFCAANEANSPIIVQASEGAIKYMGIDMAVGMVKILSKRYPHIPVALNLDHGTSFESCQKAVEAGFTSVMIDASHHPFEENLQLTQKVVEMAHAKGVSVEAELGRLMGIEDNISVSEKDAVLINPDEAEEFVSKTKVDYLAPAIGTSHGAFKFKGEPKLDFERLQEVKRRTNIPLVLHGASSIPEYVREAFLATGGDLKGSKGVPFDFLKEAIKGGINKINIDTDLRIAFIAEVRRVANEDPTQFDLRKFFAPAMESITKVMVERMNILGSANKI

SEQ ID NO: 154 - protein, 310 aa, Clostridium intestinale
MALVTTKEMFKKAYEGGYAIGAFNINNLEIIQGVLRGAKAKNSAVILQCSTGAIKYAGAAYLKAMVDAAIEETGIDVALHLDHGPSLDAVKEVIDAGFTSVMFDGSHYDYEENVRLTKEVVEYAHARGVVVEAELGVLAGVEDDVVAAEHIYTDPEQAVDFVNRTGVDSLAIAIGTSHGAFKFPLDFKPQLRFDILEEIQAKLPGFPIVLHGASAVDPKAVETCNQYGGDIAGAKGIPVDMLRKASGMAVCKINMDTDLRLAFTAAVRKTFGDKPKEFDPRAYLGAGRNAVQTTVESKIDEVLGSIDSMK

SEQ ID NO: 155 - protein, 326 aa, Fusobacterium mortiferum
MGYNYKDLGLSNTKEMFAKANANGYAVPAFNFNNMEMALAIVEACAEMGSPVILQCSKGALSYMGPEVTPLLAKAAVDRARSMGSDIPVALHLDHGPDLATVKTCIEAGFSSVMIDGSHYDFAKNIEVSKEVVEFAHAKDVTVEAELGVLAGIEDDVKAESHTYTNPDEVEEFVTKTGVDSLAIAIGTSHGAHKFKPGEDPKLRLDILEEIERRIPGFPIVLHGSSAVPQQYTTMIKEFGGEVKDAIGIPDSELRKAAKSAVAKINVDTDGRLAFTAAIRRVLGTTPKEFDPRKYLGAAKEEMKAYYKTKIVDVFGSEGAYKKGTK

SEQ ID NO: 156 - protein, 285 aa, Bacillus vireti
MPLVSMKEMLNKAKAEGYAVGQFNINNLEFTQAILQAAVAEKSPVILGVSEGAGRYIGGFKTVVKMVEGLMEDYNVTVPVAIHLDHGSSFEKCKEAIDAGFTSVMIDASHHPFEENIEITSKVVDYAHSKGVSVEAELGTVGGQEDDVVAEGVIYADPKECEELVKRTGIDCLAPALGSVHGPYKGEPNLGFAEMEEIGKITGMPLVLHGGTGIPTKDIQRSVSLGTAKINVNTENQIASAKTVREVLAAKPNEYDPRKYLGPARDAIKETVIGKMREFGSSGRA

SEQ ID NO: 157 - protein, 286 aa, Bacillus sp.
MNVSFVTPKEIVMDAFENGYAIGAFAVHNLEIMKAVIHGAERMNSPVILQTTPDTVRYMGLDYTVAAVKNLAEKAKIPVALHLDHGDTFHIAMQCLRAGYTSIMIDGSSLDFEENVHLVKKVTEASHAMGIPVEAELGSIARNEGNGEKTDRLMYTDPSLAGEFAKRTGIDFLAPSFGTVHGVYADEPDLDFQLLEAIKDASGIPLVMHGASGVSNEDIRKAINCGIAKINYSTELKLAFAAELRHYLQSHPTASDPRKYFMSARENVEELVKEKISVLIEKQRVL

SEQ ID NO: 158 - protein, 285 aa, Bacillus sp
MALVSMKEMLEKGKKEGYAVGQFNINNLEFTQAILQAAEEEKSPVILGVSEGAAKYMGGFTTVVHMVKGLMEDYKTSVPVAIHLDHGSSFDKCKAAIDAGFTSVMIDASHHPFEENVEITSKVVDYAHAHNVSVEAELGTVGGQEDDVIADGVIYADPAECAELVKRTAIDCLAPALGSVHGPYKGEPNLGFEEMEEISKLADLPLVLHGGTGIPTHDIKRSISLGTAKINVNTENQISATKAIRAYLDENPNQYDPRKYLTPARDAIKTTVIGKMREFGSSNKA

SEQ ID NO: 159 - DNA, 1044 bp, Artificial Sequence
  Synthetic
atgactccgaccagtcctgttcactctcgtcgggaggcccccgaccgaaatttagcattggaacttgtgcgcgtcacggaagcgggagcgatggcttccggccgttgggtagggcgcggcgataaggaaggtggtgatggcgccgcagtggacgctatgagacagctcgtgtcgagcgtttcaatgaaaggtattgttgtcatcggcgagggtgaaaaagatgaagcgccaatgctgtacaacggggagctggtcggcgatggtacaggtccggaagtggacttcgccgtggatccggtagacggaaccactctgatgagcaaaggtagtccgggcgcgatttccgtactggctgttgccgaacgcggcgcaatgtttgatcctagtgcggtgttttatatgcataaaatcgcagtgggcccagacgcggcagggagcatagatattacggcccccatcggagaaaacattcggcgcgttgcgaaggctaaacgtctctcggtttctgatctaaccgtgtgcatcctggaccgtccgcgccatgaggataccattcaacaggcacgtgatgccggagcgcggatccgcttgattagcgacggtgatgtcgccggcgctatagccgcggctcgtccggaatctggggtcgatattctcgttggcatcggaggcacgccagaaggtattattgctgcggcagcgctgcgctgtctgggcggcgaacttcaagggatgctggcgcccaaagacgatgaggaaaggcagaaagccatcgacgctggtcacgacttagatagggtattatcgacgacagatttagtgtcaggagataatgtattcttttgcgcaaccggggtcaccgatggtgacctgctccgtggcgttcgctattacgccggtggggcgtctactcagagcatcgtgatgcgctccaaatccggtaccgtgcgtatgattgacgcgtatcatcggctgactaagctgcgtgagtacagcagcgtggattttgatggcgatgattcagcaaacccgccgcttccgtag

SEQ ID NO: 160 - DNA, 1008 bp, Artificial Sequence
  Synthetic
atgactacgaataacaaccatggagatcgtaatctggccatggagcttgtccgcgcaaccgaagctgcggcgattgccgcagggccatgggttggcgccggtgaaaaaaacctcgcggacggtgcagcggtggatgctatgcggtaccgattaagcaccgtaaactttaatggcacagtggttataggcgaaggggagaaggataaagcacccatgctgtataacggtgaaaatgtcggtgacggctctggcccttcgttggacgtggcggttgatccgatcgatgggacgcgcttaaccgccctgggcatggacaacgccctgtccgtaatcgcggtcgctgatggtggcactatgttcgacccgtcagccgtgttttatatggaaaaactggttaccgggccggatgcggcggagttcgtggatcttcgtctaccagttaagcagaatctccacctggtggctaaagccaaaggcaaaaaagtgagtgaattgacagtatgcgtgctggacagaccgcgtcatgcgaagttgattcaagaaattcgcgaggctggtgcacgcacgcgtatcattttagacggagatgtcgcaggagctattgccgcatgtagggaaaacaccggtgtcgatctgatgctgggcacgggcggtacccctgaaggtgtagttgcggcgtgcgcgatcaaagcaaccggcggggtcatccagggacgcctggccccgacggatgaagcggaacgtgagaaggcattggaagcggggcacgatctcgaccgtgtactgacaactaacgacctggtgacgtcagataattgttttttcgccgctaccgggattaccgacggcaaattattgcgcggcgttcgctactccaaaaatgttgtcactacgcagtctctcgtcatgcgaagctcgtccggtactgttcgcacagtggaggctgagcatcgtctaagccgacttcgcgaaattctgagccacacgaaatcacctgaagagcaatag

SEQ ID NO: 161 - DNA, 972 bp, Artificial Sequence
  Synthetic
atggaacggtccctatcaatggagttagttcgagtgaccgaagcggcagctttggcctctgcgcgttggatgggtcgcggaaagaaagacgaagccgatgatgcagcgacaagcgctatgcgtgacgtctttgatacgatcccaatgaaaggcactgtagtgattggggagggcgaaatggatgaggcccctatgctgtatataggggaaaaacttggtaacggctacggcccgcgcgttgacgtggcagttgatcccctcgaaggtaccaatatcgtcgcgtcgggcggttggaacgcgctggccgttctggcgattgcggatcatggaaatctccttcacgctccggatatgtatatggacaaaattgcggtggggccggaagccgtaggtacgatcgatattaacgcaccagtgatagacaatctgcgcgccgtcgcaaaggctaaaaacaaagacgttgaggatattgtagctaccgtgctgaatcgtccgaggcatgaacacatcatcgcccaaatcagagaagcgggtgctcgtattaaattaatcaacgatggcgatgtggcgggcgccattaatacagctttcgatcatactggtgtcgatattctgtttggcagtggtggagccccggagggggtcattgcagccgttgccctgaaatgcctcggcggggaactgcaaggcaagttgctgcctcagaccgacgaagagctacagcgctgtaaagaaatggggatcgcagacataacgcgtgtattctacatggaagatttagtgaagggggacgacgccatctttgcggcaaccggtgtcaccgacggcgaactgcttaaaggtgttcagttcaaaggcagcgtcggcactacccattccctggtgatgcgcgccaagtcgggaacggtgcgttttgttgatggtagacacagcttaaaaaaaaaacccaacctggttattaagccaagttag

SEQ ID NO: 162 - DNA, 987 bp, Artificial Sequence
  Synthetic
atgactagcaatacgtccgatgcaccttttcacgaccgcatgctgtcgttgggtcttgctcgtgtagcggagcaggccgcgttagcctcagcatctctgattgggcgaggagatgaaaaggcggcagaccaagcggccgttaacgctatgcgcgaacagctcaacctgctggatatagcgggcgtcgtggtgatcggtgaaggcgagcgtgacgaagcaccgatgctatatattggcgaagaagttggtacaggtaaaggcccaggggtcgatattgccctggatcccttagaggggaccacgttgaccgcgaaagatatgccgaatgccctcaccgtgatcgctatgggcccgcggggaagtatgctgcatgccccagacacttacatggacaaactggcgatcggtccgggctatgctgagggagttgtaagcctggatatgagtcctcgcgaacgtgtggaagctttggcagcggcaaaggggtgcgcgccgtcggatattacggtgtgtatcttagaacgcccacgacatgaggcaatgattgcagaagtccgtgagacaggtgccgccatccgtctgattaccgatggtgacgtagctggggttatgcactgcgcggaaagcgatgtgaccggcatcgatatgtacatgggtcagggcggcgcgccggagggtgtgcttgccgccgcggccctcaaatgtatgggcggtcagatattcggccgcctgctatttcggaacgacgatgaaaaagggcgtgcagcgaaagctggaatcacggacctggatagaatttatacccgcgatgaaatggtgacacaagacgtcatttttgctgccacgggcgttaccggtggctctttattgcccgcgataaaacgcactccgggctgggttgagactaccactttactaatgcgctcaaaaacggggtctgtccggcgtatgtcctaccgtaccccgctggaaccacatcaaaaatag

SEQ ID NO: 163 - DNA, 963 bp, Artificial Sequence
  Synthetic
atgcctagcaccgactttaatgatcgtatgctcagtttgggtctggcacgcgtttcagaagctgccgcgcacgcctcggcgcggctgataggccgaggagatgagaaagcagcggatcaggctgcggtaaacgccatgcgtgaacaacttaacctgttagacatcaagggcgtggtcgtgattggggaaggtgagcgcgatgaagcaccaatgctgtacattggcgaggaagttggttctggcaatggtcccgaagtggatattgcgttggacccgctggaggggacaacgttaactgcgaaagatatgccgaacgccctgaccgtcatcgcaatggctccgcgcggcacgctcctacatgctcctgacgtgtatatggataaactggccatcggcccaggatacccgaaggacattgttaatctggaaatgaccccgtccgaacgtgtacatgccttggcgaaagcaaggggtgtcgcggcgagcgacattacttgttgcatcttagaacgcccccgtcacgaggatttggtggaggaagtccggtccacaggtgcgggcatccgtttaattaccgatggggatgtggcaggcgttattcatgttgcagaagcagaattgacgggtattgatatgtatatggggagtggaggtgcgccggaaggcgtgctagccgctagcgccctgaaatgcatgggtggtcagatgtggggcagactgcttttccgcaacgatgacgaacggggccgcgcgcacaaagcagggataaccgaccttaaccgtatctattcgcgcgatgaactggtaacagcggatgtgatttttgccgcaaccggcgtaactaatggttctatcgttcagggggttaaacgtcaaccacattatctgcaaactgaaaccatactgatgcgcagcaagaccggcagtatccgtcgcatgatttacaggaacccgatccgttag

SEQ ID NO: 164 - DNA, 999 bp, Artificial Sequence
  Synthetic
atgtctgacgccaagaaacctggaccctcccaggtgatcgaacggatattgactctcgaattagtacgcgttacggagcgagcggcagtcgctgcggcccgtcttagaggtcaaggcaacgaaaaagcagcggatcaggccgcggtggatgctatgcgccgtgagctgaatcgcctgccaattgacggcaccgtcgttattggggaaggtgaacgtgatgaggcaccgatgctgttcatcggcgaatcactgggtaacggctcgggaccgaaagtggacattgcggtggatccgctggaagggaccacactatgcgccaaagatatgcccggtagtgtagcagttatggctatggccgaaggcggaacgttattggcggcgccggacgtatatatgcataaaatcgcgattggtccagggtacccggcgggcaccgttcacctggatgcaagccctgaagagaatatccatgcacttgccaaggctaaaggagtcccgccagcggagatcacagcactcgtgctggaccgcccgcgtcacaccgatctgattgccgccattcggcgcactggtgctggggtgcgtttgatcagcgacggtgatgttgcgggtgttatttttactacgatgccggaggaaaccggtatcgatatatatctgggcattggcgccgctcctgaaggcgtgctggcggcgggcgcgctccgctgtatcggcggccaaatgcaggggcgtctgattttagatacacaggaaaaaagggatcgtgccgcgaagatgggcgtcgcggatccaaaccgcttatacgcactggacgacttggcgcgaggagatgtggtagtcgccctgacgggtgtgaccgacggtgctcttgtaaaaggtgtgcgctttggtcgtcaaaccataagaactgaaaccgtagtctatcgctcgcataccggtactgtcaggcgtattgaagcggagcatcgcgacttcgataaatttcacctaatctag

SEQ ID NO: 165 - DNA, 999 bp, Artificial Sequence
  Synthetic
atgtctgcggaaacgaatactccatcctatgtggtatcggatcggaacttggctctcgaattagtccgcgttacagaggcagccgcggtggcctcagcgcgttggaccgggcgcggaaaaaagaacgacgcagatggcgccgcagtcgaagctatgcgaaaagcgttcgacaccgttgccattgatggtacggttgtgatcggtgagggcgaaatggatgaagcacccatgctatacataggcgagaaagtcggtgcgggtggccctgcaatggacattgcggtagatccgcttgaagggaccaatttgtgtgcgaaggatatgccgaacgctatcactgtggtggccctggctgaacgtggcaattttctgcacgctccagacgtgtatatggataaactgattgttggcgcgggtctgccggacgatgtaatcgatctcgatgccagcattggggagaacctgcgcaacctggctaaagcccgtggccgtcatatcggtgatattaccctttgcgcgctggaaagagagcgccatgaagagttaatcgccaaaacacgggaagctggagcgcgcgtccgtctgattagtgacggagatgtcgcagccggcattgcggcatgcttagaaacgagcagcgttgacatctacgccggttcaggtggggcaccggaaggtgtgcttgcagcggcggccgtgagatgtatgggcggccaaatgcaggctcggttgatgtttgaagatgacgctcagcgcgagcgcgcccaaaagatgaatcctaataaacagccggaccgtaaactggggctgcacgacttagcgtcgggagatgtactgttcagtgcgaccggcgtgaccacgggttttcttctgaaaggtgtaaaacgtatgccccatcgcagtgtgactcattctctagttatgcgctccaaatctggtactctcaggttcatcgaagggtatcacaactacaatacgaaaacatggagcgtctcgtag

SEQ ID NO: 166 - protein, 347 aa, Nocardia sp.
MTPTSPVHSRREAPDRNLALELVRVTEAGAMASGRWVGRGDKEGGDGAAVDAMRQLVSSVSMKGIVVIGEGEKDEAPMLYNGELVGDGTGPEVDFAVDPVDGTTLMSKGSPGAISVLAVAERGAMFDPSAVFYMHKIAVGPDAAGSIDITAPIGENIRRVAKAKRLSVSDLTVCILDRPRHEDTIQQARDAGARIRLISDGDVAGAIAAARPESGVDILVGIGGTPEGIIAAAALRCLGGELQGMLAPKDDEERQKAIDAGHDLDRVLSTTDLVSGDNVFFCATGVTDGDLLRGVRYYAGGASTQSIVMRSKSGTVRMIDAYHRLTKLREYSSVDFDGDDSANPPLP

SEQ ID NO: 167 - protein, 335 aa, Mycobacterium tuberculosis
MTTNNNHGDRNLAMELVRATEAAAIAAGPWVGAGEKNLADGAAVDAMRYRLSTVNFNGTVVIGEGEKDKAPMLYNGENVGDGSGPSLDVAVDPIDGTRLTALGMDNALSVIAVADGGTMFDPSAVFYMEKLVTGPDAAEFVDLRLPVKQNLHLVAKAKGKKVSELTVCVLDRPRHAKLIQEIREAGARTRIILDGDVAGAIAACRENTGVDLMLGTGGTPEGVVAACAIKATGGVIQGRLAPTDEAEREKALEAGHDLDRVLTTNDLVTSDNCFFAATGITDGKLLRGVRYSKNVVTTQSLVMRSSSGTVRTVEAEHRLSRLREILSHTKSPEEQ

SEQ ID NO: 168 - protein, 323 aa, Bacillus koreensis
MERSLSMELVRVTEAAALASARWMGRGKKDEADDAATSAMRDVFDTIPMKGTVVIGEGEMDEAPMLYIGEKLGNGYGPRVDVAVDPLEGTNIVASGGWNALAVLAIADHGNLLHAPDMYMDKIAVGPEAVGTIDINAPVIDNLRAVAKAKNKDVEDIVATVLNRPRHEHIIAQIREAGARIKLINDGDVAGAINTAFDHTGVDILFGSGGAPEGVIAAVALKCLGGELQGKLLPQTDEELQRCKEMGIADITRVFYMEDLVKGDDAIFAATGVTDGELLKGVQFKGSVGTTHSLVMRAKSGTVRFVDGRHSLKKKPNLVIKPS

SEQ ID NO: 169 - protein, 328 aa, Leisingera sp.
MTSNTSDAPFHDRMLSLGLARVAEQAALASASLIGRGDEKAADQAAVNAMREQLNLLDIAGVVVIGEGERDEAPMLYIGEEVGTGKGPGVDIALDPLEGTTLTAKDMPNALTVIAMGPRGSMLHAPDTYMDKLAIGPGYAEGVVSLDMSPRERVEALAAAKGCAPSDITVCILERPRHEAMIAEVRETGAAIRLITDGDVAGVMHCAESDVTGIDMYMGQGGAPEGVLAAAALKCMGGQIFGRLLFRNDDEKGRAAKAGITDLDRIYTRDEMVTQDVIFAATGVTGGSLLPAIKRTPGWVETTTLLMRSKTGSVRRMSYRTPLEPHQK

SEQ ID NO: 170 - protein, 320 aa, Paracoccus aminophilus
MPSTDFNDRMLSLGLARVSEAAAHASARLIGRGDEKAADQAAVNAMREQLNLLDIKGVVVIGEGERDEAPMLYIGEEVGSGNGPEVDIALDPLEGTTLTAKDMPNALTVIAMAPRGTLLHAPDVYMDKLAIGPGYPKDIVNLEMTPSERVHALAKARGVAASDITCCILERPRHEDLVEEVRSTGAGIRLITDGDVAGVIHVAEAELTGIDMYMGSGGAPEGVLAASALKCMGGQMWGRLLFRNDDERGRAHKAGITDLNRIYSRDELVTADVIFAATGVTNGSIVQGVKRQPHYLQTETILMRSKTGSIRRMIYRNPIR

SEQ ID NO: 171 - protein, 332 aa, Methylobacterium aquaticum
MSDAKKPGPSQVIERILTLELVRVTERAAVAAARLRGQGNEKAADQAAVDAMRRELNRLPIDGTVVIGEGERDEAPMLFIGESLGNGSGPKVDIAVDPLEGTTLCAKDMPGSVAVMAMAEGGTLLAAPDVYMHKIAIGPGYPAGTVHLDASPEENIHALAKAKGVPPAEITALVLDRPRHTDLIAAIRRTGAGVRLISDGDVAGVIFTTMPEETGIDIYLGIGAAPEGVLAAGALRCIGGQMQGRLILDTQEKRDRAAKMGVADPNRLYALDDLARGDVVVALTGVTDGALVKGVRFGRQTIRTETVVYRSHTGTVRRIEAEHRDFDKFHLI

SEQ ID NO: 172 - protein, 332 aa, Acetobacter aceti
MSAETNTPSYVVSDRNLALELVRVTEAAAVASARWTGRGKKNDADGAAVEAMRKAFDTVAIDGTVVIGEGEMDEAPMLYIGEKVGAGGPAMDIAVDPLEGTNLCAKDMPNAITVVALAERGNFLHAPDVYMDKLIVGAGLPDDVIDLDASIGENLRNLAKARGRHIGDITLCALERERHEELIAKTREAGARVRLISDGDVAAGIAACLETSSVDIYAGSGGAPEGVLAAAAVRCMGGQMQARLMFEDDAQRERAQKMNPNKQPDRKLGLHDLASGDVLFSATGVTTGFLLKGVKRMPHRSVTHSLVMRSKSGTLRFIEGYHNYNTKTWSVS

SEQ ID NO: 173 - DNA, 1413 bp, Artificial Sequence
  Synthetic
atggaaaagcaacagattggtgtaatcggcctcgcggtcatggggaaaaatttagcctggaacattgagtcgaaaggatatacagtgagcgttttcaaccgatcccgctcaaaaactgaccagatgttgaaagaaagtgagggcaagaatatatttggttactttaccatggaagaatttgtgaactctcttgaaaaacctcgtaaaatcctgctgatggttaaagctggcgaggcaacggatgcgaccattgaacaattgaagcccttcctagataaaggggatatactgatcgacggtggcaatacgttctttaaagatacccagcgcagaaacaaagagctgagtgcccttggtattcattttatcgggactggtgtcagcggcggagaagaaggcgcactgaaggggccatccattatgccgggcggacagaaagaagcgtatgatctggtggctccgattctgaaggatattgccgcgaaagtaaacggtgaaccgtgtaccacgtacatcggcccggacggtgccgggcactatgtgaaaatggttcataatggtatcgagtacggcgacatggaattaataagcgaatcgtataatctgttaaagaacattttaggtctgggcgctaacgaactgcacgaggtctttgcagattggaataaaggcgaactcgattcttatctgatcgagattacagcggatattttcaccaaaaaagaccctgagacgggtaagccattggttgacgttatcctcgacaccgccggccagaagggtaccggcaaatggacaagccaatctgcgctggatctcggggtcccgcttccgcttatcacggaatcagtgttcgcaaggtttatttctgctatgaaagaagaacgcaaagcagcctccaaactcctgaaaggtcccgaaaagccagcgtttagtggtgataaaaaagccttcattgaggccgtgcggaaagcgctgtacatgagtaagatttgcagctacgcgcagggttttgctcagatgcgtgcagcgagcgaagagtataactgggatttgaactatggcgaaatagcaatgatcttccgtggcggatgcattatccgcgcgcaatttttacagaaaattaaagacgcgtacgaccgtgatcgcaatttaaagaatctgctattggatccgtattttaaagagatcgtagagtcctaccaagatgctctgcgggaagtgatcgctactgcggtgcgatttggcgtcccggctccagcactgtcggccgcactggcatattatgattcataccgttcggaagtattaccggcgaatctcattcaagcccagcgcgattatttcggtgcgcatacgtatcagcgtgtggacaaagagggcattttccacaccgaatggcttgaactgtag

SEQ ID NO: 174 - DNA, 1419 bp, Artificial Sequence
  Synthetic
atgtctaagcaacagattggtgtaatcggcctcgcggtcatggggaaaaatttagcctggaacattgagtcgcgtggatatagtgtgagcgttttcaaccgatcctcagataaaactgaacagatggtggcagaaagcacgggcaaaaatatatttcccacatacaccatcgaagagtttgtttccagccttgaaaaaccgcgcaaaatcttgctgatggtaaaggctggtaaagcgaccgacgccacgattgattcactgaaaccatatctggaagagggcgacattctgatagatgggggaaacacctttttccaggacaccattcggagaaataaggaattgagtgagcttggtctacattttatcggcacgggtgtctctgggggcgaagaaggtgcactgactggcccgtcaattatgccgggcggacaaaaagaagcgtacgagttggtggcacctatcctgaaggatattgcggctaaagtcgatggtgaggcctgtaccacctatatcgggccggacggcgcgggtcactacgtgaaaatggttcataacggcattgaatatggcgatatgcagttaattgcggaatcctacttcctcctgaaaaacgttctgggtttatcggccgatgagctacacgaagtgtttgctgaatggaataaaggagaattagactcgtatttgatcgaaataacggcagacatcttcacaaaaaaagatgatgaaactggaaaaccaatggtggacgtcattctggataaggcagggcaaaaaggtacggggaaatggaccagccagagtgcgctggatctgggagtgagcctgcctgtgatcacagaaagtgtatttgcccgcttcattagcgccatcaaagatgagcgcgttgctgcgtctaaggttttggctggcccgaacgctgaatcttacaccggcgatcgtaaagccttaattgaagcgatccgtaaagcgctgtatatgagcaagattgtcagctatgcacaggggttcgcacaaatgcgcgcggcctcggaggaatacaattgggacctgcaatatggcgatattgctatgatctttcgtggcggttgcatcatacgtgcgcagttccttcagaaaattaaagaagcctacgaccgcgacccagccttgcgaaatctgctactggattcctattttaaagaaattgtggagggttaccaaggcgcattacgcgaggtgatcagtgtcgctgttcagcagggcattccggtaccgggtttttcgagcgcgctggcatattatgattcttatcgcacagcaacccttcccgctaacctgattcaggctcaacgtgactactttggtgcacatacatacgagcgcgtggataaggagggaatctttcatacagaatggatcgaactcgaacggtag

SEQ ID NO: 175 - DNA, 1422 bp, Artificial Sequence
  Synthetic
atgtctaagaaaagtgattttggattaattgggctggccgttatgggccaaaatcttgtcttgaacgtggagtcccgaggtttccaggtgtcagtatataaccgcaccgaagcgactacggaagcatttatcgctgacaatcccggcaaaaaactcgttggtgcgaaaacactggaggaatttgtgcagtcgttggccaaacctaggaagatccaaattatggtcaaagcgggcgcaccggtagatcaggttataaaacagttaattccactgctggaaaaagacgatattgtgatcgacggtggcaacagcctatacaccgatacggagcgtcgtgatgcatatctctcgtccaaaggactgcggttcattggggcgggtgtgagcggcggcgaagaaggtgcccgcaaggggccgagcatcatgccgggcggtccactgtccacctgggaagttatgaagccgattttcgagtctatcgctgcaaaagtcgatggcgaaccgtgcgtgatacacatcggacctggcggggcgggtcattacgttaaaatggtacataatggcattgaatatggagacatgcagttaatttgtgaagcctatagcctatttaaagctgccggttttacgaccgaggagatggcggctatcttcaacgaatggaatgatggagaactccaaagttacctgatacagatcactgcgaaggccctggagcaaaaagatccggaaacaggtaagccaattgttgacttaattctggacaaagccggccagaagggtaccggccagtggacactgatcaacgcggcggagaatgcggtcgtgatttcaaccatcaacgcagccgtggaagcaagagtcctttcttcccaaaaaaaagctcgcgttgcagcttcaaaagtcctgcaaggtcctaaagtagaattgagcttggaaaaaaaagccctggtggcgaaagtgcacgatgccctgtacgcttcgaaggtcattagctatacgcagggttttgatctgattaaaaccatgggggataagaaagagtggaaacttgaccttggcggtatagcatcgatctggcgtggcgggtgcattatacgcgcgcgtttcttaaaccgcattactgacgcgtttcgaacagatccagccttagcgaatctgatgttggatccgttttttaaagacctgctgaaccgtacccagcaaaattggcgggaggtggtagctttggcggtgagtaatggcatcccggttcccgcattcagtgcaagtctggcatattatgattcataccgcacggaacgtttaccggcgaaccttttacaggcacagcgggattttttcggtgcgcatacgtatgaacgtaccgacaagccggaaggccagttctttcacacggattggccagaagtaatcggttag

SEQ ID NO: 176 - DNA, 1458 bp, Artificial Sequence
  Synthetic
atgtataactccaattcatactgcaacgatagcagtcgccaagagttcattatgacaaaacagcagataggagttgtgggcatggcagtaatggggcgtaatcttgccttgaacatcgaatctcggggttataccgtcagcgtgtttaaccgatcccgcgaaaagactgaggaagtaatcgctgaaaatcccggtaaaaaattagttccgtactataccgtccaagaatttattgagtcgctggaaacgcctcgtcgcattctcctgatggtgaaagcgggcgcgggcacggactcggcaatcgatagcttaaaaccgtacctggataagggggacatcattattgacggcggtaataccttctttcaggatacaatacgtcgtaacagggagctgagtgccgaaggctttaatttcattggtaccggggtgtcagggggtgaagaaggcgcgttgaaaggaccatctatcatgccgggtggccagaaagaggcttatgagctagttgccccaatcctgaagcagattgcggccgtcgcggaagatggagaaccttgtgtaacttatattggcgcagatggtgcaggccattacgtgaaaatggtccacaacggtatcgaatacggtgatatgcaattgatagctgaggcgtatgccttactgaaaggaggcctggcattgagtaatgaagaactggctcagacgttcaccgaatggaacgaaggcgagctgagcagctatctcattgacatcaccaaagacatttttacaaagaaagatgaagaggggaaataccttgtggatgttatactggatgaggcggcgaacaagggtacgggcaaatggacgtcgcaatccagcctagacctgggcgaacctttatcactgattaccgagtctgtatttgctcgctatatcagttctcttaaagaccagagagttgccgcttctaaagttctaagcggcccgcaagcgcagcccgccggggataaagcagaatttattgaaaaggtgcgccgtgctttgtacctgggaaaaatcgtgtcgtacgcacagggtttctcacagctccgcgccgcgagtgatgaatataattgggacctgaattacggcgagattgcaaaaatcttccgtgcaggatgcattatccgggcgcaatttttacagaaaatcaccgatgcttatgcgcaaaacgcgggcattgcgaatctgctgttagccccgtacttcaagcagattgctgacgactatcaacaggccctgcgtgatgtggtggcgtatgcagtccagaacggtattccggtcccgactttttcggctgcgatcgcctattatgattcgtaccggtctgccgttttaccggcgaacctcatccaagcgcagcgagactattttggagcacatacgtacaaacgcaccgataaagaaggtgtattccacaccgaatggatggtctag

SEQ ID NO: 177 - DNA, 1413 bp, Artificial Sequence
  Synthetic
atggaaaagcaacagattggtgtaatcggcctcgcggtcatggggaaaaatttagcctggaacattgagtcgaaaggatatacagtgagcgttttcaaccgatcccgctcaaaaactgaacagatgttgaaagaaagtgagggcaagaatatatttggttactttaccatggaagagttcgtgcatagccttgaaaaaccacgtaaaatcctgctgatggttaaagcaggcgaagctacggacgcgaccattgaacaactgaaaccctttctggataagggtgatattctgatcgacgggggcaatactttctttaaagatacccagcggcgcaacaaagaattgtctgccctcggaatccactttattgggacgggcgtatcaggtggtgaagagggagctttaaaggggccttccattatgccgggcggccagaaagaagcatatgacttagtggcgccgatccttaaagatattgccgcgaaagtcaacggcgatccgtgcaccacatacataggacccgacggtgctggtcattatgttaaaatggtgcacaatggcatcgaatacggcgatatggagctgatctctgagtcgtataatttgctgaagaacatcctaggcctgacggccgatgaactccatgaagtgttcgccgactggaacaaaggcgaactggacagctaccttatagagattaccgcggatatttttacgaaaaaggatccggagactggaaaaccactggtggatgtcattctggacactgcgggtcaaaaggggacgggtaaatggacaagtcagtccgcactcgatctaggggtaccgctgcctctgattaccgaaagcgtttttgcgcgtttcatttctgctatgaaggaggaacgcaaagcagcaagcaaactattaaaaggtcctgaaaagccggcatttagcggggataaaaaagcctttatcgaggccgtcaggaaggcgctgtatatgtccaaaatttgttcatatgcgcagggattcgcgcaaatgcgtgcggcttcggaagagtacaattgggacttaaactacggcgaaatagcaatgatcttccgtggtggctgtatcatccgcgcccagtttctccaaaaaattaaagatgcgtatgatcgtgaccgcaatttgaagaacctgctgttggatccgtattttaaagaaatcgtggaatcttatcaggacgcgttgcgagaagtaattgcaaccgcggtgcggttcggcgttcccgttccagccctgagtgccgctctggcttactacgattcgtatcgcagtgaggtgttaccagccaatctgctgcaagcgcagagagactacttcggtgcccacacctatcagagagtcgataaagaaggcatctttcatacggagtggctcgaactttag

SEQ ID NO: 178 - DNA, 1464 bp, Artificial Sequence
  Synthetic
atgattacgtttaagttgcgtacattccgcagtgaccatactcggcaggaatatgtaatgtccaaacaacagatcggagtcgtggggatggccgttatgggccgcaatcttgcgttaaacatcgagtcacgaggttacaccgtgtcggtctttaaccgtagcagagaaaaaaccgaggaagttattgcagaaaatcctggcaaaaaactggtgccctattacacggtacaagagttcgtgaagagcctggaaaccccacgccgtatactcctgatggttaaagcgggtgccgggaccgatagtgctattgattctctgaaaccgtatctagacaaaggcgatattatcattgatggtggcaatacttttttccaggacacaatccgccgtaaccgagaattgtccgcggagggatttaactacattggtacgggcgttagcggaggtgaagaaggggcattaaagggcccgtcgatcatgccgggcggtcagaaagaagcgtatgagctggtggcccccattctgaagcaaatcgctgctgtcgcagaagatggcgaaccgtgcgtaacctacattggggcggatggtgccggtcactatgtgaaaatggttcataatggcattgagtatggggacatgcagttaatagccgaggcatacgcgttgctgaaaggtggtctggccctgtcgaacgaagaactggcacagaccttcaccgaatggaacgaaggcgaactgtcatcttatctcattgatataacgaaagacatcttcactaaaaaagacgaagatgggaaatatcttgtggatgtaatcttagacgaggcggctaacaagggcaccgggaagtggacgagccagtctagtctggatttgggcgaaccattgtcccttattacggagtctgtctttgcgcgctacatcagctcccttaaagatcaaagggtcgcagctagcaaagttctaagcggcccccaggcgcaaccggcgggagacaaggctgaatttatcgaaaaagtgcgtagagccctgtacctgggtaaaattgtgtcatatgctcagggcttttcccagttacgtgcggcgtctgacgaatacaattgggatctaaattatggtgagatcgccaagatttttcgcgcaggatgtattattcgggcccaatttctgcaaaaaattaccgatgcttatgcgcagaacgcgggcattgctaacctgctgttagccccatacttcaaacagatcgcggatgattatcagcaagcccttcgtgatgtcgtagcctacgctgtgcagaatggcattcctgtaccgacgttttccgcagccatcgcgtactatgactcataccgcagcgcggttctcccggcgaatctgatacaagcccagcgtgattacttcggcgcacacacctataaacgcaccgacaaggaaggtgtctttcataccgaatggctcgaatag

SEQ ID NO: 179 - protein, 470 aa, Bacillus coagulans
MEKQQIGVIGLAVMGKNLAWNIESKGYTVSVFNRSRSKTDQMLKESEGKNIFGYFTMEEFVNSLEKPRKILLMVKAGEATDATIEQLKPFLDKGDILIDGGNTFFKDTQRRNKELSALGIHFIGTGVSGGEEGALKGPSIMPGGQKEAYDLVAPILKDIAAKVNGEPCTTYIGPDGAGHYVKMVHNGIEYGDMELISESYNLLKNILGLGANELHEVFADWNKGELDSYLIEITADIFTKKDPETGKPLVDVILDTAGQKGTGKWTSQSALDLGVPLPLITESVFARFISAMKEERKAASKLLKGPEKPAFSGDKKAFIEAVRKALYMSKICSYAQGFAQMRAASEEYNWDLNYGEIAMIFRGGCIIRAQFLQKIKDAYDRDRNLKNLLLDPYFKEIVESYQDALREVIATAVRFGVPAPALSAALAYYDSYRSEVLPANLIQAQRDYFGAHTYQRVDKEGIFHTEWLEL

SEQ ID NO: 180 - protein, 472 aa, Bacillus coahuilensis
MSKQQIGVIGLAVMGKNLAWNIESRGYSVSVFNRSSDKTEQMVAESTGKNIFPTYTIEEFVSSLEKPRKILLMVKAGKATDATIDSLKPYLEEGDILIDGGNTFFQDTIRRNKELSELGLHFIGTGVSGGEEGALTGPSIMPGGQKEAYELVAPILKDIAAKVDGEACTTYIGPDGAGHYVKMVHNGIEYGDMQLIAESYFLLKNVLGLSADELHEVFAEWNKGELDSYLIEITADIFTKKDDETGKPMVDVILDKAGQKGTGKWTSQSALDLGVSLPVITESVFARFISAIKDERVAASKVLAGPNAESYTGDRKALIEAIRKALYMSKIVSYAQGFAQMRAASEEYNWDLQYGDIAMIFRGGCIIRAQFLQKIKEAYDRDPALRNLLLDSYFKEIVEGYQGALREVISVAVQQGIPVPGFSSALAYYDSYRTATLPANLIQAQRDYFGAHTYERVDKEGIFHTEWIELER

SEQ ID NO: 181 - protein, 473 aa, Variovorax paradoxus
MSKKSDFGLIGLAVMGQNLVLNVESRGFQVSVYNRTEATTEAFIADNPGKKLVGAKTLEEFVQSLAKPRKIQIMVKAGAPVDQVIKQLIPLLEKDDIVIDGGNSLYTDTERRDAYLSSKGLRFIGAGVSGGEEGARKGPSIMPGGPLSTWEVMKPIFESIAAKVDGEPCVIHIGPGGAGHYVKMVHNGIEYGDMQLICEAYSLFKAAGFTTEEMAAIFNEWNDGELQSYLIQITAKALEQKDPETGKPIVDLILDKAGQKGTGQWTLINAAENAVVISTINAAVEARVLSSQKKARVAASKVLQGPKVELSLEKKALVAKVHDALYASKVISYTQGFDLIKTMGDKKEWKLDLGGIASIWRGGCIIRARFLNRITDAFRTDPALANLMLDPFFKDLLNRTQQNWREVVALAVSNGIPVPAFSASLAYYDSYRTERLPANLLQAQRDFFGAHTYERTDKPEGQFFHTDWPEVIG

SEQ ID NO: 182 - protein, 485 aa, Klebsiella sp.
MYNSNSYCNDSSRQEFIMTKQQIGVVGMAVMGRNLALNIESRGYTVSVFNRSREKTEEVIAENPGKKLVPYYTVQEFIESLETPRRILLMVKAGAGTDSAIDSLKPYLDKGDIIIDGGNTFFQDTIRRNRELSAEGFNFIGTGVSGGEEGALKGPSIMPGGQKEAYELVAPILKQIAAVAEDGEPCVTYIGADGAGHYVKMVHNGIEYGDMQLIAEAYALLKGGLALSNEELAQTFTEWNEGELSSYLIDITKDIFTKKDEEGKYLVDVILDEAANKGTGKWTSQSSLDLGEPLSLITESVFARYISSLKDQRVAASKVLSGPQAQPAGDKAEFIEKVRRALYLGKIVSYAQGFSQLRAASDEYNWDLNYGEIAKIFRAGCIIRAQFLQKITDAYAQNAGIANLLLAPYFKQIADDYQQALRDVVAYAVQNGIPVPTFSAAIAYYDSYRSAVLPANLIQAQRDYFGAHTYKRTDKEGVFHTEWMV

SEQ ID NO: 183 - protein, 470 aa, Bacillus coagulans
MEKQQIGVIGLAVMGKNLAWNIESKGYTVSVFNRSRSKTEQMLKESEGKNIFGYFTMEEFVHSLEKPRKILLMVKAGEATDATIEQLKPFLDKGDILIDGGNTFFKDTQRRNKELSALGIHFIGTGVSGGEEGALKGPSIMPGGQKEAYDLVAPILKDIAAKVNGDPCTTYIGPDGAGHYVKMVHNGIEYGDMELISESYNLLKNILGLTADELHEVFADWNKGELDSYLIEITADIFTKKDPETGKPLVDVILDTAGQKGTGKWTSQSALDLGVPLPLITESVFARFISAMKEERKAASKLLKGPEKPAFSGDKKAFIEAVRKALYMSKICSYAQGFAQMRAASEEYNWDLNYGEIAMIFRGGCIIRAQFLQKIKDAYDRDRNLKNLLLDPYFKEIVESYQDALREVIATAVRFGVPVPALSAALAYYDSYRSEVLPANLLQAQRDYFGAHTYQRVDKEGIFHTEWLEL

SEQ ID NO: 184 - protein, 487 aa, lebsiella pneumoniae
MITFKLRTFRSDHTRQEYVMSKQQIGVVGMAVMGRNLALNIESRGYTVSVFNRSREKTEEVIAENPGKKLVPYYTVQEFVKSLETPRRILLMVKAGAGTDSAIDSLKPYLDKGDIIIDGGNTFFQDTIRRNRELSAEGFNYIGTGVSGGEEGALKGPSIMPGGQKEAYELVAPILKQIAAVAEDGEPCVTYIGADGAGHYVKMVHNGIEYGDMQLIAEAYALLKGGLALSNEELAQTFTEWNEGELSSYLIDITKDIFTKKDEDGKYLVDVILDEAANKGTGKWTSQSSLDLGEPLSLITESVFARYISSLKDQRVAASKVLSGPQAQPAGDKAEFIEKVRRALYLGKIVSYAQGFSQLRAASDEYNWDLNYGEIAKIFRAGCIIRAQFLQKITDAYAQNAGIANLLLAPYFKQIADDYQQALRDVVAYAVQNGIPVPTFSAAIAYYDSYRSAVLPANLIQAQRDYFGAHTYKRTDKEGVFHTEWLE

SEQ ID NO: 185 - DNA, 987 bp, Artificial Sequence
  Synthetic
atgtctccgaaaacgactaagaaaattgctatactgacctccgggggagatgcccccggtatgaatgcgacattagtatatctcacccggtacgcaaccagttcggaaatcgaggttttctttgtgaaaaacggctattacggcctttatcacgacgaactggtccctgcgcatcagttggatctgtcaaactcgctgtttagcgcgggtacggtgattggcagcaaacgattcgttgagtttaaggaattaaaagtccgtgaacaagccgctcagaatctgaaaaagaggcaaatcgactacctagttgtgattggaggtgatggcagctatatgggtgcaaaactactttctgaattgggggtaaactgctactgtttgccagggacaatcgataatgacattaacagtagtgaatttaccataggcttcctgactgccctggagtccattaaagtgaatgtccaggcggtgtatcatacgaccaaatctcacgagcgtgtggcgatcgtagaagttatgggacgtcattgcggcgatttagccatctttggtgcactggctactaacgcggatttcgtcgttaccccgagcaataagatggatctcaaacagttggaatcagccgtcaaaaaaattctgcaacatcaaaaccactgtgtggtgattgtgagtgaaaacatctatggctttgacggttacccgagcctgaccgctatcaaacagcacttcgacgccaataacatgaaatgcaatctggtttcgctgggccatacgcagagaggattcgccccgacatcgttggagttagtccagatttcgctgatggcgcaacataccatcaatcttattggtcagaacaaagttaatcaggtgattggtaacaaggcaaacgtcccagttaattatgattttgaccaggcatttaacatgcctccggtggatcgctccgcgttgatcgcggtgataaacaaaaatattatctag

SEQ ID NO: 186 - DNA, 1059 bp, Artificial Sequence
  Synthetic
atgttactgaatatccttactctgaaaaccacgataaaggctctcgacttgtatggagaaaaaggtaacaaaattctgaactgcctgggggtcgcattagtaatgaccaaaatcggcgtgcttacatccggcggtgatgcgcccggcatgaatgccgttattcgggcggtggttaaggccgcatcacactaccatttggaggtcatggggattcaatgtggtttccagggcctgctggaaggaaaaatccatcgtctcacgcctctggaagtggaggatattgcggatagagggggtaccatactcaaaacttcgcgaagcatggaatttatggaagagattggccgcaagaaagctgttgaaatcctaaaaaaccagggtattaatagcctgatcgtaattggcggcggtggcagtttgaaaggagcggaaaagctgcacgagttgggaatcaaagtggtgggtattccagggacaattgacaacgatctggcctttacggattattctatcggcttcgacaccaccctgaacaccgtcctggaatgcatcggtaaaattaaagatactgacttttcccatgataaaacgactatagtagaagtcatgggtcgctactgtggcgacttagctctttattctgcgttggcaggaggcggtgaaatcattagcaccccggagaaaccgcttgatgttaataccatctgctcgaaactgcgccttcgtatgagtaatggtaagaaagacaacatagtgattgttacggaacgtatgtacgaactccaagatttacagcgctatattgaggagaaattaaacatcagcgtgaggactacggtactgggcttcatccagcgtgggggaaatccgtcagcctttgatcgcgtgctagccagtaatatgggtgttaccgccgtggaattactgatgaacggctactccggacaagccgttggtattaaggaaaacaaaatcatccataaagagctgggcaatatcaatgcggggatcgcggacaaacaggataagtatcgtctgctggaaaaactgctcagctag

SEQ ID NO: 187 - DNA, 963 bp, Artificial Sequence
  Synthetic
atggaaataaatcggattggtgtattaactagcggaggcgacgcacccggtatgaacgctgccgtgcgcgcgatcgttcgagcggggcttgccgctggcaaagagatgttcgtcgtgtatgatggctacaagggtctggttgaaaacaaaattatgcaggtcgatcgtctgtttgtgtccgagatcattacccgcggcggtacgatcattcattcagcgcgtttgccggaatttaaagacccagaagttcgcaaaattgcagtcaagaatctgaaagagcgtgggatagatgcgctggtagtgattggcggggacggctcttatatgggtgcgaaagccctcacagaaatgggtatcaactgtatcggactacctggtaccatagataacgatattgcctcgacggatttcaccatcggctttgacacatgcctgaataccatttgcgaagcagtggataaacttagggacactagcttcagtcaccatcgctgttctgttatcgaagtaatggggagatactgcggcgatttggcgatctatgcaggtattggctgtggcgctgatctgattatcagtagcgaccacccgctctccaaggataaagcgattgagcaaatccgtaaaatgcatgaaagcggtcggatgcacattattgtaattatcacggagcatatttgcgatgtccatgaatttgcgaaggagatagaagaaaaagccggcatcgaaacccgtgcagaagtgttagggcgcattcagcggggtggctcgccgtcggctcgtgacagggttctggccgcggaaatgggggtgaaagcaatcgacctgctgtgtgagggcaagggtggacgctgcgtcgggctccgcggacaagagttagttgattacgatattatggaagccttgtccatgaatcgagcgcctcagaaagagctgctggatgtgatttataaattacgttag

SEQ ID NO: 188 - DNA, 984 bp, Artificial Sequence
  Synthetic
atgttaaagattccgacccatatagctgttctgacgtcaggtggggacgcacctggaatgaatgccgcgatccgtgcggtagtgcgaagcgccgtctattacggcaaaaaaatcactggcatttataacggttacgagggccttattaacggtaattttcaggaattgaactccagaagtgtgaaatatatcctcaatcaaggcggtacattcctgaaatctgcacggtcggatcgctttcgcaccccagaaggccgtaagcaggcgtatgataacctggccaaaacggggatcgacgcgctgattgttattggtggggatggctctttcacaggcgcgaaaatttttagcgaagagtacgatttccaagtaatcggggttcccggcacgatcgacaatgatctttacggtaccgactttactataggatatgatacggctaccaataccgccattgaatgcattgacaaaattcgcgataccgcatccagtcacgatcgtctgttcctggtggaggtcatgggcagggactcgggttttatcgctctccgctctgcaatcgccgcgggagcgttggatgtgatcatgccggaaaacgacactacgtatgatcatttagtcgaaaccataaaccgagcaggcaaaaataagaaattcagcaacattattgtggttgctgaagggaataagctgggcaacatttttgagatttcaaactttctcaaaggcaaattcccgcacctggatataaaagtcacaatcctaggtcatctgcaacgtggtgggtcgccaacggtatatgaccgggtgctagcgtccaagcttggagttgcagccgtcgaagggctgcttatcggtcgcaataaagtgatggccggtgtgatgcaccagcagattatttacacaccttttgaagaggcaatcacccgcaaagcttatattaatccggaactgattagaatcaacaaaatactcaccatttag

SEQ ID NO: 189 - DNA, 957 bp, Artificial Sequence
  Synthetic
atgattaagaaaatagccatcctcacttccgggggagattgtccgggcatgaatgtagctttgaaagcgattgttaacgcagcgatcaacaataacattgagccctatgtcgtgtttgaaggttacaaaggcctttatgacaataacttcgaaaaaatcacgaaggaagaggtgaaatttattgatagaaaaggtggtacagttatttactcagcccgtttcccacagtttaaggaactggagatccgaaaacaagcagtcaataacttaaaagctgaaggcatagaagcgctgatttgcatcggcggggatggtacctatatgggtgcggcgaaactgaccgaaatgggcattaaaaccatcgccctaccgggaacgattgacaatgacatcagctcgaccgattacactatcgggtttaacacggcgctggagacgattgtgcgcgcagtagataacctgcgtgataccagtgaatctcacaatcgcattaatcttgtggaagttatgggccatgggtgcggcgacctggccattaacgcggcaattatcactggtgctgaggtcttaagcacacctgaacggaagttggatgtgaaacagatcatcgaaaagttaaaaaaatcggattctaaacgctccaagattgtgatgattagtgaatatatttacaaagacctgaataaagttgctcaagagattgagaaggccacaggtcaggaaaccaaagcgaccatcctcggccatatacagaggggaggttccgcgaacccgatcgagcgccttctgacgatacgtatggccaactatgcaataaaaatgctgatcaagggcaaaaatggggtagcagttaacattaccgataacaaactcaatacgaaagatattctggaaattgttaaaatgaagcgtccctcaaaagaagagttgctgaaagaatatgataaaagcatctag

SEQ ID NO: 190 - DNA, 1113 bp, Artificial Sequence
  Synthetic
atgttagacgccatgaaagttggaattttgacgggtggcggggattgtcctggcctcaatgcggtaatacgagcagcggtcaagactggcatcgctcgtcacggtttcgagatgctgggcattgaagatgcctttcatgggcttgtggacctgggttaccaatccccccatggtaacaggtggctaaccgaaatggatgtgcggggaatccagacacgcggcggtaccattttgggcaccagtaaccgcggcgacccatttcactatgtagtgaaatcggaatctgggaaagagattgaaacggatatttcagatcgcgttctggaaaatatgcatcgtatcgggttagatgcaataatcagcatcggtggcgacggtagcatgcgtattgcgcagcgcttctttgagaaaggtatgccgattgtcggagttccgaaaactatcgataacgacctcggcgccaccgatcagacgttcgggtttgacaccgctgtgtgcattgcgactgaagccatcgatcgtctgtcggatacagcagcatcccatgaccgggttatgctggtcgaggttatgggtcgcgatgctggctggattgcgctgcacgcgggcctcgctggcggtgcggatgccatcttaatcccggaaattccgtatagaatagacgcgattgcgaagatgattgcacaacgttcagccgccaaacagaagtacagtattatcgtcgtgagcgaaggagctaaaccactgggtggcgatcggtctatcggggaaacccgcgcgggggcaatgcctcggctgatgggtgcaggctcccgtgtggcggaggggctgcgcgaattggtaagcgccgatattcgcgttaccgtccttggacacattcaacgtggcggcccgcccagttcttttgatcgtaatctggccacgcgctatgggcgtgctgcggcagatttagtggcgacgaaacagttcggtcgtatggtagcactacgcgacggccagatcgtgactctgccgatagccgacgctatagcaaaacccaagttggtcgatcctaaatcggagatggtcgaaaccgcccgtgccctgggcacattctttggtgatgaaccatag

SEQ ID NO: 191 - protein, 328 aa, Mycoplasma pneumoniae
MSPKTTKKIAILTSGGDAPGMNATLVYLTRYATSSEIEVFFVKNGYYGLYHDELVPAHQLDLSNSLFSAGTVIGSKRFVEFKELKVREQAAQNLKKRQIDYLVVIGGDGSYMGAKLLSELGVNCYCLPGTIDNDINSSEFTIGFLTALESIKVNVQAVYHTTKSHERVAIVEVMGRHCGDLAIFGALATNADFVVTPSNKMDLKQLESAVKKILQHQNHCVVIVSENIYGFDGYPSLTAIKQHFDANNMKCNLVSLGHTQRGFAPTSLELVQISLMAQHTINLIGQNKVNQVIGNKANVPVNYDFDQAFNMPPVDRSALIAVINKNII

SEQ ID NO: 192 - protein, 352 aa, Bacillus bataviensis
MLLNILTLKTTIKALDLYGEKGNKILNCLGVALVMTKIGVLTSGGDAPGMNAVIRAVVKAASHYHLEVMGIQCGFQGLLEGKIHRLTPLEVEDIADRGGTILKTSRSMEFMEEIGRKKAVEILKNQGINSLIVIGGGGSLKGAEKLHELGIKVVGIPGTIDNDLAFTDYSIGFDTTLNTVLECIGKIKDTDFSHDKTTIVEVMGRYCGDLALYSALAGGGEIISTPEKPLDVNTICSKLRLRMSNGKKDNIVIVTERMYELQDLQRYIEEKLNISVRTTVLGFIQRGGNPSAFDRVLASNMGVTAVELLMNGYSGQAVGIKENKIIHKELGNINAGIADKQDKYRLLEKLLS

SEQ ID NO: 193 - protein, 320 aa, Coprobacillus sp
MEINRIGVLTSGGDAPGMNAAVRAIVRAGLAAGKEMFVVYDGYKGLVENKIMQVDRLFVSEIITRGGTIIHSARLPEFKDPEVRKIAVKNLKERGIDALVVIGGDGSYMGAKALTEMGINCIGLPGTIDNDIASTDFTIGFDTCLNTICEAVDKLRDTSFSHHRCSVIEVMGRYCGDLAIYAGIGCGADLIISSDHPLSKDKAIEQIRKMHESGRMHIIVIITEHICDVHEFAKEIEEKAGIETRAEVLGRIQRGGSPSARDRVLAAEMGVKAIDLLCEGKGGRCVGLRGQELVDYDIMEALSMNRAPQKELLDVIYKLR

SEQ ID NO: 194 - protein, 327 aa, Schleiferia thermophila
MLKIPTHIAVLTSGGDAPGMNAAIRAVVRSAVYYGKKITGIYNGYEGLINGNFQELNSRSVKYILNQGGTFLKSARSDRFRTPEGRKQAYDNLAKTGIDALIVIGGDGSFTGAKIFSEEYDFQVIGVPGTIDNDLYGTDFTIGYDTATNTAIECIDKIRDTASSHDRLFLVEVMGRDSGFIALRSAIAAGALDVIMPENDTTYDHLVETINRAGKNKKFSNIIVVAEGNKLGNIFEISNFLKGKFPHLDIKVTILGHLQRGGSPTVYDRVLASKLGVAAVEGLLIGRNKVMAGVMHQQIIYTPFEEAITRKAYINPELIRINKILTI

SEQ ID NO: 195 - protein, 318 aa, Candidatus Hepatoplasma crinochetorum
MIKKIAILTSGGDCPGMNVALKAIVNAAINNNIEPYVVFEGYKGLYDNNFEKITKEEVKFIDRKGGTVIYSARFPQFKELEIRKQAVNNLKAEGIEALICIGGDGTYMGAAKLTEMGIKTIALPGTIDNDISSTDYTIGFNTALETIVRAVDNLRDTSESHNRINLVEVMGHGCGDLAINAAIITGAEVLSTPERKLDVKQIIEKLKKSDSKRSKIVMISEYIYKDLNKVAQEIEKATGQETKATILGHIQRGGSANPIERLLTIRMANYAIKMLIKGKNGVAVNITDNKLNTKDILEIVKMKRPSKEELLKEYDKSI

SEQ ID NO: 196 - protein, 370 aa, Sandaracinus amylolyticus
MLDAMKVGILTGGGDCPGLNAVIRAAVKTGIARHGFEMLGIEDAFHGLVDLGYQSPHGNRWLTEMDVRGIQTRGGTILGTSNRGDPFHYVVKSESGKEIETDISDRVLENMHRIGLDAIISIGGDGSMRIAQRFFEKGMPIVGVPKTIDNDLGATDQTFGFDTAVCIATEAIDRLSDTAASHDRVMLVEVMGRDAGWIALHAGLAGGADAILIPEIPYRIDAIAKMIAQRSAAKQKYSIIVVSEGAKPLGGDRSIGETRAGAMPRLMGAGSRVAEGLRELVSADIRVTVLGHIQRGGPPSSFDRNLATRYGRAAADLVATKQFGRMVALRDGQIVTLPIADAIAKPKLVDPKSEMVETARALGTFFGDEP

SEQ ID NO: 197 - DNA, 747 bp, Artificial Sequence
  Synthetic
atgttacggtatctgcaaattcgcactcatcagaacccctttgcgatgacaaaaacgaataagtctaccgtaatcagtccatcgatactctccgccgatttctcacgtcttggggacgagattcgagctgtcgatgcagcgggcgccgactggattcacgtggatgttatggatggacgctttgtgccgaacatcaccgtcggtcctctggttgtagatgcaatccgtccggtgacgaaaaaaccgctagacgttcatttgatgattgtcgaacctgaaaaatacgtggaggacttcgcgaaggccggcgctgatattatctctgtgcactgtgaacataatgcgagcccacatctctatcgcaccctgtgccagattcgtgaactggacaaacaagcaggcgttgtgctgaacccgagcaccccgttggaactgatcgattacgtcttagaggtgtgcgatctgattttgatcatgagtgtgaatcccggttttggtgggcagagcttcataccggccgttgtgccgaaaatccgtaaactccgacagttatgtaacgaacgcggcctggatccttggattgaagtagacggtggattgaaggctaacaatacttggcaagttctggaagcgggcgccaattctatcgtcgcgggctcggcagtttttaaagctcctgactatgcgaaggcgatctatgatattcgcaactcgcggcgttccgcacaccagcttgcgcaggtctag

SEQ ID NO: 198 - DNA, 729 bp, Artificial Sequence
  Synthetic
atgttaaagaatccgcctgctatgactcaaaacccatcaaaaaaaccgattgttatctccccctctatactctcggcggatttcagccggttgggagacgatattcgcgccgtggataaagcaggcgcggactggatccacgtcgatgtaatggatggtcgatttgtgccgaacattacgatcggcccgcttgttgtcgaggccattaggcctattaccaccaaaccactggacgtgcatctgatgatcgttgaaccggaaaaatatgtcgaaggttttgcaaaggcgggggcggatataatcagtgtgcatgctgagcacaatgctagcccgcatctgcatcgtacactgggccagattaaagaattgggtaagaaagccggtgtagtgctgaacccaggcacgccccttgaactgattgaatacgtgctagagctgtgtgacttagtcctcattatgtcggttaatccggggttcggtggacagtcctttatcccaggagttgtcccgaaaatccgccagctccgccaaatgtgcgacgagcgtggcttagatccttggatcgaagtagatggcggcctgaaagcaaacaatacctggcaggtattagaagccggagccaacgcgatcgtggcaggttctgcggttttcaatgcgccggattatgctgaagctattagtagcattcgtaactccaagcgccccaccccggagctggccgcggtatag

SEQ ID NO: 199 - DNA, 690 bp, Artificial Sequence
  Synthetic
atgtctcagaaaagtttggttatctcccctagcatactttcagcggactttggtcgcttaggcgaagagattcgtgcagtagatgccgcgggagctgattggattcatgtcgatgtgatggacggccggttcgtgccgaatatcacaattggtcccctgatcgttgaagccgtgcgaccacacacgaagaaaccgctggatgtccatctcatgattgtcgaaccggagaaatacgtggcggactttgcaaaagccggggctgatattatctcggtacacgcggaacataacgcaagcccgcacctacatcgtactctggggcaaataaaagaactgggcaagcaggctggtgtcgttctgaacccaggcaccccccttgagttgattgaatatgtgctggagttgtgcgacctcatcttaatcatgtctgtgaatccgggcttcggaggtcaaagctttattccttccgcagtaaccaaagttgccaaactgaggcagatgtgtaacgaacgcgggctggatccgtggattgaagtagatggtggcctgaaggcgaataactcgtggcaggttattgacgccggagctaacgcgatcgttgctggcagtgccgtgtttaatgcgccagattatgcagaagcgatcaaaggtattcgcaattccaaacgcccagagctggtgacggcctag

SEQ ID NO: 200 - DNA, 708 bp, Artificial Sequence
  Synthetic
atgactcagaccagttccaaaaagcctattgtgataagcccgtcaattctttctgccgatttctcgcgtctcggcgaggaagtacgcgcagttgacgaagctggagcggattggatccacgtcgatgtgatggacgggcggtttgttcccaacatcacaatcggtccgctggtcgtggaggcgattcgtccagttaccaaaaaaattttagatgtacatttgatgatcgtggaaccggaaaaatatgtcgccgattttgctaaggcaggcgcggacattataagcgtccattgcgaacacaatgccagtccgcatttacacaggacgctgggtctgatccgagaactaggcaaacaagcgggtgtggtgctcaaccccggcacgccactgtctctgattgagaatgttctggatttgtgtgacctggttctaatcatgtcggtaaaccctggtttcgggggtcagagctttattccgaccgtggtgccgaaaattcgccagttacgccaaatgtgcgatgaacgtggcctggacccatggatcgaggttgacggaggtctgaaagcaaataacacttggcaagttcttgaagctggggccaacgcgatcgtcgctggctccgcggtatacaataccccggattataaagaggccatccatgcgattcgcaacagtaagcgtccggtccccgaactagccaaggtatag

SEQ ID NO: 201 - DNA, 717 bp, Artificial Sequence
  Synthetic
atgaaatacttggagaatcctagtatgcccaagaacatcgttgtggcaccatctattttatcagccgactttagccgactgggcgaagaaataaaagctgtcgatcaagcgggtgcggattggattcacgtagacgtgatggatggacgcttcgtcccgaacatcacgattggcccgctgatcgttgatgccattcgtccgcttactcagaaaccactagacgtgcatctgatgatcgtagaacctgagaaatatgtcgaagattttgcgaaggcaggggccgacattatttcggtgcatgttgagcacaatgcgtccccgcatctgcatcgcaccctctgtcagatccgggaattaggtaaaaaagccggcgctgtcctgaacccgagcacacctcttgatttcctggaatatgtgctcccggtatgcgacctgattttgatcatgagtgttaaccccggttttggtggccagtcttttattccggaagtgctgccgaagatacgttcgttgaggcaaatgtgcgatgaacgtgggctggatccatggattgaggtagatggcggtctgaaacctaataatacctggcaggttctcgaagctggcgcaaacgcgatcgtggcaggatcggctgtctttaatgcgccggattacgccgaagctatagcaggggtgcgcaactccaaacgccccgagccgcaacttgcaacggtttag

SEQ ID NO: 202 - DNA, 660 bp, Artificial Sequence
  Synthetic
atgattaagatcgcgccctccatattatctagcgactttgctaacctcatggccgaggttaaaaaaatcgaagatagtggcgcagattacttgcacgtcgatgtaatggacggttgcttcgtgcctaatattacaattggaccggtggttgtccaagcgctgcgtccgtattggaaacttccaatcgatgtgcatctgatgattgaagaaccgggccgccatctggagtcgtttatcgccgcgggggcagatttaattactgtacacgcagaagcggacagacatctgcacaggaccctgaaatatataaaggatcgtggtaaaaaagccggtgtcgctattaacccagcgacgcatcattcatgtctagactacgttctcccgttcgtggacttgatcgtgataatgagcgtgaatcctggctttggaggtcaggtatttattccggaggtcattccgaaaatcaaggctgttaaagaaatgatcgaaaccttcgggtataacacggagatttccgtggatggcggcattggtcccggaaccgtttttcaggtcgtagaagccggcgctaacatcgttgtggcaggtagtgccgtgttcggctctcctgatccggcccaggcggtgcgaaatattaaagaagcagcggcagggcgctag

SEQ ID NO: 203 - DNA, 645 bp, Artificial Sequence
  Synthetic
atgactttcgtcgcgccctccctcttagctgccgactacatgaatatggcaaactctataaaggaagcggagctggccggggcagattatcttcatattgatgtgatggacggtcactttgtaccaaacctgacatttggaatcgatatggttgaacaaatcggcaaaacggcgaccattcctttggatgtgcatctgatgctcgctaatccggaaaactatattgagaaattcgcggctgccggtgcacacatcattagcgttcatatagaagcggcgccgcacattcatcgggtgatccagcagatcaaacaggctggctgcaaggccggcgtcgttctgaatccgggtacccctgcctcgatgctggaggcagtacttggcgatgtggacttagtcctgcaaatgacggtgaacccagggtttggcggtcagacctttatcgaatcaaccattgaaaacatgcgttacttggataattggagacgaaaaaaccgtggcagctatagtattgaagttgatggaggtgttaataaagccacagcggagacttgtaagcaggctggcgtagacatcttagtggcagggtcttatttctttcgcgcgattgacaaagccgcctgtgtaaaaacgctgaaatcgtag

SEQ ID NO: 204 - protein, 248 aa, Richelia intracellularis HH01
MLRYLQIRTHQNPFAMTKTNKSTVISPSILSADFSRLGDEIRAVDAAGADWIHVDVMDGRFVPNITVGPLVVDAIRPVTKKPLDVHLMIVEPEKYVEDFAKAGADIISVHCEHNASPHLYRTLCQIRELDKQAGVVLNPSTPLELIDYVLEVCDLILIMSVNPGFGGQSFIPAVVPKIRKLRQLCNERGLDPWIEVDGGLKANNTWQVLEAGANSIVAGSAVFKAPDYAKAIYDIRNSRRSAHQLAQV

SEQ ID NO: 205 - protein, 242 aa, Anabaena cylindrica
MLKNPPAMTQNPSKKPIVISPSILSADFSRLGDDIRAVDKAGADWIHVDVMDGRFVPNITIGPLVVEAIRPITTKPLDVHLMIVEPEKYVEGFAKAGADIISVHAEHNASPHLHRTLGQIKELGKKAGVVLNPGTPLELIEYVLELCDLVLIMSVNPGFGGQSFIPGVVPKIRQLRQMCDERGLDPWIEVDGGLKANNTWQVLEAGANAIVAGSAVFNAPDYAEAISSIRNSKRPTPELAAV

SEQ ID NO: 206 - protein, 229 aa, Chamaesiphon minutus
MSQKSLVISPSILSADFGRLGEEIRAVDAAGADWIHVDVMDGRFVPNITIGPLIVEAVRPHTKKPLDVHLMIVEPEKYVADFAKAGADIISVHAEHNASPHLHRTLGQIKELGKQAGVVLNPGTPLELIEYVLELCDLILIMSVNPGFGGQSFIPSAVTKVAKLRQMCNERGLDPWIEVDGGLKANNSWQVIDAGANAIVAGSAVFNAPDYAEAIKGIRNSKRPELVTA

SEQ ID NO: 207 - protein, 235 aa, Calothrix sp.
MTQTSSKKPIVISPSILSADFSRLGEEVRAVDEAGADWIHVDVMDGRFVPNITIGPLVVEAIRPVTKKILDVHLMIVEPEKYVADFAKAGADIISVHCEHNASPHLHRTLGLIRELGKQAGVVLNPGTPLSLIENVLDLCDLVLIMSVNPGFGGQSFIPTVVPKIRQLRQMCDERGLDPWIEVDGGLKANNTWQVLEAGANAIVAGSAVYNTPDYKEAIHAIRNSKRPVPELAKV

SEQ ID NO: 208 - protein, 238 aa, Synechocystis sp
MKYLENPSMPKNIVVAPSILSADFSRLGEEIKAVDQAGADWIHVDVMDGRFVPNITIGPLIVDAIRPLTQKPLDVHLMIVEPEKYVEDFAKAGADIISVHVEHNASPHLHRTLCQIRELGKKAGAVLNPSTPLDFLEYVLPVCDLILIMSVNPGFGGQSFIPEVLPKIRSLRQMCDERGLDPWIEVDGGLKPNNTWQVLEAGANAIVAGSAVFNAPDYAEAIAGVRNSKRPEPQLATV

SEQ ID NO: 209 - protein, 219 aa, Desulfotomaculum sp.
MIKIAPSILSSDFANLMAEVKKIEDSGADYLHVDVMDGCFVPNITIGPVVVQALRPYWKLPIDVHLMIEEPGRHLESFIAAGADLITVHAEADRHLHRTLKYIKDRGKKAGVAINPATHHSCLDYVLPFVDLIVIMSVNPGFGGQVFIPEVIPKIKAVKEMIETFGYNTEISVDGGIGPGTVFQVVEAGANIVVAGSAVFGSPDPAQAVRNIKEAAAGR

SEQ ID NO: 210 - protein, 214 aa, Listeria ivanovii
MTFVAPSLLAADYMNMANSIKEAELAGADYLHIDVMDGHFVPNLTFGIDMVEQIGKTATIPLDVHLMLANPENYIEKFAAAGAHIISVHIEAAPHIHRVIQQIKQAGCKAGVVLNPGTPASMLEAVLGDVDLVLQMTVNPGFGGQTFIESTIENMRYLDNWRRKNRGSYSIEVDGGVNKATAETCKQAGVDILVAGSYFFRAIDKAACVKTLKS

SEQ ID NO: 211 - DNA, 702 bp, Artificial Sequence
  Synthetic
atgatttacaatgcgcgcactacgcattccctcgggaacatcatgacacaagatgagttaaaaaaggcagtaggttgggctgccctgcaatatgttcagcccggcaccatagtcggagtgggcaccggttcgacggcggcccacttcattgacgcactgggcaccatgaaagggcagatcgaaggagcggtgtctagctcagatgcgagtactgaaaaacttaaaagcctgggtattaccgtctttgatttgaacgaagttgaccgtctgggcatctatgtggatggcgcagacgagatcaatgatcatatgcagatgattaaaggcggaggtgccgctttgacgcgggaaaagattattgcctccgtagcggacaaatttatctgcatcgcggatgcctcgaaacaggtcgcgattctaggcaacttcccgctgcctgttgaagtgatcccaatggcacgcagtgccgtggcacgtgcacttgttaagttaggtgggcgcccggagtaccgacagggggtgctgacagacaatggtaacgtgattctggatgttcacggcctcgaaatcctggatccggtagctttggaaaacgcgattaatggtattccgggtgtggtcaccgttggtctgtttgctaaccgtggagcggatgtcgctctcattggcaccgcggacggtgtgaaaactattgtgaaatag

SEQ ID NO: 212 - DNA, 663 bp, Artificial Sequence
  Synthetic
atgaatctgaaacagttggctggagaatatgcggcaggctttgtgcgagatggtatgactattggcctagggaccggttcaacggtatactggacaatccaaaagcttggccaccgtgtccaggagggtctgagtatacaagccgttccaacctccaaagaaacagaggtgctggcgaaacagctctcgattcctctgatctctctgaacgaaattgacatcttagatttgacgattgatggtgccgacgaaatcaacaatgatctccagttaatcaagggcgggggcggagctttgttacgggagaaaattgttgcaaccagcagtaaagaactgattattatcgcggacgaatctaaactggtgagccatctgggcaccttccccctgccgattgagataatcccgtttagctggaaacaaactgaaaagcgcattcagtcgctgggatgtgaaacgcgtcttaggatgaaagatggtggtccgttcataaccgacaacggcaatcttatcatcgattgcatttttcccaacaaaattctcaatccgaacgatacacatactgagctgaaaatgatcaccggggttgtagaaacgggtttattcattaatatgaccagcaaggccattattggcaccaaaaacgggatcaaagagtattag

SEQ ID NO: 213 - DNA, 684 bp, Artificial Sequence
  Synthetic
atggaaaacttgaagaaaatggcaggtattaaagcggctgagttcgtaaaagatggaatggttgtcgggctcggtacaggcagtacggcgtattactttgtggaagaaatcggccgtcggatcaaagaggaaggcctacagattaccgccgtgactacctcgtctgtgacgagcaagcaagccgagggtttaaatatacctcttaaatccattgaccaggttgattttgtagacctgaccgtcgatggcgctgatgaagttgactcacaattcaacggcatcaaagggggtgggggcgcgttactgatggaaaaagttgtggcgactccgtccaaagagtatatttgggtcgtagatgaaagcaagctggttgaaaaactgggtgcatttaaactgcccgtggaagtggttcagtacggggccgagcaggtattccgccgatttgaacgcgcaggttataagccgcactttcgcgaaaaagatggccaaagattcgtcaccgatatgcagaatttcatcattgacttggccctggacgtcatcgaagatccaattgcctttggacaggagctagatcatgttgtgggagtcgtggaacatggcttattcaaccagatggttgacaaagtcatagtggcgggtcgtgatggtgtgcaaatcctgacgtctacaaaagcgaagtag

SEQ ID NO: 214 - DNA, 711 bp, Artificial Sequence
  Synthetic
atgaaaatacaagcgttgatgctcgatcatgtgcggcgctctaaggcaatggaccttaaacagattgccggagaatacgctgcgacattcgttaaagatggcatgaaaatcgggttaggcactggttcaacggcctattggaccattcagaagctaggtcagcgagtcaaagagggcctgtcgatccaagcagtacctacctccaaagaaacggaagcgctggcccagcaactgaacattccgctgatcagtttaaatgacgttcagagtctggatctcaccatcgatggggcggacgagattgatagcaatcttcagttgattaagggaggtggcggtgctctgctgcgtgaaaaaattgtggccagctcgtctaaagaactgatcataatcgtagatgagtcgaaagtggttactcgcctgggcacatttcccttgccaattgaaattatcccgtttgcatggaagcagaccgagtccaaaatccaaagcctgggttgtcagacgaccctaaggctgaaaaacaacgaaaccttcataactgacaataacaatatgattattgattgcatttttccgaaccacattccgacgccttcagacttacataaacgccttaagatgattaccggagtcgtggaaacgggcctttttgttaatatgacaagcaaagccattatcggtactaaaaacggcatccaggagctgtag

SEQ ID NO: 215 - DNA, 663 bp, Artificial Sequence
  Synthetic
atgaatgcggatgagatgaaaaagcaagctgcatgggccgcactggaatatattaaaggtgacggcatagtaggagtggggacaggcagcactgtcaaccactttatcgatgcgttagccaccattaaaggtcgcatcgaaggcgcggtttcgtctagtgaggctagcaccaagaaaatgcaggaacttggtattaaagtgttcgacttgaacgaatgtaatgaaatcgaggtttacgtggatggggccgatgaagcgaactcactcctggaactggtcaaaggcgggggaggtgcgctgacgcgggaaaaaattatcgccgctgcaagtaaacagtttgtttgcattgtcgatgccacgaagcaagtagacatattaggtaaattcccactgcccgtggaggtcattcctatggctcgttcctatgtggcgagggaaatcgttaaactcggcggccagccggtataccgagagggtgtgattaccgataatggcaacgttatccttgatgtgcatgggatggacatcatggaaccgatcaagcttgagaaaactttgaatgacattgtcggagtcgtaaccaacggcttgttcgcgatgcgtccggccgacgttctgctggtgggttctgaagatggtacgcagacggtgcatgcaaaatag

SEQ ID NO: 216 - DNA, 684 bp, Artificial Sequence
  Synthetic
atggaaaacttgaagaaaatggcaggtattaaagcggctgagttcgtaaaagatggaatggttgtcgggctcggtacaggcagtacggcgtattactttgtggaagaaatcggccgtcggatcaaagaggaaggcctacagattaccgccgtgactacctcgtctgtgacgagcaagcaagccgagggtttacagatacctcttaaatccattgaccaagttgattttgtagacctgaccgtcgatggcgctgatgaagttgactcacagttcaatggcatcaaagggggtgggggcgcgttactgatggaaaaaattgtggcgactccgtccaaagagtatatttgggttgtcgatgaaagcaagctggttgaaaaactgggtgcatttaaactgcccgtagaagtggtccagtacggggccgagcaggtctttcgacgcttcgagcgcgccggttataagccgtctttccgtgaaaaagatggccaacgctttgtgaccgacatgcagaacttcatcatcgatcttgacctgaaagtgattgaagatccaatcgctttgggacaagaactggatcatgttgtgggagttgtagaacacggcttatttaatcagatggttgacaaagtcatagtggcgggtcagaacggtctgcaaattctcacgagcactaaggcaaaatag

SEQ ID NO: 217 - protein, 233 aa, Trabulsiella guamensis
MIYNARTTHSLGNIMTQDELKKAVGWAALQYVQPGTIVGVGTGSTAAHFIDALGTMKGQIEGAVSSSDASTEKLKSLGITVFDLNEVDRLGIYVDGADEINDHMQMIKGGGAALTREKIIASVADKFICIADASKQVAILGNFPLPVEVIPMARSAVARALVKLGGRPEYRQGVLTDNGNVILDVHGLEILDPVALENAINGIPGVVTVGLFANRGADVALIGTADGVKTIVK

SEQ ID NO: 218 - protein, 220 aa, Bacillus cereus
MNLKQLAGEYAAGFVRDGMTIGLGTGSTVYWTIQKLGHRVQEGLSIQAVPTSKETEVLAKQLSIPLISLNEIDILDLTIDGADEINNDLQLIKGGGGALLREKIVATSSKELIIIADESKLVSHLGTFPLPIEIIPFSWKQTEKRIQSLGCETRLRMKDGGPFITDNGNLIIDCIFPNKILNPNDTHTELKMITGVVETGLFINMTSKAIIGTKNGIKEY

SEQ ID NO: 219 - protein, 227 aa, Streptococcus sp.
MENLKKMAGIKAAEFVKDGMVVGLGTGSTAYYFVEEIGRRIKEEGLQITAVTTSSVTSKQAEGLNIPLKSIDQVDFVDLTVDGADEVDSQFNGIKGGGGALLMEKVVATPSKEYIWVVDESKLVEKLGAFKLPVEVVQYGAEQVFRRFERAGYKPHFREKDGQRFVTDMQNFIIDLALDVIEDPIAFGQELDHVVGVVEHGLFNQMVDKVIVAGRDGVQILTSTKAK

SEQ ID NO: 220 - protein, 236 aa, Bacillus thuringiensis
MKIQALMLDHVRRSKAMDLKQIAGEYAATFVKDGMKIGLGTGSTAYWTIQKLGQRVKEGLSIQAVPTSKETEALAQQLNIPLISLNDVQSLDLTIDGADEIDSNLQLIKGGGGALLREKIVASSSKELIIIVDESKVVTRLGTFPLPIEIIPFAWKQTESKIQSLGCQTTLRLKNNETFITDNNNMIIDCIFPNHIPTPSDLHKRLKMITGVVETGLFVNMTSKAIIGTKNGIQEL

SEQ ID NO: 221 - protein, 220 aa, Methylophaga thiooxydans
MNADEMKKQAAWAALEYIKGDGIVGVGTGSTVNHFIDALATIKGRIEGAVSSSEASTKKMQELGIKVFDLNECNEIEVYVDGADEANSLLELVKGGGGALTREKIIAAASKQFVCIVDATKQVDILGKFPLPVEVIPMARSYVAREIVKLGGQPVYREGVITDNGNVILDVHGMDIMEPIKLEKTLNDIVGVVTNGLFAMRPADVLLVGSEDGTQTVHAK

SEQ ID NO: 222 - protein, 227 aa, Streptococcus infantis
MENLKKMAGIKAAEFVKDGMVVGLGTGSTAYYFVEEIGRRIKEEGLQITAVTTSSVTSKQAEGLQIPLKSIDQVDFVDLTVDGADEVDSQFNGIKGGGGALLMEKIVATPSKEYIWVVDESKLVEKLGAFKLPVEVVQYGAEQVFRRFERAGYKPSFREKDGQRFVTDMQNFIIDLDLKVIEDPIALGQELDHVVGVVEHGLFNQMVDKVIVAGQNGLQILTSTKAK

SEQ ID NO: 223 - DNA, 954 bp, Artificial Sequence
  Synthetic
atgactgacaaactaacctccctccgtcaatacacgaccgttgtggcagatacaggagatattgctgcgatgaagctttatcagccacaggatgccaccacgaatccctcactgatcctgaacgcggcccaaataccggagtatcgaaaattgattgacgacgcggtcgcatgggcgaaacagcagagcagtgatcgcgctcagcaaatcgtagatgccaccgataagctggcagtgaacattggtttagaaatcttaaaattggttcctgggcgcatctctacggaagtagacgcgcgtctgtcatacgacaccgaagctagcattgccaaagctaaacggctgattaaactttataatgatgcaggcatatctaacgataggatcctgattaagctggcgagcacgtggcagggcattcgcgccgcagagcaactagaaaaagaaggtatcaactgtaatctcactctgttattcagttttgcgcaggcccgtgcgtgcgcggaggcaggcgtctacctgatctcgccgtttgtcggtcgcattttagattggtataaagccaataccgataagaaagaatacgcaccggcggaagatccgggtgtggtgtcggtttccgaaatctatcagtattacaaagaacacggctatgagacagttgtgatgggggcgtccttccgcaacatgggagagattcttgagcttgcaggctgcgaccgtttgacgattgccccagcgctgctcaaagaactggctgaaagcgagggtgccgtggaacgtaagctgagctttagcggtgaagtaaaagctcggccggaacgcataaccgaaagtgaatttttgtggcagcataatcaggatcccatggccgttgataagctggctgacggcatccgaaaattcgcggttgatcaagaaaaactggagaaaatgatcggggaattgctgtag

SEQ ID NO: 224 - DNA, 954 bp, Artificial Sequence
  Synthetic
atgactgacaaactaacctccctccgtcaattcacgaccgttgtggcagatacaggagatattgctgcgatgaagctttatcagccacaggatgccaccacgaatccctcactgatcctgaacgcggcccaaataccggagtaccgaaaattgattgacgacgcggtcgcatgggcgaaacagcagagcagtgatcgcgctcagcaaatcgtagatgccaccgataagctggcagtgaacattggtttagaaatcttaaaattggttcctgggcgcatctctacggaagtagacgcgcgtctgtcatatgacaccgaagctagcattgccaaagctaaacggattattaaactctacaatgatgcaggcatctctaacgataggatcctgatcaagctggcgagcacgtggcagggcattcgcgccgcagagcaactggaaaaagaaggtataaactgtaatcttactctgttatttagttttgcgcaggcccgtgcgtgcgcggaggcaggcgtctatctgatctcgccgttcgtcggtcgcattttagattggtacaaagccaataccgataagaaagaatatgcaccggcggaagatccgggtgtggtgtcggttacagaaatttatgagtactacaaacaacatggctatgagactgtggtaatgggggctagctttcgtaacataggcgaaattctagaactggccgggtgcgaccgtctgactattgcaccggcattgcttaaggagttagccgaatcggaaggcgcggtcgaacgaaaactgtccttctctggagaagttaaagcgcgcccagaaagaatcaccgagtcggagtttttgtggcagcacaatcaggatcccatggctgtcgataagctggctgacggtatccgcaaatttgcggttgatcaagaaaaactggaaaaaatgatcggggatcttctgtag

SEQ ID NO: 225 - DNA, 984 bp, Artificial Sequence
  Synthetic
atggctaacttgctggatcaactcaaacagatgacggtcgttgtggcggacactggagatattcaggcaatcgaaaagtatacaccacgggatgccaccaccaatccctcactgataacggcggcagcccaaatgccgcagtaccaggggattgtggacgacaccttaaaagcggcccgtcaaagtcttggtgcggatgctcctgcatcggaggtagtatccctggcgttcgatcgcttggccgtttcttttggtctgaaaatcctggaaattatcccaggccgcgtgagcaccgaagtcgatgcgcgtcttagctatgatactgaggctacaattgcaaagggccgtgacctcatagcgcagtacgaagccgccggcgtcagtcgcgatagaatcctgattaaaattgcctccacgtgggaaggtatccaagctgccgcagttttagagaaagaaggcattcattgcaacctgaccctgctatttggtttgcaccaggcagtggcttgtgcggaaaatggtatcacactaatcagcccgttcgttgggcgaattttagactggtataaaaaggatactggccgcgatagctatccgtcgaacgaagatccgggcgtgctgtcagtaactgagatttactcttactataaaaaatttgggtataacacggaagtcatgggcgcgtccttccgtaatgtcggggagattaccgagttagcaggagtggacctcctgacaatatctcctgcactgcttgacgaactgcaaaacacggaaggaaccctggaacggaaactaagtccggaagtggcggcacagtcggacgttgctgaactgaatttggacaaagcgacctttgatgccatgcatgctgaaaatcgcatggcggccgagaaattatctgaaggtatcgatggctttgcgaaggctcttgagagcttggaagagcttctggcgacgaggctggctaaccttgagtcgtag

SEQ ID NO: 226 - DNA, 990 bp, Artificial Sequence
  Synthetic
atggctaagaatctattggaacagttacgtgagatgaccgttgtggtagcagatacaggtgacattcaagcgatcgaaactttcaaaccgcgcgatgccacgaccaaccccagccttataaccgcggcagcccagatgcctcaataccagggcatcgtcgatgacacgctgaaaggagctagagtgactctcggcgcgggggcgtcagcagccgaggttgcgtcgctggcttttgatcgcctggccgtgtcttttggtctgaaaattctggaaattatcgaaggccgtgtcagtacagaagttgacgcgcgactgtcctatgatgtggaaggtaccattgccaaaggacgggacattattgcacagtataaggcagccggcatcgatacggagaaacgcatcctgatcaaaatagcggccacctgggaaggtattcaggctgcggcagtactcgaaaaggagaacattcatacaaatttaaccttgcttttcgggatccaccaagcgatcgcttgtgcggagaacggcattcaacttatcagcccatttgtaggccgtattctggattggtacaaaaaagacacgggtcgagatagctatgcaccttctgaagatccgggggttctgtcggtcactgaaatctataactactacaaaaaattcggttataaaaccgaagtgatgggcgcgtcatttcgcaatattggagaaattaccgagttagcgggttgcgacttgttgacgattgccccgagcctgctcgccgagctgcaatccgtggaaggcgagctgccacgtaagctggatgcggctaaggcagcatcggcgaatattgaaaaaatcagtgtggataaagctacttttgaacgcatgcatgaagaaaaccgtatggccaacgacaaattgaaagagggcatagatgggttcgctaaagctcttgaggcactagaaaagctgttagccgaccggttggccgtgcttgaagcgtag

SEQ ID NO: 227 - DNA, 990 bp, Artificial Sequence
  Synthetic
atggctaagaatctattggaacagttacgtgagatgaccgttgtggtagcagatacaggtgacattcaagcgatcgaaactttcaaaccgcgcgatgccacgaccaaccccagccttataaccgcggcagcccagatgcctcaataccagggcatcgtcgatgacacgctgaaatccgctcgggcgactctgggagcctcagcgtcgccggcagaggtggcgagtctggcatttgatcgactcgctgtttcttttggcctgaaaattcttgaaatcattgaagggcgtgtgtctaccgaggtcgatgccaggctcagctatgacacggaaggtaccttggccaaagcgcgcgacattattgctcagtataaggcggcaggcatcgataccgaaaaacgtattctgataaaaatcgcggccacatgggaaggtattcaggcggctgccgtgttagaaaaagaaaacatccacacgaatctgacactcctgttcgggatgcatcaagctattgcatgtgctgagaacggcatccagttgattagcccatttgttggacgcatcttagactggtacaaaaaagataccggtagagatagttatgcaccgcatgaggatccgggcgtactgtccgtgactgaaatttacaattattacaagaagtttgggtataaaaccgaggtcatgggtgcgtcattccgtaacatcggcgaaataactgaactggcgggctgcgacctgctgactattgccccgtcgctcctggcagaactacagagcgtagagggtgaccttccacgcaaactggatcctgcgaaggcagcgtcagccgatattgaaaaaatttccgtggataaagctacatttgatcggatgcatgaagaaaaccgcatggccaatgaaaaattaaaagaagggatcgacggtttcgcgaaagccctggagacgctggaaaaactgctggcggaccgtttagctgcgcttgaggcctag

SEQ ID NO: 228 - DNA, 1446 bp, Artificial Sequence
  Synthetic
atgaaacaggaagagtgtcaaatgactaaggcgaactttggtgtggtaggaatggccgttatgggcaggaatttagcacttaacatcgaatcccgcggctacacagtcgctatatataatcgttcgaaagaaaaaacggaggatgtgattgcgtgccatccggaaaaaaacttcgtaccatcatatgacgttgaatcttttgtcaatagcattgaaaaacctcgacgcatcatgctcatggtgcaggccggtcccggcaccgatgctaccattcaggcactgttgccgcacctggacaagggggatattctgatcgacggtggtaacacgttctacaaagataccatccgtcgcaatgaggaactagcgaacagcggcattaattttatcgggaccggcgtcagtggtggcgagaaaggcgcgctggaagggccgtcaattatgccaggaggtcaaaaggaagcctatgagctggttagcgatgtgttagaagagatttccgcaaaagcaccggaagatggaaagccttgcgtgacgtatatcggtcccgatggcgccggtcattacgtcaaaatggtacacaacgggatcgaatacggcgacatgcagttgatagctgaatcgtatgatctgatgcagcatcttctcggtctgtctgcggaagatatggcggaaatttttaccgaatggaacaaaggggaactggacagttatctgattgagattacagccgacatcctgagtcgtaaagacgatgaggatcaagatggcccgatagtggattacattctagatgcagcgggcaataaggggacgggcaaatggaccagccagtccagtcttgatttgggggttccgctgtcactaattactgaaagcgttttcgcgcgctatatctctacttataaagaggaacgggttcacgccagtaaagtgttacctaaacccgctgcgtttaacttcgagggagacaaagcagaattgattgagaaaatcagacaggcgctgtatttttccaagattatctcgtacgcgcaaggattcgcacaactgcgtgtggcctcgaaagagaataattggaacttaccgttcgcggatatagccagcatttggcgtgacggttgtatcatccgctcacggtttcttcagaaaattacggacgcatacaatcgtgacgctgatttggcgaacctgctgttagatgaatattttctggacgtgaccgccaaatatcagcaggcggttcgcgatattgtagcactggcagtccaagccggcgttccagtcccgacattttcggctgcaattacgtactttgattcttatcgaagcgcggatttaccagctaacctaatacaagcgcagcgggactacttcggtgctcatacctaccagcgaaaagataaggaaggcacatttcactactcctggtatgacgagaagtag

SEQ ID NO: 229 - protein, 317 aa, Escherichia fergusonii
MTDKLTSLRQYTTVVADTGDIAAMKLYQPQDATTNPSLILNAAQIPEYRKLIDDAVAWAKQQSSDRAQQIVDATDKLAVNIGLEILKLVPGRISTEVDARLSYDTEASIAKAKRLIKLYNDAGISNDRILIKLASTWQGIRAAEQLEKEGINCNLTLLFSFAQARACAEAGVYLISPFVGRILDWYKANTDKKEYAPAEDPGVVSVSEIYQYYKEHGYETVVMGASFRNMGEILELAGCDRLTIAPALLKELAESEGAVERKLSFSGEVKARPERITESEFLWQHNQDPMAVDKLADGIRKFAVDQEKLEKMIGELL

SEQ ID NO: 230 - protein, 317 aa, Citrobacter sp.
MTDKLTSLRQFTTVVADTGDIAAMKLYQPQDATTNPSLILNAAQIPEYRKLIDDAVAWAKQQSSDRAQQIVDATDKLAVNIGLEILKLVPGRISTEVDARLSYDTEASIAKAKRIIKLYNDAGISNDRILIKLASTWQGIRAAEQLEKEGINCNLTLLFSFAQARACAEAGVYLISPFVGRILDWYKANTDKKEYAPAEDPGVVSVTEIYEYYKQHGYETVVMGASFRNIGEILELAGCDRLTIAPALLKELAESEGAVERKLSFSGEVKARPERITESEFLWQHNQDPMAVDKLADGIRKFAVDQEKLEKMIGDLL

SEQ ID NO: 231 - protein, 327 aa, Methylophaga nitratireducenticrescens
MANLLDQLKQMTVVVADTGDIQAIEKYTPRDATTNPSLITAAAQMPQYQGIVDDTLKAARQSLGADAPASEVVSLAFDRLAVSFGLKILEIIPGRVSTEVDARLSYDTEATIAKGRDLIAQYEAAGVSRDRILIKIASTWEGIQAAAVLEKEGIHCNLTLLFGLHQAVACAENGITLISPFVGRILDWYKKDTGRDSYPSNEDPGVLSVTEIYSYYKKFGYNTEVMGASFRNVGEITELAGVDLLTISPALLDELQNTEGTLERKLSPEVAAQSDVAELNLDKATFDAMHAENRMAAEKLSEGIDGFAKALESLEELLATRLANLES

SEQ ID NO: 232 - protein, 329 aa, Methylomonas koyamae
MAKNLLEQLREMTVVVADTGDIQAIETFKPRDATTNPSLITAAAQMPQYQGIVDDTLKGARVTLGAGASAAEVASLAFDRLAVSFGLKILEIIEGRVSTEVDARLSYDVEGTIAKGRDIIAQYKAAGIDTEKRILIKIAATWEGIQAAAVLEKENIHTNLTLLFGIHQAIACAENGIQLISPFVGRILDWYKKDTGRDSYAPSEDPGVLSVTEIYNYYKKFGYKTEVMGASFRNIGEITELAGCDLLTIAPSLLAELQSVEGELPRKLDAAKAASANIEKISVDKATFERMHEENRMANDKLKEGIDGFAKALEALEKLLADRLAVLEA

SEQ ID NO: 233 - protein, 329 aa, Methylomonas koyamae
MAKNLLEQLREMTVVVADTGDIQAIETFKPRDATTNPSLITAAAQMPQYQGIVDDTLKSARATLGASASPAEVASLAFDRLAVSFGLKILEIIEGRVSTEVDARLSYDTEGTLAKARDIIAQYKAAGIDTEKRILIKIAATWEGIQAAAVLEKENIHTNLTLLFGMHQAIACAENGIQLISPFVGRILDWYKKDTGRDSYAPHEDPGVLSVTEIYNYYKKFGYKTEVMGASFRNIGEITELAGCDLLTIAPSLLAELQSVEGDLPRKLDPAKAASADIEKISVDKATFDRMHEENRMANEKLKEGIDGFAKALETLEKLLADRLAALEA

SEQ ID NO: 234 - protein, 481 aa, Streptococcus pneumoniae
MKQEECQMTKANFGVVGMAVMGRNLALNIESRGYTVAIYNRSKEKTEDVIACHPEKNFVPSYDVESFVNSIEKPRRIMLMVQAGPGTDATIQALLPHLDKGDILIDGGNTFYKDTIRRNEELANSGINFIGTGVSGGEKGALEGPSIMPGGQKEAYELVSDVLEEISAKAPEDGKPCVTYIGPDGAGHYVKMVHNGIEYGDMQLIAESYDLMQHLLGLSAEDMAEIFTEWNKGELDSYLIEITADILSRKDDEDQDGPIVDYILDAAGNKGTGKWTSQSSLDLGVPLSLITESVFARYISTYKEERVHASKVLPKPAAFNFEGDKAELIEKIRQALYFSKIISYAQGFAQLRVASKENNWNLPFADIASIWRDGCIIRSRFLQKITDAYNRDADLANLLLDEYFLDVTAKYQQAVRDIVALAVQAGVPVPTFSAAITYFDSYRSADLPANLIQAQRDYFGAHTYQRKDKEGTFHYSWYDEK

SEQ ID NO: 235 - DNA, 1995 bp, Artificial Sequence
  Synthetic
atgtttgacaaaatcgatcaactcggtgttaacacgattcgtacactttcagtcgatgctgtacagaaggcaaatagtggacacccagggttacccatgggcgccgcgcctatggcgtacgccctgtggaccaaacatctgaaagtgaacccgaaaactagcaagaattgggcagaccgggatcgcttcgtgctatcggccggtcatggctctgcgatgctgtattccctgttgcacctggcgggctatcaggttaccattgatgatcttaaacagtttaggcaatgggagagcaaaacgccgggtcatccggaagtgaaccataccgacggcgtagaagctacaaccggtcccttaggacaggggatagcaatggctgttggcatggcgatggccgaagcacacctcgccgcgacttacaacaaggatcagttcaatgtcgtagaccactatacgtacgccttgtgtggggacggtgatctgatggagggtgtgagccaagaagcatcctcgatggcgggacatatgaaactcggcaaactgatcgtattatatgatagtaatgatatttcactggacggcccgacctctaaggcgtttaccgaaaacgtgggtgcgcgttacgaagcttatggctggcagcatatcctggtcaaagatggcaatgaccttgaggccattagtaaagctattgaggaagcgaaagcagaaactgacaagccaacgctgatcgaagttaaaaccgtgattgggttcggtgctccgaaccaaggcacgagcgccgtccacggggctcctcttgggcttgaggggatccagaaagcgaaggaaatatatggctgggagtatccggattttaccgtgccggaagaggtcgcggaacgctttcgacaaaccatggttgaagaaggtgaaaaagcggagaatgcctggcgcgaaatgttcgcagcttacaaagctgcctaccccgaattggcgcagcaatttgaggatgccttcgcgggtaaactgccggagaactgggatgccgaactgccaacctatgacgaaggagaaagccaggcatccagagtttcatctaaggaagtgattcaggaacttagtaaagctatcccaagtttttggggtggctcggctgatctgagcggcagtaacaatactatggttacggcagacaaagattttacgccggaacattacgagggccgcaatatctggtttggtgtgcgcgagttcgcaatggccagcgcgatgaacggcattcagttacacggagggacacgtatctatggcggtacctttttcgtattcgtagattatttgcggccggccgtccgtctagcagcgatccaaaatactcctgtgattttcgttctgacccacgactcggtggccgtcggcgaggatggaccgacccatgaacctgtagagcaactcgcgagcgtccgttccatgccaggagtgcatgttctgcgcccggcagatggtaacgaaacacgggcggcctggaaggtggcaatggagtcaacggataccccgacaattctggtgctatcgcgccagaacctgccagtactgccgacgactaaagaagtcgcggatgatatggtcaaaaaaggggcttatgtactcagcccggcgaagggagaacagcccgagggcatactgatcgcgaccggttccgaagtagaccttgcggtgaaagcccagaaagttctagccgaacagggcaaggacgtttctgttgtgagcatgccatcattcgacttgtttgaacagcaatcggcagagtaccaggaatccgtcttacccaaaagtgtgactaaacgagtagcaattgaagcggcggccagctttggctgggagcgttatgtaggaattgagggccagacgataactatagatcatttcggtgcctccgcaccgggaaataaaattctggaagaatttggttttacggtcgataacgtggtcaacgtgttcaaccagttgtag

SEQ ID NO: 236 - DNA, 1995 bp, Artificial Sequence
  Synthetic
atgtttgacaaaatcgatcaactcggtgttaacacgattcgtacactttcaattgaggctgtccagaaggcaaatagtggacacccagggttacccatgggcgccgcgcctatggcgtacgccctgtggaccaaacatctgaaagtgaacccggtaactagccggaattgggtggatcgagatcgcttcgttttgtctgcgggtcatgggtccgccatgctgtatagtctgctgcacctcagcggctatcaggtcaccatcgacgatttaaaacaatttcgtcagtggggctcgaaaacgccgggccatcctgaagtgcatcacaccgatggtgtagaagcaactaccggcccgctaggtcagggtattggcatggcggtgggaatggctatggccgaagcgcatctcgcagcgacgtacaacaaggagaatttcaacgttgtggaccactatacctacgcattatgcggcgatggcgatctgatggaaggtgtctcccaagaggcgagcagtatggctggccacatgaaactgggtaaattgatagtcttatatgactctaatgacatctcgttggacgggccaacctcgaaagcatttacggaaaacgttggtgcccgctatgaagcctacgggtggcagcatattcttgtgaaggatggcaatgatctagaagctatctcaaacgcgattgaggccgcgaaggccgaaacaaccaaaccgacgctaatagaagtgaaaactgttatcggttatggagcgccgaaagaggggacgtctgccgtacacggtgcaccgctgggtgcagacgggattaagattgcgaaagaggtctacggctgggattacccagatttcaccgtgcctgaagaagtagctactcgctttcatgaaaaaatggttgaggacggtgaaaaagcggaagcgcaatggaatgaaaaatttgccaactataaaaatgcgtaccccgaactggcacagcagttcgaagatgcgttcgcgggcaaattaccagagaactgggatgccgagatgccgagctatgatgaaggccactcccaggctagccgcgtctccagcaaagatatgatccaagcgatcagtaacgccgttccgtcattgtggggaggatcggcagacctgtctggctctaacaatacaatggtagctgctgagacagactttgaaccgggtaattacgaggggcgtaacatttggttcggagtgcgtgaatttgcaatggcaaccgcgatgaacggcatccagcttcatggtggcacacggatttatggcggtacgttctttgtctttaccgattacctgcgtcctgctattcgcctggcgtcaatccaaaaggcaccggtgatttatgtactgacccacgactcggtcgccgttggcgaggatggcccgacgcatgaacccattgaacagcttgctagcgtgcgatgtatgcccggcgtgcatgtggtgcgcccggcggacggcaatgagacacgcgccgcatggaaaatagcgatggaaagtaccgaaacgccaaccatcctggtgctctccagacagaacttacccgttctaccgagcacgaaagaaaaggccgacgagatggtgaagaaaggggcatacgtcctgagcccggcgcaaggtgaaactccagaaggcatactgatcgccaccggttcggaggttgatctggcagtgaaggctcagaaagtcctggcggaaaatgggaaagatgtttcggtagttagtatgccgtcgttcgatctttttgaagcccagagtgcggaatataaggaatcagtccttccgaaagccgtaactaaaagagtagcgattgaagctgcggcaccgttcggatgggaaaggtatgtcgggactgaaggcaccacgatcaccattaatcattttggtgcctctgccccaggcaacaaaatcctggaggagttcggatttaccgtggaaaatgtagtcaagacatacgaagagctgtag

SEQ ID NO: 237 - DNA, 2076 bp, Artificial Sequence
  Synthetic
atgactgacaccaatacggcgatccatgaggatggctctcttgaacgtttaacaattgataccatacggacgctgtcaatggatgccgtccaaaaagcaaacagcggtcaccccggaaccccgatggctctggcgcctgtagggtacactctatggagtcagtttttgaggtatgacccagccaagccggactggccgaaccgcgatcgcttcgtgctctcggttggccatgcatccatgctgttatattcactgattcacctagcgggtatcgaagaaattgatgccgacggtaataaaacaggccgtccggcgctgagcttggatgacctgaaaggctttcgccagctctcgtctcgtacccccggccatccagagttccgacacacgaccggggtggaaaccactacgggtcctctgggagctggttgtagcaactctgtcggcatggcaattgcagagcgctggctggctgcgagatacaaccgcccggaatttaccctgttcgatcatgatgtttatacattgtgcggcgatggcgacatgatggaaggtgtggccgctgaagcggccagtttagcgggtcacttaaaactttccaatctgtgctggatctacgattctaatcatatcagcattgagggtgggaccgatttagcgtttgacgaagatgttgggctgcgttttcaggcctatggctggaacgtgattcacctggatgatgcgaatgacacgaaggcattcgccaaagcgattgaaaccttcaaagccacggacgataagccgacgtttatagtcgtgcatagtgtaatcggatggggtagcccgaaagcgggcagtgaaaaagcccacggcgaaccattgggagaagataacgttcgggcgactaaaaaagcatacgggtggccggaggataaagatttttatatcccagaaggggtggctgaacatttccatgacgcgattgcagggagaggaggcgctttgcgtgaggagtgggaagcaacgtttgcgcgctaccgtgaagccaaccctgagcttggagcagaactcgcgttgatgctgaaggatgagctgccggaaggttgggacgccgatattccggactttccggccgatgaaaaaggtatggcatcgcgcgattccggcggcaaagttctgaatgccctggctaaacgtgtcccttggctgatcggaggttctgctgacctaagcccttcaaccaagactgacatcaagggcgcaccatcgttcgaagccaataactatggcggtcaaaactttcacttcggtgtacgtgaacatgggatgggtggtgtagtgaatggcatgaccctatcccatgtacgcggctacgggtcaacctttttggtattcgctgattatatgcgagcgccgattcgcctgagcgcaattatggaacttgcatcggtctgggtgtttacgcacgatagcatcggggtcggcgaggacggacccacccaccagcccatagagcatctggcgaccctgagagcaatcccaggcctggatactattcgtccgggagacgctaatgaagtcgcgtacagttggcgcgctgcgctcgaagatgcgagccgtccgacagctctcatctttagtcggcaggccttgcccaccctggatcgaagcaaatatgcgtctgcggagggcacactgaaaggtggttatgtgttagcggactgtgaaggaactccggaagttattcttatcgcaactggtagtgaactctcacttgtggttcaagcacatgagaagctgagcgcagatggcatcaaatctcgcgtggtgagtatgccgagttggtataggtacgaactgcaatccgaagattacaaagaatcggttcttccatcctcagttcctagccgcctggcagtggagcaggcgggggagatgggctggcatcgttatgtcgggctcaagggtcggaccattaccatgagcacattcggtgcatcggcgcccatttcgaaattacaggataaatatggcttcacgctggataacgtagttaaagttgccagagaaatgctggaatccaacaacggctag

SEQ ID NO: 238 - DNA, 1992 bp, Artificial Sequence
  Synthetic
atgcctagccgtaaggaattggcaaatgctatcagagtcttaagtatggatgccgtacaaaaagcgaaatcaggtcacccaggggcgccgatgggaatggccgacattgcagaggttctgtggcgagattacctcaaacataacccgacaaaccccgaatgggcggatagggaccggttcatactttcgaatggccatggctctatgctgatttattccctgctgcacttgagcggttatgacctgccgatcgatgaaattaaaaactttcgccagatgcatagcaaaacgccgggccacccggagtacggttatgcgccaggcattgaaaccactacgggtcctctagggcagggcatcaccaatgctgtgggaatggctttagccgagaaggcgctggcagcccaatttaaccgcgaaggtcatgatattgtggatcactatacctacgctttcatgggcgatggctgcctgatggaaggcatctcccatgaagcgtgttcacttgccgggacgctgggactaggtaaattggttgcgttttgggacgataatggtatctcgattgacggagaggtagaaggatggtttagcgacgataccccagcccgcttcaaggcatacggttggcatgtgattagtggcgtcgatggtcatgattctgacgcaatatcagcggccatcgcggaggcgaaaagcgtgactgataaaccgacccttatctgctgtaaaacggtcattggctatggttccccaaacaaatctggcagccacgattgccacggggctccgctgggcgatgacgaaataacagcgtctcgcgaatttctcggatggaccggggaggcattcgaaattcctgaagatatttacgctcagtgggatggtaaagcgaagggtcagcaactggaaagttcgtgggatgaaaaatttgccgcgtatgcagacgcgtaccctgaactggcagccgagttcaagcggcgtactgctggcgaccttccggccgactgggcacagaaaagccaagaatatatcgaacagttacaggcaaatcccgcgaacccggcaagtcgtaaggcaagtcagaacgctctcaatgcttttgggccgattctgccagaatttatgggtggctcggccgatttggctgggtccaatttaacgatctgggacggctcaaaaggtctgacagcggacgatgcttctggaaactacgtttattatggcgttcgcgagttcggcatgtcggcaatcatgaatggtattgccctgcataaaggctttataccgtatggcgctaccttcctgatgtttatggaatatgcgcgcaacgccgtgcgtatggcggcgctcatgaaacaaccgtcgatcttcgtctacacccatgatagcattggcctaggggaggatggccccacccaccagccagttgaacaaattgcctcgatgcgtctgaccccgaacttgtacaactggcgtccctgcgatcaggtggaaagtgcaattgcgtggcaacaggcgatcgagagaaaagacggcccgacgtcccttatctttacgcgtcaaggtctagagcagcagtctcgcgatgcccagcagctcgcggatgtgaaaaagggtgggtacatactgtcatgtgacggtaatccagaactgattatcattgccactggcagcgaagtgcagctcgcgcaagattccgcaaaggagctgcgcagccagggtaaaaaagtacgtgtagtcagtatgccgtgtaccgatgctttcgaagagcagtctgccgagtataaagaatccgtgctcccttcggccgtaacacgaaggctggccgttgaggctggtatcgcggactactggtacaagtatgttgggctgaacggggctgttgtcggcatgacaacttttggtgaaagcgcccccgccaatgaactttttgaatttttcggattcacggtggaaaacattgtcaataaagcgaacgcgttattctag

SEQ ID NO: 239 - DNA, 1981 bp, Artificial Sequence
  Synthetic
tgtcgcgacaatccgtaccttatccattgacgccatcgaaaaagcaaaaagcggccaccctggaatgccaatgggggctgcgcccatggcctacgcactatggactaaaatgatgaatgtaaacccggaaaacccgaattggtttaacagagatcgcttcgtgctttctgcgggtcatggttcaatgctgctctattcgatgctgcatctgagcggctatgatgtttcaatggacgatctgaagaactttcggcagtggggcagcaaaacccctggtcacccggaatttgggcatacgccgggtgtggacgcaaccactggcccactgggccaaggaatagctatggccgtgggaatggcgcttgcagagcgtcacctggctgaaacatacaatcgagatgaatatcgcgttgtcgatcattacacctattcaatttgcggtgacggcgatttgatggaggggatttcgtccgaagcggcgagcctggcaggccacttaaaactgggacgtctcatcgttttgtacgattctaatgacattagtctggatggtgaactgaaccgctccttctctgagaatgtgaaacagcgttttgaagccatgaactgggaggtactttatgttgaagatggcaacaacatcgctgagattaccgctgcgttggaaaaggccaaacaaaatgaaaaacagccgacgctcatcgaggtcaagaccacgatcggttatgggtcgcccaacagggctggcaccagcggtgtgcatggcgccccgctggggagtgaagaagcgaaactaactaaagaagcctatgagtggacatacgaagaggatttctacgtgccctccgaagtttatgatcattttcgcgagacggttaaagaagatgggaaacgcaaagaacaggaatggaacgaactgttcagcgcgtataaaaaggcatatccggacttagcagagcagctcgaattaggtataaaaggcgacctgccgtcggggtgggacaaagaaattccggtctacgaaaagggctcctccctggcttcacgcgcgtctagcggtgaggtacttaatggtattgctaaacaagtgccattcttttttggcggctctgccgatttagcgggttccaataagacaaccatcaaaaatggcggtgatttcagtgcgaaggactatgccggacgaaacatttggtttggagttcgtgagttcgcgatgggcgcagcattgaatggtatggcactgcacggtggattaagagtgtttgccggtacttttttcgtgttttcagattatctgcggccggccatccgtctggcggcgctgatgggcctcccagtaacctacgtctttactcatgactccattgcggtgggagaagatggccctacgcacgaacctatcgaacagcttgcatcgctgcgcgccctgccgaatctgagcgtgattcgtccggccgacggcaacgagacagcggcggcttggaaattggcgctgcaaagtaaagaccagcccaccgcgctagtgttaacccgccagaacctgccgactattgatcaaagcgggcaggcggcatatgagggcgtagaacgaggagcgtacgttgtctcgaaaagtcagaacgagaagccggccgccatccttctagccagcgggagtgaagtgggtttggcagtggacgcccaaagcgaactccgtaaagaaggtatcgatgtatcggtagtttcagtcccttcatgggaccggtttgataagcagccacaagattacaaaaatgcagttctgccgtcggacgtaacgaaacgcttagctatcgagatgggaagcccgctggggtgggataaatatacgggtaccgaaggcgacatattggcaattgatcagtttggcgcttccgcgccaggcgaaacgattatgaaggagtacggattcaccgccgaaaacgtcgcggatagagttaaaaaactgcttcagaagtag

SEQ ID NO: 240 - DNA, 1998 bp, Artificial Sequence
  Synthetic
atgactaacaaagtggaagagttagctgtaaatacaattcggacgctttctatcgattcaattgaaaaggccaactcgggacaccccggcatgccgatgggggcagcgcctatggcgctaaatctctggaccaaacatatgaaccataatccggccaacccaaaatggagcaatcgtgaccgatttgttctgtccgctggtcacggcagtatgctgctgtacagcctgttgcatttatcaggttatgatgtcacccttgacgatctgaaaagcttccgccagttgggctctcgtacgccgggtcatccggagtatgggcacaccgacggcgtggaagcaactaccggcccactgggacaaggtatcgcgatggcggttggcatggccatggcagaacgccatctggcggccacgtacaatacagataaatatcccatagtggatcactttacctacgctatttgcggtgatggcgatctaatggagggggtaagtcaggaagccgcgagcttggcgggtcatctcaagctggaacgcctgatcgtcctctatgactccaacgacatttcgctggatggagatttacacgaatctttcagtgaaagcgttgaggaccgttttaaagcatatggatggcacgtggttagagtcgaagatggcaccgacatggaggagattcatcgcgccatcgaagaagcaaaacgagtagaccgtccgacgcttattgaggttaagaccgtgatcggttacgggagccctaacaaagcggcttcaagcgcatcccacggaagtccgctgggtacggaagaagtaaagctgactaaagaggcgtataaatggacatttgaagaagatttctatatccctgaagaagtcaaagcttacttcgctgccgtcaaggaagagggcgcggctaaagaagctgaatggaacgatttatttgcggcctataaagcagaatacccggaactggcggcgcagtacgaacgtgccttctcgggcgagctaccggaggggtttgaccaagcacttccggtgtatgaacatggtacctccctggctactcgggcgtctagcggcgaggcattgaatagcctggccgcgcataccccagaattattcggcggctcagccgatctggccggttctaacaaaaccacgttgaaaggcgaatcaaactttagtcgcgataattatgcggggagaaatatttggttcggtgtgcgcgagtttgcaatgggcgcagctctcaatggtatggcactgcatggcggtctgaaggtttttggtggcacattcttcgtcttttcagattacctgaggcccgcgattcgcctctcggcgttaatgggagtgccagtgacgtatgtcctcactcacgactctgtcgcggtgggcgaagatggcccgacccacgaacctgtagaacatctggccgcccttcgtgccatgccgggtctgagtgtggttcgtccgggcgacggcaacgagacagccgcggcgtggaaaatagccctggagtcgtcggatcgcccgaccgttctggtactgtctcgtcagaacgtggacacgttaaaaggaaccgacaagaaagcgtacgaaggggtaaagaaaggggcgtacatagtttccgaacctcaagataaaccggaggtggtccttttggcaacaggtagcgaggtaccgctggctgtgaaagcacaggcggcactcgcggacgaaggtatcgatgctagtgtcgtgtcgatgccttcctgggatcgctttgaggagcaaccccaggaatataaagatgcggttattccacgtgacgtgaaagcgcggttggccatcgaaatgggcagcagcttcgggtgggcaaagtatgtgggcgatgagggtgatgttcttggaattgatacctttggcgcctccggtgccggcgaagccgtaatcgcggaatttgggttcacggtggataacgttgttagtcgcgcgaaagcgttactgaaaaagtag

SEQ ID NO: 241 - protein, 664 aa, Enterococcus mundtii
MFDKIDQLGVNTIRTLSVDAVQKANSGHPGLPMGAAPMAYALWTKHLKVNPKTSKNWADRDRFVLSAGHGSAMLYSLLHLAGYQVTIDDLKQFRQWESKTPGHPEVNHTDGVEATTGPLGQGIAMAVGMAMAEAHLAATYNKDQFNVVDHYTYALCGDGDLMEGVSQEASSMAGHMKLGKLIVLYDSNDISLDGPTSKAFTENVGARYEAYGWQHILVKDGNDLEAISKAIEEAKAETDKPTLIEVKTVIGFGAPNQGTSAVHGAPLGLEGIQKAKEIYGWEYPDFTVPEEVAERFRQTMVEEGEKAENAWREMFAAYKAAYPELAQQFEDAFAGKLPENWDAELPTYDEGESQASRVSSKEVIQELSKAIPSFWGGSADLSGSNNTMVTADKDFTPEHYEGRNIWFGVREFAMASAMNGIQLHGGTRIYGGTFFVFVDYLRPAVRLAAIQNTPVIFVLTHDSVAVGEDGPTHEPVEQLASVRSMPGVHVLRPADGNETRAAWKVAMESTDTPTILVLSRQNLPVLPTTKEVADDMVKKGAYVLSPAKGEQPEGILIATGSEVDLAVKAQKVLAEQGKDVSVVSMPSFDLFEQQSAEYQESVLPKSVTKRVAIEAAASFGWERYVGIEGQTITIDHFGASAPGNKILEEFGFTVDNVVNVFNQL

SEQ ID NO: 242 - protein, 664 aa, Enterococcus thailandicus
MFDKIDQLGVNTIRTLSIEAVQKANSGHPGLPMGAAPMAYALWTKHLKVNPVTSRNWVDRDRFVLSAGHGSAMLYSLLHLSGYQVTIDDLKQFRQWGSKTPGHPEVHHTDGVEATTGPLGQGIGMAVGMAMAEAHLAATYNKENFNVVDHYTYALCGDGDLMEGVSQEASSMAGHMKLGKLIVLYDSNDISLDGPTSKAFTENVGARYEAYGWQHILVKDGNDLEAISNAIEAAKAETTKPTLIEVKTVIGYGAPKEGTSAVHGAPLGADGIKIAKEVYGWDYPDFTVPEEVATRFHEKMVEDGEKAEAQWNEKFANYKNAYPELAQQFEDAFAGKLPENWDAEMPSYDEGHSQASRVSSKDMIQAISNAVPSLWGGSADLSGSNNTMVAAETDFEPGNYEGRNIWFGVREFAMATAMNGIQLHGGTRIYGGTFFVFTDYLRPAIRLASIQKAPVIYVLTHDSVAVGEDGPTHEPIEQLASVRCMPGVHVVRPADGNETRAAWKIAMESTETPTILVLSRQNLPVLPSTKEKADEMVKKGAYVLSPAQGETPEGILIATGSEVDLAVKAQKVLAENGKDVSVVSMPSFDLFEAQSAEYKESVLPKAVTKRVAIEAAAPFGWERYVGTEGTTITINHFGASAPGNKILEEFGFTVENVVKTYEEL

SEQ ID NO: 243 - protein, 691 aa, Sphingomonas sp.
MTDTNTAIHEDGSLERLTIDTIRTLSMDAVQKANSGHPGTPMALAPVGYTLWSQFLRYDPAKPDWPNRDRFVLSVGHASMLLYSLIHLAGIEEIDADGNKTGRPALSLDDLKGFRQLSSRTPGHPEFRHTTGVETTTGPLGAGCSNSVGMAIAERWLAARYNRPEFTLFDHDVYTLCGDGDMMEGVAAEAASLAGHLKLSNLCWIYDSNHISIEGGTDLAFDEDVGLRFQAYGWNVIHLDDANDTKAFAKAIETFKATDDKPTFIVVHSVIGWGSPKAGSEKAHGEPLGEDNVRATKKAYGWPEDKDFYIPEGVAEHFHDAIAGRGGALREEWEATFARYREANPELGAELALMLKDELPEGWDADIPDFPADEKGMASRDSGGKVLNALAKRVPWLIGGSADLSPSTKTDIKGAPSFEANNYGGQNFHFGVREHGMGGVVNGMTLSHVRGYGSTFLVFADYMRAPIRLSAIMELASVWVFTHDSIGVGEDGPTHQPIEHLATLRAIPGLDTIRPGDANEVAYSWRAALEDASRPTALIFSRQALPTLDRSKYASAEGTLKGGYVLADCEGTPEVILIATGSELSLVVQAHEKLSADGIKSRVVSMPSWYRYELQSEDYKESVLPSSVPSRLAVEQAGEMGWHRYVGLKGRTITMSTFGASAPISKLQDKYGFTLDNVVKVAREMLESNNG

SEQ ID NO: 244 - protein, 663 aa, Pseudoalteromonas sp.
MPSRKELANAIRVLSMDAVQKAKSGHPGAPMGMADIAEVLWRDYLKHNPTNPEWADRDRFILSNGHGSMLIYSLLHLSGYDLPIDEIKNFRQMHSKTPGHPEYGYAPGIETTTGPLGQGITNAVGMALAEKALAAQFNREGHDIVDHYTYAFMGDGCLMEGISHEACSLAGTLGLGKLVAFWDDNGISIDGEVEGWFSDDTPARFKAYGWHVISGVDGHDSDAISAAIAEAKSVTDKPTLICCKTVIGYGSPNKSGSHDCHGAPLGDDEITASREFLGWTGEAFEIPEDIYAQWDGKAKGQQLESSWDEKFAAYADAYPELAAEFKRRTAGDLPADWAQKSQEYIEQLQANPANPASRKASQNALNAFGPILPEFMGGSADLAGSNLTIWDGSKGLTADDASGNYVYYGVREFGMSAIMNGIALHKGFIPYGATFLMFMEYARNAVRMAALMKQPSIFVYTHDSIGLGEDGPTHQPVEQIASMRLTPNLYNWRPCDQVESAIAWQQAIERKDGPTSLIFTRQGLEQQSRDAQQLADVKKGGYILSCDGNPELIIIATGSEVQLAQDSAKELRSQGKKVRVVSMPCTDAFEEQSAEYKESVLPSAVTRRLAVEAGIADYWYKYVGLNGAVVGMTTFGESAPANELFEFFGFTVENIVNKANALF

SEQ ID NO: 245 - protein, 667 aa, Bacillus sonorensis
MKTIELKSVATIRTLSIDAIEKAKSGHPGMPMGAAPMAYALWTKMMNVNPENPNWFNRDRFVLSAGHGSMLLYSMLHLSGYDVSMDDLKNFRQWGSKTPGHPEFGHTPGVDATTGPLGQGIAMAVGMALAERHLAETYNRDEYRVVDHYTYSICGDGDLMEGISSEAASLAGHLKLGRLIVLYDSNDISLDGELNRSFSENVKQRFEAMNWEVLYVEDGNNIAEITAALEKAKQNEKQPTLIEVKTTIGYGSPNRAGTSGVHGAPLGSEEAKLTKEAYEWTYEEDFYVPSEVYDHFRETVKEDGKRKEQEWNELFSAYKKAYPDLAEQLELGIKGDLPSGWDKEIPVYEKGSSLASRASSGEVLNGIAKQVPFFFGGSADLAGSNKTTIKNGGDFSAKDYAGRNIWFGVREFAMGAALNGMALHGGLRVFAGTFFVFSDYLRPAIRLAALMGLPVTYVFTHDSIAVGEDGPTHEPIEQLASLRALPNLSVIRPADGNETAAAWKLALQSKDQPTALVLTRQNLPTIDQSGQAAYEGVERGAYVVSKSQNEKPAAILLASGSEVGLAVDAQSELRKEGIDVSVVSVPSWDRFDKQPQDYKNAVLPSDVTKRLAIEMGSPLGWDKYTGTEGDILAIDQFGASAPGETIMKEYGFTAENVADRVKKLLQK

SEQ ID NO: 246 - protein, 665 aa, Bacillus clausii
MTNKVEELAVNTIRTLSIDSIEKANSGHPGMPMGAAPMALNLWTKHMNHNPANPKWSNRDRFVLSAGHGSMLLYSLLHLSGYDVTLDDLKSFRQLGSRTPGHPEYGHTDGVEATTGPLGQGIAMAVGMAMAERHLAATYNTDKYPIVDHFTYAICGDGDLMEGVSQEAASLAGHLKLERLIVLYDSNDISLDGDLHESFSESVEDRFKAYGWHVVRVEDGTDMEEIHRAIEEAKRVDRPTLIEVKTVIGYGSPNKAASSASHGSPLGTEEVKLTKEAYKWTFEEDFYIPEEVKAYFAAVKEEGAAKEAEWNDLFAAYKAEYPELAAQYERAFSGELPEGFDQALPVYEHGTSLATRASSGEALNSLAAHTPELFGGSADLAGSNKTTLKGESNFSRDNYAGRNIWFGVREFAMGAALNGMALHGGLKVFGGTFFVFSDYLRPAIRLSALMGVPVTYVLTHDSVAVGEDGPTHEPVEHLAALRAMPGLSVVRPGDGNETAAAWKIALESSDRPTVLVLSRQNVDTLKGTDKKAYEGVKKGAYIVSEPQDKPEVVLLATGSEVPLAVKAQAALADEGIDASVVSMPSWDRFEEQPQEYKDAVIPRDVKARLAIEMGSSFGWAKYVGDEGDVLGIDTFGASGAGEAVIAEFGFTVDNVVSRAKALLKK

SEQ ID NO: 247 - DNA, 1533 bp, Artificial Sequence
  Synthetic
atggaagtggccatgcccttgcgaatggatgcgacgggctctagctcgaaaattcacgctggtggaaagcgcgacaactcaggggcagtagcgttcgattttgttatcgtcggcgccacaggtgaccttaccatgcggaaactcctgccggcattttatgagtgcttcaggcgtcgccagatagaaaaatccactaaaatcattggcgtggcgcgtagtggtctgagcgttgaggattaccgcgcacgtgctcatgaagccttaaagggttttgtcgcgaccagctcctatgacgatgcgacgattcaagattttctgggactggttgaatacgtgtctttagatatgtcggataaagacgcggattggaccgggctgagagcccagctcagtactgaacgcgatcgtccaagagtgttctatgtagccaccgcaccgaaactatacgtccctacagcggacgctatcgcccataatgaactgatcaccgagtcatcacgcattgtgctggagaagccgattggcacggaccaagcaactgctgccgaaatcaatgatggcgtcggccagcactttaccgaggaacagattttccgtatcgatcattatttgggtaaacaaacggttcagaacatactagcgcttcgttttgccaacccaattctggaacgcgtctggaatacggatagcatcgcgcacgtacagattaccgccgcggaaaccgtaggggtcggaaaaaggggcccctattacgattcagcaggggcattgcgcgacatggttcaaaaccatcttctgcaagtcctgagcctggtggcgatggagccgccgaccgcgttctccgctatggacctccgggatgaaaaattaaaaatcctccgtgcattgaagcctatgtctgatcacgacattgctactgacacagtgcgcgcgcagtatggtgaaggccatgtgaatggtaaactgattccgggatacttggatgaccttggcgcgccgacgagtactactgaaacatatctggccatccgggccgagatccgaaccgcacgttgggctggtgttccgttttatattcgaaccggtaagcagatggcgcgcaaagaaacaaccgtggtaattcaattccgcccccagccatgggccatttttacggataacccagaacctagtcagttggttctgcgtatccagcccaatgaaggtgtaagcctgagtctggcatctaaagacccggcgtccgagcagtaccgtctacgcgaggcggtgctggatgtagattatgttaaagcctttaacacccgctatccggactcttacgaagatttattaatggctgcggtgagaggcgaccaagtgctgttcatccgtcgtgatgaggtcgaagcgtcgtggcgctggatcgagcctattctccacggatgggaagaaaacatacggccgttagaaatttacccggccggcacccagggcccggcatcaagcgacgagctgctggcacgtgacggctttgtgtggaaagaaaacacctag

SEQ ID NO: 248 - DNA, 1470 bp, Artificial Sequence
  Synthetic
atgcaaacgtgtacaattatcatatttggtgcgaccggagatttgtctaagaaaaaattactgccagctctgtatcacctcgacgccgagcagcgacttactgcggataccaaaattatctgcctgggccgccgggaaatgccccaggcagaatggctggagcaggtcacggaatacgtttccgacaaagccaggggcggtgtagatgcagcgaccctggaacgcttcctcgcacgtgtgtcgtttttcaagcatgatattaacaccccggaagattataaagcgatggccgatttgctgaaaaaacctgagaatagcttttcaagcaacatcgtgttttaccttagtatttcgccgtctttattcggggtcgtgggcgaccaactggctgccgttggtcttaataacgaacaggacggctggcgtcatctggttgtggagaaaccgtttgggtatgatcagaagtcagccgaacaactggaacaaattttgcgcaagaacttcacggagcagcagacttacagaatcgaccactatttgggaaaaggtaccgtacagaatatctttgtctttaggttcgctaatctactcctggaaccgctctggaatcataaatacattgaccatgtgcagatcacccatgcggaacagcaaggcgtcggtgggcgtgccggttattatgatggcagcggagcactgcgcgatatgatacaatcgcacctgttacaggttatggcgcttgttgcgatggaaccaccggcagatttagatgacgagtccctgcgggatgaaaaagtgaaggtactgaaaagcattcgccctatcacgtcagatatggtggaccagcacgcgtttcgtggccagtattccgcaggcgaagtcaacgggcaaaaaattccgggttacttggaggatgaagaagttcccaaggacagtgttacggagacttatgcggccatgaaaatatatattgacaactggcgctggcgtggtgtgccattctacctgagaacagggaaatgcatgccggaaagcaaagctatgatcgcaattcgtttcaaaaaaccgccgttagagctgttcaaagataccaaaattggtgatagtcacgccaactggatcgtcatgggtctgcaacccgataatacgttgcgtattgagctacaggcgaaacagccaggtctggaaatcaaggcacatactgtggcgctggaaaccgtagagtctgaagataagaaacataaactcgatgcttatgaagcacttatcttagacgctatacagggcgaccgttcactgtttctgcgctctgatgaagtgaacctggcctggaaagcggtggacccgattttggaaaagtgggcgcaggataaagattttgtacacacttaccctgcgggcacctggggccccgacgcagtctccacattgatggatgatccatgtcacgtctggcgaaataacctatag

SEQ ID NO: 249 - DNA, 1521 bp, Artificial Sequence
  Synthetic
atgaaaaactatacgactcctaagtgtattatagtgatctttggggcaaccggtgacttggctaaaaggaaattattcccaagtctgtttcgtctcttccgacaaggcaaaatctccgagaattttgccgtcgtaggagttgcgcgccgcccgctttcaacagaagaatttcgggagaacgtgaagcagtctattcacaatctgcaagaagaaaacatgacccatgatacgttcgcgagccatttttactatcaccccttcgatgttaccaacctgagcagttaccaggagctgaaatcgttactcattacactagatggcagatatttcactgaaggtaatcgtatgttttatctggccatggcgccggactttttcgggaccatcgcaacgaatctgaaatcagaaggtttgaccagcacagagggatggattcgtctggtaattgaaaagccgtttggccatgactatgaatcggctcaggtcctcaacgatcagatccgccacgcgttcacggaggatgaaatttaccgaatagatcattacttaggcaaagaaatggtgcaaaatatcaaagtgattcgtttcgccaacgccatctttgagcctctgtggaacaatcagtatatcgctaacattcagatcacctcttctgaaactctgggtgtcgaagaacgcggccgttattacgaagattcgggggcactgcgcgacatggtacaaaatcatatgttgcagatggtggcgcttttagcgatggagccgccgattaaactgaccgcgaatgaaattcggtccgaaaaggttaaagtgctgagggcactgcaaccacttagcgaagagacagttgaacacaactttgtgcgcggtcaatatggccccggtatgattgatgaggagaaagttattagttaccgcgaagagaatgctgttgattccgaaagcaatacggaaacctttgtgtccggcaagctgatgatcgaagatttccgttggtcgggcgtaccgttctacatacgtacaggcaaacgcatgcaggagaaatccaccgagattgtcatccagtttaaggacctaccaatgaacctttattttaacaaagaaaaaaaagtacatcccaacttactggtgatccacattcagccggaagaaggtataacccttcacttgaacgcccaaaaaacggacagcgggaccacttctacgccgatccagctaagttactgcaataactgcatggataaaatgaatactcctgaagcctatcaggtccttctgtatgactgtatgcgtggtgattcgacgaactttacccattgggacgaggtgtgcctgtcctggaagttcgtagataccatcagctcagtgtggcgcaataaaccagcaaagcattttccgaactacgaatcaggctcgatgggaccgaaagaaagtgatgcactgttagaacgggaccggttccattggtggccgaccattacgagccaccttaaaggagaatcctacaacgaaaatacatag

SEQ ID NO: 250 - DNA, 1545 bp, Artificial Sequence
  Synthetic
atgactacgtccgcgcccccttgggctggtcagataattcaagacggggtcggctgccatttggaaggagcaccagatccgtgtgtggtagttatctttggcgcctcaggtgatttatgccaccgcaaactcatgccggcgctttacgacctgttcgtgaaccatggcctgcaagagtcgctggcggttgtcggttgtgcccgtacagcatatgatgatgaccagtttagagaactgatggcacaggctgttgccgaagctggcttagatttggcgcgctgggacgcattcgcgcgtcggttgttttatcagccgttaacctacgatgaccctgccagcttcgccccactacgccaccgtctggaggtgattgatcgagactgcgggggatgtggtaatcgcatctataacctggcgatcccgccgcagctttatgcggatgtcgcacgctctctgagtgcggcaggtatgaatcaaagcgatggccccggatggctgcgtctggtagtggaaaagccatttggtgatgatctccagtctgcccggcaactcaacgcagccttggcggagggctttgccgaagaacagattttccgcattgatcattacttggcgaaagacaccgtccaaaatctgatgctgtttcggttcgctaacgctgtatttgagccgctgtgggaccgaaaatacgtggatttcgtagccatcaccgcggctgaaacgctgggcgttgaacaccgtgcaggctattatgaacaggcgggggttcttcgtgacatgtttcagaatcatatgctgcaactgttagcgctcgtggccggggaggccccgccgaacatggacgcagagcgtgtccgcgatgaaaaaattcgcctctttcgttgcttgaggccgttacctgctgacaatctggatggtactttggttttaggtcagtacgcggctgggagagttgccggccaggaagtggtggcctatagagacgagccaggtgtcgcaccgggcagcctgacgcctaccttcgcggccctacgtgtgtttgtcgataactggcgctggcagggtgtgccattctacctgtgttcaggcaaacgcctggcgaagaaacgtacctcgattgatatacagtttaaacaagtgccacattccctgttccgccaggctcttggcgaacacatcacgagcaaccgattatcactgggaatccaaccggaagagactattacactgagtatccagaccaagaaacccggtccgaaactctgcttgcgcactgtgggaatgggctttgattttcgggcgggtggtgaacctatgcacgacgcctacgaaaaggtactgctagatgccatgctaggagatcataccctgttctggcgtcaggacggcgtcgaactttgctggcagtggttagaaccgctgctgcgtgcctgtgaggcatgcgcggatagggggaagcgccttcacttttatcccgccggaggctgggggccgccccaagcgcgtgacgtagcaccgctcctggcggatcgcaacgaagattag

SEQ ID NO: 251 - DNA, 1593 bp, Artificial Sequence
  Synthetic
atgaataaccccacgaaacctgactctttaatcctggtcattttcggagcctccggcgatttgactaagcgcaaactgataccgagtctctatcagctttttaaacaagcaaagctgccgaaacgatttgcggtactggggttgggtcggacagcttacgatagcgcgagctatagaccacatctagacgaatcattaaaaaaatacctggccgagggtgaatatgatccgtcgctggcggagcagttccttgcttcagttcactacttgagtatggacccagcgctcgaagaagaatatccgaaactgaaatcacgcctgcaagaactggatgagcagattgataacccggcaaattatatctactatctcagcacccctccttccctgtacggcgtggtgccgcttcatcttgcatctgttggcctgaaccgtgaggaatgtgattcgccagatggtcgctgccaccttaacgcccatcgtggcgaagatggagtgccccgtccgattcgcaggatcattatcgagaagccgtttgggtacgacctgaaatctgccgaagaattaaatgaaatttatcgtagctgctttagggagcatcagttataccgtatagatcactttttaggtaaagaaacggtccaggacattatggctctgcgcttcgcgaacggcattttcgaacccttatggaatcggaactatatcgatagaatcgaagtcaccgccgtagaaaacatgggagttgagagtcgtggtggcttttatgacgagactggcgcgctgcgtgatatggtgcaaaatcacctgtctcagctagtagcgttggtggcaatggaaccgccagttcaattcaacgcagacctgttccgtaatgaagtggttaaagtgtatcaggcttttcgcccaatgagcgaagaagatattagccgctcggttattcgtggtcaatacaccgagtccgagtggaaaggtgagtatcatcgcgggtatcgcgaagaggacaagatcaatcctgaatcacgaaccgaaacgtttgtggcaatgaaactgcatatagataactggagatggcatggcgtacccttttacatccgtacgggcaagatgatgccaaccaaagttaccgagattgtcatccactttaaaccgactccgcacaagatgttcgctggggccgatggtcggagtattccgaatcagctcattattaggatccagccgaacgaaggtatcgtgctgaaattcggcgcgaaagtaccggggagtggctttgaagtcaaaaaagtctcaatgaatttcacctacgatcagctaggtggcttagcctcgggggacgcttattcacgtctgctggaggatagcatgctgggagactcgacattgtttacgcgcagtgacgcggtagaaatgagctggcgttttttcgacccaatccttcgcgcatggcaggatgaacattttcccctctatggttacccggccgggacatggggaccgaagcaatccgacgaaatcatggatggcgattgttacaactggaccaacccttgcaagaatctgaccaacagcgaattgtactgtgagttatag

SEQ ID NO: 252 - DNA, 1470 bp, Artificial Sequence
  Synthetic
atgaatacgattaacaacaaactccctactacaataatcattttcggagcctctggcgatttgacccagcgcaagctgatcccgagtctgtttaatttatttcgtaaacgaaaaaccccaaaacaacttcagattatcgggtgtggtacgaccgaatttagcaacgagtcattccggaaacatctgctagaaggtatgaagaatttcgctacttataaatttacccaagaggaatggaacattttcgcatccaatctgcgttacttaacgggcacatatagcgaagtggaggactttaagaaactggcggaacagttgaaaaagtacgaagataacgaaaacaccaatcgcctttattacatggcggtaccgcccaaaattttcccgtcgatcatcgagaacctgcacaaaactgatcagctcgaagagcgcaaaggctattggcgtagagtcgttattgaaaagccgtttggaacctccctggaaacggcaattaccctgaataaacaggtgcataaagccctacacgaaaaccaagtttaccgtattgaccattatttaggtaaagaaacagtacagaatatcctgttcactcgctttgccaatactatctatgaaccgatttggaaccgcaattatatcgatcacgtccagatcaccgtggcggaaaaagtgggcctggagcatagggctgggtactacgacggcgttggtgtcctacgtgatatgttccagaaccatctgttacaactcctgacgttggtcgcgatggaaccacccgcgtcttttagcgcctcacacctgagaaacgagaaagtgaaagtgctgagtgcaattaagcctctcagcccggaggaagttcttacaaataccgtacgcgcccaatataaaggttactcgcaagaaaaaggggtaggagctgagtctaccactgctacgttcgcggcgttaagactgtttattaacaactggcgttggcagggcgtgccgttctacttgcgttccggcaaaaatctcagtgagaagcagtcgcagattataatccagtttaaagaaccgccacttgcaatgtttcctatgcagaccatgaaaccgaacatgttggtcctgtttctccagccagatgagggtgttcatctccgtttcgaagcaaaagctcctgacaaagttaatgaaacgcgcagcgtcgatatggaatttcactatgacgaggcatttggtaagagtgcgattccggaagcatatgaacgcctgctgctggatgccatccaaggcgatgcctcgctgttcacccgcgctgatgaagtggagactgcctggtctatcatagaccccatattgcagacgtgggacacccatcaaacgccgccgctggcggtctataaaccaagctcttggggaccggcggaatcagatatgctgctagccaaagatggtcggcgatggttaaacgaggaaagcgacgcctag

SEQ ID NO: 253 - protein, 510 aa, Acetobacter aceti
MEVAMPLRMDATGSSSKIHAGGKRDNSGAVAFDFVIVGATGDLTMRKLLPAFYECFRRRQIEKSTKIIGVARSGLSVEDYRARAHEALKGFVATSSYDDATIQDFLGLVEYVSLDMSDKDADWTGLRAQLSTERDRPRVFYVATAPKLYVPTADAIAHNELITESSRIVLEKPIGTDQATAAEINDGVGQHFTEEQIFRIDHYLGKQTVQNILALRFANPILERVWNTDSIAHVQITAAETVGVGKRGPYYDSAGALRDMVQNHLLQVLSLVAMEPPTAFSAMDLRDEKLKILRALKPMSDHDIATDTVRAQYGEGHVNGKLIPGYLDDLGAPTSTTETYLAIRAEIRTARWAGVPFYIRTGKQMARKETTVVIQFRPQPWAIFTDNPEPSQLVLRIQPNEGVSLSLASKDPASEQYRLREAVLDVDYVKAFNTRYPDSYEDLLMAAVRGDQVLFIRRDEVEASWRWIEPILHGWEENIRPLEIYPAGTQGPASSDELLARDGFVWKENT

SEQ ID NO: 254 - protein, 489 aa, Methylophaga lonarensis
MQTCTIIIFGATGDLSKKKLLPALYHLDAEQRLTADTKIICLGRREMPQAEWLEQVTEYVSDKARGGVDAATLERFLARVSFFKHDINTPEDYKAMADLLKKPENSFSSNIVFYLSISPSLFGVVGDQLAAVGLNNEQDGWRHLVVEKPFGYDQKSAEQLEQILRKNFTEQQTYRIDHYLGKGTVQNIFVFRFANLLLEPLWNHKYIDHVQITHAEQQGVGGRAGYYDGSGALRDMIQSHLLQVMALVAMEPPADLDDESLRDEKVKVLKSIRPITSDMVDQHAFRGQYSAGEVNGQKIPGYLEDEEVPKDSVTETYAAMKIYIDNWRWRGVPFYLRTGKCMPESKAMIAIRFKKPPLELFKDTKIGDSHANWIVMGLQPDNTLRIELQAKQPGLEIKAHTVALETVESEDKKHKLDAYEALILDAIQGDRSLFLRSDEVNLAWKAVDPILEKWAQDKDFVHTYPAGTWGPDAVSTLMDDPCHVWRNNL

SEQ ID NO: 255 - protein, 506 aa, Bacillus pseudomycoides
MKNYTTPKCIIVIFGATGDLAKRKLFPSLFRLFRQGKISENFAVVGVARRPLSTEEFRENVKQSIHNLQEENMTHDTFASHFYYHPFDVTNLSSYQELKSLLITLDGRYFTEGNRMFYLAMAPDFFGTIATNLKSEGLTSTEGWIRLVIEKPFGHDYESAQVLNDQIRHAFTEDEIYRIDHYLGKEMVQNIKVIRFANAIFEPLWNNQYIANIQITSSETLGVEERGRYYEDSGALRDMVQNHMLQMVALLAMEPPIKLTANEIRSEKVKVLRALQPLSEETVEHNFVRGQYGPGMIDEEKVISYREENAVDSESNTETFVSGKLMIEDFRWSGVPFYIRTGKRMQEKSTEIVIQFKDLPMNLYFNKEKKVHPNLLVIHIQPEEGITLHLNAQKTDSGTTSTPIQLSYCNNCMDKMNTPEAYQVLLYDCMRGDSTNFTHWDEVCLSWKFVDTISSVWRNKPAKHFPNYESGSMGPKESDALLERDRFHWWPTITSHLKGESYNENT

SEQ ID NO: 256 - protein, 514 aa, Desulfarculus baarsii
MTTSAPPWAGQIIQDGVGCHLEGAPDPCVVVIFGASGDLCHRKLMPALYDLFVNHGLQESLAVVGCARTAYDDDQFRELMAQAVAEAGLDLARWDAFARRLFYQPLTYDDPASFAPLRHRLEVIDRDCGGCGNRIYNLAIPPQLYADVARSLSAAGMNQSDGPGWLRLVVEKPFGDDLQSARQLNAALAEGFAEEQIFRIDHYLAKDTVQNLMLFRFANAVFEPLWDRKYVDFVAITAAETLGVEHRAGYYEQAGVLRDMFQNHMLQLLALVAGEAPPNMDAERVRDEKIRLFRCLRPLPADNLDGTLVLGQYAAGRVAGQEVVAYRDEPGVAPGSLTPTFAALRVFVDNWRWQGVPFYLCSGKRLAKKRTSIDIQFKQVPHSLFRQALGEHITSNRLSLGIQPEETITLSIQTKKPGPKLCLRTVGMGFDFRAGGEPMHDAYEKVLLDAMLGDHTLFWRQDGVELCWQWLEPLLRACEACADRGKRLHFYPAGGWGPPQARDVAPLLADRNED

SEQ ID NO: 257 - protein, 530 aa, Porphyromonas sp.
MNNPTKPDSLILVIFGASGDLTKRKLIPSLYQLFKQAKLPKRFAVLGLGRTAYDSASYRPHLDESLKKYLAEGEYDPSLAEQFLASVHYLSMDPALEEEYPKLKSRLQELDEQIDNPANYIYYLSTPPSLYGVVPLHLASVGLNREECDSPDGRCHLNAHRGEDGVPRPIRRIIIEKPFGYDLKSAEELNEIYRSCFREHQLYRIDHFLGKETVQDIMALRFANGIFEPLWNRNYIDRIEVTAVENMGVESRGGFYDETGALRDMVQNHLSQLVALVAMEPPVQFNADLFRNEVVKVYQAFRPMSEEDISRSVIRGQYTESEWKGEYHRGYREEDKINPESRTETFVAMKLHIDNWRWHGVPFYIRTGKMMPTKVTEIVIHFKPTPHKMFAGADGRSIPNQLIIRIQPNEGIVLKFGAKVPGSGFEVKKVSMNFTYDQLGGLASGDAYSRLLEDSMLGDSTLFTRSDAVEMSWRFFDPILRAWQDEHFPLYGYPAGTWGPKQSDEIMDGDCYNWTNPCKNLTNSELYCEL

SEQ ID NO: 258 - protein, 489 aa, Chloroflexi bacterium
MNTINNKLPTTIIIFGASGDLTQRKLIPSLFNLFRKRKTPKQLQIIGCGTTEFSNESFRKHLLEGMKNFATYKFTQEEWNIFASNLRYLTGTYSEVEDFKKLAEQLKKYEDNENTNRLYYMAVPPKIFPSIIENLHKTDQLEERKGYWRRVVIEKPFGTSLETAITLNKQVHKALHENQVYRIDHYLGKETVQNILFTRFANTIYEPIWNRNYIDHVQITVAEKVGLEHRAGYYDGVGVLRDMFQNHLLQLLTLVAMEPPASFSASHLRNEKVKVLSAIKPLSPEEVLTNTVRAQYKGYSQEKGVGAESTTATFAALRLFINNWRWQGVPFYLRSGKNLSEKQSQIIIQFKEPPLAMFPMQTMKPNMLVLFLQPDEGVHLRFEAKAPDKVNETRSVDMEFHYDEAFGKSAIPEAYERLLLDAIQGDASLFTRADEVETAWSIIDPILQTWDTHQTPPLAVYKPSSWGPAESDMLLAKDGRRWLNEESDA

SEQ ID NO: 259 - protein, 180 aa, Artificial Sequence
  Synthetic
MSKLEELDIVSNNILILKKFYTNDEWKNKLDSLIDRIIKAKKIFIFGVGRSGYIGRCFAMRLMHLGFKSYFVGETTTPSYEKDDLLILISGSGRTESVLTVAKKAKNINNNIIAIVCECGNVVEFADLTIPLEVKKSKYLPMGTTFEETALIFLDLVIAEIMKRLNLDESEIIKRHCNLL

(19) United States
(12) Patent Application Publication
(10) Pub. No.: US 2022/0213492 A1
(43) Pub. Date: Jul. 7, 2022

(54) METHANOL UTILIZATION

(71) Applicant: Ginkgo Bioworks, Inc., Boston, MA (US)

(72) Inventors: Hui Zhou, Boston, MA (US); Massimo Merighi, Boston, MA (US); Micjael G. Napolitano, Boston, MA (US); Kennji Abe, Kanagawa (JP); Yoshihiro Ito, Kanagawa (JP); Takayuki Asahara, Kanagawa (JP); Thomas Perli, Boston, MA (US); Sergio L. Florez, Boston, MA (US); Ryan J. Putman, Boston, MA (US); Ryo Takeshita, Kanagawa (JP); Yuri Uehara, Kanagawa (JP); Akito Chinen, Kanagawa (JP); Kazuteru Yamada, Kanagawa (JP)

(73) Assignee: Ginkgo Bioworks, Inc., Boston, MA (US)

(21) Appl. No.: 17/604,737

(22) PCT Filed: Apr. 17, 2020
(86) PCT No.: PCT/US2020/028746

§ 371 Date: Oct. 18, 2021

Related U.S. Application Data
(60) Provisional application No. 62/836,152, filed on Apr. 19, 2019.

(51) Int. Cl. C12N 15/70; C12N 15/52; C12N 9/04; C12N 9/88; C12N 9/90; C12P 13/08

(52) U.S. Cl. CPC C12N 15/70; C12N 15/52; C12N 9/0006; C12N 9/88; C12Y 503/01027; C12P 13/08; C12Y 101/01244; C12Y 401/02043; C12N 9/90

(57) ABSTRACT

Described herein are enzymes, such as for example, methanol dehydrogenase (MDH), 3-hexulose-6-phosphate isomerase (PHI), 3-hexulose-6-phosphate synthase (HPS), ribose-5-phosphate isomerase (RPI), ribulose 5-phosphate 3-epimerase (RPE), transketolase (TKT), transaldolase (TAL) enzymes, phosphofructokinase (PFK), Sedoheptulose 1,7-Bisphosphatase (GLPX), fructose-bisphosphate aldolase (FBA), 6-phosphogluconate dehydrogenase (GND), and glucose-6-phosphate dehydrogenase (ZWF); recombinant host cells expressing the enzymes; methods of producing methylotrophic cells; and methods of producing amino acids (e.g., lysine).

This application claims priority under 35 U.S.C. § 119 to U.S. Provisional Patent Application No. 62/836,152, filed Apr. 19, 2019, the entirety of which is incorporated by reference herein. Also, the Sequence Listing filed electronically herewith is hereby incorporated by reference (File name: 2020-04-17T_US-592PCT_Seq_List; File size:537 KB; Date recorded: Apr. 16, 2020).

### BACKGROUND

### Field of the Invention

The present disclosure relates to the production of recombinant host cells that can use methanol as a carbon source.

### Background Art

Methanol is a reduced one-carbon compound with the chemical formula CH₃OH. Methanol is inexpensive and can be produced on a large scale using syngas feedstocks starting from coal, petroleum oil, natural gas, and methane. Use of methanol as a carbon source in industrial fermentation processes, however, is often limited due to inefficient methanol assimilation and low product yields by naturally occurring organisms, including bacteria.

### SUMMARY

Aspects of the invention relate to recombinant host cells that express a heterologous gene encoding a methanol dehydrogenase (MDH), wherein the MDH comprises a sequence that is at least 90% identical to a region of SEQ ID NOS: 29-56 or SEQ ID NOS: 81-88, wherein the region corresponds to residues 96 to 295 of A0A031LYD0_9GAMM (SEQ ID NO: 34).

In some embodiments, the MDH comprises a region that:

(a) corresponds to residues 256 to 295 of wild-type A0A031LYD0_9GAMM (SEQ ID NO: 34), wherein the region comprises no more than seventeen amino acid substitutions relative to residues 256 to 295 of wild-type A0A031LYD0_9GAMM (SEQ ID NO: 34);

(b) corresponds to residues 167 to 172 of wild-type A0A031LYD0_9GAMM (SEQ ID NO: 34), wherein the region comprises no more than three amino acid substitutions relative to residues 167 to 172 of wild-type A0A031LYD0_9GAMM (SEQ ID NO: 34);

(c) corresponds to residues 366 to 369 of wild-type A0A031LYD0_9GAMM (SEQ ID NO: 34), wherein the region comprises no more than two amino acid substitutions relative to residues 366 to 369 of wild-type A0A031LYD0_9GAMM (SEQ ID NO: 34);

(d) corresponds to residues 42 to 46 of wild-type A0A031LYD0_9GAMM (SEQ ID NO: 34), wherein the region comprises no more than 1 amino acid substitution relative to residues 42 to 46 of wild-type A0A031LYD0_9GAMM (SEQ ID NO: 34);

(e) corresponds to residues 101 to 112 of wild-type A0A031LYD0_9GAMM (SEQ ID NO: 34), wherein the region comprises no more than four amino acid substitutions relative to residues 101 to 112 of wild-type A0A031LYD0_9GAMM (SEQ ID NO: 34);

(f) corresponds to residues 144 to 152 of wild-type A0A031LYD0_9GAMM (SEQ ID NO: 34), wherein the region comprises no more than two amino acid substitutions relative to residues 144 to 152 of wild-type A0A031LYD0_9GAMM (SEQ ID NO: 34); and/or

(g) corresponds to residues 194 to 211 of wild-type A0A031LYD0_9GAMM (SEQ ID NO: 34), wherein the region comprises no more than three amino acid substitutions relative to residues 194 to 211 of wild-type A0A031LYD0_9GAMM (SEQ ID NO: 34).

In some embodiments, the region in (a) comprises at least one of:

(i) a leucine (L) or methionine (M) at a residue corresponding to position 256 of wild-type A0A031LYD0_9GAMM (SEQ ID NO: 34);

(ii) a valine (V) or methionine (M) at a residue corresponding to position 259 of wild-type A0A031LYD0_9GAMM (SEQ ID NO: 34);

(iii) an alanine (A) or glycine (G) at a residue corresponding to position 264 of wild-type A0A031LYD0_9GAMM (SEQ ID NO: 34);

(iv) an asparagine (N), glycine (G), or serine (S) at a residue corresponding to position 265 of wild-type A0A031LYD0_9GAMM (SEQ ID NO: 34);

(v) a phenylalanine (F), tyrosine (Y), or leucine (L) at a residue corresponding to position 268 of wild-type A0A031LYD0_9GAMM (SEQ ID NO: 34);

(vi) an alanine (A) or serine (S) at a residue corresponding to position 271 of wild-type A0A031LYD0_9GAMM (SEQ ID NO: 34);

(vii) (vii) a isoleucine (I) or methionine (M) at a residue corresponding to position 272 of wild-type A0A031LYD0_9GAMM (SEQ ID NO: 34);

(viii) (viii) an alanine (A) or serine (S) at a residue corresponding to position 273 of wild-type A0A031LYD0_9GAMM (SEQ ID NO: 34);

(ix) (ix) a leucine (L) or valine (V) at a residue corresponding to position 276 of wild-type A0A031LYD0_9GAMM (SEQ ID NO: 34);

(x) (x) a phenylalanine (F), leucine (L), or valine (V) at a residue corresponding to position 279 of wild-type A0A031LYD0_9GAMM (SEQ ID NO: 34);

(xi) (xi) an asparagine (N), aspartic acid (D), glycine (G), or lysine (K) at a residue corresponding to position 281 of wild-type A0A031LYD0_9GAMM (SEQ ID NO: 34);

(xii) (xii) a leucine (L), methionine (M), or phenylalanine (F) at a residue corresponding to position 282 of wild-type A0A031LYD0_9GAMM (SEQ ID NO: 34);

(xiii) (xiii) a proline (P) or glutamine (Q) at a residue corresponding to position 283 of wild-type A0A031LYD0_9GAMM (SEQ ID NO: 34);

(xiv) (xiv) a valine (V) or isoleucine (I) at a residue corresponding to position 286 of wild-type A0A031LYD0_9GAMM (SEQ ID NO: 34);

(xv) (xv) an alanine (A) or cysteine (C) at a residue corresponding to position 287 of wild-type A0A031LYD0_9GAMM (SEQ ID NO: 34);

(xvi) (xvi) an alanine (A) or serine (S) at a residue corresponding to position 289 of wild-type A0A031LYD0_9GAMM (SEQ ID NO: 34);

(xvii) (xvii) a leucine (L), valine (V), or isoleucine (I) at a residue corresponding to position 290 of wild-type A0A031LYD0_9GAMM (SEQ ID NO: 34);

(xviii) (xviii) a leucine (L) or valine (V) at a residue corresponding to position 291 of wild-type A0A031LYD0_9GAMM (SEQ ID NO: 34); and

(xix) (xix) a methionine (M) or leucine (L) at a residue corresponding to position 292 of wild-type A0A031LYD0_9GAMM (SEQ ID NO: 34).

In some embodiments, the MDH comprises a region that:

(a) corresponds to residues 256 to 295 of wild-type A0A031LYD0_9GAMM (SEQ ID NO: 34), wherein the region comprises no more than three amino acid substitutions relative to residues 256 to 295 of wild-type A0A031LYD0_9GAMM (SEQ ID NO: 34);

(b) corresponds to residues 167 to 172 of wild-type A0A031LYD0_9GAMM (SEQ ID NO: 34), wherein the region comprises no more than one amino acid substitution relative to residues 167 to 172 of wild-type A0A031LYD0_9GAMM (SEQ ID NO: 34); and/or

(c) corresponds to residues 366 to 369 of wild-type A0A031LYD0_9GAMM (SEQ ID NO: 34), wherein the region comprises no more than one amino acid substitution relative to residues 366 to 369 of wild-type A0A031LYD0_9GAMM (SEQ ID NO: 34).

In some embodiments, the region in (b) comprises an alanine (A), proline (P), or valine (V) at a residue corresponding to position 169 of wild-type A0A031LYD0_9GAMM (SEQ ID NO: 34). In some embodiments, the region in (b) comprises a valine (V) at a residue corresponding to position 169 of wild-type A0A031LYD0_9GAMM (SEQ ID NO: 34). In some embodiments, the region in (c) comprises an alanine (A), valine (V), glycine (G), or arginine (R) at a residue corresponding to position 368 of wild-type A0A031LYD0_9GAMM (SEQ ID NO: 34).

In some embodiments, the MDH comprises an arginine (R) at a residue corresponding to position 368 of wild-type A0A031LYD0_9GAMM (SEQ ID NO: 34). In some embodiments, the MDH further comprises an alanine (A), aspartic acid (D), glutamic acid (E), asparagine (N), proline (P), glutamine (Q), serine (S), threonine (T), valine (V), or glycine (G) at an amino acid residue corresponding to position 31 in A0A031LYD0_9GAMM (SEQ ID NO: 34).

In some embodiments, the MDH comprises a valine (V) at an amino acid residue corresponding to position 31 in A0A031LYD0_9GAMM (SEQ ID NO: 34). In some embodiments, the MDH further comprises an alanine (A), a isoleucine (I), a leucine (L), or valine (V) at an amino acid residue corresponding to position 26 in A0A031LYD0_9GAMM (SEQ ID NO: 34).

In some embodiments, the MDH further comprises a valine (V) at an amino acid residue corresponding to position 26 in A0A031LYD0_9GAMM (SEQ ID NO: 34). In some embodiments, the MDH comprises more than one amino acid substitution relative to the sequence of wild-type A0A031LYD0_9GAMM (SEQ ID NO: 34), wherein at least one of the amino acid substitutions is a conservative substitution.

In some embodiments, the MDH has at least 25% of the NAD reductase activity as compared to cnMDHm3 as measured by XTT enzyme assay. In some embodiments, the MDH is capable of catalyzing conversion of methanol to formaldehyde. In some embodiments, the MDH has a k_{cat} of at least 20 s⁻¹ as calculated using total protein and optical density of NADH. In some embodiments, the MDH has a Kₘ that is lower than 1.2 M as calculated using total protein and optical density of NADH. In some embodiments, the MDH has a k_{cat}/Kₘ ratio of between 300 L/(mol*s) and 1,000 L/(mol*s) as calculated by total protein and optical density of NADH. In some embodiments, the MDH has a k_{cat} of at least 0.3 s⁻¹ as calculated using target protein concentration and concentration of NADH. In some embodiments, the MDH has a Kₘ that is lower than 1.3 M as calculated using target protein concentration and concentration of NADH. In some embodiments, the MDH has a k_{cat}/Kₘ ratio of between 1 L/(mol*s) and 30 L/(mol*s).

In some embodiments, the recombinant host cell further comprises a heterologous gene encoding a 3-hexulose-6-phosphate synthase (HPS) selected from SEQ ID NOS: 106-122 or HPS amino acid sequences in Table 3. In some embodiments, the recombinant host cell further comprises a heterologous gene encoding a 3-hexulose-6-phosphate isomerase (PHI) selected from SEQ ID NOS: 135-146 or PHI amino acid sequences in Table 4.

Aspects of the invention relate to recombinant host cells that express a heterologous gene encoding a methanol dehydrogenase (MDH), wherein the MDH comprises a sequence that is at least 90% identical to a region that corresponds to residues 96 to 295 of A0A031LYD0_9GAMM (SEQ ID NO: 34) and wherein the MDH comprises:

(a) a valine (V) at an amino acid residue corresponding to position 26 in A0A031LYD0_9GAMM (SEQ ID NO: 34);

(b) a valine (V) at an amino acid residue corresponding to position 31 in A0A031LYD0_9GAMM (SEQ ID NO: 34);

(c) a valine (V) at an amino acid residue corresponding to position 169 in A0A031LYD0_9GAMM (SEQ ID NO: 34); and/or

(d) an arginine (R) at an amino acid residue corresponding to position 368 in A0A031LYD0_9GAMM (SEQ ID NO: 34).

In some embodiments, the MDH comprises (a), (c), and (d). In some embodiments, the MDH comprises (b), (c), and (d). In some embodiments, the MDH comprises (a), (b), (c), and (d). In some embodiments, the MDH comprises (a) and (b); (a) and (c); (a) and (d); (b) and (c); (b) and (d); or (c) and (d). In some embodiments, the MDH comprises more than one amino acid substitution relative to the sequence of wild-type A0A031LYD0_9GAMM (SEQ ID NO: 34), wherein at least one of the amino acid substitution(s) is a conservative amino acid substitution.

In some embodiments, the MDH has at least 25% of the NAD reductase activity as compared to cnMDHm3 as measured by XTT enzyme assay. In some embodiments, the MDH is capable of catalyzing conversion of methanol to formaldehyde. In some embodiments, the MDH has a k_{cat} of at least 20 s⁻¹ as calculated using total protein and optical density of NADH. In some embodiments, the MDH has a Kₘ of at least 0.04 M as calculated using total protein and optical density of NADH. In some embodiments, the MDH has a k_{cat}/Kₘ ratio of at least 300. In some embodiments, the MDH has a k_{cat} of at least 0.3 s⁻¹ as calculated using target protein concentration and concentration of NADH. In some embodiments, the MDH has a Kₘ of at least 0.04 M as calculated using target protein concentration and concentration of NADH. In some embodiments, the MDH has a k_{cat}/Kₘ ratio of at least 1.1. In some embodiments, the recombinant host cell further comprises a heterologous gene encoding a 3-hexulose-6-phosphate synthase (HPS) selected from SEQ ID NOS: 106-122 or HPS amino acid sequences in Table 3. In some embodiments, the recombinant host cell further comprises a heterologous gene encoding a 3-hexulose-6-phosphate isomerase (PHI) selected from SEQ ID NOS: 135-146 or PHI amino acid sequences in Table 4.

Aspects of the invention relate to recombinant host cells that express a heterologous gene encoding a methanol dehydrogenase (MDH), wherein the MDH comprises a sequence that is at least 90% identical to a sequence selected from SEQ ID NOS: 29-56, SEQ ID NOS: 81-88, or MDH amino acid sequences in Table 2. In some embodiments, the MDH comprises at least one amino acid substitution relative to the sequence of wild-type A0A031LYD0_9GAMM (SEQ ID NO:34). In some embodiments, the MDH comprises more than one amino acid substitution relative to the sequence of wild-type A0A031LYD0_9GAMM (SEQ ID NO:34), wherein at least one of the amino acid substitutions is a conservative amino acid substitution. In some embodiments, the MDH has at least 25% of the NAD reductase activity as compared to cnMDHm3 as measured by XTT enzyme assay. In some embodiments, the MDH is capable of catalyzing conversion of methanol to formaldehyde. In some embodiments, the MDH has a k_{cat} of at least 20 s⁻¹ as calculated using total protein and optical density of NADH. In some embodiments, the MDH has a Kₘ of at least 0.04 M as calculated using total protein and optical density of NADH. In some embodiments, the MDH has a k_{cat}/Kₘ ratio of at least 300. In some embodiments, the MDH has a k_{cat} of at least 0.3 s⁻¹ as calculated using target protein concentration and concentration of NADH. In some embodiments, the MDH has a Kₘ of at least 0.04 M as calculated using target protein concentration and concentration of NADH. In some embodiments, the MDH has a k_{cat}/Kₘ ratio of at least 1.1. In some embodiments, the recombinant host cell further comprises a heterologous gene encoding a 3-hexulose-6-phosphate synthase (HPS) selected from SEQ ID NOS: 106-122 or HPS amino acid sequences in Table 3. In some embodiments, the recombinant host cell further comprises a heterologous gene encoding a 3-hexulose-6-phosphate isomerase (PHI) selected from SEQ ID NOS: 135-146 or PHI amino acid sequences in Table 4.

Aspects of the invention relate to recombinant host cells that express a heterologous gene encoding a 3-hexulose-6-phosphate (HPS), wherein the HPS comprises a sequence that is at least 90% identical to a region of SEQ ID NOS: 106-122, wherein the region corresponds to residues 26 to 151 of wild-type A0A0M4M0F0 (SEQ ID NO: 106).

In some embodiments, the HPS comprises a region that comprises:

(a) a glutamine (Q) at a residue corresponding to position 4 of wild-type A0A0M4M0F0 (SEQ ID NO: 6);

(b) an alanine (A) at a residue corresponding to position 6 of wild-type A0A0M4M0F0 (SEQ ID NO: 6);

(c) an aspartic acid (D) at a residue corresponding to position 8 of wild-type A0A0M4M0F0 (SEQ ID NO: 6);

(d) an aspartic acid (D) at a residue corresponding to position 27 of wild-type A0A0M4M0F0 (SEQ ID NO: 6);

(e) a glutamic acid (E) at a residue corresponding to position 30 of wild-type A0A0M4M0F0 (SEQ ID NO: 6);

(f) a glycine (G) at a residue corresponding to position 32 of wild-type A0A0M4M0F0 (SEQ ID NO: 6);

(g) a threonine (T) at a residue corresponding to position 33 of wild-type A0A0M4M0F0 (SEQ ID NO: 6);

(h) a proline (P) at a residue corresponding to position 34 of wild-type A0A0M4M0F0 (SEQ ID NO: 6);

(i) a glycine (G) at a residue corresponding to position 40 of wild-type A0A0M4M0F0 (SEQ ID NO: 6);

(j) an aspartic acid (D) at a residue corresponding to position 59 of wild-type A0A0M4M0F0 (SEQ ID NO: 6);

(k) a lysine (K) at a residue corresponding to position 61 of wild-type A0A0M4M0F0 (SEQ ID NO: 6);

(1) a methionine (M) at a residue corresponding to position 63 of wild-type A0A0M4M0F0 (SEQ ID NO: 6);

(m) an aspartic acid (D) at a residue corresponding to position 64 of wild-type A0A0M4M0F0 (SEQ ID NO: 6);

(n) a glutamic acid (E) at a residue corresponding to position 69 of wild-type A0A0M4M0F0 (SEQ ID NO: 6);

(o) an glycine (G) at a residue corresponding to position 77 of wild-type A0A0M4M0F0 (SEQ ID NO: 6);

(p) an alanine (A) at a residue corresponding to position 78 of wild-type A0A0M4M0F0 (SEQ ID NO: 6);

(q) a leucine (L) at a residue corresponding to position 84 of wild-type A0A0M4M0F0 (SEQ ID NO: 6);

(r) an isoleucine (I) at a residue corresponding to position 92 of wild-type A0A0M4M0F0 (SEQ ID NO: 6);

(s) an alanine (A) at a residue corresponding to position 99 of wild-type A0A0M4M0F0 (SEQ ID NO: 6);

(t) a valine (V) at a residue corresponding to position 108 of wild-type A0A0M4M0F0 (SEQ ID NO: 6);

(u) an aspartic acid (D) at a residue corresponding to position 109 of wild-type A0A0M4M0F0 (SEQ ID NO: 6);

(v) an alanine (A) at a residue corresponding to position 120 of wild-type A0A0M4M0F0 (SEQ ID NO: 6);

(w) a glycine (G) at a residue corresponding to position 127 of wild-type A0A0M4M0F0 (SEQ ID NO: 6);

(x) a histidine (H) at a residue corresponding to position 134 of wild-type A0A0M4M0F0 (SEQ ID NO: 6);

(y) a glycine (G) at a residue corresponding to position 136 of wild-type A0A0M4M0F0 (SEQ ID NO: 6);

(z) an aspartic acid (D) at a residue corresponding to position 138 of wild-type A0A0M4M0F0 (SEQ ID NO: 6);

(aa) a glutamine (Q) at a residue corresponding to position 140 of wild-type A0A0M4M0F0 (SEQ ID NO: 6);

(bb) an alanine (A) at a residue corresponding to position 141 of wild-type A0A0M4M0F0 (SEQ ID NO: 6);

(cc) an alanine (A) at a residue corresponding to position 164 of wild-type A0A0M4M0F0 (SEQ ID NO: 6);

(dd) a glycine (G) at a residue corresponding to position 165 of wild-type A0A0M4M0F0 (SEQ ID NO: 6);

(ee) a glycine (G) at a residue corresponding to position 166 of wild-type A0A0M4M0F0 (SEQ ID NO: 6);

(ff) a glycine (G) at a residue corresponding to position 186 of wild-type A0A0M4M0F0 (SEQ ID NO: 6);

(gg) an isoleucine (I) at a residue corresponding to position 189 of wild-type A0A0M4M0F0 (SEQ ID NO: 6); and/or

(hh) an alanine (A) at a residue corresponding to position 199 of wild-type A0A0M4M0F0 (SEQ ID NO: 6).

In some embodiments, the HPS is capable of converting formaldehyde and ribulose 5-phosphate into hexulose-6-P. In some embodiments, the HPS has an activity that is at least 50% of a control enzyme, wherein the control enzyme is HPS from Methylococcus capsulatus (UniProtKB-Q602L4) (SEQ ID NO: 122). In some embodiments, the recombinant host cell further comprises a heterologous gene encoding a methanol dehydrogenase (MDH) selected from SEQ ID NOS: 29-56, SEQ ID NOS: 81-88, or an MDH amino acid sequence in Table 2. In some embodiments, the recombinant host cell further comprises a heterologous gene encoding a 3-hexulose-6-phosphate isomerase (PHI) selected from SEQ ID NOS: 135-146 or PHI amino acid sequences in Table 4.

Aspects of the invention relate to recombinant host cells that express a heterologous gene encoding a 3-hexulose-6-phosphate (HPS), wherein the HPS comprises a sequence that is at least 90% identical to an HPS in SEQ ID NOS: 106-122 or HPS amino acid sequences in Table 3. In some embodiments, the HPS comprises at least one amino acid substitution relative to the sequence of HPS from Methylococcus capsulatus (UniProtKB-Q602L4) (SEQ ID NO: 122). In some embodiments, the HPS is capable of converting formaldehyde and ribulose 5-phosphate into hexulose-6-P. In some embodiments, the HPS has an activity that is at least 50% of a control enzyme, wherein the control enzyme is HPS from Methylococcus capsulatus (UniProtKB-Q602L4) (SEQ ID NO: 122). In some embodiments, the recombinant host cell further comprises a heterologous gene encoding a methanol dehydrogenase (MDH) selected from SEQ ID NOS: 29-56, SEQ ID NOS: 81-88, or an MDH amino acid sequence in Table 2. In some embodiments, the recombinant host cell further comprises a heterologous gene encoding a 3-hexulose-6-phosphate isomerase (PHI) selected from SEQ ID NOS: 135-146 or PHI amino acid sequences in Table 4.

Aspects of the invention relate to recombinant host cells that express a heterologous gene encoding a 3-hexulose-6-phosphate isomerase (PH), wherein the PHI comprises a sequence that is at least 90% identical to a PHI selected from SEQ ID NOS: 135-146 or PHI amino acid sequences in Table 4. In some embodiments, the PHI comprises at least one amino acid substitution relative to PHI from Methylococcus capsulatus (SEQ ID NO: 146).

In some embodiments, the PHI is capable of converting hexulose-6-phosphate to fructose-6-phosphate. In some embodiments, the PHI has an activity that is at least 50% of a control enzyme, wherein the control enzyme is PHI from Methylococcus capsulatus (SEQ ID NO: 146). In some embodiments, the recombinant host cell further comprises a heterologous gene encoding a methanol dehydrogenase (MDH) selected from SEQ ID NOS: 29-56, SEQ ID NOS: 81-88, or an MDH amino acid sequence in Table 2.

In some embodiments, the recombinant host cell further comprises a heterologous gene encoding a 3-hexulose-6-phosphate synthase (HPS) selected from SEQ ID NOS: 106-122 or HPS amino acid sequences in Table 3. In some embodiments, the recombinant host cell further comprises a sequence that is at least 90% identical to an RPI enzyme selected from SEQ ID NOS: 217-222 or RPI amino acid sequences in Table 5. In some embodiments, the recombinant host cell further comprises a sequence that is at least 90% identical to an RPE enzyme selected from SEQ ID NOS: 204-210 or RPE amino acid sequences in Table 5. In some embodiments, the recombinant host cell further comprises a sequence that is at least 90% identical to a TKT enzyme selected from SEQ ID NOS: 241-246 or TKT amino acid sequences in Table 5. In some embodiments, the recombinant host cell further comprises a sequence that is at least 90% identical to a TAL enzyme selected from SEQ ID NOS: 229-234 or TAL amino acid sequences in Table 5. In some embodiments, the recombinant host cell further comprises a sequence that is at least 90% identical to a PFK enzyme selected from SEQ ID NOS: 191-196 or PFK amino acid sequences in Table 5. In some embodiments, the recombinant host cell further comprises a sequence that is at least 90% identical to a GLPX enzyme selected from SEQ ID NOS: 166-172 or GLPX amino acid sequences in Table 5. In some embodiments, the recombinant host cell further comprises a sequence that is at least 90% identical to an FBA enzyme selected from SEQ ID NOS: 153-158 or FBA amino acid sequences in Table 5. In some embodiments, the recombinant host cell further comprises a sequence that is at least 90% identical to a GND enzyme selected from SEQ ID NOS: 179-184 or GND amino acid sequences in Table 5. In some embodiments, the recombinant host cell further comprises a sequence that is at least 90% identical to a ZWF enzyme selected from SEQ ID NOS: 253-258 or ZWF amino acid sequences in Table 5.

In some embodiments, the recombinant host cell is capable of producing an organic compound with at least one carbon derived from methanol in a feedstock comprising substitution of a saccharide with methanol. In some instances, the organic compound is an amino acid. In some instances, the organic compound is a lysine. In some embodiments, the % weight per weight (% w/w) substitution of the saccharide with methanol is at least 5%. In some embodiments, at least 25% of the methanol provided in feedstock is consumed by the recombinant host cell. In some embodiments, the saccharide is sucrose, glucose, lactose, dextrose, or fructose. In some embodiments, the recombinant host cell is an Escherichia coli (E. coli) cell. In some embodiments, the recombinant host cell further comprises a knockout of a gene encoding S-(hydroxymethyl)glutathione dehydrogenase. In some embodiments, the gene is frmA gene. In some embodiments, at least one heterologous gene is expressed from a J23104 promoter, an Ec-TTL-P041 promoter, and/or a P_{gal} promoter. In some embodiments, at least two heterologous genes are driven by the J23104 promoter, the Ec-TTL-P041 promoter, or the P_{gal} promoter.

Aspects of the invention relate to methods of producing methanol-derived lysine comprising culturing recombinant host cells described herein in feedstock comprising substitution of a saccharide with methanol, thereby producing methanol-derived lysine.

In some embodiments, the % weight per weight (% w/w) substitution of the saccharide with methanol in the feedstock is at least 5%. In some embodiments, at least 25% of the methanol provided in feedstock is consumed by the recombinant host cell. In some embodiments, the saccharide is sucrose, glucose, lactose, dextrose, or fructose.

Further aspects of the disclosure relate to vectors comprising a sequence that is at least 90% identical to a sequence selected from SEQ ID NOs: 1-28, 73-80, 89-105, 123-134, 147-152, 159-165, 173-178, 185-190, 197-203, 211-216, 223-228, 235-240 and 247-252.

Further aspects of the disclosure relate to expression cassettes comprising a sequence that is at least 90% identical to a sequence selected from SEQ ID NOs: 1-28, 73-80, 89-105, 123-134, 147-152, 159-165, 173-178, 185-190, 197-203, 211-216, 223-228, 235-240 and 247-252.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways.

### BRIEF DESCRIPTION OF DRAWINGS

The accompanying drawings are not intended to be drawn to scale. The drawings are illustrative only and are not required for enablement of the disclosure. For purposes of clarity, not every component may be labeled in every drawing. In the drawings:

FIG. 1 shows a non-limiting example of a ribulose monophosphate pathway (RuMP) for methanol assimilation.

FIG. 2 shows a diagram of a sequence similarity network (SSN) of approximately 6,000 proteins in a screening library to identify methanol dehydrogenases (MDHs).

FIGS. 3A-3G show a sequence logo of a Hidden Markov Model (HMM).

FIGS. 4A-4C show an alignment of twenty-eight MDHs (SEQ ID NOs: 29-56) that were identified as disclosed herein. The alignment was generated with ClustalW.

FIG. 5 is a chart showing a list of candidate MDHs with formaldehyde production activity as determined by a Nash assay and methanol-dependent NAD+ reductase activity as determined by an NAD assay. In the Nash assay, the absorbance at 412 nm by optical density compared to a positive control is shown. The NAD assay is depicted in FIG. 6.

FIG. 6 shows results of screening of MDHs with methanol-dependent NAD⁺ reductase activity. Values were normalized to the positive control CnMDHm3 (SEQ ID NO: 30). The colorimetric assay measures reduction of the XTT tetrazolium dye (colorless) by the generated NADH from the enzymatic reaction to form a brightly colored orange formazan derivative.

FIGS. 7A-7B show enzyme activity of engineered methanol dehydrogenase variants as determined by the Nash assay. Variants of Acinetobacter sp. Ver3 Uniprot A0A031LYD0_9GAMM (1) A26V, S31V, A169V, and A368R; (2) A26V, A169V, and A368R; (3) A26V and A368R; or (4) S31V, A169V, and A368R) demonstrated improved catalytic activity on average compared to CnMDHm3 and wild-type A0A031LYD0_9GAMM as measured by net NAD reductase activity. CnMDHm3 was used as a positive control. FIG. 7B provides a list of mutations for each of the four MDH native enzymes from the hits in FIG. 6.

FIG. 8 shows results of an in vivo Nash assay for formaldehyde production indicative of methanol dehydrogenase activity. CnMDHm3 (SEQ ID NO: 30) was used as a positive control.

FIGS. 9A-9B include data showing a lack of correlation between in vitro NAD reductase activity (rate per mg protein) with methanol dehydrogenase activity in vivo as determined by the NASH assay. CnMDHm3 was used as a positive control. FIG. 9A is a graph comparing the NAD reductase activity of cell extracts (rate per mg protein) comprising a recombinant MDH variant with the Nash activity in intact cells expressing the same recombinant MDH for variants shown in FIG. 9B. The value for MDH_m3 is shown. FIG. 9B shows the NADH reductase activity and Nash activity values for the MDH variants tested.

FIGS. 10A-10B show kinetic characterization for seven active MDH enzymes calculated based on concentration of target protein and signal of generated NADH during reaction as shown in FIG. 6. FIG. 10A shows the k_{cat} (s⁻¹), Kₘ (M), and k_{cat}/Kₘ ratios for each of the indicated MDHs from cell extracts as calculated using total protein and optical absorption of XTT formazan coupled with NADH production. FIG. 10B shows the k_{cat} (s⁻¹), Kₘ (M), and k_{cat}/Kₘ ratios for each of the indicated MDHs from cell extracts as calculated using target protein concentration and concentration of NADH. The NADH concentration for FIG. 10B is calculated by standard curve of fluorescent absorption of NADH (Ex=340 nm, Em=445 nm). The target protein concentrations are obtained by absolute quantification proteomics using internal standard 13C-peptides. * indicates that isotope labeled peptide was not available for A0A031LYDO_9GAMM-A26V-A169V-A368R.

FIG. 11 depicts diagrams of sequence similarity networks (SSNs) of approximately 1,400 proteins in two separate screening libraries to identify (1) 3-hexulose-6-phosphate synthase (HPS) enzymes (left) and (2) 3-hexulose-6-phosphate isomerase (PHI) enzymes.

FIG. 12 is a schematic of a tetrazolium dye-based assay to screen for HPS and PHI enzyme activity in the RuMP pathway. The colorimetric assay measures reduction of the XTT tetrazolium dye (colorless) to form a brightly colored orange formazan derivative.

FIG. 13 shows HPS enzyme hits having a z-score greater than 2 in the screening assay.

FIG. 14 shows PHI enzyme hits having a z-score greater than 2 in the screening assay.

FIG. 15 shows the protein normalized reaction rate of HPS (left) and PHI enzymes as compared to Methylococcus capsulatus controls. * indicates a cell growth reduction in strain.

FIG. 16 shows 1,152 synthons generated using combinations of promoters, operators, mRNA stability cassettes, ribosomal binding sites, and terminators, with genes encoding 8 different MDH enzymes, 4 different HPS enzymes, and 4 different PHI enzymes. Assimilation of ¹³C-methanol into biomass and product was measured (not shown).

FIG. 17 shows the individual MDH, HPS, and PHI enzymes used to synthesize the pathways.

FIG. 18 shows a non-limiting example of a host cell expressing a heterologous MDH, a heterologous HPS and a heterologous PHI that was capable of producing up to 95% lysine titer fed with 90% glucose+10% methanol, as compared to 88% lysine titer detected with only 90% glucose feeding. The lysine titer ratio % is calculated against a control strain that does not express a heterologous RuMP pathway enzyme.

FIG. 19 shows a list of fifty-six additional RuMP cycle enzymes with enzyme activity.

FIG. 20 shows reactions that were used to assay for activity of an indicated enzyme and non-limiting examples of assays to determine enzyme activity.

FIG. 21 shows a schematic of construction of plasmids encoding RuMP cycle modules. The plasmids encode MDH, HPS, and PHI in one expression cassette under one promoter and two to five other RuMP cycle genes from FIG. 19 under a separate promoter.

### DETAILED DESCRIPTION

Methanol (CH₃OH) is an inexpensive feedstock and can be synthesized from a variety of sources including methane, which is the most abundant fossil fuel compound on Earth. However, use of methanol as a carbon source in industrial fermentation processes often has high production costs and low yield, especially in the production of more complex compounds with multiple carbon to carbon bonds. This disclosure is premised, at least in part, on the unexpected finding that recombinant host cells may be engineered to efficiently use methanol as a carbon source, for example to produce lysine. Accordingly, provided herein are recombinant host cells engineered to express methanol dehydrogenase (MDH) enzymes, 3-hexulose-6-phosphate synthase (hexulose phosphate synthase, HPS) enzymes, and 3-hexulose-6-phosphate isomerase (phosphohexuloisomerase, PHI) enzymes, or combinations thereof. The present disclosure also provides methods for making amino acids, including lysine (e.g., using recombinant host cells expressing MDHs, HPSs, and/or PHIs).

As used herein, a methylotroph is an organism that is capable of methanol assimilation, (i.e., capable of using methyl compounds that do not include carbon-carbon bonds as the source of carbon). Methyl compounds without carbon-carbon bonds include methane and methanol.

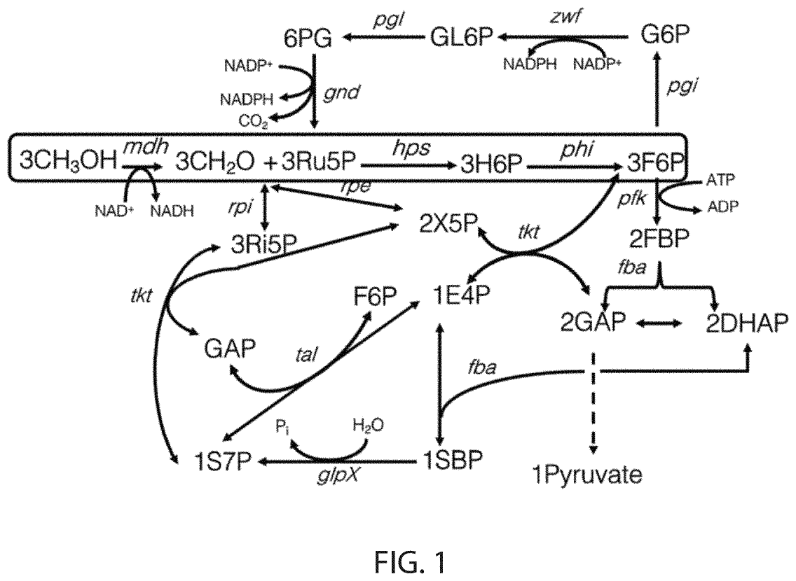

FIG. 1 is a non-limiting example of a ribulose monophosphate pathway (RuMP) in the methylotroph Bacillus methanolicus. In the RuMP pathway, methanol is converted into formaldehyde by methanol dehydrogenase (MDH) and formaldehyde is fixed with ribulose 5-phosphate (Ru-5-P) to form hexulose-6-phosphate (H-6-P) by 3-hexulose-6-phosphate synthase (HPS). Hexulose-6-phosphate (H-6-P) is then isomerized to fructose 6-phosphate (F-6-P) by 3-hexulose-6-phosphate isomerase (PHI). F-6-P is converted into fructose-1,6-bisphosphate (F-1,6-dp) by phosphofructokinase (pfk). Fructose biphosphate aldolase (fba) forms dihydroxy acetone phosphate (DHAP) from F-1,6-dp. DHAP can be used to form phospho-enol-pyruvate and pyruvate. Pyruvate is then converted into acetyl-CoA, which can enter the Kreb's cycle (citric acid cycle, TCA) to produce intermediates including oxaloacetate, which is a precursor to lysine. Concurrently pyruvate or phospho-enol-pyruvate can also be carboxylated to OAA, which is a precursor to lysine. By the assimilation of three formaldehyde molecules condensed into 3 molecules of ribulose-5-phosphate, three molecules of β-D-fructofuranose-6-phosphate (FMP) are created, for the net production of one molecule of triosophosphate (GA3P or DHAP).

Methanol Dehydrogenase (MDH) Enzymes

Aspects of the present disclosure provide methanol dehydrogenase (MDH) enzymes, which may be useful, for example, in increasing methanol assimilation in organisms including bacteria and yeast. As used herein, MDHs are capable of converting methanol into formaldehyde. In some embodiments, a MDH may be capable of converting ethanol or butanol into formaldehyde.

As a non-limiting example, one type of MDH uses a nicotinamide adenine (NAD) cofactor (e.g., nicotinamide adenine dinucleotide (NAD)+ or nicotinamide adenine dinucleotide phosphate (NADP+)) as substrates. As a non-limiting example, a NAD-dependent MDH may bind metal ions, including iron and magnesium or zinc and magnesium. See, e.g., Hektor, et al., J Biol Chem. 2002 Dec. 6; 277(49):46966-73. In some embodiments, a MDH is a type III iron-dependent alcohol dehydrogenase.

As a non-limiting example, an alcohol dehydrogenase may be identified by searching for a sequence with a conserved alcohol dehydrogenase domain (e.g., Pfam Family identification No. PF00465). Then, the putative alcohol dehydrogenase may be tested for MDH activity using the methods described herein or any method known in the art.

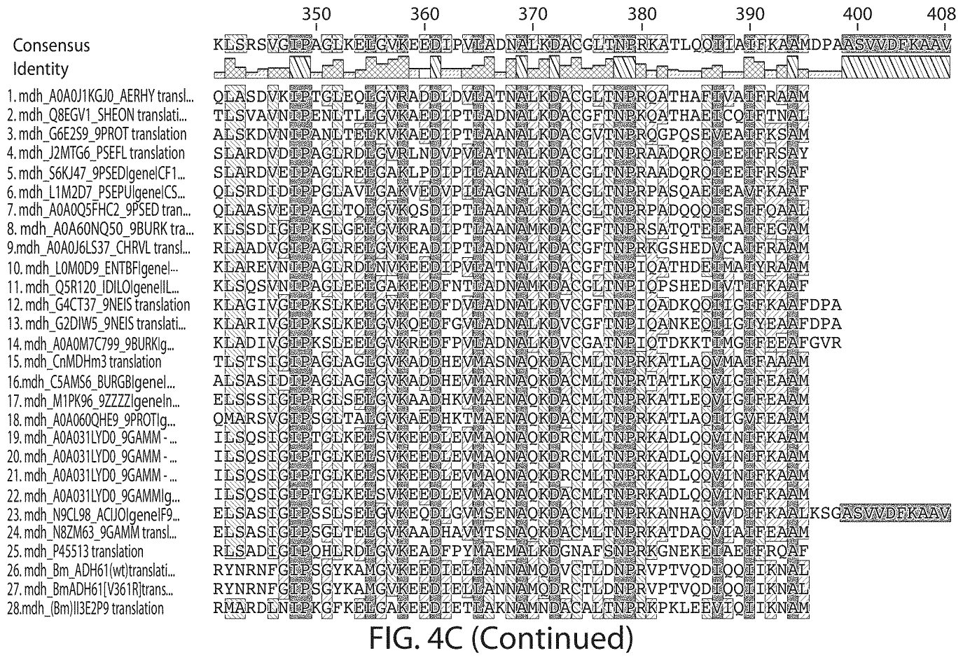

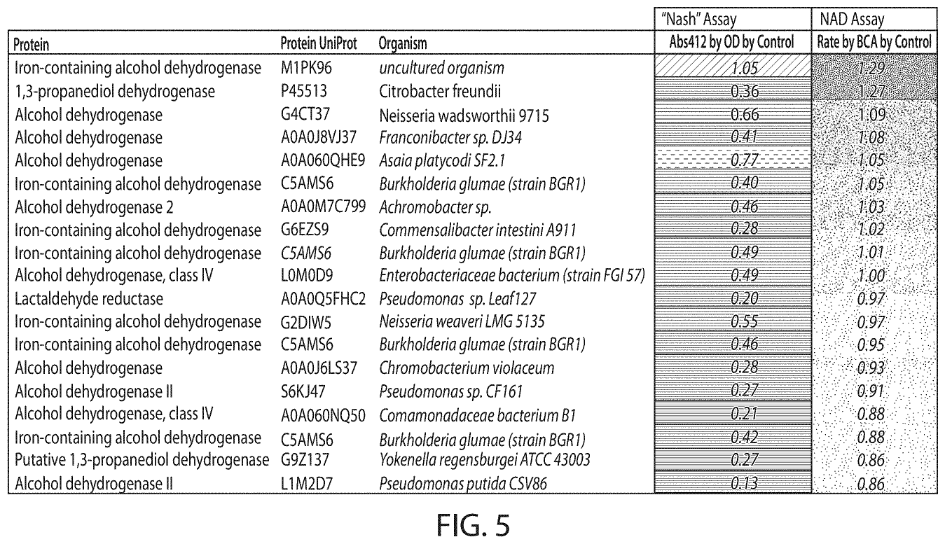

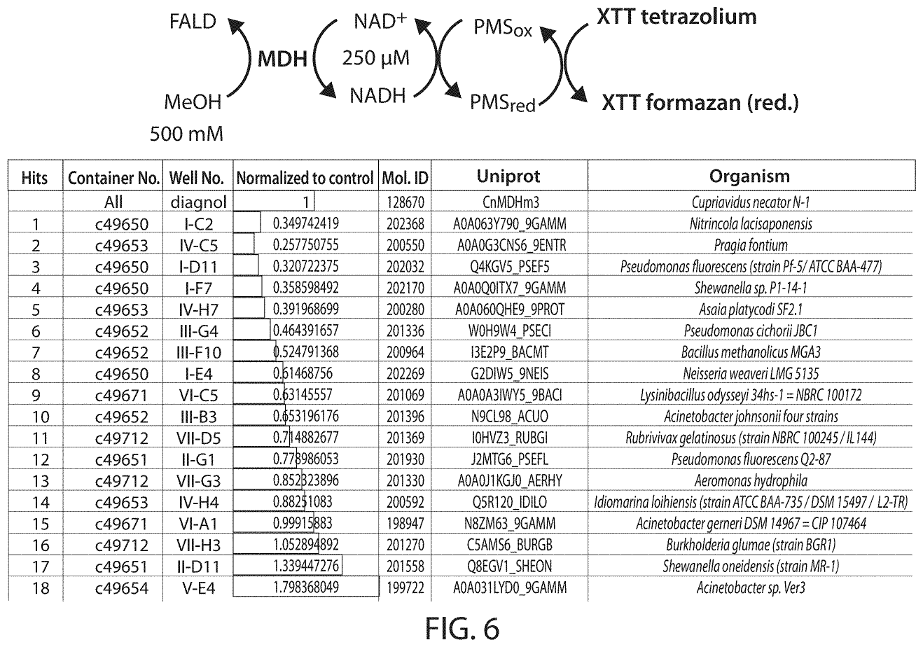

MDH enzymes of the present disclosure may include a sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or is 100% identical, including all values in between, to a sequence (e.g., nucleic acid or amino acid sequence) set forth as SEQ ID NOS: 1-28, SEQ ID NOS: 73-80, SEQ ID NOS: 29-56, or SEQ ID NOS: 81-88, or to a sequence in Table 2, or in FIGS. 5-6.

In some embodiments, a nucleic acid sequence encoding an MDH enzyme may be codon-optimized (e.g., for expression in a particular host cell, including bacteria).

MDH enzymes compatible with aspects of the invention may be derived from any species. Non-limiting examples of suitable species include Citrobacter freundii, Neisseria wadsworthii, Franconibacter, Ralstonia eutropha, Burkholderia glumae, Achromobacter, Commensalibacter intestini, Enterobacteriaceae bacterium, Pseudomonas, Comamonadaceae bacterium, Yokenella regensburgei, Pseudomonas putida, Cupriavidus necator, Nitrincola lacisaponensis, Pragia fontium, Pseudomonas fluorescens, Asaia platycodi, Pseudomonas cichorii, Shewanella sp. P1-14-1, Neisseria weaveri, Lysinibacillus odysseyi, Acinetobacter johnsonii, Chromobacterium violaceum, Rubrivivax gelatinosus, Aeromonas hydrophila, Idiomarina loihiensis, Acinetobacter gerneri, Acinetobacter sp. Ver3, Shewanella oneidensis, Brevibacterium casei, Arthrobacter methylotrophus, Mycobacterium gastri, Rhodococcus erythropolis, Amycolatopsis methanolica, Bacillus methanolicus, Acidomonas methanolica, Methylocapsa aurea, Afipia felis, Angulomicrobium tetraedrale, Methylobacterium extorquens, Methlyopila jiangsuensis, Paracoccus alkenifer, Sphingomonas melonis, Ancylobacter dichloromethanicus, Variovorax paradoxus, Methylophilus glucosoxydans, Methyloversatilis universalis, Methylibium aquaticum, Photobacterium indicum, Methylophaga thiooxydans, Methylococcus capsulatus, Klebsiella oxytoca, Gliocladium deliquescens, Paecilomyces variotii, Trichoderma lignorum, Candida boidini, Hansenula capsulatus, Pichia pastoris, Penicillium chrysogenum, and Photobacterium indicum. In some embodiments, an MDH is derived from a eukaryotic species that is capable of converting methanol into formaldehyde (e.g., Pichia spp.). Suitable species include those shown in FIGS. 5-6 and Table 2. See also, e.g., Kolb and Stacheter, Front Microbiol. 2013 Sep. 5; 4:268.

In some embodiments, an MDH of the present disclosure is capable of using methanol (MeOH or CH₃OH) and/or a longer chain alcohol as a substrate. As a non-limiting example, longer chain alcohols may include a chemical formula that is CₙH₂₊₁OH, wherein n is greater than 1. In some embodiments, an MDH of the present disclosure is capable of producing formaldehyde (CH₂O or FALD). In some embodiments, an MDH of the present disclosure catalyzes the formation of formaldehyde from methanol.

It should be appreciated that activity of an MDH can be measured by any means known to one of ordinary skill in the art. In some embodiments, the activity of an MDH may be measured by determining the methanol dehydrogenase activity of the enzyme. As a non-limiting example, methanol dehydrogenase activity may be measured using a tetrazolium dye (e.g., XTT). See, e.g., Example 1. MDH activity may also be determined by measuring the level of formaldehyde produced by an MDH enzyme, for example, using a Nash assay. See, e.g., Nash, Biochem J. 1953 October; 55(3):416-21. The activity of an MDH may be measured in cell lysate, in an intact cell, or as an isolated MDH.

In some embodiments, the activity (e.g., specific activity) of an MDH (e.g., in cell lysate, in an intact cell, or as an isolated MDH) of the present disclosure is at least 1.1 fold (e.g., at least 1.3 fold, at least 1.5 fold, at least 1.7 fold, at least 1.9 fold, at least 2 fold, at least 2.5 fold, at least 3 fold, at least 4 fold, at least 5 fold, at least 10 fold, at least 20 fold, at least 30 fold, at least 40 fold, at least 50 fold, or at least 100 fold, including all values in between) greater than that of a control. As a non-limiting example, a control may be a cell that does not include the MDH of interest. In some embodiments, a control is MDH from Bacillus methanolicus or Cupriavidus necator N−1 (e.g., SEQ ID NOS: 30 or 32) (e.g., in cell lysate, in an intact cell, or as an isolated MDH). In certain embodiments, a control is a wild-type MDH sequence. In certain embodiments, the activity of an MDH is measured in a cell or cell lysate and is compared to a control that is a cell or cell lysate does not include the MDH.

In some embodiments, the activity (e.g., specific activity) of an MDH of the present disclosure is at least 1%, at least 5%, at least 10%, at least 15%, at least 20%, at least 30%, at least 40%, at least 50%, at least 60%, at least 70%, at least 80%, at least 90%, at least 100%, at least 110%, at least 120%, at least 130%, at least 140%, at least 150%, at least 160%, at least 170%, at least 180%, at least 190%, at least 200%, at least 500%, at least 1,000%, or any values in between that of the activity (e.g., specific activity) of a control MDH (e.g., CnMDHm3, A0A031LYD0_9GAMM, and/or a wild-type MDH).

As a non-limiting example, the MDH activity of a recombinant host cell or cell lysate may be measured by determining the NAD reductase activity (e.g., using a routine XTT enzyme activity assay). See, e.g., diagram provided in FIG. 6 for an XTT enzyme activity assay. In some embodiments, a recombinant host cell comprising any of the MDHs described herein has at least 5%, at least 10%, at least 20%, at least 25%, at least 30%, at least 40%, at least 45%, at least 50%, at least 55%, at least 60%, at least 75%, at least 80%, at least 85%, at least 90%, at least 95%, at least 100%, at least 105%, at least 110%, at least 115%, at least 120%, at least 125%, at least 130%, at least 140%, at least 150%, at least 160%, at least 170%, at least 180%, at least 190%, at least 200%, at least 500%, or at least 1000% the NAD reductase activity as compared to a control cell. In some embodiments, the control cell expresses a heterologous gene encoding CnMDHm3, A0A031LYD0_9GAMM, and/or a wild-type MDH. In some embodiments, a control cell has endogenous MDH expression. In some embodiments, a control cell does not endogenously express MDH. As a non-limiting example, the NAD reductase activity may also be determined for an isolated MDH and compared to a control MDH (e.g., CnMDHm3, A0A031LYD0_9GAMM, and/or a wild-type MDH).

The catalytic constant (k_{cat}) value of an MDH enzyme in a cell lysate may be determined by routine methods. For example, the k_{cat} value may be determined based on the calculation of total cellular protein concentration and NADH optical density or based on the calculation of target protein concentration and concentration of NADH in the cell lysate. In some embodiments, the present disclosure provides MDH enzymes having a k_{cat} of at least 0.01 s⁻¹, at least 0.05 s⁻¹, at least 0.1 s⁻¹, at least 0.5 s⁻¹, at least 1 s⁻¹, at least 5 s⁻¹, at least 10 s⁻¹, at least 15 s⁻¹, at least 20 s⁻¹, at least 25 s⁻¹, at least 30 s⁻¹, at least 40 s⁻¹, at least 50 s⁻¹, at least 60 s⁻¹, at least 70 s⁻¹, at least 80 s⁻¹, at least 90 s⁻¹, at least 100 s⁻¹, at least 125 s⁻¹, at least 150 s⁻¹, at least 175 s⁻¹, at least 200 s⁻¹, at least 225 s⁻¹, at least 250 s⁻¹, at least 275 s⁻¹, at least 300 s⁻¹, at least 325 s⁻¹, at least 350 s⁻¹, at least 375 s⁻¹, at least 400 s⁻¹, at least 450 s⁻¹, at least 500 s⁻¹, at least 550 s⁻¹, at least 600 s⁻¹, at least 700 s⁻¹, at least 800 s⁻¹, at least 900 s⁻¹, or at least 1,000 s⁻¹.

The k_{cat} value of an MDH enzyme may also be measured as an isolated protein using routine methods. The k_{cat} value of an isolated MDH enzyme may be least 0.01 s⁻¹, at least 0.05 s⁻¹, at least 0.1 s⁻¹, at least 0.5 s⁻¹, at least 1 s⁻¹, at least 5 s⁻¹, at least 10 s⁻¹, at least 15 s⁻¹, at least 20 s⁻¹, at least 25 s⁻¹, at least 30 s⁻¹, at least 40 s⁻¹, at least 50 s⁻¹, at least 60 s⁻¹, at least 70 s⁻¹, at least 80 s⁻¹, at least 90 s⁻¹, at least 100 s⁻¹, at least 125 s⁻¹, at least 150 s⁻¹, at least 175 s⁻¹, at least 200 s⁻¹, at least 225 s⁻¹, at least 250 s⁻¹, at least 275 s⁻¹, at least 300 s⁻¹, at least 325 s⁻¹, at least 350 s⁻¹, at least 375 s⁻¹, at least 400 s⁻¹, at least 450 s⁻¹, at least 500 s⁻¹, at least 550 s⁻¹, at least 600 s⁻¹, at least 700 s⁻¹, at least 800 s⁻¹, at least 900 s⁻¹, or at least 1,000 s⁻¹,

The Kₘ or the concentration of substrate which permits the enzyme to achieve half Vₘₐₓ may also be calculated for any of the MDH enzymes described herein in cell lysate. The Kₘ of an MDH enzyme in a cell lysate may be determined based on the calculation of total cellular protein concentration and NADH optical density or based on the calculation of target protein concentration and concentration of NADH in the cell lysate. In some embodiments, a recombinant host cell of the present disclosure may include an MDH having a Kₘ value of less than 0.001 M, less than 0.005 M, less than 0.01 M, less than 0.02 M, less than 0.03 M less than, less than 0.04 M, less than 0.05 M, less than 0.06 M, less than 0.07 M, less than 0.08 M, less than 0.09 M, less than 0.1 M, less than 0.2 M, less than 0.3 M, less than 0.4 M, less than 0.5 V, less than 0.6 M, less than 0.7 V, less than 0.8 M, less than 0.9 V, less than 1 M, less than 1.1 M, less than 1.2 M, less than 1.3 V, less than 1.4 M, less than 1.5 V, less than 1.6 M, less than 1.7 M, less than 1.8 M, less than 1.9 M, less than 2 M, less than 3 M, less than 5 M, less than 10 v, or any values in between.

The Kₘ value of an isolated MDH may be determined using routine methods. In some embodiments, an isolated MDH of the present disclosure may have a Kₘ value of less than 0.001 M, less than 0.005 M, less than 0.01 M, less than 0.02 M, less than 0.03 M less than, less than 0.04 M, less than 0.05 M, less than 0.06 M, less than 0.07 M, less than 0.08 M, less than 0.09 M, less than 0.1 M, less than 0.2 M, less than 0.3 M, less than 0.4 M, less than 0.5 M, less than 0.6 M, less than 0.7 M, less than 0.8 M, less than 0.9 M, less than 1 M, less than 1.1 M, less than 1.2 M, less than 1.3 M, less than 1.4 M, less than 1.5 M, less than 1.6 M, less than 1.7 M, less than 1.8 M, less than 1.9 M, less than 2 M, less than 3 M, less than 5 M, less than 10 M, or any values in between.

In some embodiments, the present disclosure provides MDH enzymes having a k_{cat}/Kₘ ratio that is greater than 0.001 L/(mol*s), greater than 0.005 L/(mol*s), greater than 1 L/(mol*s), greater than 5 L/(mol*s), greater than 10 L/(mol*s), greater than 20 L/(mol*s), greater than 30 L/(mol*s), greater than 40 L/(mol*s), greater than 50 L/(mol*s), greater than 60 L/(mol*s), greater than 70 L/(mol*s), greater than 80 L/(mol*s), greater than 90 L/(mol*s), greater than 100 L/(mol*s), greater than 200 L/(mol*s), greater than 300 L/(mol*s), greater than 400 L/(mol*s), greater than 500 L/(mol*s), greater than 600 L/(mol*s), greater than 700 L/(mol*s), greater than 800 L/(mol*s), greater than 900 L/(mol*s), greater than 1,000 L/(mol*s), greater than 2,500 L/(mol*s), greater than 5,000 L/(mol*s), greater than 10,000 L/(mol*s), or any value in between. The k_{cat}/Kₘ ratio of an MDH enzyme may be calculated in cell lysate or for an isolated MDH enzyme.

In some embodiments, MDH enzymes of the present disclosure have a k_{cat}/Kₘ ratio from about 100 L/(mol*s) to about 1500 L/(mol*s). In some embodiments, a k_{cat}/Kₘ ratio is from about 250 L/(mol*s) to about 1000 L/(mol*s) as calculated based on total protein and optical density of NADH. In some embodiments, a k_{cat}/Kₘ ratio is from about 300 L/(mol*s) to about 600 L/(mol*s) as calculated based on total protein and optical density of NADH. In some embodiments, a k_{cat}/Kₘ ratio is at least 300 L/(mol*s), at least 400 L/(mol*s), at least 500 L/(mol*s), at least 600 L/(mol*s), at least 700 L/(mol*s), at least 800 L/(mol*s), at least 900 L/(mol*s), or at least 1,000 L/(mol*s) as calculated based on total protein and optical density of NADH.

In some embodiments, the present disclosure provides MDH enzymes having a k_{cat}/Kₘ ratio of from about 1 L/(mol*s) to about 75 L/(mol*s) as calculated based on concentration of target protein and NADH. In some embodiments a k_{cat}/Kₘ ratio is from about 1 L/(mol*s) to about 30 L/(mol*s) as calculated based on concentration of target protein and NADH. In some embodiments, a k_{cat}/Kₘ ratio is from about 10 L/(mol*s) to about 50 L/(mol*s) as calculated based on concentration of target protein and NADH. In some embodiments, a k_{cat}/Kₘ ratio is from about 1 L/(mol*s) to about 10 L/(mol*s) or to about 30 L/(mol*s) as calculated based on concentration of target protein and NADH. In some embodiments, a k_{cat}/Kₘ ratio is at least 1 L/(mol*s), at least 10 L/(mol*s), at least 20 L/(mol*s), at least 25 L/(mol*s), or at least 50 L/(mol*s) as calculated based on concentration of target protein and NADH.

It should be appreciated that one of ordinary skill in the art would be able to characterize a protein as an MDH enzyme based on structural and/or functional information associated with the protein. For example, in some embodiments, a protein can be characterized as an MDH enzyme based on its function, such as the ability to produce formaldehyde from methanol. In some embodiments, an MDH enzyme of the present disclosure is a decamer. In some embodiments, an MDH enzyme of the present disclosure includes an aspartic acid (D) residue at a position corresponding to position 100 of MDH from Bacillus methanolicus (UniprotKB Database Reference Number: P31005), a lysine (K) residue corresponding to position 103 from Bacillus methanolicus (UniprotKB Database Reference Number: P31005), or a combination thereof.

As used herein, a residue (such as a nucleic acid residue or an amino acid residue) in sequence “X” is referred to as corresponding to a position or residue (such as a nucleic acid residue or an amino acid residue) “a” in a different sequence “Y” when the residue in sequence “X” is at the counterpart position of “a” in sequence “Y” when sequences X and Y are aligned using amino acid sequence alignment tools known in the art, such as, for example, Clustal Omega or BLAST®.

In some embodiments, a recombinant host cell that expresses a heterologous gene encoding an MDH enzyme produces at least 10%, 20%, 30%, 40%, 50%, 60%, 70%, 80%, 90%, or 100% more formaldehyde compared to the same recombinant host cell that does not express the heterologous gene.

In some embodiments, an MDH enzyme (e.g., an isolated MDH enzyme) produces at least 10%, 20%, 30%, 40%, 50%, 60%, 70%, 80%, 90%, or 100% more formaldehyde compared to a control MDH enzyme (e.g., CnMDHm3, A0A031LYD0_9GAMM, and/or a wild-type MDH).

In other embodiments, a protein can be characterized as an MDH enzyme based on the percent identity between the protein and a known MDH enzyme. For example, the protein may be at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identical, including all values in between, to any of the MDH sequences described herein or the sequence of any other MDH enzyme. In other embodiments, a protein can be characterized as an MDH enzyme based on the presence of one or more domains (e.g., alcohol dehydrogenase domain, e.g., Fe-ADH in the Conserved Domains Database in the NCBI database under: cd08551, a NAD(P)-binding Rossman fold domain, or any combination thereof) in the protein that are associated with MDH enzymes.

In some embodiments, an MDH sequence includes at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, east least 56, at least 57, at least 58, at least 59, at least 60, at least 61, at least 62, at least 63, at least 64, at least 65, at least 66, at least 67, at least 68, at least 69, at least 70, at least 75, at least 80, at least 85, at least 90, at least 95, or at least 100 mutations, including all values in between, compared to a sequence (e.g., nucleic acid or amino acid sequence) set forth as SEQ ID NOS: 1-28, SEQ ID NOS: 73-80, SEQ ID NOS: 29-56, or SEQ ID NOS: 81-88, or compared to a sequence selected from sequences in Table 2, or a sequence selected from sequences in FIGS. 5-6.

In some embodiments, an MDH sequence includes a conservative amino acid substitution relative to one or more MDH sequences set forth as SEQ ID NOS: 29-56, or SEQ ID NOS: 81-88, or relative to MDH sequences in Table 2, or relative to MDH sequences in FIGS. 5-6. See, e.g., Table 1 for a non-limiting list of conservative amino acid substitutions.

It should be understood that an MDH may include a protein sequence that is identical to: an amino acid sequence set forth in SEQ ID NOS: 29-56 or SEQ ID NOS: 81-88; an MDH amino acid sequence in Table 2 that is encoded by a nucleic acid sequence including a synonymous mutation relative to a sequence set forth in SEQ ID NOS: 1-28 or SEQ ID NOS: 73-80; or an MDH amino acid sequence encoded by a nucleic acid sequence in Table 2.

In some embodiments, an MDH of the present disclosure may include a sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or is 100% identical, including all values in between, to SEQ ID NO: 34.

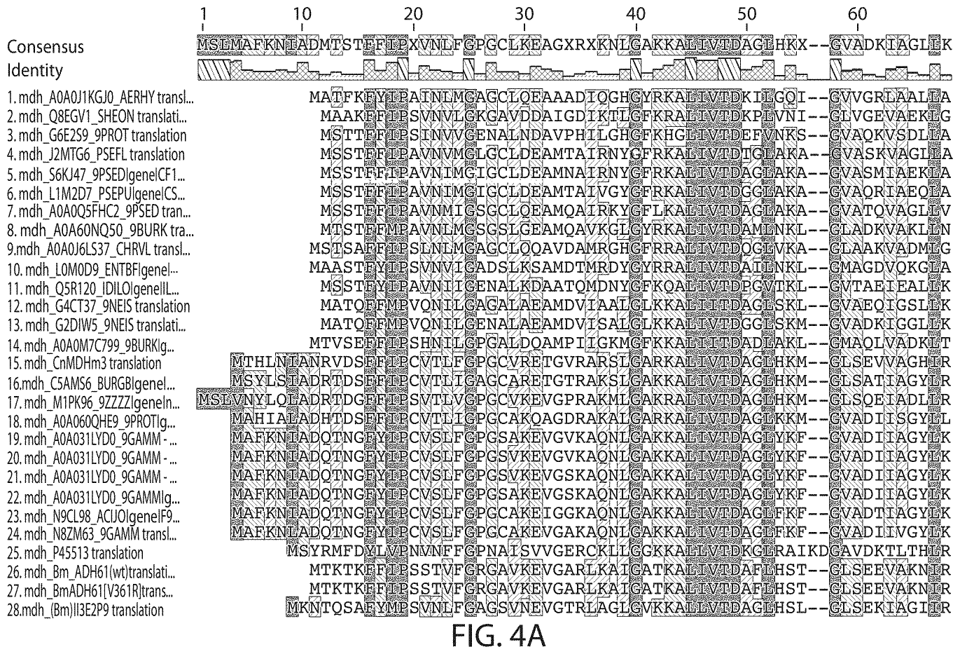

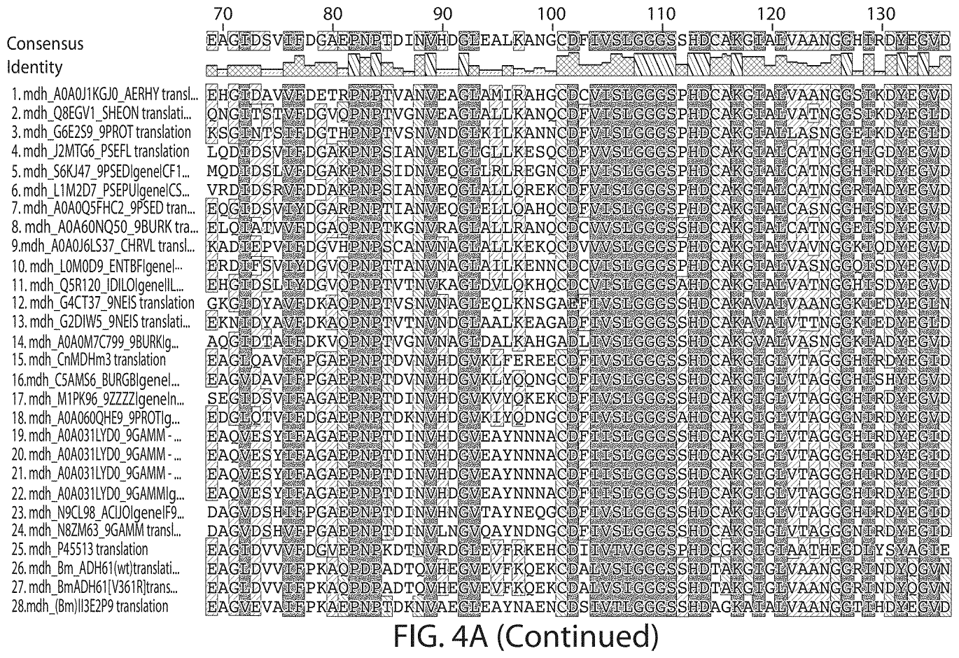

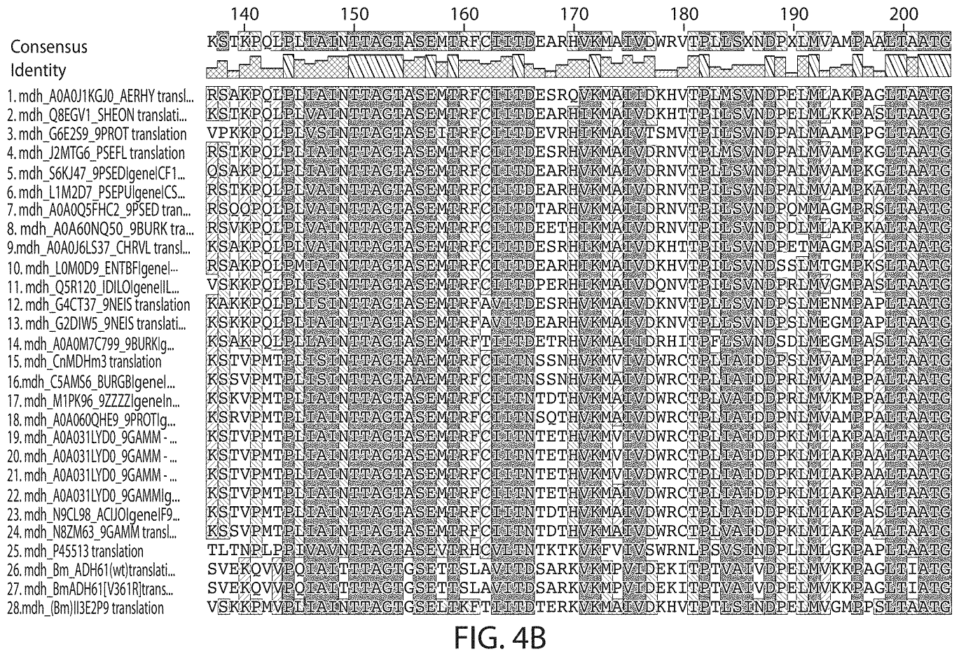

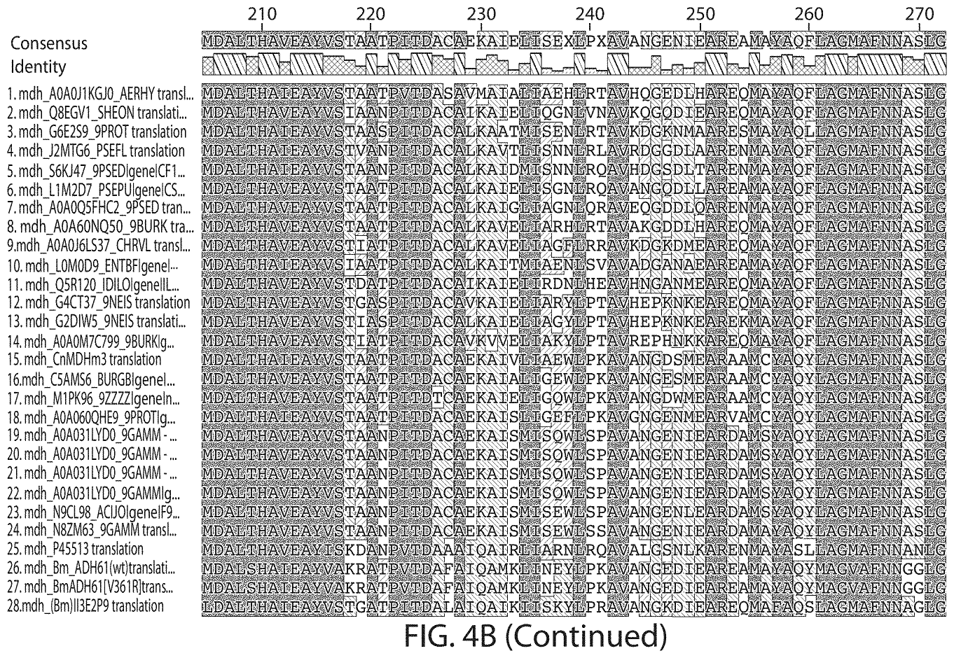

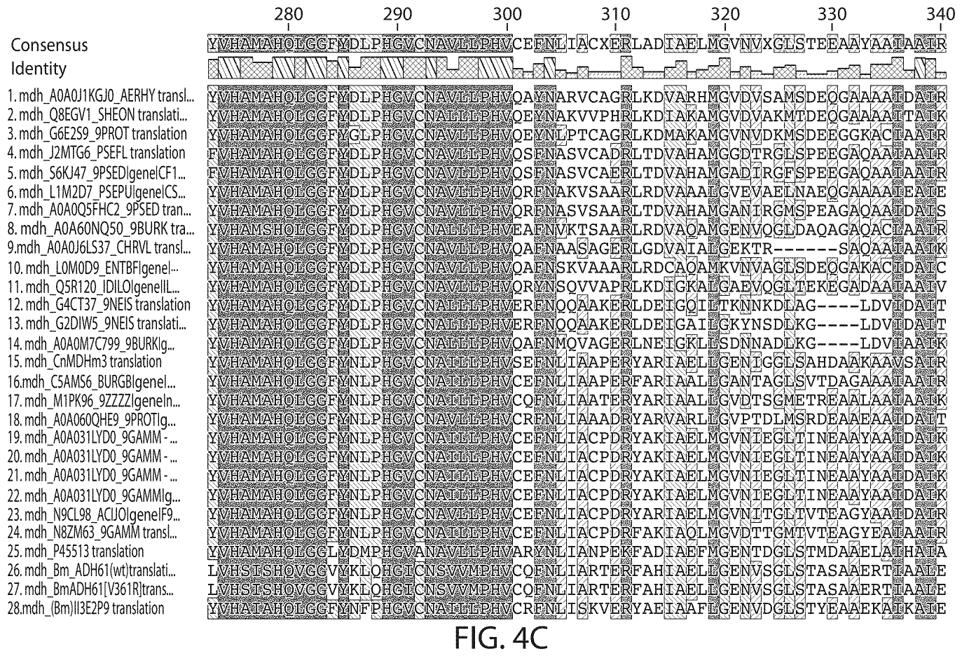

In some embodiments, an MDH of the present disclosure may include a sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or is 100% identical, including all values in between, to a highly conserved region of an MDH sequence, such as the region corresponding to residues 96 to 295 of SEQ ID NO: 34 (FIGS. 4A-4C) or to the corresponding region of any one of SEQ ID NOS: 29-33, 35-56 or 81-88 (FIGS. 4A-4C).

In some embodiments, an MDH of the present disclosure includes one or more conserved residues at a position that corresponds to one or more conserved residues depicted in FIGS. 4A-4C. In some embodiments, an MDH of the present disclosure includes at least two (e.g., at least 3, 4, 5, 6, 7, 8, 9, 10, 11, 12, 13, 14, 15, 16, 17, 18, 19, or at least 20) residues that are conserved in a region corresponding to a highly conserved region depicted in FIGS. 4A-4C.

In some embodiments, an MDH of the present disclosure includes a region that corresponds to residues 256 to 295 of wild-type A0A031LYD0_9GAMM (SEQ ID NO: 34) and the region includes no more than 1, 2, 3, 4, 5, 6, 7, 8, 9, 10, 11, 12, 13, 14, 15, 16, 17 18, 19, 20, 21, 22, 23, 24, 25, 26, 27, 28, 29, 30, 31, 32, 33, 34, 35, 36, 37, or 38 amino acid substitutions relative to residues 256 to 295 of wild-type A0A031LYD0_9GAMM (SEQ ID NO: 34). As a non-limiting example, the region corresponding to residues 256 to 295 of wild-type A0A031LYD0_9GAMM (SEQ ID NO: 34) may include a leucine (L) or methionine (M) at a residue corresponding to position 256 of wild-type A0A031LYD0_9GAMM (SEQ ID NO: 34); a valine (V) or methionine (M) at a residue corresponding to position 259 of wild-type A0A031LYD0_9GAMM (SEQ ID NO: 34); an alanine (A) or glycine (G) at a residue corresponding to position 264 of wild-type A0A031LYD0_9GAMM (SEQ ID NO: 34); an asparagine (N), glycine (G), or serine (S) at a residue corresponding to position 265 of wild-type A0A031LYD0_9GAMM (SEQ ID NO: 34); a phenylalanine (F), tyrosine (Y), or leucine (L) at a residue corresponding to position 268 of wild-type A0A031LYD0_9GAMM (SEQ ID NO: 34); an alanine (A) or serine (S) at a residue corresponding to position 271 of wild-type A0A031LYD0_9GAMM (SEQ ID NO: 34); a isoleucine (I) or methionine (M) at a residue corresponding to position 272 of wild-type A0A031LYD0_9GAMM (SEQ ID NO: 34); an alanine (A) or serine (S) at a residue corresponding to position 273 of wild-type A0A031LYD0_9GAMM (SEQ ID NO: 34); a leucine (L) or valine (V) at a residue corresponding to position 276 of wild-type A0A031LYD0_9GAMM (SEQ ID NO: 34); a phenylalanine (F), leucine (L), or valine (V) at a residue corresponding to position 279 of wild-type A0A031LYD0_9GAMM (SEQ ID NO: 34); an asparagine (N), aspartic acid (D), glycine (G), or lysine (K) at a residue corresponding to position 281 of wild-type A0A031LYD0_9GAMM (SEQ ID NO: 34); a leucine (L), methionine (M), or phenylalanine (F) at a residue corresponding to position 282 of wild-type A0A031LYD0_9GAMM (SEQ ID NO: 34); a proline (P) or glutamine (Q) at a residue corresponding to position 283 of wild-type A0A031LYD0_9GAMM (SEQ ID NO: 34); a valine (V) or isoleucine (I) at a residue corresponding to position 286 of wild-type A0A031LYD0_9GAMM (SEQ ID NO: 34); an alanine (A) or cysteine (C) at a residue corresponding to position 287 of wild-type A0A031LYD0_9GAMM (SEQ ID NO: 34); an alanine (A) or serine (S) at a residue corresponding to position 289 of wild-type A0A031LYD0_9GAMM (SEQ ID NO: 34); a leucine (L), valine (V), or isoleucine (I) at a residue corresponding to position 290 of wild-type A0A031LYD0_9GAMM (SEQ ID NO: 34); a leucine (L) or valine (V) at a residue corresponding to position 291 of wild-type A0A031LYD0_9GAMM (SEQ ID NO: 34); and/or a methionine (M) or leucine (L) at a residue corresponding to position 292 of wild-type A0A031LYD0_9GAMM (SEQ ID NO: 34). An MDH of the present disclosure may include the amino acid sequence LAGMAFNNASLGYVHAMXHQLGGFYXLPHGVCNAXLLPHV (SEQ ID NO: 57), wherein X is any amino acid. In some instances, position 18 in SEQ ID NO: 57 is alanine (A) or serine (S), position 26 in SEQ ID NO: 57 is asparagine (N) or aspartic acid (D), and/or position 35 in SEQ ID NO: 57 is leucine (L), valine (V), or isoleucine (I). See also, e.g., SEQ ID NO: 58.

An MDH of the present disclosure may include a region corresponding to residues 167 to 172 of wild-type A0A031LYD0_9GAMM (SEQ ID NO: 34) and in some embodiments, the region includes no more than 1, 2, 3, 4, or 5 amino acid substitutions relative to residues 167 to 172 of wild-type A0A031LYD0_9GAMM (SEQ ID NO: 34). As a non-limiting example, an MDH of the present disclosure may include a region corresponding to residues 167 to 172 of wild-type A0A031LYD0_9GAMM (SEQ ID NO: 34) and includes a valine (V) at a residue corresponding to position 169 of wild-type A0A031LYD0_9GAMM (SEQ ID NO: 34). In some instances, an MDH includes an alanine (A), proline (P), or valine (V) at a residue corresponding to position 169 of wild-type A0A031LYD0_9GAMM (SEQ ID NO: 34). In some instances, an MDH of the present disclosure includes the amino acid sequence KMAIVD (SEQ ID NO: 59), KMAIID (SEQ ID NO: 60), KFVIVS (SEQ ID NO: 61), KMAIVT (SEQ ID NO: 62), KMPVID (SEQ ID NO: 63), KMPVID (SEQ ID NO: 64), or KMVIVD (SEQ ID NO: 65). See also, e.g., FIGS. 4A-4C.

An MDH of the present disclosure may include a region corresponding to residues 366 to 369 of wild-type A0A031LYD0_9GAMM (SEQ ID NO: 34) and in some embodiments, the region includes no more than 1, 2, or 3 amino acid substitutions relative to residues 366 to 369 of wild-type A0A031LYD0_9GAMM (SEQ ID NO: 34). In some instances, the region includes an alanine (A), valine (V), glycine (G), or arginine (R) at a residue corresponding to position 368 of wild-type A0A031LYD0_9GAMM (SEQ ID NO: 34). In some instances, the region includes an arginine (R) at a residue corresponding to position 368 of wild-type A0A031LYD0_9GAMM (SEQ ID NO: 34). As a non-limiting example, an MDH of the present disclosure may in some instances include the sequence KDAC (SEQ ID NO: 66), KDVC (SEQ ID NO: 67), KDGN (SEQ ID NO: 68), QDVC (SEQ ID NO: 69), QDRC (SEQ ID NO: 70), NDAC (SEQ ID NO: 71), or KDRC (SEQ ID NO: 72). See also, e.g., FIGS. 4A-4C.

An MDH of the present disclosure may include a region corresponding to residues 42 to 46 of wild-type A0A031LYD0_9GAMM (SEQ ID NO: 34). In some instances, the region corresponding to residues 42 to 46 includes 1, 2, 3, or 4 amino acid substitutions relative to residues 42 to 46 of wild-type A0A031LYD0_9GAMM (SEQ ID NO: 34). In some instances, the region includes no more than 4 (e.g., no more than 3, no more than 2, or no more than 1) amino acid substitutions relative to residues 42 to 46 of wild-type A0A031LYD0_9GAMM (SEQ ID NO: 34). See also, e.g., FIGS. 4A-4C.

An MDH of the present disclosure may include a region corresponding to residues 101 to 112 of wild-type A0A031LYD0_9GAMM (SEQ ID NO: 34). In certain instances, the region includes 1, 2, 3, 4, 5, 6, 7, 8, 9, 10, or 11 amino acid substitutions relative to residues 101 to 112 of wild-type A0A031LYD0_9GAMM (SEQ ID NO: 34). In certain instances, the region includes no more than 11 (e.g., no more than 10, no more than 9, no more than 8, no more than 7, no more than 6, no more than 5, no more than 4, no more than 3, no more than 2, no more than 1) amino acid substitutions relative to residues 101 to 112 of wild-type A0A031LYD0_9GAMM (SEQ ID NO: 34). See also, e.g., FIGS. 4A-4C.

An MDH of the present disclosure may include a region corresponding to residues 144 to 152 of wild-type A0A031LYD0_9GAMM (SEQ ID NO: 34). In certain instances, the region includes no more than 8 (e.g., no more than 7, no more than 6, no more than 5, no more than 4, no more than 3, no more than 2, no more than 1) amino acid substitutions relative to residues 144 to 152 of wild-type A0A031LYD0_9GAMM (SEQ ID NO: 34). In certain instances, the region includes 1, 2, 3, 4, 5, 6, 7, or 8 amino acid substitutions relative to residues 144 to 152 of wild-type A0A031LYD0_9GAMM (SEQ ID NO: 34). See also, e.g., FIGS. 4A-4C.

An MDH of the present disclosure may include a region corresponding to residues 194 to 211 of wild-type A0A031LYD0_9GAMM (SEQ ID NO: 34). In some instances, the region includes no more than 17 (e.g., no more than 16, no more than 15, no more than 14, no more than 13, no more than 12, no more than 11, no more than 10, no more than 9, no more than 8, no more than 7, no more than 6, no more than 5, no more than 4, no more than 3, no more than 2, or no more than 1) amino acid substitutions relative to residues 194 to 211 of wild-type A0A031LYD0_9GAMM (SEQ ID NO: 34). In some instances, the region includes 1, 2, 3, 4, 5, 6, 7, 8, 9, 10, 11, 12, 13, 14, 15, 16, or 17 amino acid substitutions relative to residues 194 to 211 of wild-type A0A031LYD0_9GAMM (SEQ ID NO: 34). See also, e.g., FIGS. 4A-4C.

In some instances, an MDH includes an alanine (A), aspartic acid (D), glutamic acid (E), asparagine (N), proline (P), glutamine (Q), serine (S), threonine (T), valine (V), or glycine (G) at an amino acid residue corresponding to position 31 in A0A031LYD0_9GAMM.

In some instances, an MDH includes an alanine (A), a isoleucine (I), a leucine (L), or valine (V) at an amino acid residue corresponding to position 26 in A0A031LYD0_9GAMM. See also, e.g., FIGS. 4A-4C.

In some embodiments, an MDH of the present disclosure includes 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 130, 140, 150, 160, 170, 180, 190, 200, including any values in between, or more, mutations, relative to Acinetobacter sp. Ver3 Uniprot A0A031LYD0_9GAMM (SEQ ID NO: 34). In some embodiments, an MDH of the present disclosure includes a mutation at a residue corresponding to position 31, position 26, position 169, position 368, or any combination thereof in A0A031LYD0_9GAMM (SEQ ID NO: 34). In some embodiments, a residue in an MDH corresponding to position 26 in A0A031LYD0_9GAMM (SEQ ID NO: 34) is a valine (V) or a conservative amino acid substitution of valine (V). In some embodiments, an alanine (A) residue in an MDH corresponding to residue 26 in A0A031LYD0_9GAMM (SEQ ID NO: 34) is mutated to a valine (V) or a conservative amino acid substitution of valine (V). In some embodiments, a residue in an MDH corresponding to position 26 in A0A031LYD0_9GAMM (SEQ ID NO: 34) includes a nonpolar aliphatic R group. In some embodiments, a residue in an MDH corresponding to position 169 in A0A031LYD0_9GAMM (SEQ ID NO: 34) is a valine or a conservative amino acid substitution of valine. In some embodiments, an alanine residue in an MDH corresponding to residue 169 in A0A031LYD0_9GA1/MM (SEQ ID NO: 34) is mutated to a valine or a conservative amino acid substitution of valine. In some embodiments, a residue in an MDH corresponding to position 169 in A0A031LYD0_9GAMM (SEQ ID NO: 34) includes a nonpolar aliphatic R group. In some embodiments, a residue in an MDH corresponding to position 31 in A0A031LYD0_9GAMM (SEQ ID NO: 34) is a valine or a conservative amino acid substitution of valine. In some embodiments, a serine residue in an MDH corresponding to residue 31 in A0A031LYD0_9GAMM (SEQ ID NO: 34) is mutated to a valine or a conservative amino acid substitution of valine. In some embodiments, a residue in an MDH corresponding to position 31 in A0A031LYD0_9GAMM (SEQ ID NO: 34) includes a nonpolar aliphatic R group.

In some embodiments, a residue in an MDH corresponding to position 368 in A0A031LYD0_9GAMM (SEQ ID NO: 34) is an arginine or a conservative amino acid substitution of arginine. In some embodiments, an alanine residue in an MDH corresponding to residue 368 in A0A031LYD0_9GAMM (SEQ ID NO: 34) is mutated to an arginine or a conservative amino acid substitution of arginine. In some embodiments, a residue in an MDH corresponding to position 368 in A0A031LYD0_9GAMM (SEQ ID NO: 34) includes a positively charged R group. See also, e.g., FIGS. 4A-4C.

In some embodiments, an MDH of the present disclosure includes the following mutations relative to A0A031LYD0_9GAMM (SEQ ID NO: 34): A26V, S31V, A169V, A368R or a combination thereof. In some embodiments, an MDH of the present disclosure includes the following mutations relative to A0A031LYD0_9GAMM (SEQ ID NO: 34): (1) A26V, S31V, A169V, and A368R; (2) A26V, A169V, and A368R; (3) A26V and A368R; or (4) S31V, A169V, and A368R. See also, e.g., FIGS. 4A-4C.

In some embodiments, an MDH of the present disclosure includes 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more mutations relative to J2MTG6_PSEFL (SEQ ID NO: 48). In some embodiments, an MDH of the present disclosure includes a mutation at a residue corresponding to position 18, position 23, position 161, position 360, or any combination thereof in J2MTG6_PSEFL (SEQ ID NO: 48). In some embodiments, a residue in an MDH corresponding to position 18 in J2MTG6_PSEFL (SEQ ID NO: 48) is a valine or a conservative amino acid substitution of valine. In some embodiments, a leucine residue in an MDH corresponding to residue 18 in J2MTG6_PSEFL (SEQ ID NO: 48) is mutated to a valine or a conservative amino acid substitution of valine. In some embodiments, a residue in an MDH corresponding to position 18 in J2MTG6_PSEFL (SEQ ID NO: 48) includes a nonpolar aliphatic R group. In some embodiments, a residue in an MDH corresponding to position 23 in J2MTG6_PSEFL (SEQ ID NO: 48) is a valine or a conservative amino acid substitution of valine. In some embodiments, an threonine residue in an MDH corresponding to residue 23 in J2MTG6_PSEFL (SEQ ID NO: 48) is mutated to a valine or a conservative amino acid substitution of valine. In some embodiments, a residue in an MDH corresponding to position 23 in J2MTG6_PSEFL (SEQ ID NO: 48) includes a nonpolar aliphatic R group. In some embodiments, a residue in an MDH corresponding to position 161 in J2MTG6_PSEFL (SEQ ID NO: 48) is a valine or a conservative amino acid substitution of valine. In some embodiments, an alanine residue in an MDH corresponding to residue 161 in J2MTG6_PSEFL (SEQ ID NO: 48) is mutated to a valine or a conservative amino acid substitution of valine. In some embodiments, a residue in an MDH corresponding to position 161 in J2MTG6_PSEFL (SEQ ID NO: 48) includes a nonpolar aliphatic R group. In some embodiments, a residue in an MDH corresponding to position 360 in J2MTG6_PSEFL (SEQ ID NO: 48) is an arginine or a conservative amino acid substitution of arginine. In some embodiments, an alanine residue in an MDH corresponding to residue 360 in J2MTG6_PSEFL (SEQ ID NO: 48) is mutated to an arginine or a conservative amino acid substitution of arginine. In some embodiments, a residue in an MDH corresponding to position 360 in J2MTG6_PSEFL (SEQ ID NO: 48) includes a positively charged R group.

In some embodiments, an MDH of the present disclosure includes 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more mutations relative to Q5R120_IDILO (SEQ ID NO: 38). In some embodiments, an MDH of the present disclosure includes a mutation at a residue corresponding to position 18, position 23, position 161, position 360, or any combination thereof in Q5R120_IDILO (SEQ ID NO: 38). In some embodiments, a residue in an MDH corresponding to position 18 in Q5R120_IDILO (SEQ ID NO: 38) is a valine or a conservative amino acid substitution of valine. In some embodiments, a leucine residue in an MDH corresponding to residue 18 in Q5R120_IDILO (SEQ ID NO: 38) is mutated to a valine or a conservative amino acid substitution of valine. In some embodiments, a residue in an MDH corresponding to position 18 in Q5R120_IDILO (SEQ ID NO: 38) includes a nonpolar aliphatic R group. In some embodiments, a residue in an MDH corresponding to position 23 in Q5R120_IDILO (SEQ ID NO: 38) is a valine or a conservative amino acid substitution of valine. In some embodiments, a threonine residue in an MDH corresponding to residue 23 in Q5R120_IDILO (SEQ ID NO: 38) is mutated to a valine or a conservative amino acid substitution of valine. In some embodiments, a residue in an MDH corresponding to position 23 in Q5R120_IDILO (SEQ ID NO: 38) includes a nonpolar aliphatic R group. In some embodiments, a residue in an MDH corresponding to position 161 in Q5R120_IDILO (SEQ ID NO: 38) is a valine or a conservative amino acid substitution of valine. In some embodiments, an alanine residue in an MDH corresponding to residue 161 in Q5R120_IDILO (SEQ ID NO: 38) is mutated to a valine or a conservative amino acid substitution of valine. In some embodiments, a residue in an MDH corresponding to position 161 in Q5R120_IDILO (SEQ ID NO: 38) includes a nonpolar aliphatic R group. In some embodiments, a residue in an MDH corresponding to position 360 in Q5R120_IDILO (SEQ ID NO: 38) is an arginine or a conservative amino acid substitution of arginine. In some embodiments, an alanine residue in an MDH corresponding to residue 360 in Q5R120_IDILO (SEQ ID NO: 38) is mutated to an arginine or a conservative amino acid substitution of arginine. In some embodiments, a residue in an MDH corresponding to position 360 in Q5R120_IDILO (SEQ ID NO: 38) includes a positively charged R group.

In some embodiments, an MDH of the present disclosure includes 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more mutations relative to Uniprot C5AMS6_BURGB (SEQ ID NO: 43). In some embodiments, an MDH of the present disclosure includes a mutation at a residue corresponding to position 26, position 31, position 169, or position 368, or any combination thereof in C5AMS6_BURGB (SEQ ID NO: 43). In some embodiments, a residue in an MDH corresponding to position 26 in C5AMS6_BURGB (SEQ ID NO: 43) is a valine or a conservative amino acid substitution of valine. In some embodiments, an alanine residue in an MDH corresponding to residue 26 in C5AMS6_BURGB (SEQ ID NO: 43) is mutated to a valine or a conservative amino acid substitution of valine. In some embodiments, a residue in an MDH corresponding to position 26 in C5AMS6_BURGB (SEQ ID NO: 43) includes a nonpolar aliphatic R group. In some embodiments, a residue in an MDH corresponding to position 31 in C5AMS6_BURGB (SEQ ID NO: 43) is a valine or a conservative amino acid substitution of valine. In some embodiments, a threonine residue in an MDH corresponding to residue 31 in C5AMS6_BURGB (SEQ ID NO: 43) is mutated to a valine or a conservative amino acid substitution of valine. In some embodiments, a residue in an MDH corresponding to position 31 in C5AMS6_BURGB (SEQ ID NO: 43) includes a nonpolar aliphatic R group. In some embodiments, a residue in an MDH corresponding to position 169 in C5AMS6_BURGB (SEQ ID NO: 43) is a valine or a conservative amino acid substitution of valine. In some embodiments, an alanine residue in an MDH corresponding to residue 169 in C5AMS6_BURGB (SEQ ID NO: 43) is mutated to a valine or a conservative amino acid substitution of valine. In some embodiments, a residue in an MDH corresponding to position 169 in C5AMS6_BURGB (SEQ ID NO: 43) includes a nonpolar aliphatic R group. In some embodiments, a residue in an MDH corresponding to position 368 in C5AMS6_BURGB (SEQ ID NO: 43) is a arginine or a conservative amino acid substitution of arginine. In some embodiments, an alanine residue in an MDH corresponding to residue 368 in C5AMS6_BURGB (SEQ ID NO: 43) is mutated to a arginine or a conservative amino acid substitution of arginine. In some embodiments, a residue in an MDH corresponding to position 368 in C5AMS6_BURGB (SEQ ID NO: 43) includes a positively charged R group.

In some embodiments, an MDH of the present disclosure includes 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more mutations relative to Q8EGV1_SHEON (SEQ ID NO: 46). In some embodiments, an MDH of the present disclosure includes a mutation at a residue corresponding to position 23, position 161, position 360, or any combination thereof in Q8EGV1_SHEON (SEQ ID NO: 46). In some embodiments, a residue in an MDH corresponding to position 18 in Q8EGV1_SHEON (SEQ ID NO: 46) is a valine or a conservative amino acid substitution of valine. In some embodiments, a residue in an MDH corresponding to position 18 in Q8EGV1_SHEON (SEQ ID NO: 46) includes a nonpolar aliphatic R group. In some embodiments, a residue in an MDH corresponding to position 23 in Q8EGV1_SHEON (SEQ ID NO: 46) is a valine or a conservative amino acid substitution of valine. In some embodiments, a glycine residue in an MDH corresponding to residue 23 in Q8EGV1_SHEON (SEQ ID NO: 46) is mutated to a valine or a conservative amino acid substitution of valine. In some embodiments, a residue in an MDH corresponding to position 23 in Q8EGV1_SHEON (SEQ ID NO: 46) includes a nonpolar aliphatic R group. In some embodiments, a residue in an MDH corresponding to position 161 in Q8EGV1_SHEON (SEQ ID NO: 46) is a valine or a conservative amino acid substitution of valine. In some embodiments, an alanine residue in an MDH corresponding to residue 161 in Q8EGV1_SHEON (SEQ ID NO: 46) is mutated to a valine or a conservative amino acid substitution of valine. In some embodiments, a residue in an MDH corresponding to position 161 in Q8EGV1_SHEON (SEQ ID NO: 46) includes a nonpolar aliphatic R group. In some embodiments, a residue in an MDH corresponding to position 360 in Q8EGV1_SHEON (SEQ ID NO: 46) is a arginine or a conservative amino acid substitution of arginine. In some embodiments, an alanine residue in an MDH corresponding to residue 360 in Q8EGV1_SHEON (SEQ ID NO: 46) is mutated to a arginine or a conservative amino acid substitution of arginine. In some embodiments, a residue in an MDH corresponding to position 360 in Q8EGV1_SHEON (SEQ ID NO: 46) includes a positively charged R group.

In some embodiments, an MDH of the present disclosure includes 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more mutations relative to I3DX19_BACMT (BmADH61) (SEQ ID NO:31). In some embodiments, an MDH of the present disclosure includes a mutation at a residue corresponding to position 361 in BmADH61 (SEQ ID NO:31). In some embodiments, a residue in an MDH corresponding to position 361 in BmADH61 (SEQ ID NO:31) is an arginine or a conservative amino acid substitution of arginine. In some embodiments, a valine residue in an MDH corresponding to position 361 in BmADH61 (SEQ ID NO:31) is mutated to arginine or a conservative amino acid substitution of arginine. In some embodiments, a residue in an MDH corresponding to position 361 in BmADH61 (SEQ ID NO:31) includes a positively charged R group.

In other embodiments, a protein can be characterized as an MDH enzyme based on a comparison of the three-dimensional structure of the protein compared to the three-dimensional structure of a known MDH enzyme (e.g., UniprotKB Database Reference Number: P31005, corresponding to MDH from Bacillus methanolicus). It should be appreciated that an MDH enzyme can be a synthetic protein.

### 3-hexulose-6-phosphate Synthase (Hexulose Phosphate Synthase, HPS) Enzymes

Aspects of the present disclosure provide 3-hexulose-6-phosphate synthase (hexulose phosphate synthase, HPS) enzymes, which may be useful, for example, in increasing methanol assimilation in organisms including bacteria and yeast.

As used herein, an HPS enzyme refers to an enzyme that is capable of converting formaldehyde and ribulose 5-phosphate into hexulose-6-P. HPS enzymes may use Mn(2+) or Mg(2+) as co-factors. Any suitable assay for measurement of HPS activity may be used. See, e.g., Quayle, Methods Enzymol. 1982; 90 Pt E:314-9.

In some embodiments, an HPS of the present disclosure is capable of producing at least 1%, at least 5%, at least 10%, at least 20%, at least 30%, at least 40%, at least 50%, at least 60%, at least 70%, at least 80%, at least 90%, at least 100%, at least 110%, at least 120%, at least 130%, at least 140%, at least 150%, at least 160%, at least 170%, at least 200%, at least 300%, at least 400%, at least 500%, at least 600%, at least 700%, at least 800%, at least 900%, at least 1,000%, or any value in between, more hexulose-6-P as compared to a control enzyme. The control HPS enzyme may be from Methylococcus capsulatus (e.g., UniProtKB-Q602L4) (SEQ ID NO: 122).

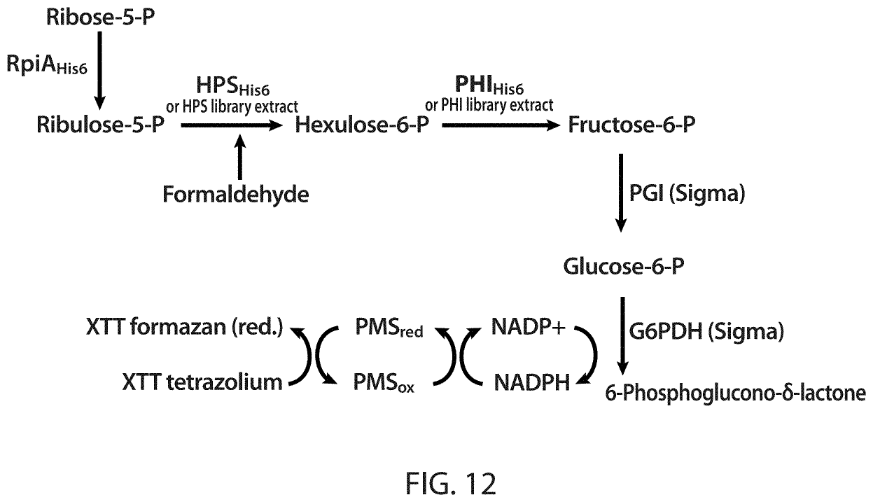

As a non-limiting example, a multi-enzyme linked assay may be used to determine HPS activity. For example, ribose phosphate isomerase (RPI) can be used to convert ribose-5-phosphate to ribulose-5-phosphate, and an isolated HPS enzyme of interest or lysate from a recombinant host cell expressing an HPS of interest may be introduced along with formaldehyde. If the HPS enzyme is capable of producing hexulose-6-phosphate from ribulose-5-phosphate and formaldehyde, hexulose-6-phosphate can serve as a substrate for 3-hexulose-6-phosphate isomerase (PHI). A PHI can be used, which could convert hexulose-6-phosphate to fructose-6-phosphate. Phosphoglucose isomerase (PGI) can be used to convert fructose-6-phosphate to glucose-6-phosphate. Finally, glucose-6-phosphate dehydrogenase (G6PDH) can be used to convert glucose-6-phosphate to 6-phosphoglucono-δ-lactone and produce NADPH from NADP+. NADPH production can be measured using absorbance at 340 nm or a solution including the electron transfer catalyst phenazine methosulfate (PMS) may be used along with XTT tetrazolium. If PMS solution and XTT tetrazolium are used, conversion of XTT tetrazolium to XTT formazan can be measured as a colorimetric readout (see also FIG. 12).

In some embodiments, an HPS enzyme (e.g., an isolated HPS, an HPS in an intact cell, or an HPS in cell lysate) has an activity that is at least 1%, at least 5%, at least 10%, at least 20%, at least 30%, at least 40%, at least 50%, at least 60%, at least 70%, at least 80%, at least 90%, at least 100%, at least 110%, at least 120%, at least 130%, at least 140%, at least 150%, at least 160%, at least 170%, at least 200%, at least 300%, at least 400%, at least 500%, at least 600%, at least 700%, at least 800%, at least 900%, at least 1,000%, or any value in between, compared to the activity of a control. A control may be an isolated control HPS enzyme, a cell or cell lysate including a control HPS enzyme, or a cell or cell lysate not including the HPS enzyme of interest. Non-limiting examples of HPS control enzymes include HPS from Methylococcus capsulatus.

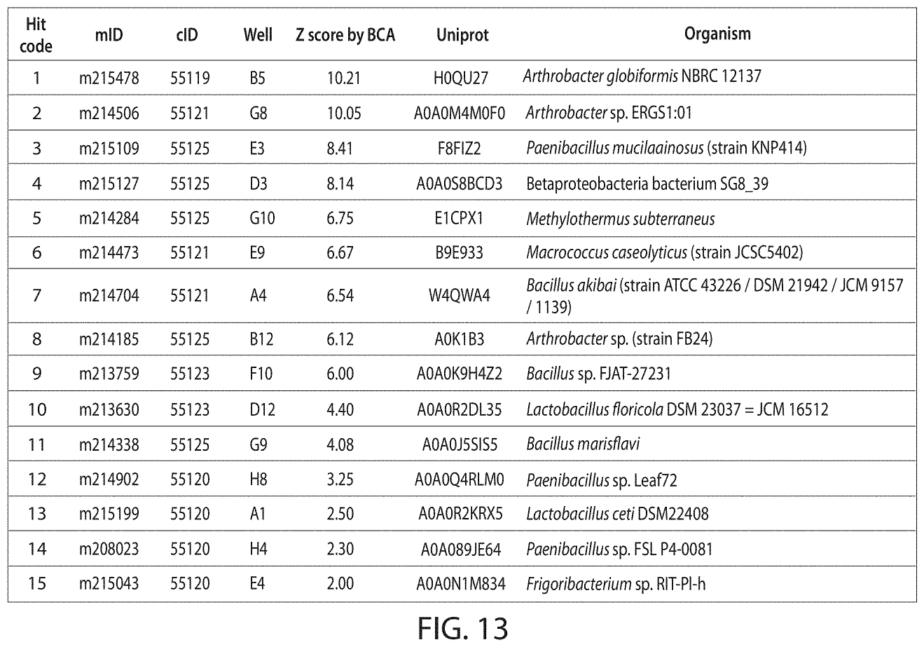

HPS enzymes may be from any species, including but not limited to, Methylococcus capsulatus, Arthrobacter globiformis, Arthrobacter sp. ERS1:01, Paenibacillus mucilaginosus, Betaproteobacteria bacterium, Methylothermus subterraneus, Macrococcus caseolyticus, Bacillus akibai, Arthrobacter sp. (strain FB24), Arthrobacter sp. (strain FB24), Bacillus sp. FJAT-27231, Lactobacillus floricola, Bacillus marisflavi, Paenibacillus sp. Leaf72, Lactobacillus ceti DSM 22408, Paenibacillus sp. FSL P4-0081, and Frigoribacterium sp. RIT-PI-h. In some embodiments, an HPS enzyme is from Brevibacterium casei, Arthrobacter methylotrophus, Mycobacterium gastri, Rhodococcus erythropolis, Amycolatopsis methanolica, Bacillus methanolicus, Acidomonas methanolica, Methylocapsa aurea, Afipia felis, Angulomicrobium tetraedrale, Methylobacterium extorquens, Methlyopila jiangsuensis, Paracoccus alkenifer, Sphingomonas melonis, Ancylobacter dichloromethanicus, Variovorax paradoxus, Methylophilus glucosoxydans, Methyloversatilis universalis, Methylibium aquaticum, Photobacterium indicum, Methylophaga thiooxydans, Methylococcus capsulatus, Klebsiella oxytoca, Gliocladium deliquescens, Paecilomyces variotii, Trichoderma lignorum, Candida boidini, Hansenula capsulatus, Pichia pastoris, Penicillium chrysogenum, or Photobacterium indicum. In some embodiments, an HPS enzyme is from a species shown in FIG. 13, or in Table 3. In some embodiments, an HPS enzyme is derived from a eukaryotic species that is capable of converting methanol into formaldehyde (e.g., Pichia spp.).

In some embodiments, an HPS of the present disclosure includes a sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or is 100% identical, including all values in between, compared to a sequence (e.g., nucleic acid or amino acid sequence) set forth as SEQ ID NOS: 89-105 or SEQ ID NOS: 106-122, or compared to an HPS sequence in Table 3, or an HPS sequence in FIG. 13.

In some embodiments, an HPS sequence includes a conservative amino acid substitution relative to one or more HPS sequences set forth in SEQ ID NOS: 106-122, or relative to one or more HPS sequences in FIG. 13, or relative to one or more HPS amino acid sequences in Table 3. See, e.g., Table 1 for a non-limiting list of conservative amino acid substitutions.

It should be understood that an HPS may include a protein sequence that is identical to: an amino acid sequence set forth in SEQ ID NOS: 106-122; an HPS amino acid sequence in Table 3 that is encoded by a nucleic acid sequence including a synonymous mutation relative to a sequence selected from SEQ ID NOS: 89-105; or compared to an HPS amino acid sequence encoded by a nucleic acid sequence in Table 3.

In some embodiments, an HPS enzyme includes a glutamine (Q) at a residue corresponding to position 4 of wild-type A0A0M4M0F0 (SEQ ID NO: 106); an alanine (A) at a residue corresponding to position 6 of wild-type A0A0M4M0F0 (SEQ ID NO: 106); an aspartic acid (D) at a residue corresponding to position 8 of wild-type A0A0M4M0F0 (SEQ ID NO: 106); an aspartic acid (D) at a residue corresponding to position 27 of wild-type A0A0M4M0F0 (SEQ ID NO: 106); a glutamic acid (E) at a residue corresponding to position 30 of wild-type A0A0M4M0F0 (SEQ ID NO: 106); a glycine (G) at a residue corresponding to position 32 of wild-type A0A0M4M0F0 (SEQ ID NO: 106); a threonine (T) at a residue corresponding to position 33 of wild-type A0A0M4M0F0 (SEQ ID NO: 106); a proline (P) at a residue corresponding to position 34 of wild-type A0A0M4M0F0 (SEQ ID NO: 106); a glycine (G) at a residue corresponding to position 40 of wild-type A0A0M4M0F0 (SEQ ID NO: 106); an aspartic acid (D) at a residue corresponding to position 59 of wild-type A0A0M4M0F0 (SEQ ID NO: 106); a lysine (K) at a residue corresponding to position 61 of wild-type A0A0M4M0F0 (SEQ ID NO: 106); a methionine (M) at a residue corresponding to position 63 of wild-type A0A0M4M0F0 (SEQ ID NO: 106); an aspartic acid (D) at a residue corresponding to position 64 of wild-type A0A0M4M0F0 (SEQ ID NO: 106); a glutamic acid (E) at a residue corresponding to position 69 of wild-type A0A0M4M0F0 (SEQ ID NO: 106); an glycine (G) at a residue corresponding to position 77 of wild-type A0A0M4M0F0 (SEQ ID NO: 106); an alanine (A) at a residue corresponding to position 78 of wild-type A0A0M4M0F0 (SEQ ID NO: 106); a leucine (L) at a residue corresponding to position 84 of wild-type A0A0M4M0F0 (SEQ ID NO: 106); an isoleucine (I) at a residue corresponding to position 92 of wild-type A0A0M4M0F0 (SEQ ID NO: 106); an alanine (A) at a residue corresponding to position 99 of wild-type A0A0M4M0F0 (SEQ ID NO: 106); a valine (V) at a residue corresponding to position 108 of wild-type A0A0M4M0F0 (SEQ ID NO: 106); an aspartic acid (D) at a residue corresponding to position 109 of wild-type A0A0M4M0F0 (SEQ ID NO: 106); an alanine (A) at a residue corresponding to position 120 of wild-type A0A0M4M0F0 (SEQ ID NO: 106); a glycine (G) at a residue corresponding to position 127 of wild-type A0A0M4M0F0 (SEQ ID NO: 106); a histidine (H) at a residue corresponding to position 134 of wild-type A0A0M4M0F0 (SEQ ID NO: 106); a glycine (G) at a residue corresponding to position 136 of wild-type A0A0M4M0F0 (SEQ ID NO: 106); an aspartic acid (D) at a residue corresponding to position 138 of wild-type A0A0M4M0F0 (SEQ ID NO: 106); a glutamine (Q) at a residue corresponding to position 140 of wild-type A0A0M4M0F0 (SEQ ID NO: 106); an alanine (A) at a residue corresponding to position 141 of wild-type A0A0M4M0F0 (SEQ ID NO: 106); an alanine (A) at a residue corresponding to position 164 of wild-type A0A0M4M0F0 (SEQ ID NO: 106); a glycine (G) at a residue corresponding to position 165 of wild-type A0A0M4M0F0 (SEQ ID NO: 106); a glycine (G) at a residue corresponding to position 166 of wild-type A0A0M4M0F0 (SEQ ID NO: 106); a glycine (G) at a residue corresponding to position 186 of wild-type A0A0M4M0F0 (SEQ ID NO: 106); an isoleucine (I) at a residue corresponding to position 189 of wild-type A0A0M4M0F0 (SEQ ID NO: 106); and/or an alanine (A) at a residue corresponding to position 199 of wild-type A0A0M4M0F0 (SEQ ID NO: 106).

In some embodiments, an HPS enzyme includes at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least at least 34, at least 35, at least 36, 3 at least 7, at least 38, at least 39, at least 40, at least 50, at least 60, at least 70, at least 80, at least 90, at least 100, at least 110, at least 120, at least 130, at least 140, at least 150, at least 160, at least 170, at least 180, at least 190, or at least 200 amino acid substitutions relative to A0A0M4M0F0 (SEQ ID NO: 106).

In some embodiments, an HPS enzyme includes at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least at least 34, at least 35, at least 36, 3 at least 7, at least 38, at least 39, at least 40, at least 50, at least 60, at least 70, at least 80, at least 90, at least 100, at least 110, at least 120, at least 130, at least 140, at least 150, at least 160, at least 170, at least 180, at least 190, or at least 200 amino acid substitutions relative to A0A0M4M0F0 (SEQ ID NO: 106) at one or more residues that does not correspond to positions 4, 6, 8, 27, 30, 32, 33, 34, 40, 59, 61, 63, 64, 69, 77, 78, 84, 92, 99, 108, 109, 120, 127, 134, 136, 138, 140, 141, 164, 165, 166, 186, 189, and/or 199 of A0A0M4M0F0 (SEQ ID NO: 106).

### 3-hexulose-6-phosphate Isomerase (PHI) Enzymes

Another aspect of the present disclosure provides 3-hexulose-6-phosphate isomerase (PHI) enzymes. As used herein, a 3-hexulose-6-phosphate isomerase (PHI) enzyme is an enzyme that is capable of converting 3-hexulose-6-phosphate to fructose-6-phosphate. In some embodiments, a PHI includes a glycine (G) at a residue corresponding to position 73 of MJ1247 from Methanococcus jannaschii, a proline (P) at a residue corresponding to position 78 of MJ1247 from Methanococcus jannaschii, and/or an aspartic acid (D) at a residue corresponding to position 84 of MJ1247 from Methanococcus jannaschii, an aspartic acid (D) or glutamic acid (E) at a residue corresponding to position 74 of MJ1247 from Methanococcus jannaschii, a threonine (T), valine (V), or isoleucine (I) at a residue corresponding to position 75 of MJ1247 from Methanococcus jannaschii. See, e.g., Martinez-Cruz et al., Structure. 2002 February; 10(2):195-204.

The PHI sequence for MJ1247 from Methanococcus jannaschii corresponding to UniProt No. Q58644 is:

(SEQ ID NO: 259)
MSKLEELDIVSNNILILKKFYTNDEWKNKLDSLIDRIIKAKKIFIFGVGR
SGYIGRCFAMRLMHLGFKSYFVGETTTPSYEKDDLLILISGSGRTESVLI
VAKKAKNINNNIIAIVCECGNVVEFADLTIPLEVKKSKYLPMGTTFEETA
LIFLDLVIAEIMKRLNLDESEIIKRHCNLL

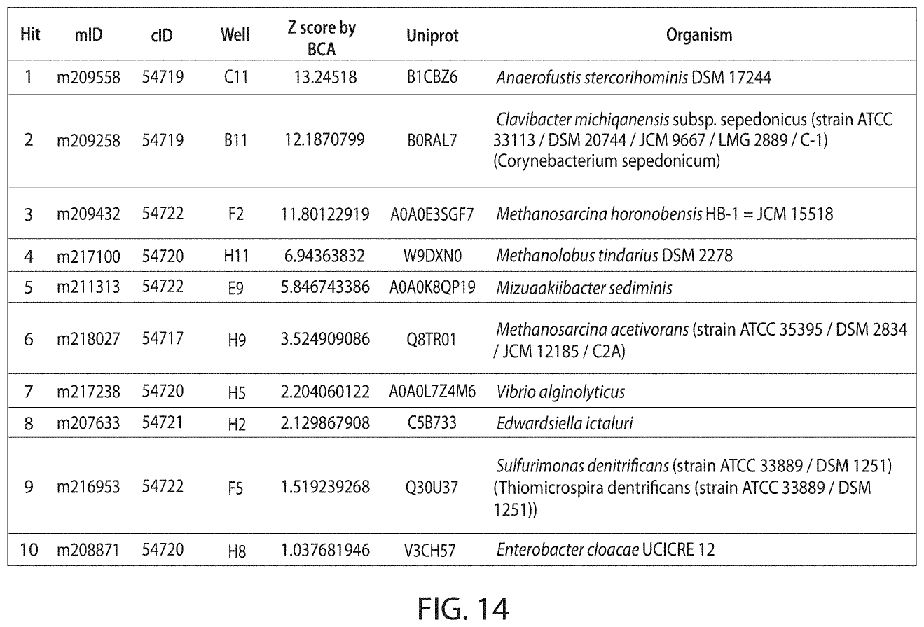

A PHI enzyme of the present disclosure may be from any suitable species, including but not limited to Anaerofustis stercorihoiminis, Clavibacter michiganensis, Methanosarcina horonobensis HB-1, Methanolobus tindarius, Mizuaakiibacter sediminis, Methanosarcina acetivorans, Vibrio alginolyticus, Edwardsiella ictaluri, Sulfurimonas denitrificans, and Enterobacter cloacae. In certain embodiments, a PHI enzyme is derived from a species shown in FIG. 14.

Any suitable method may be used to measure the activity of a PHI enzyme. As a non-limiting example, a multi-enzyme linked assay may be used to determine PHI activity. For example, ribose phosphate isomerase (RPI) can be used to convert ribose-5-phosphate to ribulose-5-phosphate, and an HPS enzyme may be introduced along with formaldehyde to produce hexulose-6-phosphate. An enzyme of interest (e.g., an isolated candidate PHI of interest or in cell lysate) can be added to determine whether the enzyme is capable of converting hexulose-6-phosphate to fructose-6-phosphate. If the enzyme is capable of converting hexulose-6-phosphate to fructose-6-phosphate, phosphoglucose isomerase (PGI) will have a substrate for further processing. PGI can be used to convert fructose-6-phosphate to glucose-6-phosphate. Finally, glucose-6-phosphate dehydrogenase (G6PDH) can be used to convert glucose-6-phosphate to 6-phosphoglucono-δ-lactone and produce NADPH. NADPH production can be measured using absorbance at 340 nm (see, e.g., Taylor et al., Acta Crystallogr D Biol Crystallogr. 2001 August; 57(Pt 8):1138-40) or a solution including the electron transfer catalyst phenazine methosulfate (PMS) may be used along with XTT tetrazolium. If PMS solution and XTT tetrazolium are used, conversion of XTT tetrazolium to XTT formazan can be measured as a colorimetric readout (see also FIG. 12).

In some embodiments, a PHI enzyme (e.g., an isolated PHI, an PHI in an intact cell, or an PHI in cell lysate) has an activity that is at least 1%, at least 5%, at least 10%, at least 20%, at least 30%, at least 40%, at least 50%, at least 60%, at least 70%, at least 80%, at least 90%, at least 100%, at least 110%, at least 120%, at least 130%, at least 140%, at least 150%, at least 160%, at least 170%, at least 200%, at least 300%, at least 400%, at least 500%, at least 600%, at least 700%, at least 800%, at least 900%, at least 1,000%, or any value in between, compared to the activity of a control. A control may be an isolated control PHI enzyme, a cell or cell lysate including a control PHI enzyme, or a cell or cell lysate not including the PHI enzyme of interest. A non-limiting example of PHI control enzymes includes PHI from Methylococcus capsulatus (SEQ ID NO: 146).

In some embodiments, a PHI enzyme of the present disclosure includes a sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or is 100% identical, including all values in between, compared to a sequence (e.g., nucleic acid or amino acid sequence) set forth as SEQ ID NOS: 123-134 or SEQ ID NOS: 135-146, or compared to a PHI sequence in Table 4, or a PHI sequence in FIG. 14.

In some embodiments, a PHI sequence includes a conservative amino acid substitution relative to one or more PHI sequences set forth as SEQ ID NOS: 135-146, relative to one or more PHI amino acid sequences in Table 4, or relative to one or more PHI sequences in FIG. 14. See, e.g., Table 1 for a non-limiting list of conservative amino acid substitutions.

It should be understood that a PHI may include a protein sequence that is identical to: an amino acid sequence selected from SEQ ID NOS: 135-146; a PHI amino acid sequence in Table 4 that is encoded by a nucleic acid including a synonymous mutation relative to a sequence selected from SEQ ID NOS: 123-134; or a PHI amino acid sequence encoded by a nucleotide sequence in Table 4.

Additional RuMP Pathway Enzymes

Additional RuMP pathway enzymes are also encompassed by the present disclosure, including ribose-5-phosphate isomerase (RPI) enzymes, ribulose 5-phosphate 3-epimerase (RPE) enzymes, transketolase (TKT) enzymes, transaldolase (TAL) enzymes, phosphofructokinase (PFK) enzymes, Sedoheptulose 1,7-Bisphosphatase (GLPX), fructose-bisphosphate aldolase (FBA) enzymes, 6-phosphogluconate dehydrogenase (GND) enzymes, and glucose-6-phosphate dehydrogenase (ZWF) enzymes.

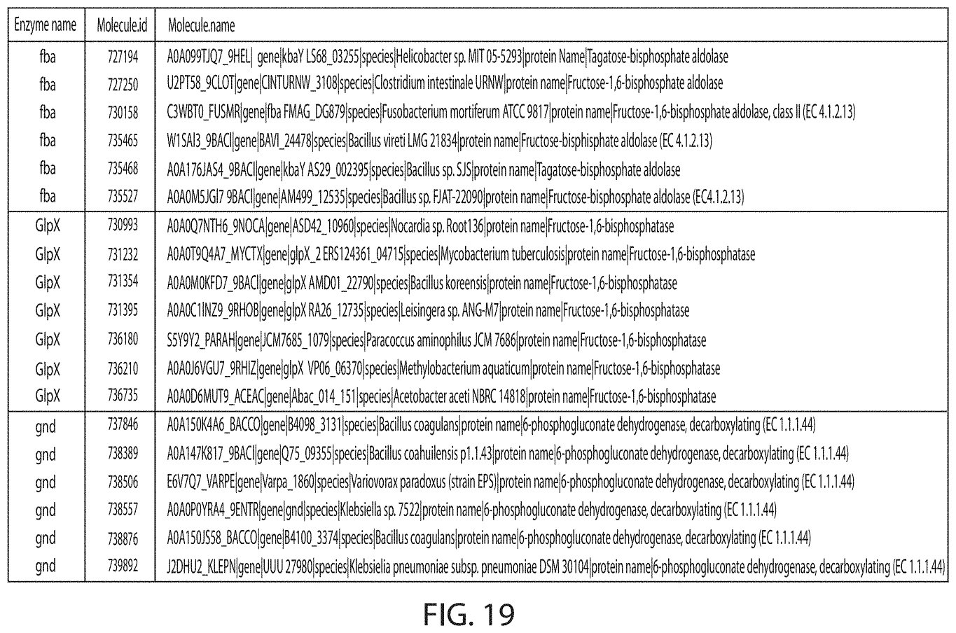

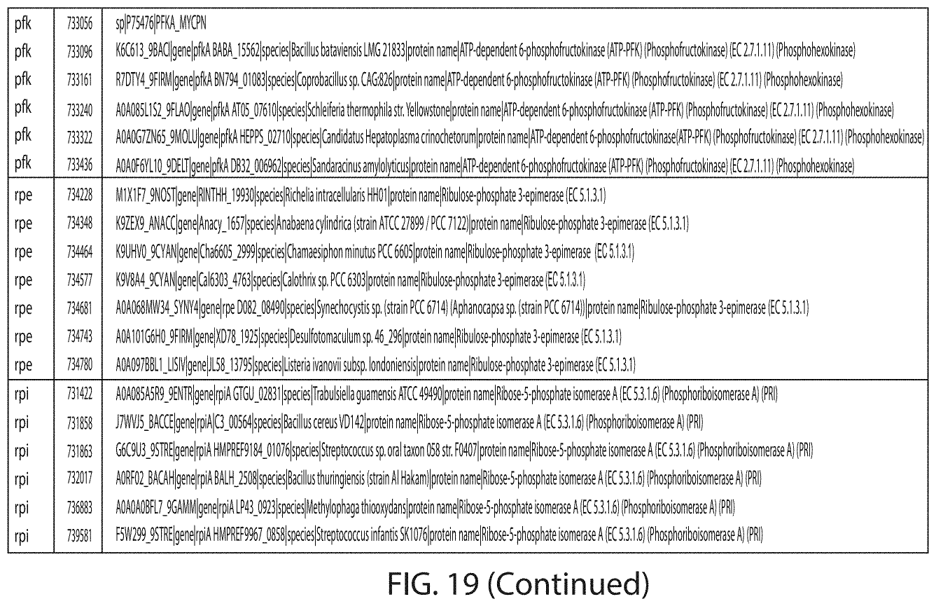

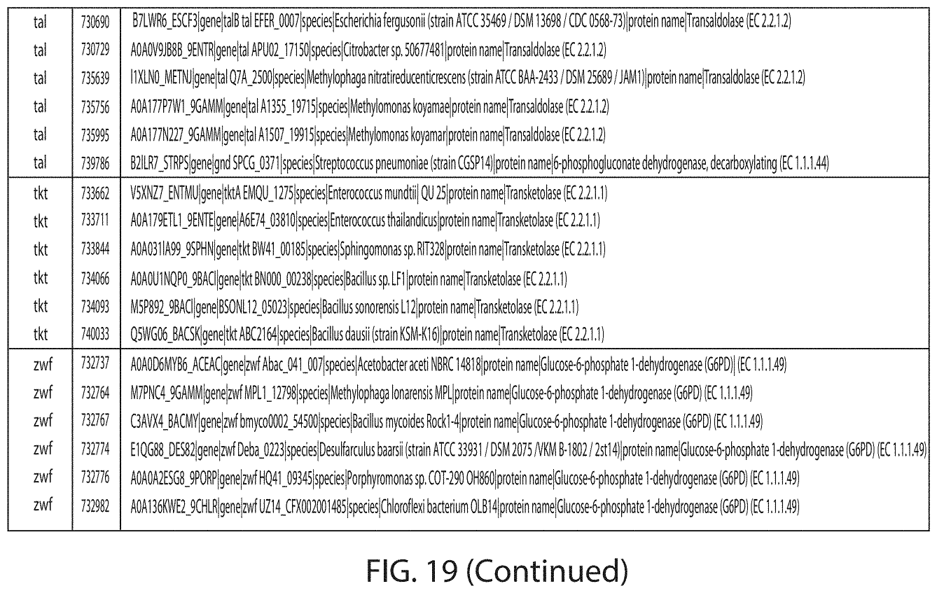

RPI enzymes are capable of catalyzing the conversion of ribose-5-phosphate to ribulose-5-phosphate. In some embodiments, an RPI enzyme may include a sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or is 100% identical, including all values in between, compared to a sequence (e.g., nucleic acid or amino acid sequence) set forth as SEQ ID NOs: 211-216 or SEQ ID NOS: 217-222, or compared to an RPI sequence in Table 5, or compared to an RPI sequence in FIG. 19.

In some embodiments, an RPI sequence includes a conservative amino acid substitution relative to one or more RPI sequences set forth as SEQ ID NOS: 217-222, relative to one or more RPI amino acid sequences in Table 5, or relative to one or more RPI sequences in FIG. 19. See, e.g., Table 1 for a non-limiting list of conservative amino acid substitutions.

It should be understood that an RPI may include a protein sequence that is identical to: an amino acid sequence selected from SEQ ID NOS: 217-222; an RPI amino acid sequence in Table 5 that is encoded by a nucleic acid including a synonymous mutation relative to a sequence selected from SEQ ID NOs: 211-216; or an RPI amino acid sequence that is encoded by an RPI nucleotide sequence in Table 5.

RPE enzymes are capable of catalyzing the epimerization of D-ribulose 5-phosphate to D-xylulose 5-phosphate. In some embodiments, an RPE enzyme includes a sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or is 100% identical, including all values in between, compared to a sequence (e.g., nucleic acid or amino acid sequence) set forth as SEQ ID NOs: 197-203 or SEQ ID NOS: 204-210, or compared to an RPE sequence in Table 5, or compared to an RPE sequence in FIG. 19.

In some embodiments, an RPE sequence includes a conservative amino acid substitution relative to one or more RPE sequences set forth as SEQ ID NOS: 204-210, relative to an RPE amino acid sequence in Table 5, or relative to an RPE sequence in FIG. 19. See, e.g., Table 1 for a non-limiting list of conservative amino acid substitutions.

It should be understood that an RPE may include a protein sequence that is identical to: an amino acid sequence selected from SEQ ID NOS: 204-210; an RPE amino acid sequence in Table 5 that is encoded by a nucleic acid including a synonymous mutation relative to a sequence selected from SEQ ID NOs: 197-203; or an RPE amino acid sequence encoded by an RPE nucleotide sequences in Table 5.

TKT enzymes are capable of transferring a 2-carbon fragment from D-xylulose-5-P to ribose-5-phosphate to produce seduheptulose-7-phosphate and glyceraldehyde-3-P and vice versa; capable of transferring a 2-carbon fragment from D-xylulose-5-P to the aldose erythrose-4-phosphate to produce fructose 6-phosphate and glyceraldehyde-3-P; or any combination thereof. A TKT enzyme may use the cofactor thiamine diphosphate. In some embodiments, a TKT enzyme includes a sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or is 100% identical, including all values in between, compared to a sequence (e.g., nucleic acid or amino acid sequence) set forth as SEQ ID NOs: 235-240 or SEQ ID NOS: 241-246, or compared to a TKT sequence in Table 5, or compared to a TKT sequence in FIG. 19.

In some embodiments, a TKT sequence includes a conservative amino acid substitution relative to one or more TKT sequences set forth as SEQ ID NOS: 241-246, relative to a TKT amino acid sequence in Table 5, or relative to a TKT amino acid sequence in FIG. 19. See, e.g., Table 1 for a non-limiting list of conservative amino acid substitutions.

It should be understood that a TKT may include a protein sequence that is identical to: an amino acid sequence selected from SEQ ID NOS: 241-246; a TKT amino acid sequence in Table 5 that is encoded by a nucleic acid including a synonymous mutation relative to a sequence selected from SEQ ID NOS: 235-240; or a TKT amino acid sequence encoded by a TKT nucleotide sequence in Table 5.

TAL enzymes are capable of catalyzing the interconversion of sedoheptulose 7-phosphate and D-glyceraldehyde 3-phosphate to D-erythrose 4-phosphate and D-fructose 6-phosphate. In some embodiments, a TAL enzyme include a sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or is 100% identical, including all values in between, compared to a sequence (e.g., nucleic acid or amino acid sequence) set forth as SEQ ID NOS: 223-228 or SEQ ID NOS: 229-234, compared to a TAL sequence in Table 5, or compared to a TAL sequence in FIG. 19.

In some embodiments, a TAL sequence includes a conservative amino acid substitution relative to one or more TAL sequences set forth as SEQ ID NOS: 229-234, relative to a TAL amino acid sequence in Table 5, or relative to a TAL amino acid sequence in FIG. 19. See, e.g., Table 1 for a non-limiting list of conservative amino acid substitutions.

It should be understood that a TAL may include a protein sequence that is identical to: an amino acid sequence set forth as SEQ ID NOS: 229-234; a TAL amino acid sequence in Table 5 that is encoded by nucleic acid including a synonymous mutation relative to a sequence set forth as SEQ ID NOS: 223-228; or a TAL amino acid sequence encoded by a TAL nucleotide sequence in Table 5.

PFK enzymes are capable of converting fructose-6-phosphate to fructose-1,6-bisphosphate. In some embodiments, a PFK enzyme include a sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or is 100% identical, including all values in between, compared to a sequence (e.g., nucleic acid or amino acid sequence) set forth as SEQ ID NOs: 185-190 or SEQ ID NOS: 191-196, compared to a PFK sequence in Table 5, or compared to a PFK sequence in FIG. 19.

In some embodiments, a PFK sequence includes a conservative amino acid substitution relative to one or more PFK sequences set forth as SEQ ID NOS: 191-196, relative to a PFK amino acid sequence in Table 5, or relative to a PFK sequence in FIG. 19. See, e.g., Table 1 for a non-limiting list of conservative amino acid substitutions.

It should be understood that a PFK may include a protein sequence that is identical to: an amino acid sequence selected from SEQ ID NOS: 191-196; a PFK amino acid sequence in Table 5 that is encoded by nucleic acid including a synonymous mutation relative to a sequence selected from SEQ ID NOS: 185-190; or a PFK amino acid sequence encoded by a PFK nucleotide sequences in Table 5.

GLPX enzymes are capable of hydrolyzing a phosphate from sedoheptulose 1,7-bisphosphate to produce sedoheptulose 7-phosphate. In some embodiments, a GLPX enzyme include a sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or is 100% identical, including all values in between, to a sequence (e.g., nucleic acid or amino acid sequence) selected from SEQ ID NOS: 159-165 or SEQ ID NOS: 166-172, compared to a GLPX sequences in Table 5, or compared to a GLPX sequence in FIG. 19.

In some embodiments, a GLPX sequence includes a conservative amino acid substitution relative to one or more GLPX sequences set forth as SEQ ID NOS: 166-172, relative to a GLPX amino acid sequence in Table 5, or relative to a GLPX sequence in FIG. 19. See, e.g., Table 1 for a non-limiting list of conservative amino acid substitutions.

It should be understood that a GLPX may include a protein sequence that is identical to: an amino acid sequence set forth in SEQ ID NOS: 166-172; a GLPX amino acid sequence in Table 5 that is encoded by nucleic acid including a synonymous mutation relative to a sequence set forth in SEQ ID NOS: 159-165; or a GLPX amino acid sequence encoded by a GLPX nucleotide sequences in Table 5.

FBA enzymes are capable of producing dihydroxyacetone phosphate and D-glyceraldehyde 3-phosphate from β-D-fructose 1,6-bisphosphate. In some embodiments, an FBA enzyme includes a sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or is 100% identical, including all values in between, compared to a sequence (e.g., nucleic acid or amino acid sequence) set forth as SEQ ID NOs: 147-152 or SEQ ID NOS: 153-158, compared to an FBA sequence in Table 5, or compared to an FBA sequence in FIG. 19.

In some embodiments, an FBA sequence includes a conservative amino acid substitution relative to one or more FBA sequences set forth as SEQ ID NOS: 153-158, relative to one or more FBA amino acid sequences in Table 5, or relative to one or more FBA sequences in FIG. 19. See, e.g., Table 1 for a non-limiting list of conservative amino acid substitutions.

It should be understood that an FBA may include a protein sequence that is identical to: an amino acid sequence set forth in SEQ ID NOS: 153-158; an FBA amino acid sequence in Table 5 that is encoded by nucleic acid sequence including a synonymous mutation relative to a sequence set forth in SEQ ID NOS: 147-152; or an FBA amino acid sequence that is encoded by an FBA nucleotide sequences in Table 5.

GND enzymes are capable of producing D-ribulose 5-phosphate, NADPH, and CO₂ from 6-phospho-D-gluconate and NADP+. In some embodiments, a GND enzyme includes a sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or is 100% identical, including all values in between, compared to a sequence (e.g., nucleic acid or amino acid sequence) set forth in SEQ ID NOs: 173-178 or SEQ ID NOS: 179-184, compared to a GND sequence in Table 5, or compared to a GND sequence in FIG. 19.

In some embodiments, a GND sequence includes a conservative amino acid substitution relative to one or more GND sequences set forth in SEQ ID NOS: 179-184, relative to one or more GND amino acid sequences in Table 5, or relative to one or more GND sequences in FIG. 19. See, e.g., Table 1 for a non-limiting list of conservative amino acid substitutions.

It should be understood that a GND may include a protein sequence that is identical to: an amino acid sequence set forth in SEQ ID NOS: 179-184; a GND amino acid sequence in Table 5 that is encoded by nucleic acid including a synonymous mutation relative to a sequence set forth in SEQ ID NOS: 173-178; or a GND amino acid sequence that is encoded by a GND nucleic acid sequence in Table 5.

ZWF enzymes are capable of producing 6-phospho-D-glucono-1,5-lactone, H+, and NADPH from D-glucose 6-phosphate and NADP+. In some embodiments, a ZWF enzyme includes a sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or is 100% identical, including all values in between, compared to a sequence (e.g., nucleic acid or amino acid sequence) set forth in SEQ ID NOs: 247-252 or SEQ ID NOS: 253-258, compared to a ZWF sequence in Table 5, or compared to a ZWF sequence in FIG. 19.

In some embodiments, a ZWF sequence includes a conservative amino acid substitution relative to one or more ZWF sequences set forth in SEQ ID NOS: 253-258, relative to one or more ZWF amino acid sequences in Table 5, or relative to one or more ZWF sequences in FIG. 19. See, e.g., Table 1 for a non-limiting list of conservative amino acid substitutions.

It should be understood that a ZWF may include a protein sequence that is identical to: an amino acid sequence set forth in SEQ ID NOS: 253-258; a ZWF amino acid sequence in Table 5 that is encoded by a nucleic acid including a synonymous mutation relative to a sequence set forth in SEQ ID NOs: 247-252; or a ZWF amino acid sequence encoded by a ZWF nucleotide sequence in Table 5.

Variants

Variants of the sequences (e.g., MDH, HPS, PHI, or other RuMP cycle enzyme), including nucleic acid or amino acid sequences) described herein are also encompassed by the present disclosure. A variant may share at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with a reference sequence, including all values in between.

The term “sequence identity,” as known in the art, refers to a relationship between the sequences of two polypeptides or polynucleotides, as determined by sequence comparison (alignment). In some embodiments, sequence identity is determined across the entire length of a recombinant sequence (e.g., MDH, HPS, PHI, or other RuMP cycle enzyme). In some embodiments, sequence identity is determined over a region (e.g., a stretch of amino acids or nucleic acids) of a recombinant sequence (e.g., MDH, HPS, PHI, or other RuMP cycle enzyme).

Identity can also refer to the degree of sequence relatedness between two sequences as determined by the number of matches between strings of two or more residues (e.g., nucleic acid or amino acid residues). Identity measures the percent of identical matches between the smaller of two or more sequences with gap alignments (if any) addressed by a particular mathematical model or computer program (e.g., “algorithms”).

Identity of related polypeptides or nucleic acid sequences can be readily calculated by any of the methods known to one of ordinary skill in the art. The “percent identity” of two sequences (e.g., nucleic acid or amino acid sequences) may, for example, be determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST® and XBLAST® programs (version 2.0) of Altschul et al., J. Mol. Biol. 215:403-10, 1990. BLAST® protein searches can be performed, for example, with the XBLAST program, score=50, wordlength=3 to obtain amino acid sequences homologous to the protein molecules of the invention. Where gaps exist between two sequences, Gapped BLAST® can be utilized, for example, as described in Altschul et al., Nucleic Acids Res. 25(17):3389-3402, 1997. When utilizing BLAST® and Gapped BLAST® programs, the default parameters of the respective programs (e.g., XBLAST® and NBLAST®) can be used, or the parameters can be adjusted appropriately as would be understood by one of ordinary skill in the art.

Another local alignment technique which may be used, for example, is based on the Smith-Waterman algorithm (Smith, T. F. & Waterman, M. S. (1981) “Identification of common molecular subsequences.” J. Mol. Biol. 147:195-197). A general global alignment technique which may be used, for example, is the Needleman-Wunsch algorithm (Needleman, S. B. & Wunsch, C. D. (1970) “A general method applicable to the search for similarities in the amino acid sequences of two proteins.” J. Mol. Biol. 48:443-453), which is based on dynamic programming.

More recently, a Fast Optimal Global Sequence Alignment Algorithm (FOGSAA) was developed that purportedly produces global alignment of nucleic acid and amino acid sequences faster than other optimal global alignment methods, including the Needleman-Wunsch algorithm. In some embodiments, the identity of two polypeptides is determined by aligning the two amino acid sequences, calculating the number of identical amino acids, and dividing by the length of one of the amino acid sequences. In some embodiments, the identity of two nucleic acids is determined by aligning the two nucleotide sequences and calculating the number of identical nucleotide and dividing by the length of one of the nucleic acids.

For multiple sequence alignments, computer programs including Clustal Omega (Sievers et al., Mol Syst Biol. 2011 Oct. 11; 7:539) may be used.

As used herein, variant sequences may be homologous sequences. As used herein, homologous sequences are sequences (e.g., nucleic acid or amino acid sequences) that share a certain percent identity (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% percent identity, including all values in between). Homologous sequences include but are not limited to paralogous or orthologous sequences. Paralogous sequences arise from duplication of a gene within a genome of a species, while orthologous sequences diverge after a speciation event.

In some embodiments, a polypeptide variant (e.g., MDH, HPS, PHI, or other RuMP cycle enzyme variant) includes a domain that shares a secondary structure (e.g., alpha helix, beta sheet) with a reference polypeptide (e.g., a reference MDH, HPS, PHI, or other RuMP cycle enzyme). In some embodiments, a polypeptide variant (e.g., MDH, HPS, PHI, or other RuMP cycle enzyme variant) shares a tertiary structure with a reference polypeptide (e.g., a reference MDH, HPS, PHI, or other RuMP cycle enzyme). As a non-limiting example, a variant polypeptide (e.g., MDH, HPS, PHI, or other RuMP cycle enzyme) may have low primary sequence identity (e.g., less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, or less than 5% sequence identity) compared to a reference polypeptide, but share one or more secondary structures (e.g., including but not limited to loops, alpha helices, or beta sheets, or have the same tertiary structure as a reference polypeptide. For example, a loop may be located between a beta sheet and an alpha helix, between two alpha helices, or between two beta sheets. Homology modeling may be used to compare two or more tertiary structures.

Any suitable method, including circular permutation (Yu and Lutz, Trends Biotechnol. 2011 January; 29(1):18-25), may be used to produce such variants. In circular permutation, the linear primary sequence of a polypeptide can be circularized (e.g., by joining the N-terminal and C-terminal ends of the sequence) and the polypeptide can be severed (“broken”) at a different location. Thus, the linear primary sequence of the new polypeptide may have low sequence identity (e.g., less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less or less than 5%, including all values in between) as determined by linear sequence alignment methods (e.g., Clustal Omega or BLAST). Topological analysis of the two proteins, however, may reveal that the tertiary structure of the two polypeptides is similar or dissimilar. Without being bound by a particular theory, a variant polypeptide created through circular permutation of a reference polypeptide and with a similar tertiary structure as the reference polypeptide can share similar functional characteristics (e.g., enzymatic activity, enzyme kinetics, substrate specificity or product specificity). In some instances, circular permutation may alter the secondary structure, tertiary structure or quaternary structure and produce an enzyme with different functional characteristics (e.g., increased or decreased enzymatic activity, different substrate specificity, or different product specificity). See, e.g., Yu and Lutz, Trends Biotechnol. 2011 January; 29(1):18-25.

It should be appreciated that in a protein that has undergone circular permutation, the linear amino acid sequence of the protein would differ from a reference protein that has not undergone circular permutation. However, one of ordinary skill in the art would be able to readily determine which residues in the protein that has undergone circular permutation correspond to residues in the reference protein that has not undergone circular permutation by, for example, aligning the sequences and detecting conserved motifs, and/or by comparing the structures or predicted structures of the proteins, e.g., by homology modeling.

Functional variants of the recombinant MDH, HPS, PHI, or other RuMP cycle enzyme disclosed herein are also encompassed by the present disclosure. For example, functional variants may bind one or more of the same substrates (e.g., methanol, ribulose-5-P, or hexulose-6-P) or produce one or more of the same products (e.g., formaldehyde, hexulose-6-P, or fructose-6-P). Functional variants may be identified using any method known in the art. For example, the algorithm of Karlin and Altschul Proc. Natl. Acad. Sci. USA 87:2264-68, 1990 described above may be used to identify homologous proteins with known functions.

Putative functional variants may also be identified by searching for polypeptides with functionally annotated domains. Databases including Pfam (Sonnhammer et al., Proteins. 1997 July; 28(3):405-20) may be used to identify polypeptides with a particular domain.

Homology modeling may also be used to identify amino acid residues that are amenable to mutation without affecting function. A non-limiting example of such a method may include use of position-specific scoring matrix (PSSM) and an energy minimization protocol.

Position-specific scoring matrix (PSSM) uses a position weight matrix to identify consensus sequences (e.g., motifs). PSSM can be conducted on nucleic acid or amino acid sequences. Sequences are aligned and the method takes into account the observed frequency of a particular residue (e.g., an amino acid or a nucleotide) at a particular position and the number of sequences analyzed. See, e.g., Stormo et al., Nucleic Acids Res. 1982 May 11; 10(9):2997-3011. The likelihood of observing a particular residue at a given position can be calculated. Without being bound by a particular theory, positions in sequences with high variability may be amenable to mutation (e.g., PSSM score ≥0) to produce functional homologs.

PSSM may be paired with calculation of a Rosetta energy function, which determines the difference between the wild-type and the single-point mutant. The Rosetta energy function calculates this difference as (ΔΔG_{calc}). With the Rosetta function, the bonding interactions between a mutated residue and the surrounding atoms are used to determine whether a mutation increases or decreases protein stability. For example, a mutation that is designated as favorable by the PSSM score (e.g. PSSM score ≥0), can then be analyzed using the Rosetta energy function to determine the potential impact of the mutation on protein stability. Without being bound by a particular theory, potentially stabilizing mutations are desirable for protein engineering (e.g., production of functional homologs). In some embodiments, a potentially stabilizing mutation has a ΔΔG_{calc} value of less than −0.1 (e.g., less than −0.2, less than −0.3, less than −0.35, less than −0.4, less than −0.45, less than −0.5, less than −0.55, less than −0.6, less than −0.65, less than −0.7, less than −0.75, less than −0.8, less than −0.85, less than −0.9, less than −0.95, or less than −1.0) Rosetta energy units (R.e.u.). See, e.g., Goldenzweig et al., Mol Cell. 2016 Jul. 21; 63(2):337-346. doi: 10.1016/j.molcel.2016.06.012.

In some embodiments, an MDH, HPS, PHI, or other RuMP cycle enzyme coding sequence includes a mutation at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more than 100 positions corresponding to a reference (e.g., MDH, HPS, PHI, or other RuMP cycle enzyme) coding sequence. In some embodiments, the MDH, HPS, PHI, or other RuMP cycle enzyme coding sequence includes a mutation in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more codons of the coding sequence relative to a reference (e.g., MDH, HPS, PHI, or other RuMP cycle enzyme) coding sequence. As will be understood by one of ordinary skill in the art, a mutation within a codon may or may not change the amino acid that is encoded by the codon due to degeneracy of the genetic code. In some embodiments, the one or more mutations in the coding sequence do not alter the amino acid sequence of the coding sequence (e.g., MDH, HPS, PHI, or other RuMP cycle enzyme) relative to the amino acid sequence of a reference polypeptide (e.g., MDH, HPS, PHI, or other RuMP cycle enzyme).

In some embodiments, the one or more mutations in a recombinant MDH, HPS, PHI, or other RuMP cycle enzyme sequence alters the amino acid sequence of the polypeptide (e.g., MDH, HPS, PHI, or other RuMP cycle enzyme) relative to the amino acid sequence of a reference polypeptide (e.g., MDH, HPS, PHI, or other RuMP cycle enzyme). In some embodiments, the one or more mutations alters the amino acid sequence of the recombinant polypeptide (e.g., MDH, HPS, PHI, or other RuMP cycle enzyme) relative to the amino acid sequence of a reference polypeptide (e.g., MDH, HPS, PHI, or other RuMP cycle enzyme) and alters (enhances or reduces) an activity of the polypeptide relative to the reference polypeptide.

The activity (e.g., specific activity) of any of the recombinant polypeptides described herein (e.g., MDH, HPS, PHI, or other RuMP cycle enzyme) may be measured using routine methods. As a non-limiting example, a recombinant polypeptide's activity may be determined by measuring its substrate specificity, product(s) produced, the concentration of product(s) produced, or any combination thereof. As used herein, “specific activity” of a recombinant polypeptide refers to the amount (e.g., concentration) of a particular product produced for a given amount (e.g., concentration) of the recombinant polypeptide per unit time.

The skilled artisan will also realize that mutations in a recombinant polypeptide (e.g., MDH, HPS, PHI, or other RuMP cycle enzyme) coding sequence may result in conservative amino acid substitutions to provide functionally equivalent variants of the foregoing polypeptides, e.g., variants that retain the activities of the polypeptides. As used herein, a “conservative amino acid substitution” refers to an amino acid substitution that does not alter the relative charge or size characteristics or functional activity of the protein in which the amino acid substitution is made.

In some instances, an amino acid is characterized by its R group (see, e.g., Table 1). For example, an amino acid may include a nonpolar aliphatic R group, a positively charged R group, a negatively charged R group, a nonpolar aromatic R group, or a polar uncharged R group. Non-limiting examples of an amino acid including a nonpolar aliphatic R group include alanine, glycine, valine, leucine, methionine, and isoleucine. Non-limiting examples of an amino acid including a positively charged R group includes lysine, arginine, and histidine. Non-limiting examples of an amino acid including a negatively charged R group include aspartic acid and glutamic acid. Non-limiting examples of an amino acid including a nonpolar, aromatic R group include phenylalanine, tyrosine, and tryptophan. Non-limiting examples of an amino acid including a polar uncharged R group include serine, threonine, cysteine, proline, asparagine, and glutamine.

Variants can be prepared according to methods for altering polypeptide sequence known to one of ordinary skill in the art such as are found in references which compile such methods, e.g., Molecular Cloning: A Laboratory Manual, J. Sambrook, et al., eds., Fourth Edition, Cold Spring Harbor Laboratory Press, Cold Spring Harbor, N.Y., 2012, or Current Protocols in Molecular Biology, F. M. Ausubel, et al., eds., John Wiley & Sons, Inc., New York, 2010.

Non-limiting examples of functionally equivalent variants of polypeptides may include conservative amino acid substitutions in the amino acid sequences of proteins disclosed herein. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D. Additional non-limiting examples of conservative amino acid substitutions are provided in Table 1.

In some embodiments, 1, 2, 3, 4, 5, 6, 7, 8, 9, 10, 11, 12, 13, 14, 15, 16, 17, 18, 19, 20 or more than 20 residues can be changed when preparing variant polypeptides. In some embodiments, amino acids are replaced by conservative amino acid substitutions.

TABLE 1
Non-limiting Examples of Conservative Amino Acid Substitutions

| Original | | Conservative Amino |
|---|---|---|
| Residue | R Group Type | Acid Substitutions |
| Ala | nonpolar aliphatic R group | Cys, Gly, Ser |
| Arg | positively charged R group | His, Lys |
| Asn | polar uncharged R group | Asp, Gln, Glu |
| Asp | negatively charged R group | Asn, Gln, Glu |
| Cys | polar uncharged R group | Ala, Ser |
| Gln | polar uncharged R group | Asn, Asp, Glu |
| Glu | negatively charged R group | Asn, Asp, Gln |
| Gly | nonpolar aliphatic R group | Ala, Ser |
| His | positively charged R group | Arg, Tyr, Trp |
| Ile | nonpolar aliphatic R group | Leu, Met, Val |
| Leu | nonpolar aliphatic R group | Ile, Met, Val |
| Lys | positively charged R group | Arg, His |
| Met | nonpolar aliphatic R group | Ile, Leu, Phe, Val |
| Pro | polar uncharged R group | |
| Phe | nonpolar aromatic R group | Met, Trp, Tyr |
| Ser | polar uncharged R group | Ala, Gly, Thr |
| Thr | polar uncharged R group | Ala, Asn, Ser |
| Trp | nonpolar aromatic R group | His, Phe, Tyr, Met |
| Tyr | nonpolar aromatic R group | His, Phe, Trp |
| Val | nonpolar aliphatic R group | Ile, Leu, Met, Thr |

Amino acid substitutions in the amino acid sequence of a polypeptide to produce a recombinant polypeptide (e.g., MDH, HPS, PHI, or other RuMP cycle enzyme) variant having a desired property and/or activity can be made by alteration of the coding sequence of the polypeptide (e.g., MDH, HPS, PHI, or other RuMP cycle enzyme). Similarly, conservative amino acid substitutions in the amino acid sequence of a polypeptide to produce functionally equivalent variants of the polypeptide typically are made by alteration of the coding sequence of the recombinant polypeptide (e.g., MDH, HPS, PHI, or other RuMP cycle enzyme).

Mutations (e.g., substitutions) can be made in a nucleotide sequence by a variety of methods known to one of ordinary skill in the art. For example, mutations can be made by PCR-directed mutation, site-directed mutagenesis according to the method of Kunkel (Kunkel, Proc. Nat. Acad. Sci. U.S.A. 82: 488-492, 1985), or by chemical synthesis of a gene encoding a polypeptide.

Methods of Increasing Methanol Assimilation, Producing Methylotrophic Cells, and Producing Amino Acids

Aspects of the present disclosure relate to the recombinant expression of genes encoding enzymes, functional modifications and variants thereof, as well as uses relating thereto. For example, the methods described herein may be used to increase methanol assimilation, produce cells that are capable of using methanol as a carbon source, and promote amino acid production.

A nucleic acid encoding any of the recombinant polypeptides (e.g., MDHs, HPSs, PHIs, or other RuMP cycle enzymes) described herein may be incorporated into any appropriate vector through any method known in the art. For example, the vector may be an expression vector, including but not limited to a viral vector (e.g., a lentiviral, retroviral, adenoviral, or adeno-associated viral vector), any vector suitable for transient expression, any vector suitable for constitutive expression, or any vector suitable for inducible expression (e.g., a galactose-inducible vector (e.g., including a P_{gal} promoter) or doxycycline-inducible vector). A non-limiting example of a vector for expression of a recombinant polypeptide (e.g., MDH, HPS, PHI, or other RuMP cycle enzyme) is described in Example 1 below.

In some embodiments, a vector replicates autonomously in the cell. A vector can contain one or more endonuclease restriction sites that are cut by a restriction endonuclease to insert and ligate a nucleic acid containing a gene described herein to produce a recombinant vector that is able to replicate in a cell. Vectors are typically composed of DNA, although RNA vectors are also available. Cloning vectors include, but are not limited to: plasmids, fosmids, phagemids, virus genomes and artificial chromosomes. As used herein, the terms “expression vector” or “expression construct” refer to a nucleic acid construct, generated recombinantly or synthetically, with a series of specified nucleic acid elements that permit transcription of a particular nucleic acid in a host cell (e.g., microbe), such as a bacterial cell or a yeast cell. In some embodiments, the nucleic acid sequence of a gene described herein is inserted into a cloning vector such that it is operably joined to regulatory sequences and, in some embodiments, expressed as an RNA transcript. In some embodiments, the vector contains one or more markers, such as a selectable marker as described herein, to identify cells transformed or transfected with the recombinant vector. In some embodiments, the nucleic acid sequence of a gene described herein is codon-optimized. Codon-optimization may increase production of the gene product by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%, including all values in between) relative to a reference sequence that is not codon-optimized.

A coding sequence and a regulatory sequence are said to be “operably joined” when the coding sequence and the regulatory sequence are covalently linked and the expression or transcription of the coding sequence is under the influence or control of the regulatory sequence. If the coding sequence is to be translated into a functional protein, the coding sequence and the regulatory sequence are said to be operably joined if induction of a promoter in the 5′ regulatory sequence transcribes the coding sequence and if the nature of the linkage between the coding sequence and the regulatory sequence does not (1) result in the introduction of a frame-shift mutation, (2) interfere with the ability of the promoter region to direct the transcription of the coding sequence, or (3) interfere with the ability of the corresponding RNA transcript to be translated into a protein. Thus, a promoter region is operably joined to a coding sequence if the promoter region transcribes the coding sequence and the transcript can be translated into the protein or polypeptide of interest.

In some embodiments, the nucleic acid encoding any of the proteins described herein is under the control of regulatory sequences (e.g., enhancer sequences). In some embodiments, a nucleic acid is expressed under the control of a promoter. The promoter can be a native promoter, e.g., the promoter of the gene in its endogenous context, which provides normal regulation of expression of the gene. Alternatively, a promoter can be a promoter that is different from the native promoter of the gene, e.g., the promoter is different from the promoter of the gene in its endogenous context. As used herein, a “heterologous promoter” or “recombinant promoter” is a promoter that is not naturally or normally associated with or that does not naturally or normally control transcription of a DNA sequence to which it is operably joined. In some embodiments, a nucleotide sequence is under the control of a heterologous promoter.

In some embodiments, a promoter may drive expression of more than one heterologous gene. As a non-limiting example, one promoter may drive expression of heterologous genes encoding an MDH, an HPS, a PHI, and/or any other RuMP cycle enzymes (e.g., ribose-5-phosphate isomerase (RPI), ribulose 5-phosphate 3-epimerase (RPE), transketolase (TKT), transaldolase (TAL) enzymes, phosphofructokinase (PFK), Sedoheptulose 1,7-Bisphosphatase (GLPX), fructose-bisphosphate aldolase (FBA), 6-phosphogluconate dehydrogenase (GND), and glucose-6-phosphate dehydrogenase (ZWF)). In some embodiments, an MDH, an HPS, a PHI, and/or any other RuMP cycle enzymes may be encoded by one operon. In some embodiments, an MDH, an HPS, a PHI, and/or any other RuMP cycle enzymes may be encoded by separate operons. In some embodiments, separate promoters may drive expression of each heterologous gene.

In some embodiments, the promoter is a eukaryotic promoter. Non-limiting examples of eukaryotic promoters include TDH3, PGK1, PKC1, PDC1, TEF1, TEF2, RPL18B, SSA1, TDH2, PYK1, TPI1 GAL1, GAL10, GAL7, GAL3, GAL2, MET3, MET25, HXT3, HXT7, ACT1, ADH1, ADH2, CUP1-1, ENO2, and SOD1, as would be known to one of ordinary skill in the art (see, e.g., Addgene website: blog.addgene.org/plasmids-101-the-promoter-region). In some embodiments, the promoter is a prokaryotic promoter (e.g., bacteriophage or bacterial promoter). Non-limiting examples of bacteriophage promoters include Pls1con, T3, T7, SP6, and PL. Non-limiting examples of bacterial promoters include apFAB101, apFAB92 (Ec-TTL-P100), abFAB71 (Ec-TTL-P097), apFAB45 (Ec-TTL-9092), apFAB29, apFAB76(EC-TTL-P075), BBA J23104 (Ec TTL-P054), J23104, Ec-TTL-P041, apFAB436 (Ec-TTL-P046), apFAB332, Pbad, PmgrB, Ptrc2, Plac/ara, Ptac, and Pm.

In some embodiments, the promoter is an inducible promoter. As used herein, an “inducible promoter” is a promoter controlled by the presence or absence of a molecule. Non-limiting examples of inducible promoters include chemically-regulated promoters and physically-regulated promoters. For chemically-regulated promoters, the transcriptional activity can be regulated by one or more compounds, such as alcohol, tetracycline, galactose, a steroid, a metal, or other compounds. For physically-regulated promoters, transcriptional activity can be regulated by a phenomenon such as light or temperature. Non-limiting examples of tetracycline-regulated promoters include anhydrotetracycline (aTc)-responsive promoters and other tetracycline-responsive promoter systems (e.g., a tetracycline repressor protein (tetR), a tetracycline operator sequence (tetO) and a tetracycline transactivator fusion protein (tTA)). Non-limiting examples of steroid-regulated promoters include promoters based on the rat glucocorticoid receptor, human estrogen receptor, moth ecdysone receptors, and promoters from the steroid/retinoid/thyroid receptor superfamily. Non-limiting examples of metal-regulated promoters include promoters derived from metallothionein (proteins that bind and sequester metal ions) genes. Non-limiting examples of pathogenesis-regulated promoters include promoters induced by salicylic acid, ethylene or benzothiadiazole (BTH). Non-limiting examples of temperature/heat-inducible promoters include heat shock promoters. Non-limiting examples of light-regulated promoters include light responsive promoters from plant cells. In certain embodiments, the inducible promoter is a galactose-inducible promoter. In some embodiments, the inducible promoter is induced by one or more physiological conditions (e.g., pH, temperature, radiation, osmotic pressure, saline gradients, cell surface binding, or concentration of one or more extrinsic or intrinsic inducing agents). Non-limiting examples of an extrinsic inducer or inducing agent include amino acids and amino acid analogs, saccharides and polysaccharides, nucleic acids, protein transcriptional activators and repressors, cytokines, toxins, petroleum-based compounds, metal containing compounds, salts, ions, enzyme substrate analogs, hormones or any combination thereof.

In some embodiments, the promoter is a constitutive promoter. As used herein, a “constitutive promoter” refers to an unregulated promoter that allows continuous transcription of a gene. Non-limiting examples of a constitutive promoter include TDH3, PGK1, PKC1, PDC1, TEF1, TEF2, RPL18B, SSA1, TDH2, PYK1,TPI1, HXT3, HXT7, ACT1, ADH1, ADH2, ENO2, and SOD1.

Other inducible promoters or constitutive promoters known to one of ordinary skill in the art are also contemplated herein.

The precise nature of the regulatory sequences needed for gene expression may vary between species or cell types, but generally include, as necessary, 5′ non-transcribed and 5′ non-translated sequences involved with the initiation of transcription and translation respectively, such as a TATA box, capping sequence, CAAT sequence, and the like. In particular, such 5′ non-transcribed regulatory sequences will include a promoter region which includes a promoter sequence for transcriptional control of the operably joined gene. Regulatory sequences may also include enhancer sequences or upstream activator sequences. The vectors disclosed herein may include 5′ leader or signal sequences. The regulatory sequence may also include a terminator sequence. In some embodiments, a terminator sequence marks the end of a gene in DNA during transcription. The choice and design of one or more appropriate vectors suitable for inducing expression of one or more genes described herein in a heterologous organism is within the ability and discretion of one of ordinary skill in the art.

Expression vectors containing the necessary elements for expression are commercially available and known to one of ordinary skill in the art (see, e.g., Sambrook et al., Molecular Cloning: A Laboratory Manual, Fourth Edition, Cold Spring Harbor Laboratory Press, 2012).

Any of the polynucleotides and proteins of the present disclosure may be expressed in a host cell. The term “host cell” refers to a cell that can be used to express a polynucleotide, such as a polynucleotide that encodes an enzyme. A “recombinant host cell” refers to a host cell that has been genetically modified by, e.g., cloning and transformation methods, or by other methods known in the art (e.g., selective editing methods).

The term “heterologous” with respect to a polynucleotide, such as a polynucleotide comprising a gene, is used interchangeably with the term “exogenous” and the term “recombinant” and refers to: a polynucleotide that has been artificially supplied to a biological system; a polynucleotide that has been modified within a biological system, or a polynucleotide whose expression or regulation has been manipulated within a biological system. A heterologous polynucleotide that is introduced into or expressed in a host cell may be a polynucleotide that comes from a different organism or species than the host cell, or may be a synthetic polynucleotide, or may be a polynucleotide that is also endogenously expressed in the same organism or species as the host cell. For example, a polynucleotide that is endogenously expressed in a host cell may be considered heterologous when it is situated non-naturally in the host cell; expressed recombinantly in the host cell, either stably or transiently; modified within the host cell; selectively edited within the host cell; expressed in a copy number that differs from the naturally occurring copy number within the host cell; or expressed in a non-natural way within the host cell, such as by manipulating regulatory regions that control expression of the polynucleotide. In some embodiments, a heterologous polynucleotide is a polynucleotide that is endogenously expressed in a host cell but whose expression is driven by a promoter that does not naturally regulate expression of the polynucleotide. In other embodiments, a heterologous polynucleotide is a polynucleotide that is endogenously expressed in a host cell and whose expression is driven by a promoter that does naturally regulate expression of the polynucleotide, but the promoter or another regulatory region is modified. In some embodiments, the promoter is recombinantly activated or repressed. For example, gene-editing based techniques may be used to regulate expression of a polynucleotide, including an endogenous polynucleotide, from a promoter, including an endogenous promoter. See, e.g., Chavez et al., Nat Methods. 2016 July; 13(7): 563-567. A heterologous polynucleotide may comprise a wild-type sequence or a mutant sequence as compared with a reference polynucleotide sequence.

Any suitable host cell may be used to produce any of the recombinant polypeptides (e.g., MDH, HPS, PHI, or other RuMP cycle enzyme) disclosed herein, including eukaryotic cells or prokaryotic cells. Suitable host cells include bacteria cells (e.g., Escherichia coli cells) and fungal cells (e.g., yeast cells). Non-limiting examples of genera of bacteria cells include Brevibacterium spp., Achromobacter spp., Acidomonas spp., Acinetobacter spp., Aeromonas spp., Afipia spp., Amycolatopsis spp., Anaerofustis spp., Ancylobacter spp., Frigoribacterium spp., Photobacterium spp., Enterobacter spp., Angulomicrobium spp., Arthrobacter spp., Asaia spp., Bacillus spp., Betaproteobacteria spp., Burkholderia spp., Candida spp., Chromobacterium spp., Citrobacter spp., Clavibacter spp., Comamonadaceae spp., Commensalibacter spp., Cupriavidus spp., Edwardsiella spp., Escherichia spp., Franconibacter spp., Gliocladium spp., Hansenula spp., Idiomarina spp., Klebsiella spp., Lactobacillus spp., Lysinibacillus spp., Macrococcus spp., Methanolobus spp., Methanosarcina spp., Methanosarcina spp., Methlyopila spp., Methylibium spp., Methylobacterium spp., Methylocapsa spp., Methylococcus spp., Methylophaga spp., Methylophilus spp., Methylothermus spp., Methyloversatilis spp., Mizuaakiibacter spp., Mycobacterium spp., Neisseria spp., Nitrincola spp., Paecilomyces spp., Paenibacillus spp., Paracoccus spp., Penicillium spp., Pichia spp., Pragia spp., Pseudomonas spp., Ralstonia spp., Rhodococcus spp., Rubrivivax spp., Shewanella spp., Sphingomonas spp., Sulfurimonas spp., Trichoderma spp., Variovorax spp., and Yokenella spp., and Vibrio spp.

Non-limiting examples of genera of yeast for expression include Saccharomyces (e.g., S. cerevisiae), Pichia, Kluyveromyces (e.g., K. lactis), Hansenula and Yarrowia. In some embodiments, the yeast strain is an industrial polyploid yeast strain. Other non-limiting examples of fungal cells include cells obtained from Aspergillus spp., Penicillium spp., Fusarium spp., Rhizopus spp., Acremonium spp., Neurospora spp., Sordaria spp., Magnaporthe spp., Allomyces spp., Ustilago spp., Botrytis spp., and Trichoderma spp.

The term “cell,” as used herein, may refer to a single cell or a population of cells, such as a population of cells belonging to the same cell line or strain. Use of the singular term “cell” should not be construed to refer explicitly to a single cell rather than a population of cells.

The host cell may include genetic modifications relative to a wild-type counterpart. As a non-limiting example, a host cell (e.g., E. coli) may be modified to reduce or inactivate a gene encoding S-(hydroxymethyl)glutathione dehydrogenase (e.g., frmA).

Reduction of gene expression and/or gene inactivation may be achieved through any suitable method, including but not limited to deletion of the gene, introduction of a point mutation into the endogenous gene, and/or truncation of the endogenous gene. For example, polymerase chain reaction (PCR)-based methods may be used (see, e.g., Gardner et al., Methods Mol Biol. 2014; 1205:45-78). As a non-limiting example, genes may be deleted through gene replacement (e.g., with a marker, including a selection marker). A gene may also be truncated through the use of a transposon system (see, e.g., Poussu et al., Nucleic Acids Res. 2005; 33(12): e104).

A vector encoding any of the recombinant polypeptides (e.g., MDH, HPS, PHI, or other RuMP cycle enzyme) described herein may be introduced into a suitable host cell using any method known in the art.

Non-limiting examples of bacteria transformation protocols are described in Hanahan Methods Enzymol. 1991; 204:63-113; Gerhardt, P. R, Murray, R. G. E., Wood, W. A. & Krieg, N. R. (editors) (1994). Methods for General and Molecular Bacteriology. Washington, D.C.: American Society for Microbiology; and Green, P. N. & Bousfield, I. J. (1982). A taxonomic study of some Gram-negative facultatively methylotrophic bacteria. J Gen Microbiol 128, 623-638, each of which is hereby incorporated by reference in its entirety for this purpose.

Non-limiting examples of yeast transformation protocols are described in Gietz et al., Yeast transformation can be conducted by the LiAc/SS Carrier DNA/PEG method. Methods Mol Biol. 2006; 313:107-20, which is hereby incorporated by reference in its entirety for this purpose. Host cells may be cultured under any conditions suitable as would be understood by one of ordinary skill in the art. For example, any media, temperature, and incubation conditions known in the art may be used. For host cells carrying an inducible vector, cells may be cultured with an appropriate inducible agent to promote expression.

Any of the cells disclosed herein can be cultured in media of any type (rich or minimal) and any composition prior to, during, and/or after contact and/or integration of a nucleic acid. The conditions of the culture or culturing process can be optimized as would be understood by one of ordinary skill in the art. In some embodiments, the selected media is supplemented with various components. In some embodiments, the concentration and amount of a supplemental component is optimized. In some embodiments, other aspects of the media and growth conditions (e.g., pH, temperature, etc.) are optimized. In some embodiments, the frequency that the media is supplemented with one or more supplemental components, and the amount of time that the cell is cultured, is optimized.

The recombinant host cells of the present disclosure may be cultured in the presence of methanol. In some embodiments, a recombinant host cell is cultured in at least 0.01%, at least 0.05%, at least 0.1%, at least 0.5%, at least 1%, at least 5%, at least 10%, at least 15%, at least 20%, at least 25%, at least 30%, at least 35%, at least 40%, at least 45%, at least 50%, at least 55%, at least 60%, at least 70%, at least 80%, at least 90%, at least 95%, or at least 100%, or any values in between, weight per weight (w/w) substitution of saccharide in the feedstock with methanol. Non-limiting examples of saccharides in feedstock include, but are not limited to sucrose, glucose, lactose, dextrose, and fructose.

The % w/w substitution of a saccharide in the feedstock with methanol can be estimated by calculating: [net ¹³C-amino acid of interest %* titer of the amino acid of interest*(Mw of MeOH/Mw of the amino acid)]/MeOH titer ratio in feedstock (e.g., if the amino acid of interest is lysine, the following may be calculated: [net ¹³C-lysine %*lysine titer*(Mw of MeOH/Mw of lysine)]/MeOH titer feeding titer), in which Mw indicates molecular weight and ¹³C-amino acid of interest indicates a ¹³C-labeled amino acid of interest. For the % w/w calculation, a positive control and a negative control are used. The positive control is a strain fed with “normal” full dose of glucose and the negative control is a strain fed with a “deficient” dose of saccharide (e.g., glucose) and no complementing methanol dose. For the experimental treatment, the strain is fed a mix of saccharide (e.g., glucose) and methanol (i.e., the same amount of dextrose as in the negative (glucose deficient) control plus as much methanol as to reach the same amount of total fed carbon as in the positive (full glucose dose) control). The net (natural abundance-corrected) [¹³C]-mass enrichment of an amino acid (net ¹³C-amino acid of interest %) may be calculated as [¹³C-amino acid of interest]/[¹³C-amino acid of interest+¹²C-amino acid of interest]%-natural abundance of ¹³C-amino acid of interest (e.g., net ¹³C-lysine %=[¹³C-lysine]/[¹³C-lysine+¹²C-lysine]%-natural abundance of ¹³C-lysine). As a non-limiting example, LC/MS may be used to measure the amount of an amino acid.

A recombinant host cell's capability to assimilate methanol into an amino acid may also be calculated. As a non-limiting example, methanol assimilation into an amino acid (e.g., lysine) estimates may be based on the complementation of the total production of the amino acid by a methanol-saccharide (e.g., methanol-glucose) co-feed compared to “normal-dose” saccharide and minus 10%-reduced dose saccharide processes, allowing for an estimation of what fraction (or percentage) of the methanol dose was converted into the amino acid, which may be referred to as the methanol-derived amino acid fraction or methanol-derived amino acid percentage.

In some embodiments, a recombinant host cell of the present disclosure is capable of producing an amino acid including at least one carbon (e.g., at least two carbons or all carbons) derived from methanol. As a non-limiting example, ¹³C-labeled methanol may be used as described above to determine the net ¹³C-labeled amino acid percentage produced by a recombinant cell.

In some embodiments, a recombinant host cell that expresses at least one heterologous gene encoding an MDH enzyme, an HPS enzyme, a PHI enzyme, and/or other RuMP pathway enzymes of the present disclosure produces 1%, 5%, 10%, 20%, 30%, 40%, 50%, 60%, 70%, 80%, 90%, 100%, 200%, 300%, 400%, 500%, 600%, 700%, 800%, 900%, or 1,000% more of an amino acid (e.g., lysine) in the presence of methanol compared to a host cell that does not express the at least one heterologous gene encoding an MDH enzyme, an HPS enzyme, a PHI enzyme, and/or other RuMP pathway enzymes. In some embodiments, a recombinant host cell expressing one or more of the heterologous genes described herein with increased lysine production relative to a host cell that does not express the one or more heterologous genes is a methylotrophic cell.

The amount of methanol consumed by a recombinant host cell may also be measured by any suitable technique used in the art and described herein. For example, the methanol carbon mass balance may be calculated by summation of carbons from all sources after the culturing process that derived from methanol. The methanol carbon mass balance may be calculated by taking into account how much methanol is in the initial feedstock, how much methanol is left in the feedstock after culturing the recombinant cell in the feedstock, and how much methanol is lost through evaporation. Without being bound by a particular theory, after fermentation, methanol will likely be incorporated into cell biomass, into secreted end products, into gas phase in the head space, and vented out to environment.

In some embodiments, the percentage of methanol consumed by a recombinant host cell of the present disclosure is at least 0.01%, at least 0.05%, at least 0.1%, at least 0.5%, at least 1%, at least 5%, at least 10%, at least 15%, at least 20%, at least 25%, at least 30%, at least 35%, at least 40%, at least 45%, at least 50%, at least 55%, at least 60%, at least 70%, at least 80%, at least 90%, at least 95%, or at least 100%, or any values in between. In some embodiments, methanol consumption that is at least 0.01%, at least 0.05%, at least 0.1%, at least 0.5%, at least 1%, at least 5%, at least 10%, at least 15%, at least 20%, at least 25%, at least 30%, at least 35%, at least 40%, at least 45%, at least 50%, at least 55%, at least 60%, at least 70%, at least 80%, at least 90%, at least 95%, or at least 100%, or any values in between is indicative of a cell being a methylotrophic cell.

In some embodiments, the recombinant host cells of the present disclosure have at least the same or increased viability in methanol compared to a host cell that does not express a heterologous gene encoding an MDH enzyme, an HPS enzyme, a PHI enzyme, and/or other RuMP pathway enzyme. As compared to a host cell that does not express a heterologous gene encoding an MDH enzyme, an HPS enzyme, a PHI enzyme, and/or other RuMP pathway enzyme, the viability of the recombinant host cell is at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, or any value in between higher than the viability of a host cell that does not express a heterologous gene encoding an MDH enzyme, an HPS enzyme, a PHI enzyme, and/or other RuMP pathway enzyme in the presence of methanol. Non-limiting examples of cell viability assays include MTT assays, trypan blue assays, and luminescent cell viability assays. In some embodiments, cell viability in the presence of methanol is indicative of a recombinant host cell being a methylotrophic cell.

Culturing of the cells described herein can be performed in culture vessels known and used in the art. In some embodiments, an aerated reaction vessel (e.g., a stirred tank reactor) is used to culture the cells. In some embodiments, a bioreactor or fermentor is used to culture the cells. Thus, in some embodiments, the cells are used in fermentation. As used herein, the terms “bioreactor” and “fermentor” are interchangeably used and refer to an enclosure, or partial enclosure, in which a biological, biochemical and/or chemical reaction takes place, involving a living organism or part of a living organism. A “large-scale bioreactor” or “industrial-scale bioreactor” is a bioreactor that is used to generate a product on a commercial or quasi-commercial scale. Large scale bioreactors typically have volumes in the range of liters, hundreds of liters, thousands of liters, or more.

In some embodiments, a bioreactor includes a cell (e.g., a bacteria cell or a yeast cell) or a cell culture (e.g., bacteria cell culture or yeast cell culture), such as a cell or cell culture described herein. In some embodiments, a bioreactor includes a spore and/or a dormant cell type of an isolated microbe (e.g., a dormant cell in a dry state).

Non-limiting examples of bioreactors include: stirred tank fermentors, bioreactors agitated by rotating mixing devices, chemostats, bioreactors agitated by shaking devices, airlift fermentors, packed-bed reactors, fixed-bed reactors, fluidized bed bioreactors, bioreactors employing wave induced agitation, centrifugal bioreactors, roller bottles, and hollow fiber bioreactors, roller apparatuses (for example benchtop, cart-mounted, and/or automated varieties), vertically-stacked plates, spinner flasks, stirring or rocking flasks, shaken multi-well plates, MD bottles, T-flasks, Roux bottles, multiple-surface tissue culture propagators, modified fermentors, and coated beads (e.g., beads coated with serum proteins, nitrocellulose, or carboxymethyl cellulose to prevent cell attachment).

In some embodiments, the bioreactor includes a cell culture system where the cell (e.g., bacteria cell or yeast cell) is in contact with moving liquids and/or gas bubbles. In some embodiments, the cell or cell culture is grown in suspension. In other embodiments, the cell or cell culture is attached to a solid phase carrier. Non-limiting examples of a carrier system includes microcarriers (e.g., polymer spheres, microbeads, and microdisks that can be porous or non-porous), cross-linked beads (e.g., dextran) charged with specific chemical groups (e.g., tertiary amine groups), 2D microcarriers including cells trapped in nonporous polymer fibers, 3D carriers (e.g., carrier fibers, hollow fibers, multicartridge reactors, and semi-permeable membranes that can include porous fibers), microcarriers having reduced ion exchange capacity, encapsulation cells, capillaries, and aggregates. In some embodiments, carriers are fabricated from materials such as dextran, gelatin, glass, or cellulose.

In some embodiments, industrial-scale processes are operated in continuous, semi-continuous or non-continuous modes. Non-limiting examples of operation modes are batch, fed batch, extended batch, repetitive batch, draw/fill, rotating-wall, spinning flask, and/or perfusion mode of operation. In some embodiments, a bioreactor allows continuous or semi-continuous replenishment of the substrate stock, for example a carbohydrate source and/or continuous or semi-continuous separation of the product, from the bioreactor.

In some embodiments, the bioreactor or fermentor includes a sensor and/or a control system to measure and/or adjust reaction parameters. Non-limiting examples of reaction parameters include biological parameters (e.g., growth rate, cell size, cell number, cell density, cell type, or cell state, etc.), chemical parameters (e.g., pH, redox-potential, concentration of reaction substrate and/or product, concentration of dissolved gases, such as oxygen concentration and CO₂ concentration, nutrient concentrations, metabolite concentrations, concentration of an oligopeptide, concentration of an amino acid, concentration of a vitamin, concentration of a hormone, concentration of an additive, serum concentration, ionic strength, concentration of an ion, relative humidity, molarity, osmolarity, concentration of other chemicals, for example buffering agents, adjuvants, or reaction by-products), physical/mechanical parameters (e.g., density, conductivity, degree of agitation, pressure, and flow rate, shear stress, shear rate, viscosity, color, turbidity, light absorption, mixing rate, conversion rate, as well as thermodynamic parameters, such as temperature, light intensity/quality, etc.). Sensors to measure the parameters described herein are well known to one of ordinary skill in the relevant mechanical and electronic arts. Control systems to adjust the parameters in a bioreactor based on the inputs from a sensor described herein are well known to one of ordinary skill in the art in bioreactor engineering.

In some embodiments, the method involves batch fermentation (e.g., shake flask fermentation). General considerations for batch fermentation (e.g., shake flask fermentation) include the level of oxygen and glucose. For example, batch fermentation (e.g., shake flask fermentation) may be oxygen and glucose limited, so in some embodiments, the capability of a strain to perform in a well-designed fed-batch fermentation is underestimated. Also, the final product (e.g., an amino acid, including lysine) may display some differences from a naturally occurring product (e.g., an amino acid, including lysine) in terms of solubility, toxicity, chirality cellular accumulation and secretion and in some embodiments can have different fermentation kinetics.

The methods described herein encompass production of organic compounds using a recombinant host cell, cell lysate or isolated recombinant polypeptides (e.g., MDH, HPS, PHI, or other RuMP cycle enzyme). Examples of organic compounds produced in microorganism fermentation can include amino acids, organic acids, polysaccharides, proteins, antibiotics and alcohols. Examples of amino acids include alanine (A), arginine (R), asparagine (N), aspartic acid (D), cysteine (C), glutamic acid (E), glutamine (Q), glycine (G), histidine (H), isoleucine (I), leucine (L), lysine (K), methionine (M), phenylalanine (F), proline (P), serine (S), threonine (T), tryptophan (W), tyrosine (Y), and valine (V). In some embodiments, the amino acid is a D-amino acid. In some embodiments, the amino acid is a L-amino acid.

Examples of organic acids include acetic acid, lactic acid, pyruvic acid, succinic acid, malic acid, itaconic acid, citric acid, acrylic acid, propionic acid, and fumaric acid. Examples of polysaccharides include xanthan, dextran, alginate, hyaluronic acid, curdlan, gellan, scleroglucan, and pullulan. Examples of proteins include hormones, lymphokines, interferons, and enzymes, such as amylase, glucoamylase, invertase, lactase, protease, and lipase. Examples of antibiotics include antimicrobial agents, such as β-lactams, macrolides, ansamycin, tetracycline, chloramphenicol, peptidergic antibiotics, and aminoglycosides, antifungal agents, such as polyoxin B, griseofulvin, and polyenemacrolides, anticancer agents, daunomycin, adriamycin, dactinomycin, mithramycin, and bleomycin, protease/peptidase inhibitors, such as leupeptin, antipain, and pepstatin, and cholesterol biosynthesis inhibitors, such as compactin, lovastatin, and pravastatin. Examples of alcohols include ethanol, isopropanol, glycerin, propylene glycol, trimethylene glycol, 1-butanol, and sorbitol. Other examples of organic compounds produced in microorganism fermentation can include acrylamide, diene compounds (such as isoprene), carotenoids (such as astaxanthine), isoprenoids (such as limonene, farnesene) and pentanediamine.

Amino acids (e.g., lysine) produced by any of the recombinant host cells disclosed herein may be identified and extracted using any method known in the art. Mass spectrometry (e.g., LC-MS, GC-MS), amino acid biosensors, and ninhydrin assays are non-limiting examples of a method for identification and may be used to help extract an amino acid of interest.

Methods of Determining HPS and or PHI Activity

Aspects of the present disclosure also provide methods of determining whether an enzyme has HPS and/or PHI activity. The method may include adding (i) ribose-5-phosphate; (ii) a RPI enzyme; (iii) an enzyme of interest; (iv) formaldehyde; (v) a PHI enzyme; (vi) a PGI enzyme; (vii) a G6PDH enzyme; (viii) NADP+; (ix) PMSox; and (x) XTT tetrazolium; to a reaction mixture and (b) assaying for XTT formazan, wherein the presence of XTT formazan is indicative of the enzyme of interest being an HPS. In some embodiments, the method includes adding (i) ribose-5-phosphate; (ii) a RPI enzyme; (iii) an HPS; (iv) formaldehyde; (v) an enzyme of interest; (vi) a PGI enzyme; (vii) a G6PDH enzyme; (viii) NADP+; (ix) PMSox; and (x) XTT tetrazolium; to a reaction mixture and (b) assaying for XTT formazan, wherein the presence of XTT formazan is indicative of the enzyme of interest being a PHI. In some embodiments, the method includes adding (i) ribose-5-phosphate; (ii) a RPI enzyme; (iii) an enzyme of interest; (iv) formaldehyde; (v) a second enzyme of interest; (vi) a PGI enzyme; (vii) a G6PDH enzyme; (viii) NADP+; (ix) PMSox; and (x) XTT tetrazolium; to a reaction mixture and (b) assaying for XTT formazan, wherein the presence of XTT formazan is indicative of one of the two enzymes being a PHI and the other enzyme being an HPS. In some embodiments, the method is for determining the presence of PHI and/or HPS in cell lysate. In some embodiments, the method is for determining whether at least one isolated enzyme is a PHI or HIPS.

This invention is not limited in its application to the details of construction and the arrangement of components set forth in the description. The invention is capable of other

embodiments and of being practiced or of being carried out in various ways. Additionally, the phraseology and terminology used herein is for the purpose of description and should not be regarded as limiting. The use of terms such as “including,” “including,” “having,” “containing,” “involving,” and/or variations thereof herein, is meant to encompass the items listed thereafter and equivalents thereof as well as additional items.

The present invention is further illustrated by the following Examples, which in no way should be construed as further limiting. The entire contents of all of the references (including literature references, issued patents, published patent applications, and co pending patent applications) cited throughout this application are hereby expressly incorporated by reference.

### EXAMPLES

### Example 1: Identification and Characterization of Methanol Dehydrogenase (MDH) Enzymes

The present Example describes identification, development, and characterization of MDH enzymes. Those skilled in the art will appreciate that multiple sequences can encode the same polypeptide, and that codon optimization is often useful when expressing sequences in a particular host cell.

MDH Screening

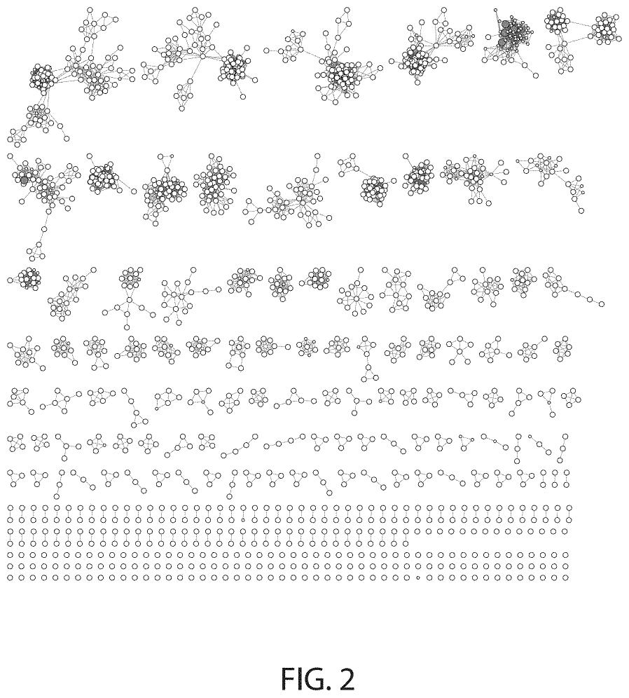

To identify MDH enzymes, a total of 5640 genes of interest were identified using bioinformatics searching and 4173 were de novo synthesized (FIG. 2). Bioinformatics searching included using three “seed” MDH sequences from Ralstonia euthropha and Bacillus methanolicus (SEQ ID NOS: 29-31). Based on sequence similarity, the largest class of enzymes screened generically belong to the broad alcohol dehydrogenase family (EC 1.1.1.1). A set of 2426 genes encoding for proteins with varying amino acid similarity to alcohol and methanol dehydrogenases (ADH/MDH) were selected from public databases as wild-type protein sequences using an alignment tool and a set of seed protein sequences. The nucleotide sequences of the corresponding genes were codon re-coded for optimal expression in E. coli and assembled as synthetic genes by de novo DNA synthesis.

A total of 1837 genes encoding the corresponding polypeptides from this protein family were synthesized. Synthetic linear double stranded DNA fragments were then cloned into suitable vectors, sequenced verified, and expressed in Escherichia coli from constitutive or inducible promoters. Any replicable plasmid for E. coli can be used as a vector. Cell extracts including the proteins were screened for methanol-dependent NAD⁺ reductase activity. Proteins were also screened for ethanol dehydrogenase and butanol dehydrogenase activity.

Cluster analysis approaches and experimental determination of activities on the set of 1837 proteins allowed for isolation of a cluster of sequences that have putative weak to strong methanol dehydrogenase activity defined as assay activity 3 standard deviations above the background negative controls. The cluster included 28 MDH enzymes (SEQ ID NOS: 29-56), which are shown in Table 2 below.

TABLE 2
Non-limiting examples of MDH enzymes.

| | | Nucleic Acid | Amino Acid |
|---|---|---|---|
| MPH | Species Source | Sequence | Sequence |
| BMMGA3_R | Bacillus methanolicus | (SEQ ID NO: 1) | (SEQ ID NO: 29); |
| S03255 | MGA3 | | see also UniprotKB |
| | | | Identifier: I3E2P9. |
| MDH_CnMD | Cupriavidus necator | (SEQ ID NO: 2) | (SEQ ID NO: 30) |
| Hm3_F8GNE | (strain ATCC 43291/ | | |
| 5_CUPNN | DSM 13513/N-1) | | |
| variant or | | | |
| CnMDHm3 | | | |
| MDH_I3DX1 | Bacillus methanolicus | (SEQ ID NO: 3) | (SEQ ID NO: 31) |
| 9 | PB1 | | |
| I3DX19_BAC | Bacillus methanolicus | (SEQ ID NO: 4) | (SEQ ID NO: 32) |
| MT (V361R) | | | |
| A0A0J6L537 | Chromobacterium | (SEQ ID NO: 5) | (SEQ ID NO: 33) |
| | violaceum | | |
| A0A031LYD0 | Acinetobacter sp. Ver3 | (SEQ ID NO: 6) | (SEQ ID NO: 34) |
| or | | | |
| A0A031LYDO | | | |
| 9GAMM | | | |
| A0A0M7C799 | Achromobacter sp. | (SEQ ID NO: 7) | (SEQ ID NO: 35) |
| A0A060QHE9 | Asaia platycodi SF2.1 | (SEQ ID NO: 8) | (SEQ ID NO: 36) |
| G4CT37 | Neisseria wadsworthii | (SEQ ID NO: 9) | (SEQ ID NO: 37) |
| | 9715 | | |
| Q5R120 | Idiomarina loihiensis | (SEQ ID NO: 10) | (SEQ ID NO: 38) |
| | (strain ATCC BAA- | | |
| | 735/DSM 15497/L2- | | |
| | TR) | | |
| A0A060NQ50 | Comamonadaceae | (SEQ ID NO: 11) | (SEQ ID NO: 39) |
| | bacterium BI | | |
| L1M2D7 | Pseudomonas putida | (SEQ ID NO: 12) | (SEQ ID NO: 40) |
| | CSV86 | | |
| LOMOD9 | Enterobacteriaceae | (SEQ ID NO: 13) | (SEQ ID NO: 41) |
| | bacterium (strain FGI | | |
| | 57) | | |
| A0A0Q5FHC | Pseudomonas sp. | (SEQ ID NO: 14) | (SEQ ID NO: 42) |
| 2 | Legf127 | | |
| C5AMS6 | Burkholderia glumae | (SEQ ID NO: 15) | (SEQ ID NO: 43) |
| | (strain BGR1) | | |
| A0A0J1KGJ0 | Aeromonas hydrophila | (SEQ ID NO: 16) | (SEQ ID NO: 44) |
| N9CL98 | Acinetobacter | (SEQ ID NO: 17) | (SEQ ID NO: 45) |
| | johnsonii ANC 3681 | | |
| Q8EGV1 | Shewanella oneidensis | (SEQ ID NO: 18) | (SEQ ID NO: 46) |
| | (strain MR-1) | | |
| G6EZS9 | Commensalibacter | (SEQ ID NO: 19) | (SEQ ID NO: 47) |
| | intestini A911 | | |
| J2MTG6 | Pseudomonas | (SEQ ID NO: 20) | (SEQ ID NO: 48) |
| | fluorescens Q2-87 | | |
| S6KJ47 | Pseudomonas sp. | (SEQ ID NO: 21) | (SEQ ID NO: 49) |
| | CF161 | | |
| M1PK96 | uncultured organism | (SEQ ID NO: 22) | (SEQ ID NO: 50) |
| G2DIW5 | Neisseria weaveri | (SEQ ID NO: 23) | (SEQ ID NO: 51) |
| | LMG 5135 | | |
| N8ZM63 | Acinetobacter gerneri | (SEQ ID NO: 24) | (SEQ ID NO: 52) |
| | DSM 14967 = CIP | | |
| | 107464 | | |
| P45513 | Citrobacter freundii | (SEQ ID NO: 25) | (SEQ ID NO: 53) |
| MDH_A0A03 | Acinetobacter sp. Ver3 | (SEQ ID NO: 26) | (SEQ ID NO: 54) |
| 1LYD0_9GA | | | |
| MM [S31V, | | | |
| A169V, | | | |
| A368R] | | | |
| MDH_A0A03 | Acinetobacter sp. Ver3 | (SEQ ID NO: 27) | (SEQ ID NO: 55) |
| 1LYD0_9GA | | | |
| MM [A26V, | | | |
| A169V, | | | |
| A368R] | | | |
| MDH_A0A03 | Acinetobacter sp. Ver3 | (SEQ ID NO: 28) | (SEQ ID NO: 56) |
| 1LYD0_9GA | | | |
| MM [A26V, | | | |
| S31V, A169V, | | | |
| A368] | | | |
| mdh_A0A0G3 | | (SEQ ID NO: 73) | (SEQ ID NO: 81) |
| CNS6 9ENTR | | | |
| mdh_I3E2P9_ | | (SEQ ID NO: 74) | (SEQ ID NO: 82) |
| BACMT | | | |
| mdh_A0A0A3 | | (SEQ ID NO: 75) | (SEQ ID NO: 83) |
| IWY5 9BACI | | | |
| mdh_W0H9W | | (SEQ ID NO: 76) | (SEQ ID NO: 84) |
| 4 PSECI | | | |
| mdh_I0HVZ3 | | (SEQ ID NO: 77) | (SEQ ID NO: 85) |
| RUBGI | | | |
| mdh_Q4KGV | | (SEQ ID NO: 78) | (SEQ ID NO: 86) |
| 5 PSEF5 | | | |
| mdh_A0A0Q0 | | (SEQ ID NO: 79) | (SEQ ID NO: 87) |
| ITX7_9GAM | | | |
| M | | | |
| mdh_A0A063 | | (SEQ ID NO: 80) | (SEQ ID NO: 88) |
| Y790_9GAM | | | |
| M | | | |

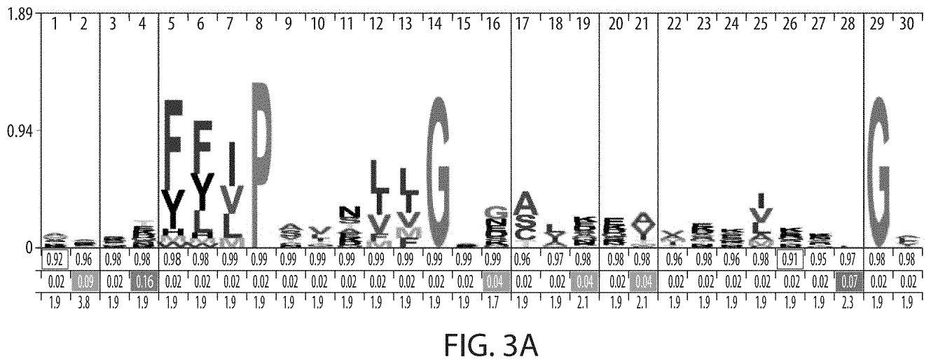

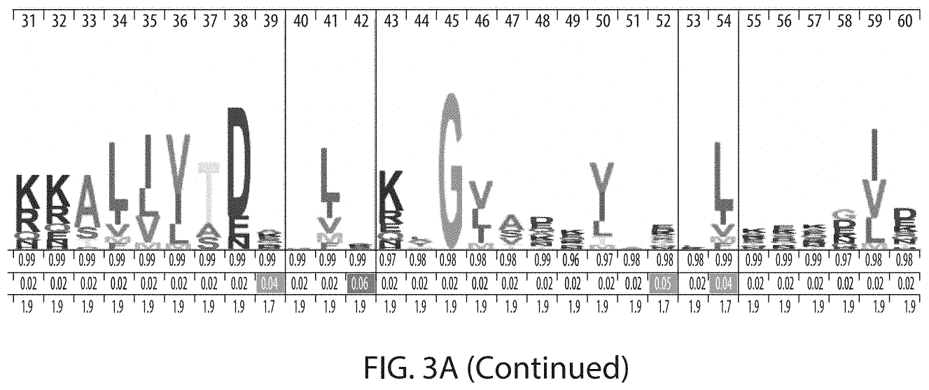

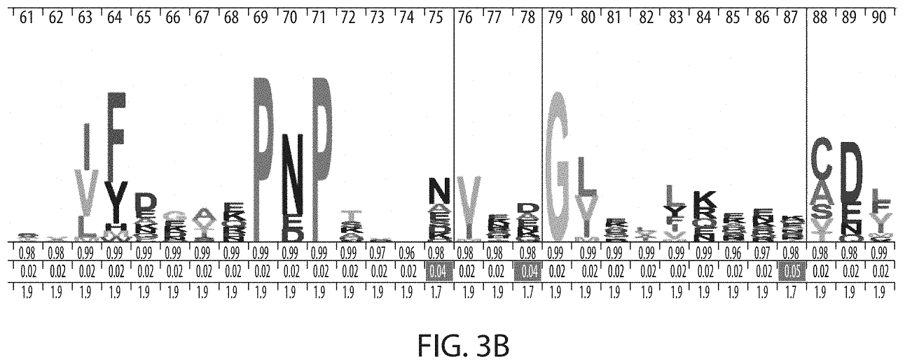

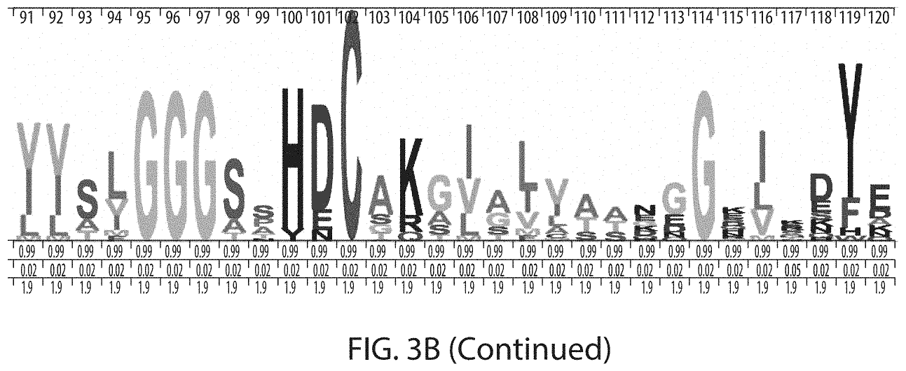

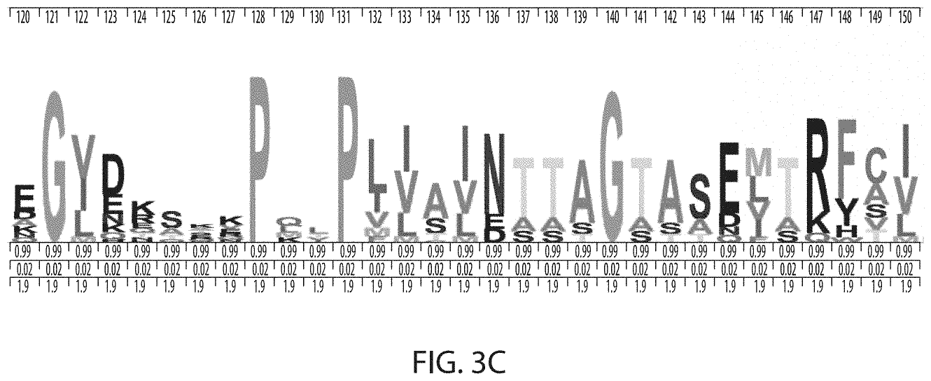

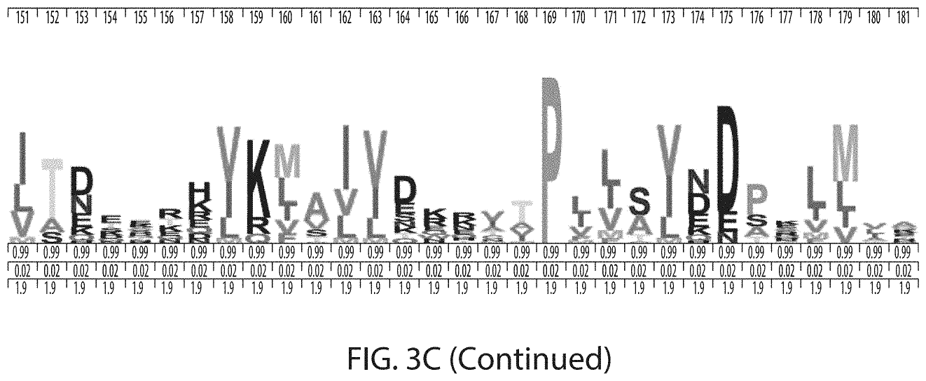

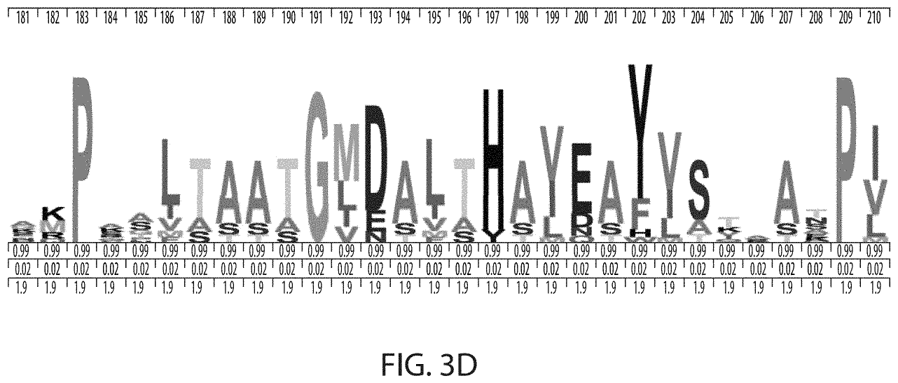

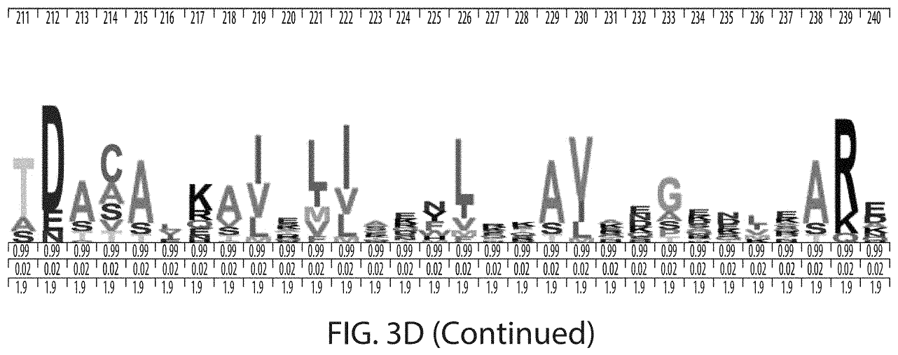

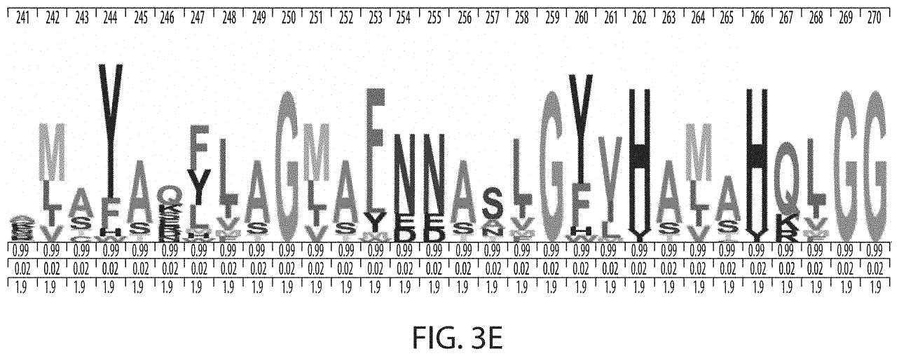

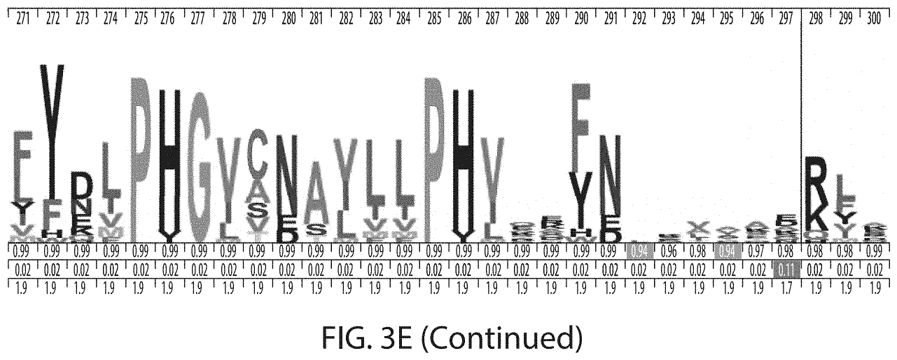

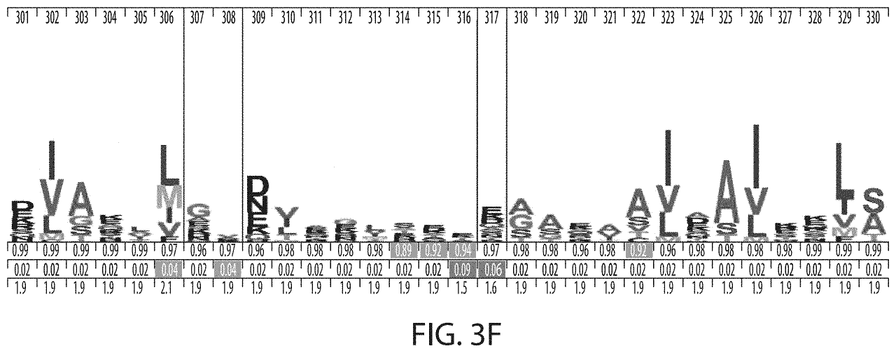

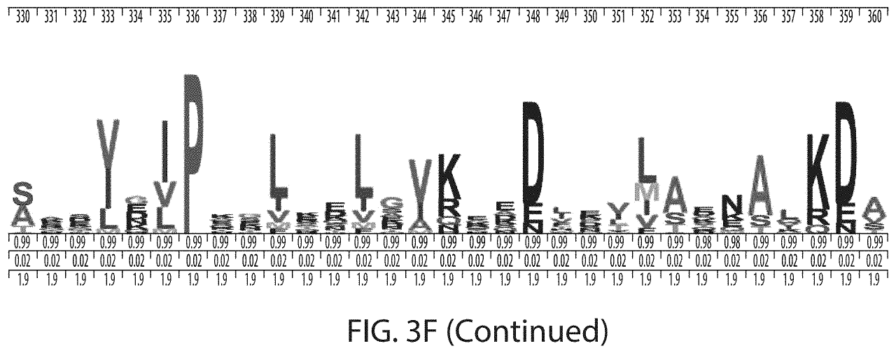

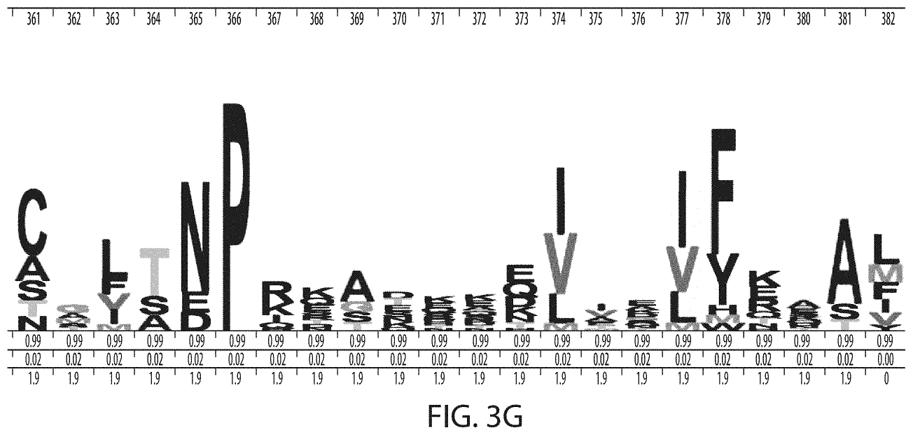

The sequence information of this identified cluster was used to generate a Hidden Markov structure model. A sequence logo of the Hidden Markov Model is shown in FIGS. 3A-3G. A ClustalW alignment of the 28 sequences is shown in FIGS. 4A-4C. In FIGS. 4A-4C, the sequences are listed as follows:

(SEQ ID NO: 44)
1. mdh_A0A0J1KGJ0_AERHY
(SEQ ID NO: 46)
2. mdh_Q8EGV1_SHEON
(SEQ ID NO: 47)
3. mdh_G6EZS9_9PROT
(SEQ ID NO: 48)
4. mdh_J2MTG6_PSEFL
(SEQ ID NO: 49)
5. mdh_S6KJ47_9PSED
(SEQ ID NO: 40)
6. mdh_L1M2D7_PSEPU
(SEQ ID NO: 42)
7. mdh_A0A0Q5FHC2_9PSED
(SEQ ID NO: 39)
8. mdh_A0A060NQ50_9BURK
(SEQ ID NO: 33)
9. mdh_A0A0J6L537_CHRVL
(SEQ ID NO: 41)
10. mdh_L0M0D9_ENTBF
(SEQ ID NO: 38)
11. mdh_Q5R120_IDILO
(SEQ ID NO: 37)
12. mdh_G4CT37_9NEIS
(SEQ ID NO: 51)
13. mdh_G2DIW5_9NEIS
(SEQ ID NO: 35)
14. mdh_A0A0M7C799_9BURK
(SEQ ID NO: 30)
15. mdh_CnMDHm3
(SEQ ID NO: 43)
16. mdh_C5AMS6_BURGB
(SEQ ID NO: 50)
17. mdh_M1PK96_9ZZZZ
(SEQ ID NO: 36)
18. mdh_A0A060QHE9_9PROT
(SEQ ID NO: 54)
19. mdh_A0A031LYD0_9GAMM-531V-A169V-A368R
(SEQ ID NO: 56)
20. mdh_A0A031LYD0_9GAMM-A26V-S31V-A169V-A368R
(SEQ ID NO: 55)
21. mdh_A0A031LYD0_9GAMM-A26V-A169V-A368R
(SEQ ID NO: 34)
22. mdh_A0A031LYD0_9GAMM
(SEQ ID NO: 45)
23. mdh_N9CL98_ACIJO
(SEQ ID NO: 52)
24. mdh_N8ZM63_9GAMM
(SEQ ID NO: 53)
25. mdh_P45513
(SEQ ID NO: 31)
26. mdh_Bm_ADH61(wt)
(SEQ ID NO: 32)
27. mdh_BmADH61[V361R]
(SEQ ID NO: 29)
28. mdh_(Bm)|I3E2P9

A subset of the expressed proteins was also screened for methanol dehydrogenase/formaldehyde production activity (FIGS. 5-6). The Nash assay (Nash Biochem J. 1953 October; 55(3):416-21) was used to determine the formaldehyde production activity, while the methanol-dependent NAD+ reductase activity was measured using the XTT tetrazolium assay shown at the top of FIG. 6. In these studies, the gene-encoded enzyme activities were screened in the context of cell extracts (lysed cells) or in vivo (whole cells).

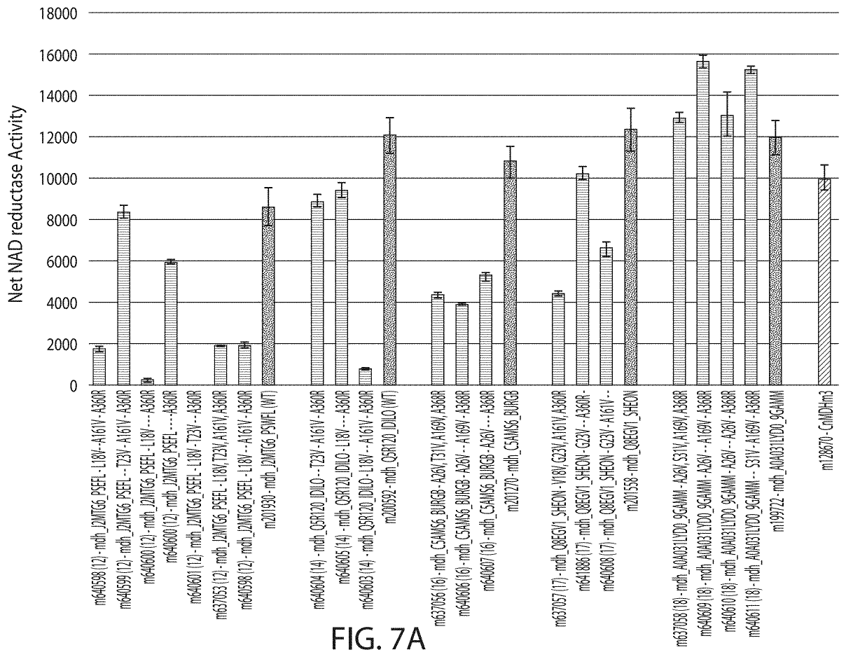

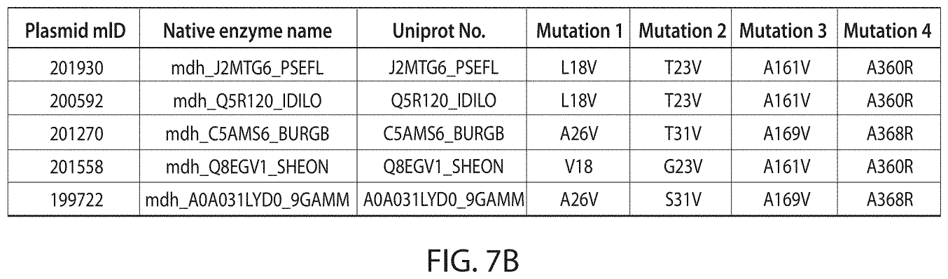

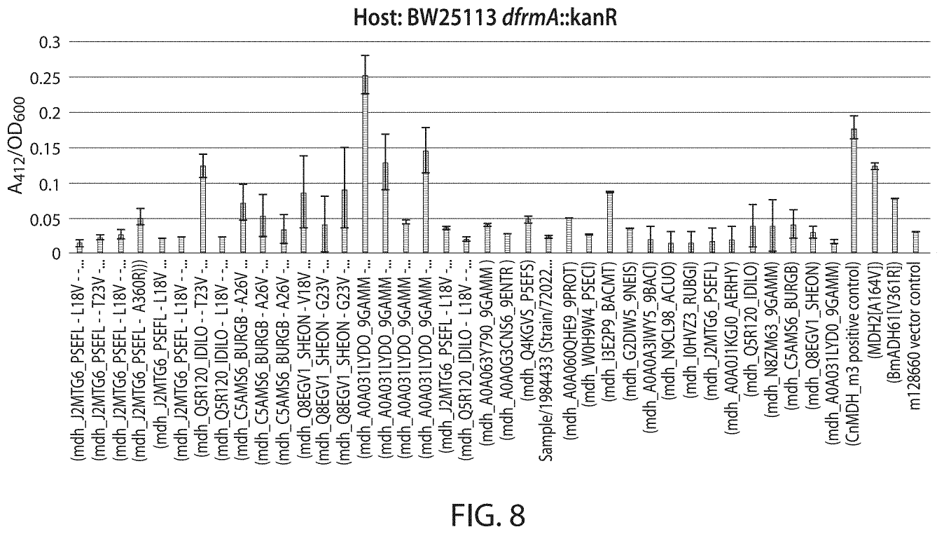

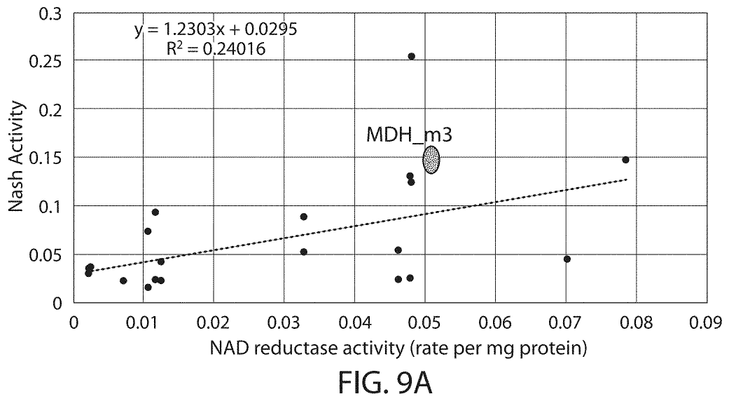

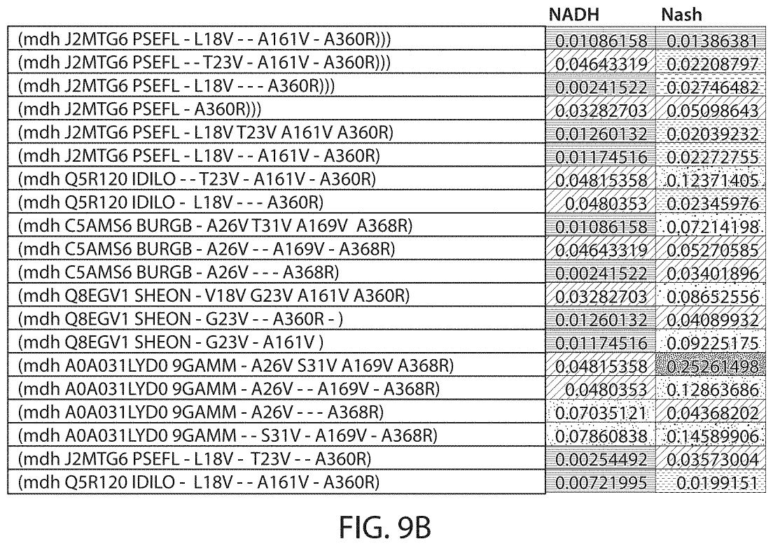

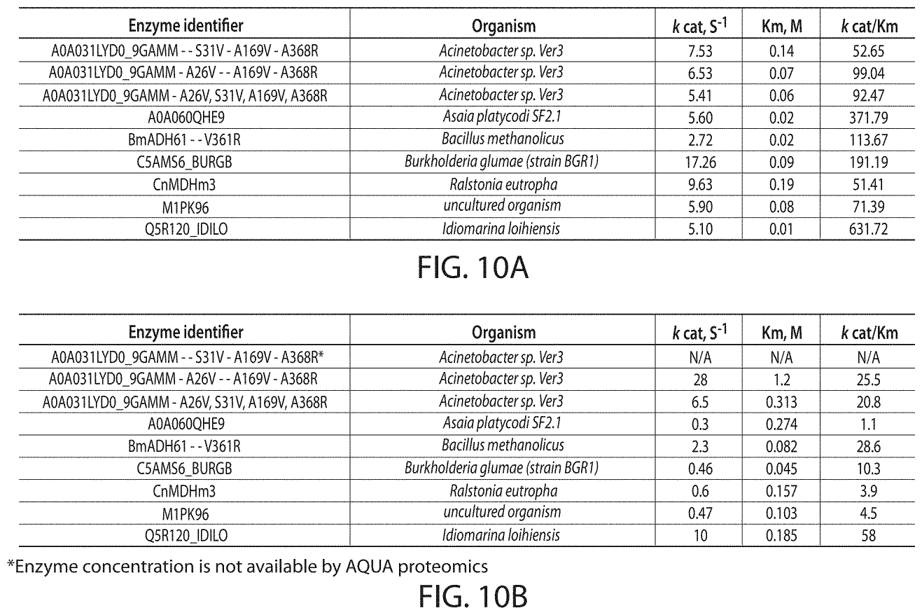

Six MDH genes were selected and subjected to site-directed mutagenesis to further improve the catalytic activity of the corresponding enzyme (FIGS. 7, 8, and 9A-9B;). A set of mutants from one of the six genes showed improved catalytic activity as measured by methanol oxidation, NADH production, and formaldehyde production (Acinetobacter sp. Ver3 Uniprot A0A031LYD0_9GAMM variants) (FIG. 8). The Acinetobacter sp. Ver3 Uniprot A0A031LYD0_9GAMM variants showing improved activity relative to wild-type A0A031LYD0_9GAMM and relative to the positive control CnMDHm3 (SEQ ID NO: 30). The variants included the following mutations: (1) A26V, S31V, A169V, and A368R; (2) A26V, A169V, and A368R; (3) A26V and A368R; or (4) S31V, A169V, and A368R. The A0A031LYD0_9GAMM variants showed at least 20% increase in net NAD reductase activity as compared to the positive control CnMDHm3 (FIG. 7). The A0A031LYD0_9GAMM variant including the A26V, A169V, and A368R mutations showed a more than 25% increase in net NAD reductase activity as compared to the wild-type A0A031LYD0_9GAMM. A complete kinetic characterization was performed for 7 of the most active enzymes identified in the MDH screenings (FIGS. 9A-9B, including 2 controls, one of which was CnMDHm3).

Therefore, MDH enzymes were identified that increased the methanol dehydrogenase activity (as determined by formaldehyde production) and methanol-dependent NAD* reductase activity of bacterial host cells.

### Example 2: Identification and Characterization of 3-hexulose-6-phosphate Synthase (HPS), and 3-hexulose-6-phosphate Isomerase (PHI) Enzymes

HPS and PHI Screening

The present Example describes identification, development, and/or characterization of certain useful HPS and PHI polypeptides and/or sequences that encode them. Those skilled in the art will appreciate that multiple sequences can encode the same polypeptide, and that codon optimization is often useful when expressing sequences in a particular host cell.

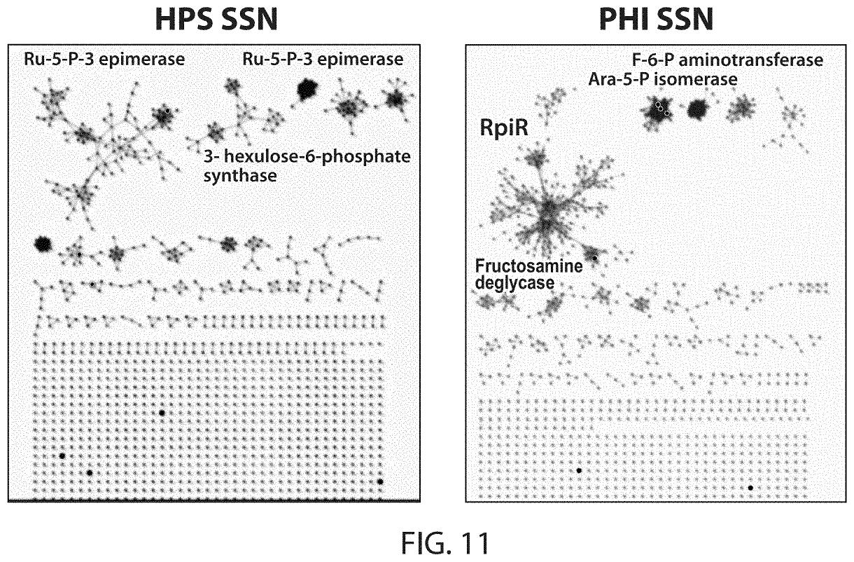

Libraries of putative 3-hexulose-6-phosphate synthase (HPS), and 3-hexulose-6-phosphate isomerase (PHI) were constructed following a similar pipeline described above for ADH/MDH genes/enzymes. A total of 2004 candidate HPS and PHI enzymes (about half from each class) were identified using seed polypeptides (FIG. 11). A total of 1346 were synthesized as individually expressed genes in the inducible expression vector m416625. Additionally, 603 synthetic two-gene (candidate HPS and candidate PHI) operons were designed taking into account syntheny/genetic linkage, taxonomy and lifestyle of the organisms the genes were derived from. A total of 460 were synthesized for expression in m416625 from a P_{L} promoter. The screening for the enzyme activities was performed on cell extracts after gene expression induction using novel enzyme assays (FIG. 12). As shown in FIG. 12, extracts from cells expressing a combination of putative HPS and putative PHI enzymes were screened in an assay that is based on reduction of the XTT tetrazolium salt.

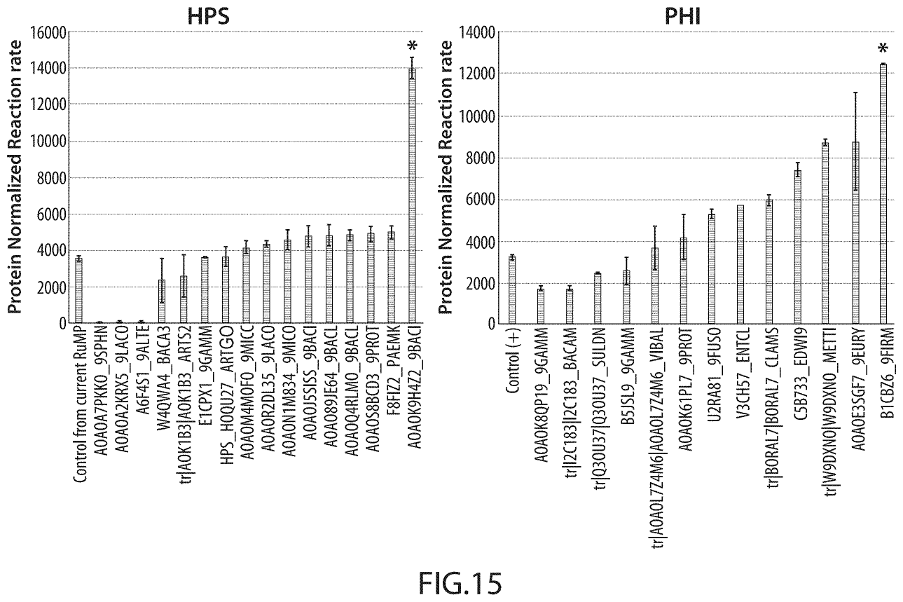

In the in vitro assay, R5P compound is converted to Ru5P as substrate for HPS together with formaldehyde. The product hexulose-6-P from HPS reaction is then isomerized to F6P by PHI. The resultant F6P is converted to NADPH by a series of enzymes including Pgi and Zwf. Flux through the pathway was determined by measuring reduction of the XTT tetrazolium salt into formazan with the presence of NADPH generated from the above enzyme coupled reaction, which was detected in a colorimetric assay. The primary screening identified at least 15 candidate HPS hits based on HPS enzyme activities (defined as Z-score greater than 2; FIG. 13, with corresponding sequences included in Table 3) and 10 candidate PHI hits based on PHI enzyme activities (defined as Z-score greater than 2; FIG. 14, with corresponding sequences included in Table 4), a subset of which was confirmed to be as active or more active than the Methylococcus capsulatus control enzymes (FIG. 15). The in vitro assay shown in FIG. 12 was used.

TABLE 3
Non-limiting examples of BPS enzymes.

| | | Nucleic acid | Amino Acid |
|---|---|---|---|
| HPS | Source | Sequence | Sequence |
| A0A0M4M | | (SEQ ID NO: 89) | (SEQ ID NO: 106) |
| 0F0 | | | |
| E1CPX1 | | (SEQ ID NO: 90) | (SEQ ID NO: 107) |
| F8FIZ2 | | (SEQ ID NO: 91) | (SEQ ID NO: 108) |
| HPS(MCA3 | | (SEQ ID NO: 92) | (SEQ ID NO:109) |
| 043) | | | |
| H0QU27 | Arthrobacter | (SEQ ID NO: 93) | (SEQ ID NO: 110) |
| | globiformis | | |
| | NBRC 12137 | | |
| A0A0S8BC | Betaproteo- | (SEQ ID NO: 94) | (SEQ ID NO: 111) |
| D3 | bacteria | | |
| | bacterium SG8 | 39 | |
| B9E933 | Macrococcus | (SEQ ID NO: 95) | (SEQ ID NO: 112) |
| | caseolyticus | | |
| | (strain | | |
| | JCSC5402) | | |
| W4QWA4 | Bacillus akibai | (SEQ ID NO: 96) | (SEQ ID NO: 113) |
| | (strain ATCC | | |
| | 43226/DS21/1 | | |
| | 21942/JC21/1 | | |
| | 9157/1139) | | |
| A0K1B3 | Arthrobacter | (SEQ ID NO: 97) | (SEQ ID NO: 114) |
| | sp. (strain | | |
| | FB24) | | |
| A0A0K9H4 | Bacillus sp. | (SEQ ID NO: 98) | A (SEQ ID NO: |
| Z2 | FJAT-27231 | | 115) |
| A0A0R2DL | Lactobacillus | (SEQ ID NO: 99) | (SEQ ID NO: 116) |
| 35 | floricola DSM | | |
| | 23037 = JCM | | |
| | | 16512 | |
| A0A0J5SIS | Bacillus | (SEQ ID NO: 100) | (SEQ ID NO: 117) |
| 5 | marisflavi | | |
| A0A0Q4RL | Paenibacillus | (SEQ ID NO: 101) | (SEQ ID NO: 118) |
| M0 | sp. Legf72 | | |
| A0A0R2KR | Lactobacillus | (SEQ ID NO: 102) | (SEQ ID NO: 119) |
| X5 | ceti DSM | | |
| | 22408 | | |
| A0A089JE6 | Paenibacillus | (SEQ ID NO: 103) | (SEQ ID NO: 120) |
| 4 | sp. FSL P4- | | |
| | 0081 | | |
| A0A0N1M8 | Frigori- | (SEQ ID NO: 104) | (SEQ ID NO: 121) |
| 34 | bacterium | | |
| | sp. RIT-PI-h | | |
| Q602L4_M | Methylococcus | (SEQ ID NO: 105) | (SEQ ID NO: 122) |
| ETCA | capsulatus | | |

TABLE 4
Non-limiting examples of PHI Enzymes.

| | | Nucleic acid | Amino Acid |
|---|---|---|---|
| PHI | Source | Sequence | Sequence |
| A0A0E3SG | Alethanosarcina | (SEQ ID NO: | (SEQ ID NO: |
| F7 | horonobensis HB- | 123) | 135) |
| | 1 | | |
| B0RAL7 | Corynebacterium | (SEQ ID NO: | (SEQ ID NO: |
| | Sepedonicum | 124) | 136) |
| B1CBZ6 | Anaerofustis | (SEQ ID NO: | (SEQ ID NO: |
| | stercorihominis | 125) | 137) |
| | DSM 17244 | | |
| PHI(MCA3 | Alethylococcus | (SEQ ID NO: | (SEQ ID NO: |
| 044) | capsulatus | 126) | 138) |
| W9DXN0 | Methanolobus | (SEQ ID NO: | (SEQ ID NO: |
| | tindarius DSM | 127) | 139) |
| | 2278 | | |
| A0A0K8QP | Mizugalciibacter | (SEQ ID NO: | (SEQ ID NO: |
| 19 | sediminis | 128) | 140) |
| Q8TRO1 | Methanosarcina | (SEQ ID NO: | (SEQ ID NO: |
| | acenvorans | 129) | 141) |
| | (strain ATCC | | |
| | 35395/DSM | | |
| | 2834/JCH | | |
| | 12185/C2.4) | | |
| A0A0L7Z4 | Vibrio | (SEQ ID NO: | (SEQ ID NO: |
| M6 | alginolyticus | 130) | 142) |
| C5B733 | Edwardsiella | (SEQ ID NO: | (SEQ ID NO: |
| ictaluri | | 131) | 143) |
| Q30U37 | Sulfurimonas | (SEQ ID NO: | (SEQ ID NO: |
| | denitrtficans | 132) | 144) |
| | (Strain ATCC | | |
| | 33889 / DSM | | |
| | 1251) | | |
| | (Thiomicrospira | | |
| | denitrtficans | | |
| | (strain ATCC | | |
| | 33889/D5M | | |
| | 1251)) | | |
| V3CH57 | Enterobacter | (SEQ ID NO: | (SEQ ID NO: |
| | cloacae UCICRE | 133) | 145) |
| | 12 | | |
| Q602L3_M | Akthylococcus | (SEQ ID NO: | (SEQ ID NO: |
| ETCA | capsulatus | 134) | 146) |

Therefore, HPS and PHI enzymes were identified that could be used to promote flux through the RuMP pathway in bacterial host cells.

### Example 3: Development of Recombinant Host Cells that are Capable of Using Methanol to Produce Lysine

This Example describes the development of recombinant host cells with increased lysine production.

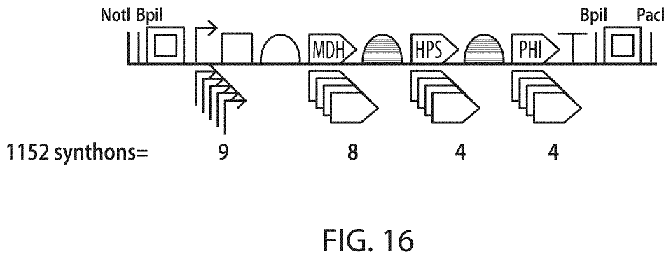

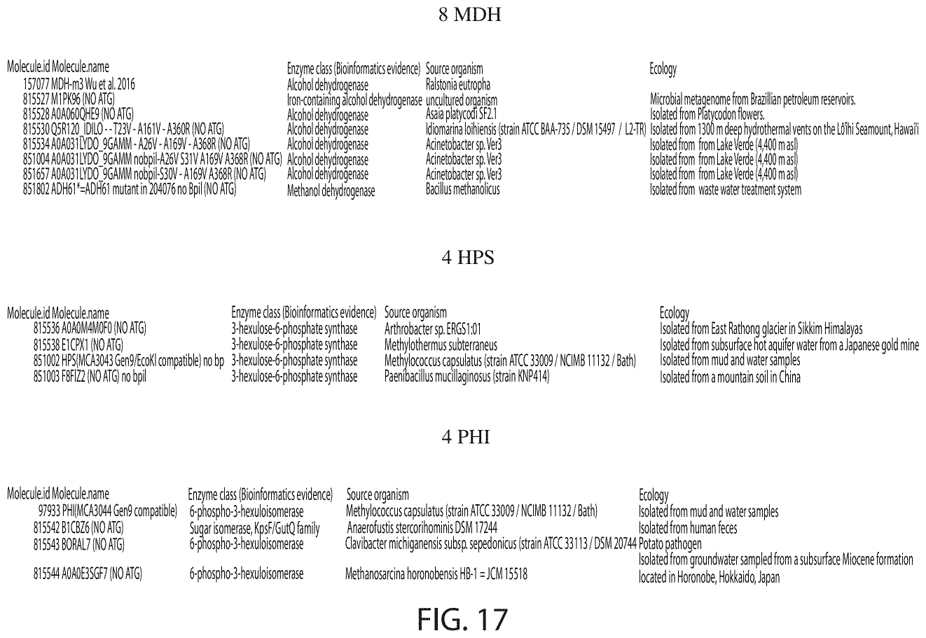

Genes expressing a subset of the MDH, HPS and PHI enzymes (FIG. 17) and a library of regulatory parts (promoters, operators, mRNA stability cassettes, ribosomal binding sites and terminators; FIG. 16) were assembled in full factorial fashion into methanol assimilation pathways of the ribulose monophosphate type by de novo techniques, cloned into low copy number vectors and tested in an E. coli strain for assimilation of ¹³C-methanol into biomass and product. The E. coli strain includes a frmA gene knockout and does not naturally undergo methanol assimilation. The frmA gene encodes S-(hydroxymethyl)glutathione dehydrogenase.

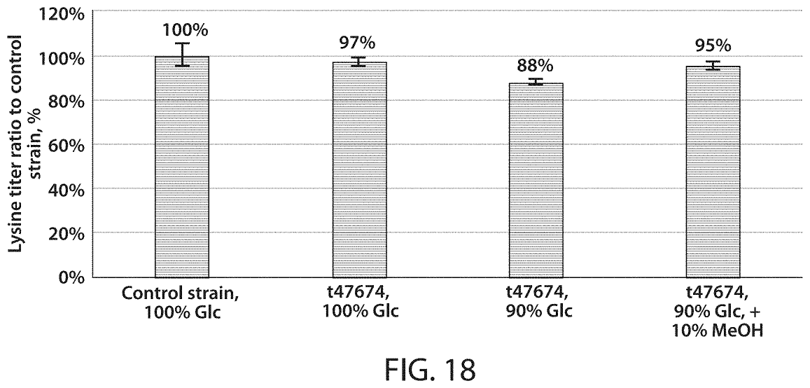

836 pathways were synthesized out of the 1,152 targeted pathways. The pathway plasmids were transformed into the E. coli strain including a frmA gene knockout and tested in a batch-growth protocol for measuring ¹³C-net enrichment in lysine using a co-feed regimen of 20 g/L of methanol and 20 g/L of glucose. Selected Reaction Monitoring LC-MS experiments were used to determine [¹³C]-lysine/[¹²C]-lysine ratios and titers. The recombinant host cells were tested for incorporation of [¹³C]-MeOH into [¹³C]-Lysine to determine a net (natural abundance-corrected) [¹³C]-mass enrichment ([M+1]/[M+M+1]). A notable fraction of these pathway plasmids showed increased fraction enrichment over the empty vector control, with at least one strain showing 26-27% fraction enrichment. The percent dextrose substitution with methanol based on lysine titers was also determined, and greater than 5% dextrose substitution with methanol based on lysine titers was identified in at least one strain (FIG. 18).

Therefore, introduction of plasmids encoding MDH, HPS, and PHI enzymes identified in the screening studies described in Examples 1 and 2 can be used to create recombinant host cells that can efficiently assimilate methanol and that can use methanol to produce lysine.

### Example 4: Identification and Characterization of Additional RuMP Cycle Enzymes

The present Example describes identification, development, and/or characterization of additional RuMP pathway enzymes including ribose-5-phosphate isomerase (rpi), D-ribulose 5-phosphate 3-epimerase (rpe), transketolase (tkt), transaldolase (tal), phosphofructokinase (pfk), sedoheptulose 1,7-Bisphosphatase (glpX), fructose-bisphosphate aldolase (fba), 6-phosphogluconate dehydrogenase (gnd), glucose-6-phosphate dehydrogenase (zwf), or a combination thereof (non-limiting examples of genes encoding the indicated enzymes in B. methanolicus are indicated in parenthesis). Those skilled in the art will appreciate that multiple sequences can encode the same polypeptide, and that codon optimization is often useful when expressing sequences in a particular host cell.

Enzyme libraries for RuMP cycle engineering were created by exploring public databases for candidate pentose phosphate pathway and glycolysis enzymes. A total of 4,677 genes belonging to 9 enzyme classes were targeted for synthesis in an expression vector and assay development was performed using E. coli native set as control enzymes, including rpe, rpiA, zwf, gnd, pfkA, tktA, talA, glpX and fbaB.

TABLE 5
Non-limiting example of additional RuMP cycle enzymes.

| RuMP | | | |
|---|---|---|---|
| Cycle | | Nucleic Acid | Amino Acid |
| Enzyme | UniProtKB | Sequence | Sequence |
| fba | A0A099TJQ7_9H | (SEQ ID NO: 147) | (SEQ ID NO: 153) |
| | ELI | | |
| fba | U2PT58_9CLOT | (SEQ ID NO: 148) | (SEQ ID NO: 154) |
| fba | C3WBT0_FUSM | (SEQ ID NO: 149) | (SEQ ID NO: 155) |
| | R | | |
| fba | W1SAI3_9BACI | (SEQ ID NO: 150) | (SEQ ID NO: 156) |
| fba | A0A176JA54_9B | (SEQ ID NO: 151) | (SEQ ID NO: 157) |
| | ACI | | |
| fba | A0A0M5JGI7_9B | (SEQ ID NO: 152) | (SEQ ID NO: 158) |
| | ACI | | |
| GlpX | A0A0Q7NTH6_9 | (SEQ ID NO: 159) | (SEQ ID NO: 166) |
| | NOCA | | |
| GlpX | A0A0T9Q4A7_M | (SEQ ID NO: 160) | (SEQ ID NO: 167) |
| | YCTX | | |
| GlpX | A0A0M0KFD7_9 | (SEQ ID NO: 161) | (SEQ ID NO: 168) |
| | BACI | | |
| GlpX | A0A0CIINZ9_9R | (SEQ ID NO: 162) | (SEQ ID NO: 169) |
| | HOB | | |
| GlpX | S5Y9Y2_PARAH | (SEQ ID NO: 163) | (SEQ ID NO: 170) |
| GlpX | A0A0J6VGU7_9 | (SEQ ID NO: 164) | (SEQ ID NO: 171) |
| | RHIZ | | |
| GlpX | A0A0D6MUT9_ | (SEQ ID NO: 165) | (SEQ ID NO: 172) |
| | ACEAC | | |
| gnd | A0A150K4A6_B | (SEQ ID NO: 173) | (SEQ ID NO:179) |
| | ACCO | | |
| gnd | A0A147K817_9B | (SEQ ID NO: 174) | (SEQ ID NO: 180) |
| | ACI | | |
| gnd | E6V7Q7_VARPE | (SEQ ID NO: 175) | (SEQ ID NO: 181) |
| gnd | A0A0P0YRA4_9 | (SEQ ID NO: 176) | (SEQ ID NO: 182) |
| | ENTR | | |
| gnd | A0A150J558_BA | (SEQ ID NO: 177) | (SEQ ID NO: 183) |
| | CCO | | |
| gnd | J2DHU2_KLEPN | (SEQ ID NO: 178) | (SEQ ID NO: 184) |
| pfk | PFKA_MYCPN | (SEQ ID NO: 185) | (SEQ ID NO: 191) |
| pfk | K6C613_9BACI | (SEQ ID NO: 186) | (SEQ ID NO: 192) |
| pfk | R7DTY4_9FIRM | (SEQ ID NO: 187) | (SEQ ID NO: 193) |
| pfk | A0A085L152_9F | (SEQ ID NO: 188) | (SEQ ID NO: 194) |
| | LAO | | |
| PR( | A0A0G7ZN65_9 | (SEQ ID NO: 189) | (SEQ ID NO: 195) |
| | MOLU | | |
| PR( | A0A0F6YL10_9 | (SEQ ID NO: 190) | (SEQ ID NO: 196) |
| | DELT | | |
| rpe | M1X1F7_ 9NOST | (SEQ ID NO: 197) | (SEQ ID NO: 204) |
| rpe | K9ZEX9_ANAC | (SEQ ID NO: 198) | (SEQ ID NO: 205) |
| | C | | |
| rpe | K9UHV0_9CYA | (SEQ ID NO: 199) | (SEQ ID NO: 206) |
| | N | | |
| rpe | K9V8A4_9CYA | (SEQ ID NO: 200) | (SEQ ID NO: 207) |
| | N | | |
| rpe | A0A068MW34_S | (SEQ ID NO: 201) | (SEQ ID NO: 208) |
| | YNY4 | | |
| rpe | A0A101G6H0_9F | (SEQ ID NO: 202) | (SEQ ID NO: 209) |
| | IRM | | |
| rpe | A0A097B8L1_LI | (SEQ ID NO: 203) | (SEQ ID NO: 210) |
| | SIV | | |
| rpi | A0A085A5R9_9E | (SEQ ID NO: 211) | (SEQ ID NO: 217) |
| | NTR | | |
| rpi | J7WVJ5_BACCE | (SEQ ID NO: 212) | (SEQ ID NO: 218) |
| rpi | G6C9U3_9STRE | (SEQ ID NO: 213) | (SEQ ID NO: 219) |
| rpi | AORF02_BACAH | (SEQ ID NO: 214) | (SEQ ID NO: 220) |
| rpi | A0A0A0BFL7_9 | (SEQ ID NO: 215) | (SEQ ID NO: 221) |
| | GAMM | | |
| rpi | F5W299_9STRE | (SEQ ID NO: 216) | (SEQ ID NO: 222) |
| tal | B7LWR6_ESCF3 | (SEQ ID NO: 223) | (SEQ ID NO: 229) |
| tal | IIXLN0_METNJ | (SEQ ID NO: 225) | (SEQ ID NO: 231) |
| tal | A0A177P7W1_9 | (SEQ ID NO: 226) | (SEQ ID NO: 232) |
| | GAMM | | |
| tal | A0A177N227_9G | (SEQ ID NO: 227) | (SEQ ID NO: 233) |
| | AMM | | |
| tal | B2ILR7_STRPS | (SEQ ID NO: 228) | (SEQ ID NO: 234) |
| tkt | V5XNZ7_ENTM | (SEQ ID NO: 235) | (SEQ ID NO: 241) |
| | U | | |
| tkt | A0A179ETL1_9E | (SEQ ID NO: 236) | (SEQ ID NO: 242) |
| | NTE | | |
| tkt | A0A0311A99_95 | (SEQ ID NO: 237) | (SEQ ID NO: 243) |
| | PHN | | |
| tkt | A0A0QOHT44_9 | (SEQ ID NO: 238) | (SEQ ID NO: 244) |
| | GAMM | | |
| tkt | M5P892_9BACI | (SEQ ID NO: 239) | (SEQ ID NO: 245) |
| tkt | Q5WG06_BACS | (SEQ ID NO: 240) | (SEQ ID NO: 246) |
| | K | | |
| zwf | A0A0D6MYB6_ | (SEQ ID NO: 247) | (SEQ ID NO: 253) |
| | ACEAC | | |
| zwf | M7PNC4_9GAM | (SEQ ID NO: 248) | (SEQ ID NO: 254) |
| | M | | |
| zwf | C3AVX4_BACM | (SEQ ID NO: 249) | (SEQ ID NO: 255) |
| | Y | | |
| zwf | EIQG88_DESB2 | (SEQ ID NO: 250) | (SEQ ID NO: 256) |
| zwf | A0A0A2ESG8_9 | (SEQ ID NO: 251) | (SEQ ID NO: 257) |
| | PORP | | |
| zwf | A0A136KWE2_9 | (SEQ ID NO: 252) | (SEQ ID NO: 258) |
| | CHLR | | |

Sourced genes were targeted broadly across phylogenetic space and, when possible, preference to known methylotrophic organisms was given. Synthesis success was on average above 80%.

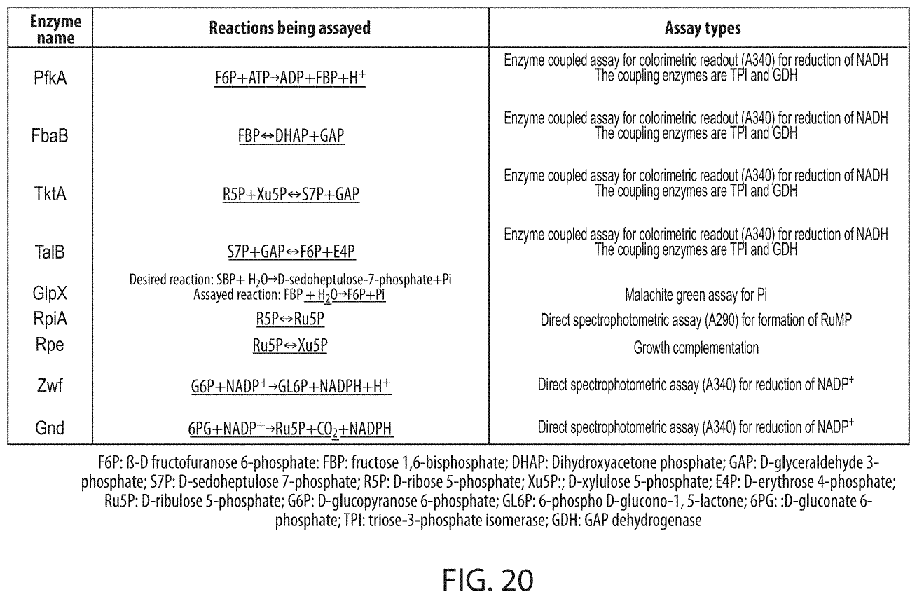

Each library was screened using a combination of methods. A set of 56 enzymes belonging to the nine enzyme activities (FIG. 19) was selected for assembly into plasmids as described below. FIG. 20 shows methods used to identify the indicated enzymes.

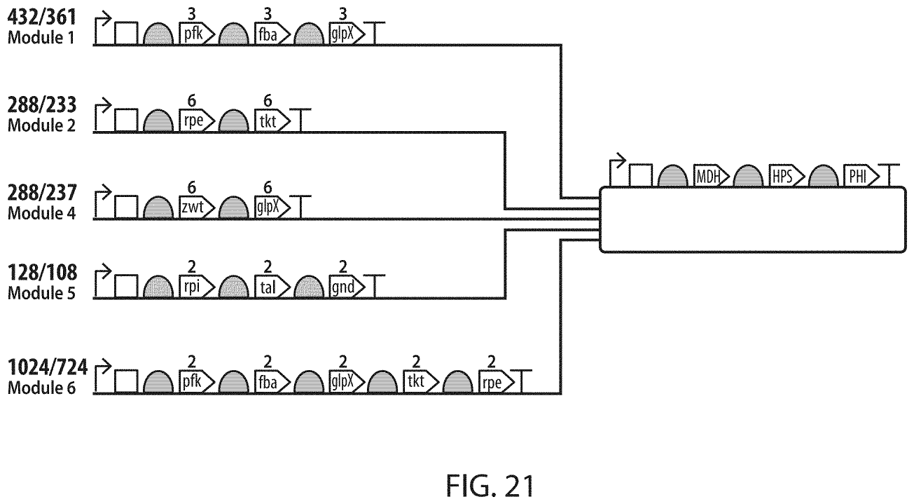

Two to five of the set of 56 genes were grouped into candidate metabolic modules and the synthon modules spanned in length from 3 to 6.2 kilobases. The synthon modules were cloned into plasmids that encode an MDH, an HPS, and a PHI. FIG. 21 is a schematic showing integration of an expression cassette including two to five of the set of 56 genes depicted in FIG. 19 under one promoter, and an expression cassette expressing MDH, HPS, and a PHI under another promoter in a plasmid. Next-generation sequencing was used to confirm the sequences encoded by the plasmids.

These plasmids were transformed into an E. coli strain that lacked frmA and tested for ¹³C-fractional enrichment in lysine. The strains were subjected to [¹³C]— MeOH-glucose co-feeds in the HTP scaled down fermentation screening, and [¹³C]-fractional enrichment showed a range from ˜35 to 6%.

Recombinant host cells including these plasmids were also tested for methanol assimilation into lysine. The methanol assimilation into lysine estimates were based on the complementation of the total lysine production by a methanol-glucose co-feed compared to “normal-dose” glucose and “minus 10%-reduced dose glucose” processes, allowing for an estimation of what fraction of the methanol dose was converted into lysine, which may be referred to as “methanol-derived” lysine %. Methanol-derived lysine of more than 5% was detected. “Methanol consumption” by various strains was also estimated by methanol carbon mass balance, in which the methanol consumed was calculated as follows: methanol added-residual methanol in culture broth—methanol evaporated. Methanol added was calculated based on feeding solution concentration and feeding volume. Residual methanol in culture broth was calculated using a quantitative enzymatic assay. Methanol evaporated is obtained by off-gas mass spectroscopy. Methanol consumption of about 35% was observed in at least one strain.

### EQUIVALENTS

Those skilled in the art will recognize, or be able to ascertain using no more than routine experimentation, many equivalents to the specific embodiments of the invention described herein. Such equivalents are intended to be encompassed by the following claims.

All references, including patent documents, disclosed herein are incorporated by reference in their entirety, particularly for the disclosure referenced herein.

What is claimed is:

1. A recombinant host cell that expresses a heterologous gene encoding a methanol dehydrogenase (MDH), wherein the MDH includes a sequence that is at least 90% identical to residues 96 to 295 of SEQ ID NO: 34 and wherein the MDH comprises:
(a) a valine (V) at an amino acid residue corresponding to position 26 in SEQ ID NO: 34;
(b) a valine (V) at an amino acid residue corresponding to position 31 in SEQ ID NO: 34;
(c) a valine (V) at an amino acid residue corresponding to position 169 in SEQ ID NO: 34; and/or
(d) an arginine (R) at an amino acid residue corresponding to position 368 in SEQ ID NO: 34.

2. The recombinant host cell of claim 1, wherein the MDH comprises (a), (c), and (d).

3. The recombinant host cell of claim 1, wherein the MDH comprises (b), (c), and (d).

4. The recombinant host cell of claim 1, wherein the MDH comprises (a), (b), (c), and (d)

5. The recombinant host cell of claim 1, wherein the MDH comprises (a) and (b); (a) and (c); (a) and (d); (b) and (c); (b) and (d); or (c) and (d).

6. The recombinant host cell of any one of claims 1-5, wherein the MDH comprises more than one amino acid substitution relative to the sequence of SEQ ID NO: 34 and wherein at least one of the amino acid substitution(s) is a conservative amino acid substitution.

7. The recombinant host cell of any one of claims 1-6, wherein the MDH has at least 25% of the NAD reductase activity as compared to cnMDHm3 (SEQ ID NO: 30) as measured by XTT enzyme assay.

8. The recombinant host cell of any one of claims 1-7, wherein the MDH is capable of catalyzing conversion of methanol to formaldehyde.

9. The recombinant host cell of any one of claims 1-8, wherein the MDH has a k_{cat} of at least 20 s⁻¹ as calculated using total protein and optical density of NADH.

10. The recombinant host cell of any one of claims 1-9, wherein the MDH has a Kₘ of at least 0.04 M as calculated using total protein and optical density of NADH.

11. The recombinant host cell of claim 9 or 10, wherein the MDH has a k_{cat}/Kₘ ratio of at least 300.

12. The recombinant host cell of any one of claims 1-11, wherein the MDH has a k_{cat} of at least 0.3 s⁻¹ as calculated using target protein concentration and concentration of NADH.

13. The recombinant host cell of any one of claims 1-8 and 12, wherein the MDH has a Kₘ of at least 0.04 M as calculated using target protein concentration and concentration of NADH.

14. The recombinant host cell of claim 12 or 13, wherein the MDH has a k_{cat}/Kₘ ratio of at least 1.1.

15. The recombinant host cell of any one of claims 1-14, wherein the MDH is at least 90% identical to SEQ ID NO: 34.

16. The recombinant host cell of any one of claims 1-15, wherein the recombinant host cell further comprises a heterologous gene encoding a 3-hexulose-6-phosphate synthase (HPS) selected from SEQ ID NOS: 106-122.

17. The recombinant host cell of any one of claims 1-16, wherein the recombinant host cell further comprises a heterologous gene encoding a 3-hexulose-6-phosphate isomerase (PHI) selected from SEQ ID NOS: 135-146.

18. A recombinant host cell that expresses a heterologous gene encoding a methanol dehydrogenase (MDH), wherein the MDH comprises a sequence that is at least 90% identical to a sequence selected from SEQ ID NOS: 32-56, and SEQ ID NOS: 81-88.

19. The recombinant host cell of claim 18, wherein the MDH comprises more than one amino acid substitution relative to the sequence of SEQ ID NO:34, and wherein at least one of the amino acid substitutions is a conservative amino acid substitution.

20. The recombinant host cell of claim 18 or 19, wherein the MDH has at least 25% of the NAD reductase activity as compared to cnMDHm3 as measured by XTT enzyme assay.

21. The recombinant host cell of any one of claims 18-20, wherein the MDH is capable of catalyzing conversion of methanol to formaldehyde.

22. The recombinant host cell of any one of claims 18-21, wherein the MDH has a k_{cat} of at least 20 s⁻¹ as calculated using total protein and optical density of NADH.

23. The recombinant host cell of any one of claims 18-22, wherein the MDH has a Kₘ of at least 0.04 M as calculated using total protein and optical density of NADH.

24. The recombinant host cell of claim 22 or 23, wherein the MDH has a k_{cat}/Kₘ ratio of at least 300.

25. The recombinant host cell of any one of claims 18-21, wherein the MDH has a k_{cat} of at least 0.3 s⁻¹ as calculated using target protein concentration and concentration of NADH.

26. The recombinant host cell of any one of claims 18-21 and 25, wherein the MDH has a Kₘ of at least 0.04 M as calculated using target protein concentration and concentration of NADH.

27. The recombinant host cell of claim 25 or 26, wherein the MDH has a k_{cat}/Kₘ ratio of at least 1.1.

28. The recombinant host cell of any one of claims 18-27, wherein the recombinant host cell further comprises a heterologous gene encoding a 3-hexulose-6-phosphate synthase (HPS) selected from SEQ ID NOS: 106-122.

29. The recombinant host cell of any one of claims 18-28, wherein the recombinant host cell further comprises a heterologous gene encoding a 3-hexulose-6-phosphate isomerase (PHI) selected from SEQ ID NOS: 135-146.

30. A recombinant host cell that expresses a heterologous gene encoding a 3-hexulose-6-phosphate (HPS), wherein the HPS comprises a sequence that is at least 90% identical to a sequence selected from SEQ ID NOS: 106-122, wherein the HPS comprises at least one amino acid substitution relative to SEQ ID NO: 122.

31. The recombinant host cell of claim 30, wherein the HPS comprises:
(a) a glutamine (Q) at a residue corresponding to position 4 of SEQ ID NO: 106;
(b) an alanine (A) at a residue corresponding to position 6 of SEQ ID NO: 106;
(c) an aspartic acid (D) at a residue corresponding to position 8 of SEQ ID NO: 106;
(d) an aspartic acid (D) at a residue corresponding to position 27 of SEQ ID NO: 106;
(e) a glutamic acid (E) at a residue corresponding to position 30 of SEQ ID NO: 106;
(f) a glycine (G) at a residue corresponding to position 32 of SEQ ID NO: 106;
(g) a threonine (T) at a residue corresponding to position 33 of SEQ ID NO: 106;
(h) a proline (P) at a residue corresponding to position 34 of SEQ ID NO: 106;
(i) a glycine (G) at a residue corresponding to position 40 of SEQ ID NO: 106;
(j) an aspartic acid (D) at a residue corresponding to position 59 of SEQ ID NO: 106;
(k) a lysine (K) at a residue corresponding to position 61 of SEQ ID NO: 106;
(l) a methionine (M) at a residue corresponding to position 63 of SEQ ID NO: 106;
(m) an aspartic acid (D) at a residue corresponding to position 64 of SEQ ID NO: 106;
(n) a glutamic acid (E) at a residue corresponding to position 69 of SEQ ID NO: 106;
(o) an glycine (G) at a residue corresponding to position 77 of SEQ ID NO: 106;
(p) an alanine (A) at a residue corresponding to position 78 of SEQ ID NO: 106;
(q) a leucine (L) at a residue corresponding to position 84 of SEQ ID NO: 106;
(r) an isoleucine (I) at a residue corresponding to position 92 of SEQ ID NO: 106;
(s) an alanine (A) at a residue corresponding to position 99 of SEQ ID NO: 106;
(t) a valine (V) at a residue corresponding to position 108 of SEQ ID NO: 106;
(u) an aspartic acid (D) at a residue corresponding to position 109 of SEQ ID NO: 106;
(v) an alanine (A) at a residue corresponding to position 120 of SEQ ID NO: 106;
(w) a glycine (G) at a residue corresponding to position 127 of SEQ ID NO: 106;
(x) a histidine (H) at a residue corresponding to position 134 of SEQ ID NO: 106;
(y) a glycine (G) at a residue corresponding to position 136 of SEQ ID NO: 106;
(z) an aspartic acid (D) at a residue corresponding to position 138 of SEQ ID NO: 106;
(aa) a glutamine (Q) at a residue corresponding to position 140 of SEQ ID NO: 106;
(bb) an alanine (A) at a residue corresponding to position 141 of SEQ ID NO: 106;
(cc) an alanine (A) at a residue corresponding to position 164 of SEQ ID NO: 106;
(dd) a glycine (G) at a residue corresponding to position 165 of SEQ ID NO: 106;
(ee) a glycine (G) at a residue corresponding to position 166 of SEQ ID NO: 106;
(ff) a glycine (G) at a residue corresponding to position 186 of SEQ ID NO: 106;
(gg) an isoleucine (I) at a residue corresponding to position 189 of SEQ ID NO: 106; and/or
(hh) an alanine (A) at a residue corresponding to position 199 of SEQ ID NO: 106.

32. The recombinant host cell of claim 30 or 31, wherein the HPS is capable of converting formaldehyde and ribulose 5-phosphate into hexulose-6-P.

33. The recombinant host cell of any one of claims 30-32, wherein the HPS has an activity that is at least 50% of a control enzyme, wherein the control enzyme is HPS from Methylococcus capsulatus (UniProtKB-Q602L4) (SEQ ID NO: 122).

34. The recombinant host cell of any one of claims 30-33, wherein the recombinant host cell further comprises a heterologous gene encoding a methanol dehydrogenase (MDH) selected from SEQ ID NOS: 29-56 and SEQ ID NOS: 81-88.

35. The recombinant host cell of any one of claims 30-34, wherein the recombinant host cell further comprises a heterologous gene encoding a 3-hexulose-6-phosphate isomerase (PHI) selected from a sequence in SEQ ID NOS: 135-146.

36. A recombinant host cell that expresses a heterologous gene encoding a 3-hexulose-6-phosphate isomerase (PHI), wherein the PHI comprises a sequence that is at least 90% identical to a sequence selected from SEQ ID NOS: 135-146, wherein the PHI comprises at least one amino acid substitution relative to SEQ ID NO: 146.

37. The recombinant host cell of claim 36, wherein the PHI is capable of converting hexulose-6-phosphate to fructose-6-phosphate.

38. The recombinant host cell of claim 36 or 37, wherein the PHI has an activity that is at least 50% of a control enzyme, wherein the control enzyme is PHI from Methylococcus capsulatus (SEQ ID NO: 146).

39. The recombinant host cell of any one of claims 36-38, wherein the recombinant host cell further comprises a heterologous gene encoding a methanol dehydrogenase (MDH) selected from SEQ ID NOS: 29-56 and SEQ ID NOS: 81-88.

40. The recombinant host cell of any one of claims 36-39, wherein the recombinant host cell further comprises a heterologous gene encoding a 3-hexulose-6-phosphate synthase (HPS) selected from SEQ ID NOS: 106-122.

41. The recombinant host cell of any one of claims 1-40 that further comprises a sequence that is at least 90% identical to an RPI enzyme selected from SEQ ID NOS: 217-222.

42. The recombinant host cell of any one of claims 1-41 that further comprises a sequence that is at least 90% identical to an RPE enzyme selected from SEQ ID NOS: 204-210.

43. The recombinant host cell of any one of claims 1-42 that further comprises a sequence that is at least 90% identical to a TKT enzyme selected from SEQ ID NOS: 241-246.

44. The recombinant host cell of any one of claims 1-43 that further comprises a sequence that is at least 90% identical to a TAL enzyme selected from SEQ ID NOS: 229-234.

45. The recombinant host cell of any one of claims 1-44 that further comprises a sequence that is at least 90% identical to a PFK enzyme selected from SEQ ID NOS: 191-196.

46. The recombinant host cell of any one of claims 1-45 that further comprises a sequence that is at least 90% identical to a GLPX enzyme selected from SEQ ID NOS: 166-172.

47. The recombinant host cell of any one of claims 1-46 that further comprises a sequence that is at least 90% identical to an FBA enzyme selected from SEQ ID NOS: 153-158.

48. The recombinant host cell of any one of claims 1-47 that further comprises a sequence that is at least 90% identical to a GND enzyme selected from SEQ ID NOS: 179-184.

49. The recombinant host cell of any one of claims 1-48 that further comprises a sequence that is at least 90% identical to a ZWF enzyme selected from SEQ ID NOS: 253-258.

50. The recombinant host cell of any one of claims 1-49, wherein the recombinant host cell is capable of producing lysine with at least one carbon derived from methanol in a feedstock comprising substitution of a saccharide with methanol.

51. The recombinant host cell of claim 50, wherein the % weight per weight (% w/w) substitution of the saccharide with methanol is at least 5%.

52. The recombinant host cell of claim 50 or 51, wherein at least 25% of the methanol provided in feedstock is consumed by the recombinant host cell.

53. The recombinant host cell of any one of claims 50-52, wherein the saccharide is sucrose, glucose, lactose, dextrose, or fructose.

54. The recombinant host cell of any one of claims 1-53, wherein the recombinant host cell is an E. coli cell.

55. The recombinant host cell of claim 54, further comprising a knockout of a gene encoding S-(hydroxymethyl)glutathione dehydrogenase.

56. The recombinant host cell of claim 55, wherein the gene is frmA gene.

57. The recombinant host cell of any one of claims 54-56, wherein the recombinant host cell expresses more than one heterologous gene and wherein at least one heterologous gene is expressed from a J23104 promoter, an Ec-TTL-P041 promoter, and/or a P_{gal} promoter.

58. The recombinant host cell of claim 55, wherein the recombinant host cell expresses more than two heterologous genes and wherein at least two heterologous genes are driven by the J23104 promoter, the Ec-TTL-P041 promoter, or the P_{gal} promoter.

59. A method of producing methanol-derived organic compounds comprising culturing the recombinant host cell of any one of claims 1-58 in feedstock comprising substitution of a saccharide with methanol, thereby producing methanol-derived organic compounds.

60. A method of producing methanol-derived amino acids comprising culturing the recombinant host cell of any one of claims 1-58 in feedstock comprising substitution of a saccharide with methanol, thereby producing methanol-derived amino acids.

61. A method of producing methanol-derived lysine comprising culturing the recombinant host cell of any one of claims 1-58 in feedstock comprising substitution of a saccharide with methanol, thereby producing methanol-derived lysine.

62. The method of any one of claims 59-61, wherein the recombinant host cell is an E. coli cell.

63. The method of any one of claims 59-62, wherein the % weight per weight (% w/w) substitution of the saccharide with methanol in the feedstock is at least 5%.

64. The method of any one of claims 59-63, wherein at least 25% of the methanol provided in feedstock is consumed by the recombinant host cell.

65. The method of any one of claims 59-63, wherein the saccharide is sucrose, glucose, lactose, dextrose, or fructose.

66. A vector comprising a sequence that is at least 90% identical to a sequence selected from SEQ ID NOs: 1-28, 73-80, 89-105, 123-134, 147-152, 159-165, 173-178, 185-190, 197-203, 211-216, 223-228, 235-240 and 247-252.

67. An expression cassette comprising a sequence that is at least 90% identical to a sequence selected from SEQ ID NOs: 1-28, 73-80, 89-105, 123-134, 147-152, 159-165, 173-178, 185-190, 197-203, 211-216, 223-228, 235-240 and 247-252.
